(19) United States
(12) Patent Application Publication
(10) Pub. No.: US 2015/0359668 A1
(43) Pub. Date: Dec. 17, 2015

(54) LIGHT DELIVERY DEVICE AND RELATED COMPOSITIONS, METHODS AND SYSTEMS

(71) Applicant: CALIFORNIA INSTITUTE OF TECHNOLOGY, PASADENA, CA (US)

(72) Inventors: Julia A. KORNFIELD, PASADENA, CA (US); Matthew S. MATTSON, PASADENA, CA (US); Joyce HUYNH, WESTMINSTER, CA (US)

(21) Appl. No.: 14/797,063

(22) Filed: Jul. 10, 2015

Related U.S. Application Data
(62) Division of application No. 13/464,950, filed on May 4, 2012, now abandoned.
(60) Provisional application No. 61/483,551, filed on May 6, 2011.

(51) Int. Cl. A61F 9/008; A61K 41/00; A61F 9/00

(52) U.S. Cl. CPC A61F 9/008; A61F 9/0008; A61K 41/0057; A61F 2009/00865; A61F 2009/00872

(57) ABSTRACT

Devices and systems for delivering light to a target are described. Methods of using such light delivery device and system are also described. A method of using a photosensitizing compound with the light delivery device is also described.

### CROSS REFERENCE TO RELATED APPLICATIONS

The present application is a Divisional of U.S. application Ser. No. 13/464,950 filed on May 4, 2012, which, in turn, claims priority to U.S. Provisional Application No. 61/483,551 filed on May 6, 2011, all of which are incorporated herein by reference in their entirety.

### STATEMENT OF GOVERNMENT GRANT

This invention was made with government support under Grant No. EY017484 and Grant No. EY019805 awarded by the National Institutes of Health. The government has certain rights in the invention.

### FIELD

The present disclosure relates to a light delivery device and related compositions methods and systems. In particular, the disclosure relates to a device to deliver light to an eye of an individual, and related compositions, methods and systems.

### BACKGROUND

Light delivery to an eye of an individual has been a challenge in the field of ophthalmology, in particular when aimed at treatment of ocular conditions. Whether for clinical applications or for fundamental anatomical or biological studies, several methods are have been developed that comprise use of light delivery to the eye alone or in combination with administration of a suitable compound or composition.

In particular, in several applications, light-activated chemical reactions in several ocular regions and in particular in the anterior segment are used to achieve diverse clinical objectives, including increasing the strength of the cornea and adjusting the power of a light adjustable lens. In some cases, light delivery is performed in combination with drug delivery to and/or through the cornea and/or sclera as an alternative to delivery by injection.

Despite the significant progresses achieved in the field, control of light delivery and of the distribution of related compounds to be used in combination with light delivery to achieve crosslinking or other desired effect, remains challenging.

### SUMMARY

Provided herein are devices, that in several embodiments, allow light delivery to the eye of an individual in a controlled fashion and related, methods systems and compositions. In particular in some embodiments provided herein are devices and related methods, systems and compositions that allow control of light delivery in combination with delivery of compounds such as drugs to the eye of the individual.

According to a first aspect, a light delivery device for delivering light to an eye of an individual is described. The device comprising a light emitting arrangement, the light emitting arrangement being configured to direct, in use, radiation towards the eye of the individual at a distance from the light delivery device along a plurality of irradiating directions, each direction of the plurality of irradiating directions being oblique to the optical axis of the eye, and being positionable at said distance from the eye to allow said radiation to be convergently directed towards a target ocular region of the individual.

According to a second aspect, a holder for a light emitting arrangement is described. The holder comprises an external region comprising a host section adapted to host a light emitting arrangement, the host section adapted to host the light emitting arrangement, the holder adapted to be positioned at a distance from a target ocular region of the eye of an individual during use to allow said radiation to be convergently directed towards the target ocular region, along a plurality of irradiating directions each direction of the plurality of irradiating directions being oblique to the optical axis.

According to a third aspect a system for light delivery, is described. The system comprises a support adapted to position a light delivery device herein described at a set distance from the target ocular region in the eye of an individual during use of the light delivery device to allow said radiation to be convergently directed towards the target ocular region, along a plurality of irradiating directions, each direction of the plurality of irradiating directions being oblique to the optical axis of the eye.

According to a fourth aspect, a method of irradiating a target ocular region of the eye of an individual is described. The method comprises providing a radiation towards a target ocular region along a plurality of irradiating directions, each direction of the plurality of irradiating directions being oblique to the optical axis, wherein the radiation is provided at a distance from the target ocular region to allow said radiation to be convergently directed towards the target ocular region of the eye.

According to a fifth aspect, a method for photodynamic cross-linking of a target tissue in an eye is described. The method comprises: applying a set quantity of a photosensitizing compound to a target ocular region of the eye for a set contact time; allowing diffusion of the photosensitizing compound in the target ocular region for a set delay time, following expiration of the contact time; and irradiating the target ocular region of the eye with a light source upon expiration of the set delay time, wherein: the contact time is set to be between approximately 0.01-10 times a diffusion time of the photosensitizing compound, wherein the diffusion time is a ratio of the square of the thickness of the target tissue divided by the diffusion coefficient of the photosensitizing compound in the target tissue; the contact time and delay time are jointly set such that the sum of the contact time and the delay time is between approximately 0.01-10 times the diffusion time of the photosensitizing compound; the set quantity of photosensitizing compound is capable of extinguishing the irradiating light by between approximately 10-99%; and the contact time, the delay time, and the quantity of photosensitizing compound are controllable to vary an effect of the photodynamic crosslinking.

According to a sixth aspect, a topical pharmaceutical composition for treatment of an ocular condition is described. The composition comprises eosin Y as an active agent to treat the ocular condition and a pharmaceutically suitable vehicle.

According to a seventh aspect, a method for providing a pharmaceutical composition suitable to be used in combination with a light emitting source for performing a photodynamic cross-linking on a target ocular region of an individual, is described. The method comprises determining a partition coefficient and a diffusion coefficient for a photosensitizing compound in the target ocular region by performing testing on a test tissue thus modifying the tissue; calculating a concentration profile of the photosensitizing compound across the target ocular region as a function of time and depth of the ocular region, based on the partition coefficient and the diffusion coefficient of the photosensitizing compound in the target ocular region for one or more set of contact time, delay time and concentration of the photosensitizing compound; calculating a light intensity profile across the target tissue as a function of time and tissue depth, at a set light dose, based on the concentration profile for the one or more set of contact time, delay time and concentration of the photosensitizing compound; quantifying an instantaneous local cross-linking rate based on the concentration profile and the light intensity profile; and selecting a concentration of the photosensitizing compound, a suitable vehicle and the related concentration based on the quantified local cross linking rate, thus providing a pharmaceutical composition comprising the photosensitizing compound and the suitable vehicle.

According to an eighth aspect a method for treating an ocular condition is described. The method comprises: administering to an individual a photosensitizing compound, the administering comprising applying the photosensitizing compound to a target ocular region for a time and under a condition to allow a suitable concentration of the photosensitizing compound throughout the target ocular region; directing a light source at the target ocular region for a time and under conditions to allow a desired extent of cross-linking of a protein to occur in the ocular tissue, wherein the compound: has a partition coefficient (k) in the target ocular region ranging from approximately 2 to 20; has a product of the partition coefficient and a diffusion coefficient (kD) in the target ocular region ranging from approximately 40 to 400 um²/sec; and is capable of generating singlet oxygen upon exposure to a light source of a suitable wavelength.

According to a ninth aspect, a compound for use in treating an ocular condition is described. The compound: is a photosensitizer, has a partition coefficient (k) in a target ocular region ranging from approximately 2 to 20; has a product of the partition coefficient and a diffusion coefficient (kD) in the target ocular region ranging from approximately 40 to 400 um²/sec; and is capable of generating singlet oxygen upon exposure to a light source of a suitable wavelength.

According to a tenth aspect, a method for selecting contact time, delay time and concentration of a photosensitizing compound for performing a photodynamic cross-linking on a target ocular region of an individual, is described, The method comprises: determining a partition coefficient and a diffusion coefficient for the photosensitizing compound in the target ocular region by performing testing on a test tissue thus modifying the tissue; calculating a concentration profile of the photosensitizing compound across the target ocular region as a function of time and depth of the ocular region, based on the partition coefficient and the diffusion coefficient of the photosensitizing compound in the target ocular region for one or more set of contact time, delay time and concentration of the photosensitizing compound; calculating a light intensity profile across the target tissue as a function of time and tissue depth, at a set light dose, based on the concentration profile for the one or more set of contact time, delay time and concentration of the photosensitizing compound; quantifying an instantaneous local cross-linking rate based on the concentration profile and the light intensity profile; selecting the contact time, delay time and concentration of the photosensitizing compound based on the quantified instantaneous local cross linking rate.

According to an eleventh aspect, a method for using a device is for applying substantially uniform irradiance to an ocular or intraocular surface of an individual is described. The device comprises light sources distributed along the devices, the method comprising: selecting a position of the light sources on the device; selecting a number of the light sources; and determining a distance of the light sources from the ocular or intraocular surface as a function of the selected radial position and the selected number of light sources.

The light delivery device, and related methods and systems, allow in several embodiments, control of light delivery to selected ocular regions of interest while substantially avoiding anti-target regions such as retinal anti-target regions such as the macula.

The light delivery device, and related methods, systems and compositions, allow in several embodiments, improvement of the safety and efficacy of treatment with respect to certain known methods by allowing a higher control of light deliver and/or related effects (such as protein crosslinking) in the eye.

The light delivery methods, systems and compositions, allow in several embodiments, control of related treatment parameters are optimized with respect to the spatial distribution of drug and light in the tissue.

The light delivery methods, systems and compositions, allow in several embodiments, identification of formulations including a selected concentration of the photoactivated compound and any auxiliary light-blocking components that are optimized with respect to the spatial distribution of drug and light in the tissue.

The light delivery device, and related methods, systems and compositions herein described can be used in connection with applications wherein control of light delivery and/or a compound distribution in the eye of an individual is desired, including but not limited to biological analysis and medical application such as, clinical applications and diagnostics, and additional applications identifiable by a skilled person.

The details of one or more embodiments of the disclosure are set forth in the accompanying drawings and the description below. Other features will be apparent from the description and drawings, and from the claims.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 show various angles associated with a light source and the eye.

FIGS. 2A-2B show schematic cross-sections of an eye showing the anti-target region.

FIG. 3 shows a simulation of a cornea being irradiated with the light delivery device. The plurality of obliquely oriented sources can treat the target (e.g., cornea) while avoiding the anti-target region (e.g., macula).

FIGS. 4A-4E show various views of a light delivery device according to the embodiments of the present disclosure.

FIGS. 5A-5E is a diagram showing discrete light sources for the light delivery device.

FIGS. 6A-6E show various views of a holder for the light emitting elements.

FIGS. 7A-7E show various views of the light delivery device and system.

FIG. 8 show various angles associated with a light source and a target.

FIG. 9 shows a cross sectional view of a cornea tissue.

FIGS. 10A-10D show various irradiation patterns on a target as a function of the distance of the light delivery device.

FIGS. 11A-11B show side views of irradiation patterns when there are a plurality of light emitting arrangements or a plurality of light delivery devices.

FIGS. 12A-12F show the effect of apertures on the light emitting elements of the light delivery device.

FIGS. 13A-13B show various angles associated with a light source and a target.

FIGS. 14A-14B show simulation illustrating intensity on ocular surfaces, including a pre-pupil plane 404 and a post-lens plane 405.

FIGS. 15A-15D show irradiance profiles for corneal surface 400, pre-pupil plane 404, post-lens plane 405, and retinal surface 406.

FIGS. 16A-16B show intensity as a function of distance from spot center. The retinal image spot intensity and size is dependent on the LED source geometry, and on the pupil size. The smaller pupil size does not allow the images of the LEDs to overlap, and has a significantly lower maximum intensity on the retina. Increasing the source geometry from the conservative 1 mm square chip to the more realistic 3.25 mm reflector blurs the image, creating a less well defined image.

FIGS. 17A-17D show a pictures of a single LED taken using a super macro lens.

FIG. 18 shows three different LED sources: a 1 mm square chip, a combination of the chip and reflector, and a 3.25 mm diameter reflector.

FIG. 19 shows that the maximum intensity incident on the retina is significantly lower with a 3 mm pupil than with a 7 mm pupil diameter. The more realistic LED source geometry of the 1 mm square chip and 3.25 mm reflector (see 3 in FIG. 18). also has a significantly lower maximum intensity than the conservative 1 mm chip (see 2 in FIG. 18).

FIG. 20 shows that the average retinal image size is approximately 1 mm for all LED source geometries when the pupil is 3 mm in diameter. When the pupil is 7 mm in diameter, the image size for the 1 mm square chip decreases while the image size for the other geometries increases (see 2-4 in FIG. 18).

FIG. 21 shows the results of the simulated exposure divided by the ISO limited for photochemical hazard. Values less than 1 indicate that the source has no hazard. All values for a 3 mm pupil size are less than 0.5. Values greater than 1 (above the dotted line) indicate that the source could be a potential hazard and should be examined for safety under Group 2 conditions.

FIGS. 22A-22B show a curved surface matching the corneal curvature for simulations of light incident on the cornea. The light incident on a 1 mm diameter white circle is the localized average. Black dotted lines indicate the cross sections taken in FIG. 22B.

FIG. 23 show that the horizontal cross section of light incident on the cornea shows that the when the distance is too close the profile has some bright spots at the outer edges, and that when it is too far the intensity between the center and edges varies too much. Distances with black lines (18.2-20.2 mm are used for the treatment).

FIG. 24A show that the retinal image intensity increases until the source is approximately 22 mm from the cornea and then the intensity decreases with increasing distance.

FIG. 24B show that the retinal image location is far from the center of the macula at all distances, but becomes closer to the macula as the source distance from the cornea increases (e.g., how far the bright spots are from the anti-target).

FIG. 25 shows a graph of a relative spectral power distribution of the 525-nm LEDs (˜30 nm FWHM). The spectral irradiance is in units of μW·cm⁻² sr⁻¹.

FIG. 26 shows a graph of a relative angular intensity distribution for a RL5-G7032 LED measured with Ocean Optics Jaz spectrometer.

FIG. 27 is a graph showing laser safety limits. A laser with a power level of 100 μW can be used for 39 seconds without being considered hazardous. Since the alignment process will most likely not exceed 10 seconds, this alignment method will not pose a hazard to the retina. Safety features will ensure that the light levels will not exceed the safe levels.

FIG. 28A show a schematic diagram of the observed fluid pocket around the eye of Eosin Y/TEOA formulations

FIG. 28B show a flexible LED source held around the eye.

FIG. 29 shows various ocular measurements.

FIG. 30A-30C shows schematically, structures of a collagen fibril (image adapted from nanobiomed.de), a proteoglycan and collagen fibrils immersed in a gel-like matrix of water and proteoglycans. (Images b and c are adapted from Oyster^{[2]})

FIG. 31A-31B comprises images showing that fibrils form parallel lamellae in the cornea and interweaving morphology in the sclera. (Images adapted from Oyster^{[2]})

FIG. 32A-32D is a schematic showing Riboflavin as a photosensitizer for inducing cross-links, displays a treatment that involves removing the epithelial cell layer, applying drops of riboflavin solution onto the cornea, and irradiating the cornea.

FIG. 33A-33D is a schematic showing Eosin Y as a visible light activated crosslinker, displays a treatment that involves removing the epithelial cell layer, applying a viscous gel containing eosin Y onto the cornea, and irradiating the cornea. (Images c & d adapted from Matthew Mattson's Thesis)

FIG. 34A-34C comprises graphs showing rate of change in storage modulus of collagen gel with riboflavin irradiated with 370 nm at 3 mW/cm² and eosin Y irradiated with 530±15 nm light at 6 mW/cm² in the presence and absence of oxygen. Rate of change in storage modulus in air for samples containing ascorbic acid (AA) and sodium azide (SA). FIG. 43B-43C comprises of graphs showing rate as a percent of the rate without oxygen quencher for riboflavin and eosin Y. The asterisk indicates a statistically significant difference compared to the samples in air with no quencher (p<0.05). (N=3 to 12)

FIG. 35A-35B comprises graphs showing change in storage modulus where ΔG′=G′ₜ−G′₁₀ as a function of time for 450 μm thick gel samples with eosin Y and riboflavin. Samples containing eosin Y were irradiated with green light at 530±15 nm and those containing riboflavin were irradiated with UV light at 370±15 nm. The presence of both drug and light are necessary for enhancing the storage modulus. (N=3 to 6)

FIG. 36A-36C comprises graphs showing rate of change of the apparent storage modulus as a function of irradiation intensity at a fixed sample thickness (450 μm) and fixed photosensitizer concentration (0.02% eosin Y, 0.1% riboflavin), as a function of photosensitizer concentration at fixed sample thickness (450 μm) and fixed irradiation intensity (6 mW/cm² for eosin Y, 3 mW/cm² for riboflavin) and as a function of sample thickness at fixed photosensitizer concentration (0.02% for eosin Y and 0.1% for riboflavin) and irradiation intensity (6 mW/cm² for eosin Y and 3 mW/cm² for riboflavin). Samples containing eosin Y were irradiated with green light at 530±15 nm and those containing riboflavin were irradiated with green light at 370±12 nm. (N=4 to 14)

FIG. 37A-37B comprises graphs showing normalized rate of change in modulus (dG′/dt)/(dG′/dt)ₘₐₓ as a function of the normalized optical penetration depth evaluated using data obtained with a fixed sample thickness (450 μm, FIG. 3.3b) and data obtained using a fixed photosensitizer concentration (0.02% eosin Y and 0.1% riboflavin, FIG. 3.3c). Eosin Y samples were irradiated with 530±15 nm light at 6 mW/cm² and riboflavin samples were irradiated with 370±12 nm light at 3 mW/cm². (N=4 to 14)

FIG. 38A-38B is a graph showing Riboflavin concentration profile inside corneal tissue after 30 minutes of applying drug using the clinical dose of 0.1%. and Keratocyte toxicity is observed in the anterior 300-350 μm of the corneal stroma after riboflavin/UVA treatment. (Image adopted from lasikcomplications.com)

FIG. 39A-39B comprises graphs showing storage modulus as a function of time for 450 μm thick gel samples with eosin Y and riboflavin. Samples containing eosin Y were irradiated with green light at 530±15 nm and those containing riboflavin were irradiated with UV light at 370±15 nm. The presence of both drug and light are necessary for enhancing the storage modulus. (To avoid over-crowding of the figures, only three samples are shown for each condition.)

FIG. 40 is a schematic showing a quantitative assay of the amount of molecules transferred to the tissue cross-section. The section was placed into 50 mL of double-distilled water for 8 hours, then transferred to a new 50 mL of double-distilled after 24 hours, then transferred again after 48 hours.

FIG. 41A-41D is a schematic showing an eye removed from eosin Y solution when contact time completed eissection to separate the cornea, trephine punch used to cut out a 9.5-mm diameter cross-section of the cornea and the section placed into a cuvette to measure the absorbance.

FIG. 42A-42B comprises graphs showing a number of drug molecules, eosin Y (EY) and riboflavin, in successive extracts from tissue specimens given a 2 hours contact time for the cornea and sclera. Approximately all of the extractable molecules were removed from the cornea after 3 extractions, and from the sclera after 5 extractions. Therefore, the sum of the number of drug molecules in all the extracts is a good approximation of all the drug molecules that had been transferred into the tissue cross-section. (N=4)

FIG. 43A-43B comprises graphs showing a total number of drug molecules delivered as a function of drug contact time for both eosin Y and riboflavin in the cornea and the sclera. Note: all data points have associated error bars, however some error bars are too small to visible on the graph. (N=4)

FIG. 44A-44B comprises graphs showing a total number of drug molecules delivered as a function of drug contact time for both eosin Y and riboflavin in the cornea and the sclera. The “best fit” curves were generated using a diffusion model with values of k and D given in Table 2.

FIG. 45A-45B comprises graphs showing the extraction and the absorbance methods yield similar results for the number of molecules delivered to the cornea for three selected delivery techniques applied for 5 minutes and the comparison of different delivery vehicles using the absorbance measurement to determine the quantity of drug delivered in 5 minutes from gels using four different viscosity-enhancing agents. (N=4)

FIG. 46A-46C is a schematic showing topical application of the drug formulation onto the cornea, the removal of the drug from the cornea at the end of contact time and irradiation after a delay time.

FIG. 47A-47B comprises graphs showing a rate of change in oscillatory storage modulus as a function of concentration for collagen gel samples with approximately uniform intensity profiles for riboflavin and eosin Y. (N=4 to 12)

FIG. 48A-48C comprises graphs showing concentration profile for 0.1% riboflavin with 30 minutes contact time, light intensity profile for 3 mW/cm² irradiation and a profile of modulus increase for 30 minutes irradiation with ΔG′_{avg} increased by 503 Pa.

FIG. 49A-49C comprises graphs showing Eosin Y concentration profile inside the tissue for three different drug concentrations after 5 minutes contact time, corresponding light intensity profiles for the three different drug concentrations and a profile of modulus increase for each drug concentration after 5 minutes irradiation at 6 mW/cm². The ΔG′_{avg} in the tissue is 80 Pa for 0.003%, 104 Pa for 0.01%, and 55 Pa for 0.03%.

FIG. 50A-50C comprises graphs showing Eosin Y concentration profile inside the tissue for three different drug contact times using 0.01% eosin Y concentration, corresponding intensity profiles for the three different drug contact times and a profile of modulus increase for each drug concentration after 5 minutes irradiation at 6 mW/cm². The ΔG′_{avg} in the tissue is 76 Pa for 1 minute, 104 Pa for 5 minutes, and 107 Pa for 10 minutes contact time.

FIG. 51A-51C comprises graphs showing Eosin Y concentration profile inside the tissue for four different drug delay times using 0.01% eosin Y concentration with 5 minutes contact time, corresponding intensity profiles for the four delay times and a profile of modulus increase for each drug concentration after 5 minutes irradiation at 6 mW/cm². The ΔG′_{avg} in the tissue is 104 Pa for 0 minute, 108 Pa for 1 minute, 115 Pa for 5 minutes, and 119 Pa for 10 minutes delay time.

FIG. 52A-52C comprises graphs showing a concentration profile for 0.01% eosin Y concentration with 5 minutes contact time and 1 minutes delay, light intensity profiles for three different irradiation intensities and a profile of modulus increase for the same light dose of 1.8 J/cm² using three pairs of intensity and irradiation duration. The ΔG′_{avg} is 198 Pa for 15 minutes at 2 mW/cm², 139 Pa for 7.5 minutes at 4 mW/cm², and 108 Pa for 5 minutes at 6 mW/cm².

FIG. 53A-53C comprises graphs showing a concentration profile for 0.01% eosin Y concentration with 5 minutes contact time and 1 minute delay, corresponding light intensity profile for 6 mW/cm² irradiation and a profile of modulus increase for three irradiation durations. The ΔG′_{avg} is 108 Pa for 5 minutes, 223 Pa for 10 minutes, and 697 Pa for 30 minutes.

FIG. 54A-54C is a schematic showing Riboflavin concentration profile after 30 minutes of topical drug application for clinical dose (0.1%) and optimal dose (0.044%), light intensity profiles for 3 mW/cm² irradiance and a profile of modulus increase after 30 minutes of irradiation (ΔG′_{avg} is 503 Pa for clinical dose and 618 Pa for optimal dose).

### DETAILED DESCRIPTION

Devices and related, methods systems that in several embodiments are described that allow light delivery to the eye of an individual in a controlled fashion. In particular in some embodiments provided herein are devices and related methods, systems and compositions that allow control of light delivery in combination with delivery of compounds such as drugs to the eye of the individual.

Eyes in the sense of the present disclosure are organs that detect light and convert it into electro-chemical impulses in neurons. Eye typically includes three coats, enclosing three transparent structures. The outermost layer is composed of the cornea and sclera. The middle layer consists of the choroid, ciliary body, and iris. The innermost is the retina, which gets its circulation from the vessels of the choroid as well as the retinal vessels, which can be seen in an ophthalmoscope. The shape of the eye is maintained by the ocular coat, which consists of the cornea and sclera. The sclera is the opaque, fibrous, protective, outer layer of the eye containing collagen and elastic fiber, also indicated as white part of the eye making up five sixth of the total surface area of the eye. Its function is to provide support and protect the eye. The cornea is the transparent front part of the eye that covers the iris, pupil, and anterior chamber. The cornea is the clear tissue in front of the eye, which provides approximately two thirds of the total focusing power. Regions of the cornea and sclera form the limbus region. The term “limbus” or “corneal limbus” as used herein is defined to mean a border of the cornea where it meets the sclera or junction between the cornea and sclera. Thus limbus is generally a thin (e.g. less than approximately 0.4 mm) region bordering the cornea. Within the corneal and scleral coats are the aqueous humor, the vitreous body, and the flexible lens connected as will be understood by a skilled person. An eye also includes an “optic axis” or an “optical axis” which the hypothetical straight line passing through the centers of curvature of the front and back surfaces of the natural lens, which will be identifiable by a skilled person.

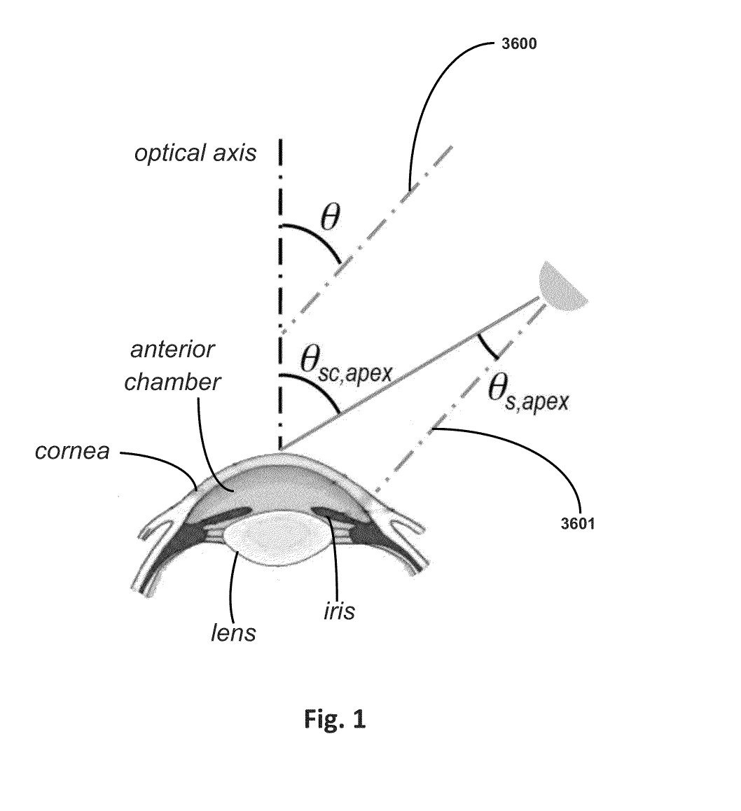

In some embodiments, light is delivered by devices, methods and systems which make reference to a target associated to the target ocular region of interest. In particular, in several embodiments, the target is a tissue in the anterior segment of the eye (see e.g., FIG. 1). In some embodiment, the target can be the corneal surface of the eye.

In some embodiments, light is delivered by a device that is configured to selectively irradiate target ocular regions of interest by providing radiation directed towards the target ocular region of interest, wherein the radiation comes towards the target from different directions oblique—slanting or inclined in direction or course or position oblique to the optical axis of the eye.

In particular, in several embodiments, the device herein described comprises a light emitting arrangement configured to direct, in use, radiation towards the target along a plurality of irradiating directions, each direction oblique to the optical axis of the eye. In some embodiments, the oblique angles are approx. 20° or higher with respect to the optic axis of the eye. In some embodiments, the oblique directions are at approximately approx. 30° or higher with respect to the optic axis of the eye.

Control of the radiation emitted and related irradiance provided on the ocular surface can be determined by design of the angular distribution of light emitted by the light delivery device and the position and orientation of the light delivery device with respect to the eye being treated.

The devices are able to meet a set of therapeutic criteria for irradiation of ocular tissues. By way of example and not of limitation, the therapeutic criteria of the device can include: 1) azimuthal uniformity of irradiation, 2) radial distribution of irradiance, and 3) overall level of irradiance.

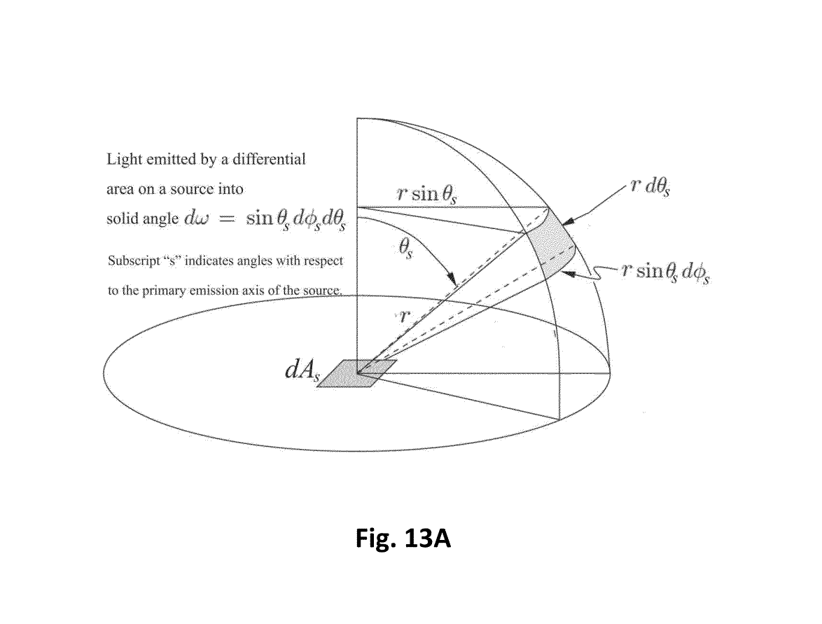

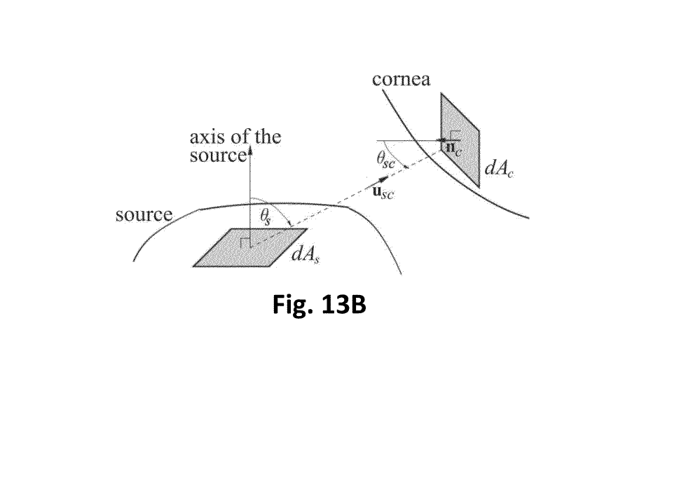

The term “Radiance” of a source describes the radiant emittance per solid angle and has SI units (W/m²sr), as indicated FIG. 13A.

The conventional notation in radiometry for these quantities is given, together with their SI units, in table 1 below:

TABLE 1
conventional notation in radiometry.

| Quantity | Symbol | Units |
|---|---|---|
| Radiant Energy | Q | J |
| Radiant Power | Φ | W |
| Irradiance | E | W/m² |
| Radiant Intensity | I | W/sr |
| Radiance | L | W/(m²sr) |

The distribution of source radiance with respect to θₛ, the angle with respect to the axis of a source, is a characteristic of a particular source. (see e.g., FIG. 1) One skilled in the art can obtain information on the angular distribution from a manufacturer at the time of selection of sources for fabrication of a device according to this invention. One skilled in the art can characterize the angular distribution of light emitted from a specific source using radiometric measurements.

For light emitted from a small area dAs of a source as light propagates out through a non-absorptive medium, the irradiance decreases in proportion to the inverse of the square of the distance from the source. (e.g. see Example 2)

The term “irradiance” can be used herein interchangeably with the term “radiative flux”, and can be defined as the power of electromagnetic radiation per unit area incident on a surface. The SI unit is watts per square meter (W/m²).

The term “radiant emittance” can be used herein interchangeably with the term “radiant exitance”, and can be defined as the power per unit area radiated by a surface. The SI unit for emittance is watts per square meter (W/m²).

A “radiant power” incident on the surface of interest can be obtained by integrating the irradiance or emittance over a surface of interest. The SI unit of radiant power is watts (W).

The term “radiant intensity” is a measure of the power of electromagnetic radiation per unit solid angle. The SI unit of radiant intensity is watts per steradian (W/sr). The radiant intensity I(θₛ) of a source is the integral of the source radiance L(θₛ) over the source area. A steradian is related to the surface area of a sphere in a similar manner as a radian is related to the circumference of a circle. One steradian intercepts an area r² of the surface of a sphere of radius r, just as a radian intercepts a length of a circle's circumference equal to its radius. A solid angle dω is measured in steradian.

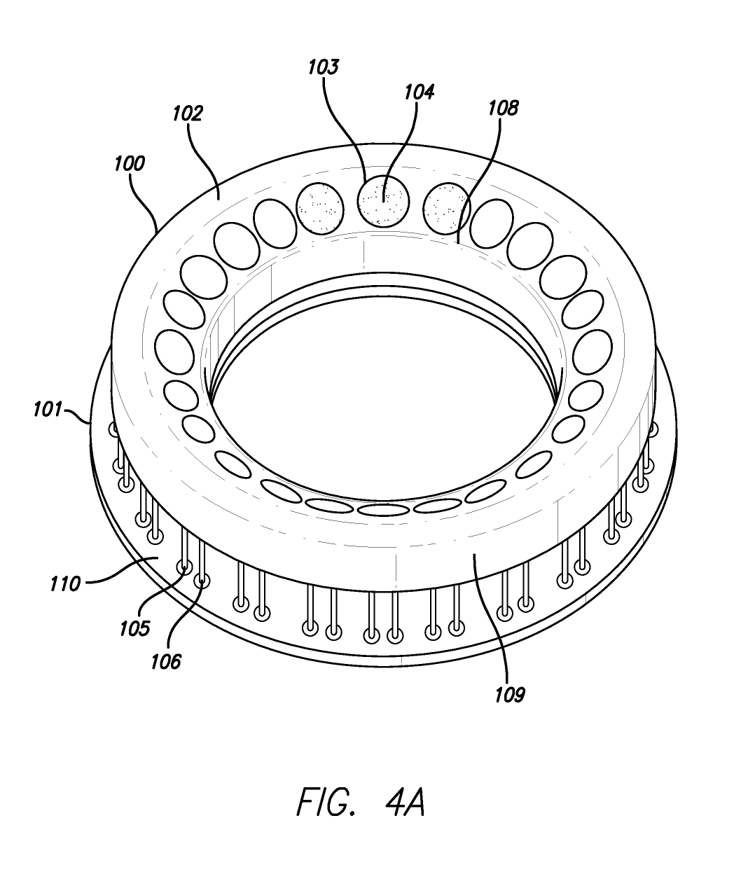

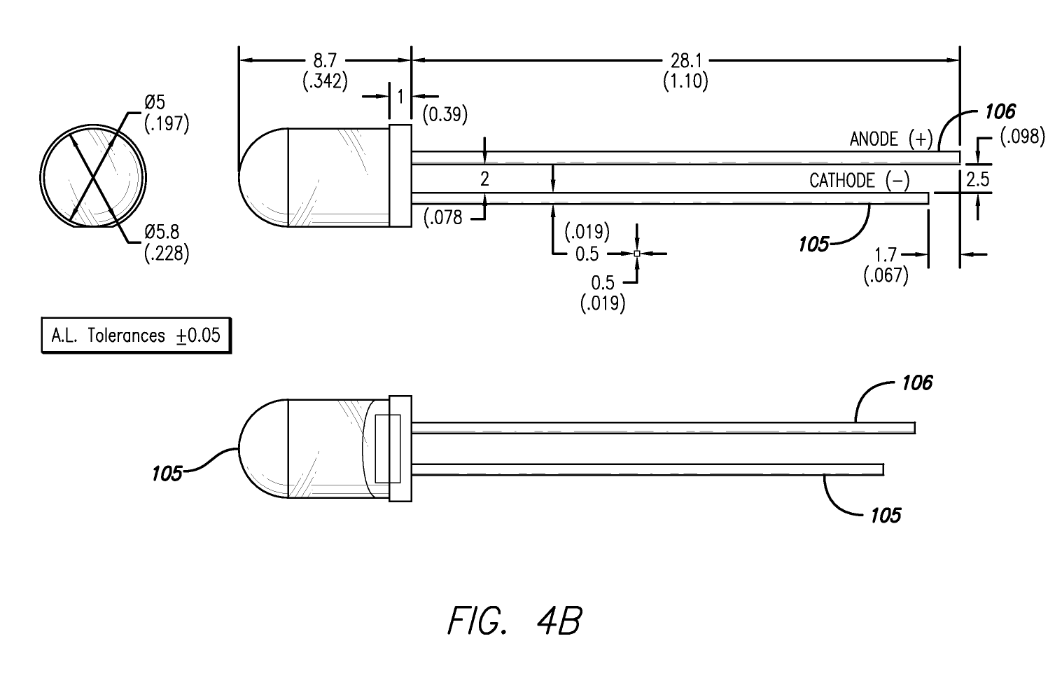

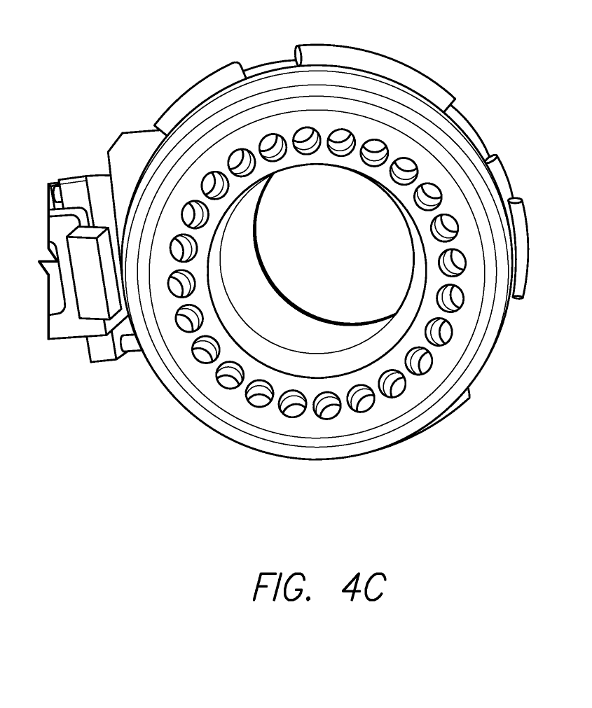

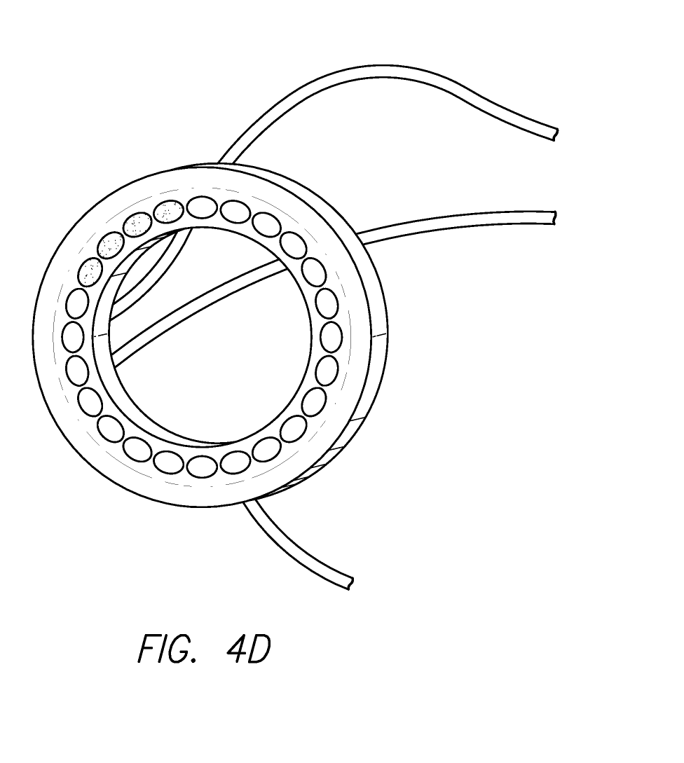

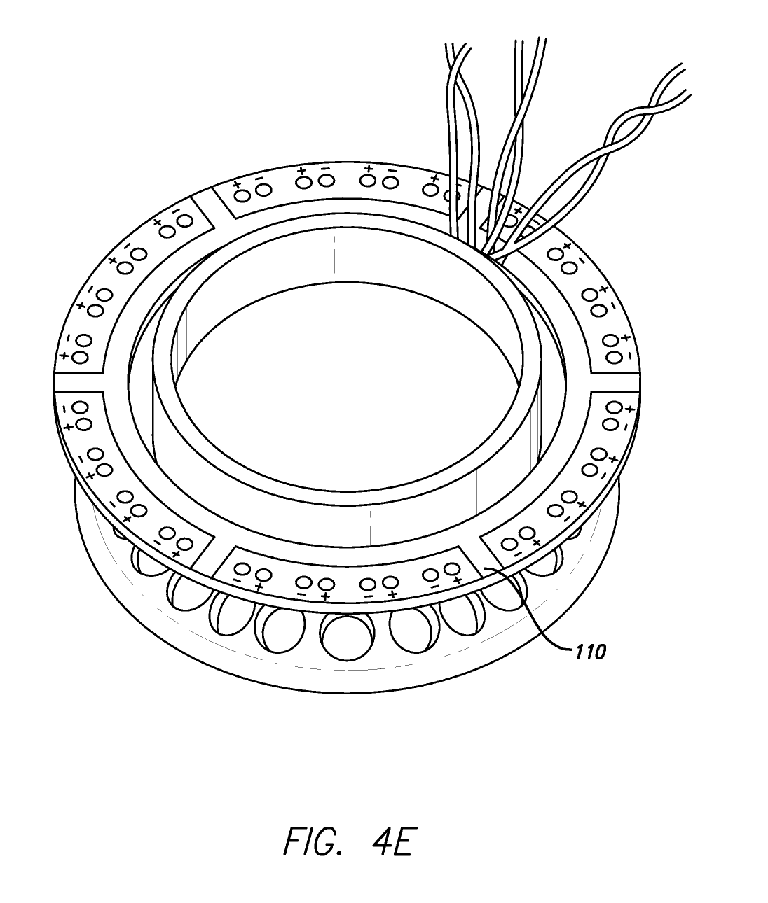

In case of a discrete number of individual light sources distributed uniformly around a ring, one skilled in the art can extend the reasoning as described herein to the cases of a source that is a uniform ring that emits light and to the case of a discrete number of individual light sources that is distributed non-uniformly on the ring or a sector of a ring. For example, a light source can comprise a circular tube containing a circular filament, or a ring can have light sources in a specified sector, such as a single quadrant. (see FIG. 4E)

By way of example and not of limitation, a light delivery device using LEDs and/or other light sources can consider the number of LEDs, N; the angular distribution of radiant intensity of the selected source characterized by, for example, angle at which the radiant intensity from a single LED falls to half the value of its radiant intensity along the axis of the beam from that LED, θ_{s,half}; the radius of the light ring at which the sources are placed, R; the angle of inclination of the axis of the beam of each LED with respect to the optical axis of the eye, θ; the height of the ring with respect to the apex of the cornea, h; and the light source power, Φ. The present teachings allow one skilled in the art to arrive at a set of parameters {N, θ_{s,half}, R, θ, Φ} based on desired design requirements for the distribution of irradiation delivered to a target in the anterior segment and substantially avoiding irradiation at the anti-target in the posterior segment.

As light from a particular source propagates out through a non-absorptive medium, the radiant intensity is unchanged. As the distance from the source increases, the radiant energy propagating out in a given solid angle is spread over an area that increases as the square of the distance from the source. Therefore, the contribution to the irradiance incident on an ocular surface of interest due to the radiant intensity emitted from a particular source into a particular solid angle depends on the distance between that source and the ocular surface element that intercepts that solid angle.

The irradiance at the ocular surface that intercepts light from a given source also depends on the orientation of the ocular surface element that intercepts the light emitted into a particular solid angle. Specifically, the irradiance varies with the cosine of the angle between the unit normal to the surface element dA and the line of sight that connects dA with a particular light source. Equivalently, the effect of the orientation of dA relative to the line of sight between it and a particular source can be described by the dot product of the unit normal of dA and the unit vector pointing from the source to the surface element dA.

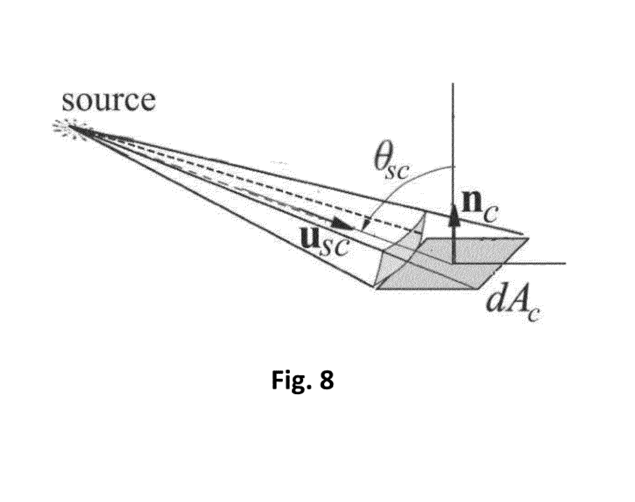

By way of example and without limitation, the surface of the cornea is used as an example of a selected area of the ocular coat (which includes the cornea, limbus and sclera) and subscript “c” is used below in Equation 1 to denote geometric quantities related to the selected area of the ocular coat, such as a differential element of surface area dA_{c} and the unit normal of dA_{c}, n_{c} as shown in FIG. 8. One skilled in the art could extend the present teachings to address light delivery to a surface inside the eye, such as the natural lens or a synthetic intraocular lens, by accounting for refraction and other optical effects associated with transmission through the cornea.

The irradiance contributed by a particular area element of a particular source incident on dA_{c} is given by:

$${dE}_{s \to c} = \frac{L ( {θ}_{s} , {φ}_{s} ) cos {θ}_{sc}}{{r}^{2}} {dA}_{s} {dA}_{c} \qquad ( 1 )$$

which is the rate energy received by dA_{c} from dAₛ. Equation 1 can be read as the energy per unit time from dAₛ entering the solid angle dω that intercepts dA_{c}: L(θₛ,φₛ) dAₛ dω, with dω=cos θ_{sc} dA_{c}/r². To evaluate the irradiance at a particular area dAc of the target surface, the contributions of all of the differential source areas that have a direct line of sight to dAc are added together. This can be accurately and conveniently accomplished using software that is commercially available for this purpose such as ZEMAX®.

By way of example and without limitation, the following useful approximations are described. For a discrete source that has minor azimuthal variations, L(θₛ,φₛ) is well approximated by L(θₛ). Further, for discrete sources that are used at a distance r much greater than their size, an entire source may be approximated using a single differential area dAₛ such that the radiant intensity of the source I(θₛ) can be approximated by L(θₛ) dAₛ. The approximate expression for the irradiance at the cornea for one such source is:

$${dE}_{s \to c} = \frac{I ( {θ}_{s} ) cos {θ}_{sc}}{{r}^{2}} {dA}_{c} \qquad ( 2 )$$

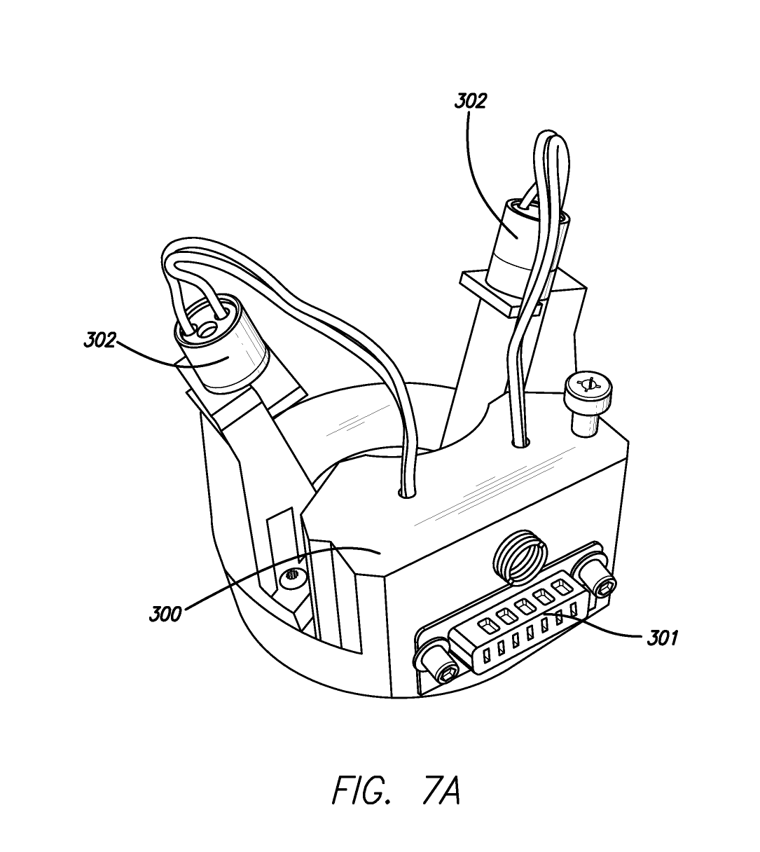

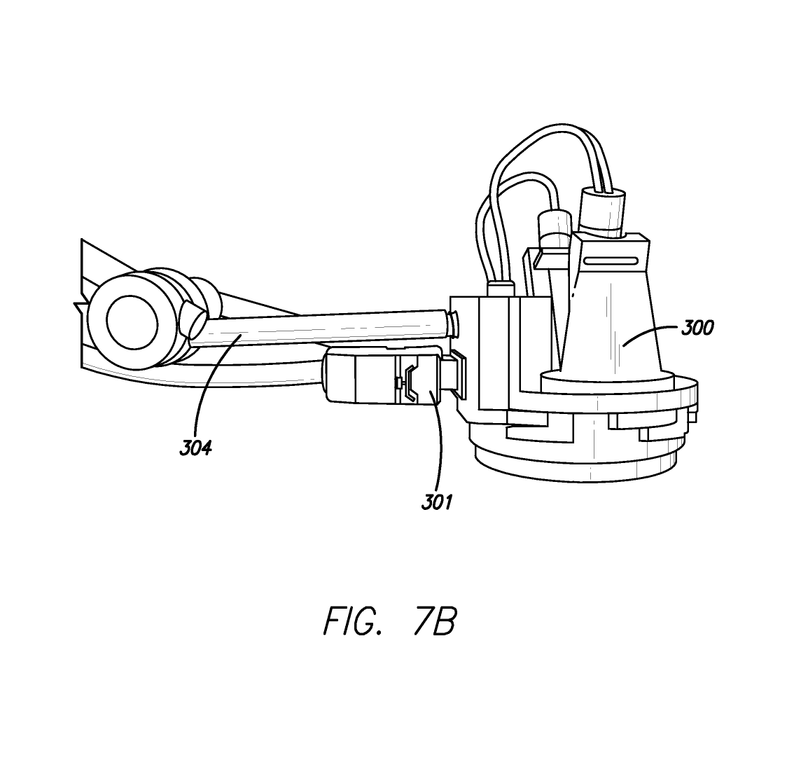

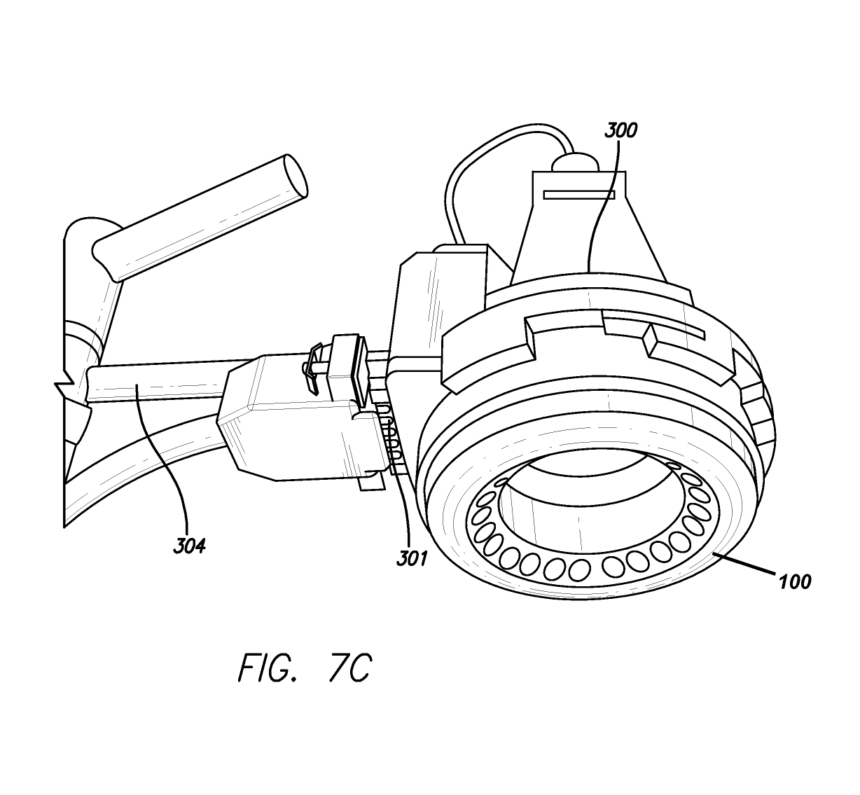

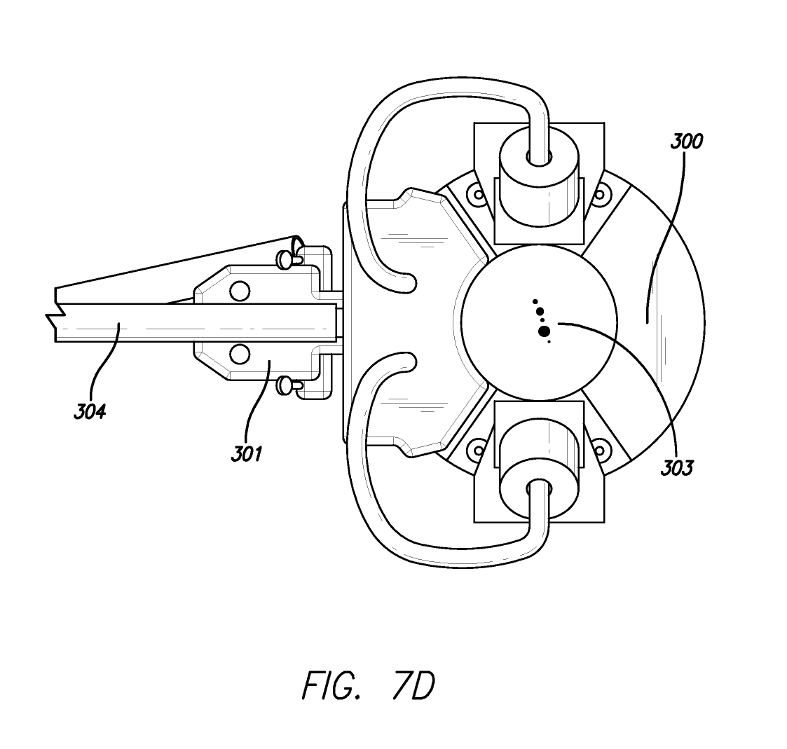

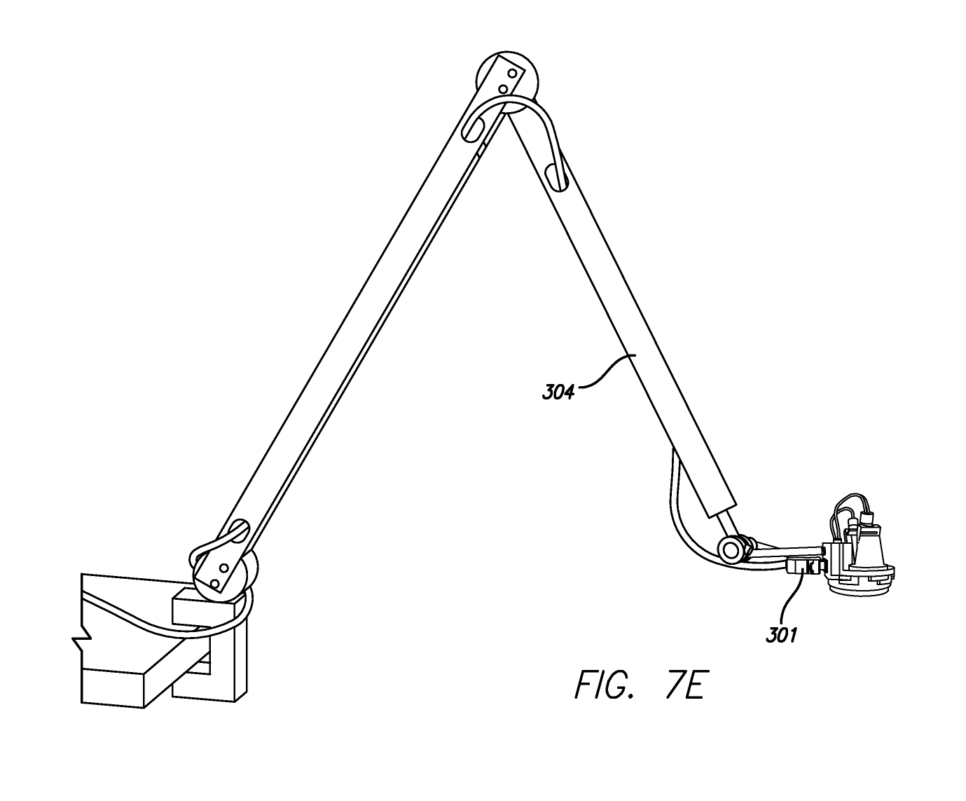

For a discrete number N of such sources distributed symmetrically on a ring (as illustrated in FIGS. 7A-7C for N=4, 8 and 16) that is aligned so that its axis of rotation coincides with the optical axis of the eye, the irradiance at the apex of the cornea when all N sources are operated with substantially the same output power is equal to the product of N and the irradiance at the apex of the cornea due to one of the sources. The irradiance at the apex of the cornea due to one of the sources is given by Equation 2 with the angles being specified for the ray connecting the source to the corneal apex. Consider a point on the cornea near the limbus that is intercepted by the source axis for one specific source. The irradiance there is dominated by that one source and it is desired that this be N-fold greater than the irradiance received by the corneal apex due to one source:

$$\frac{I ( {θ}_{s} ) cos {θ}_{sc , apex}}{{r}_{apex}^{2}} = \frac{1}{N} \frac{I ( 0 ) cos {θ}_{sc , limbus}}{{r}_{limbus}^{2}} \qquad ( 3 )$$

One can use this approximation as a starting point for selecting the components for constructing a device according to the various embodiments of the present disclosure. By way of example and without limitation, calculations are described for the case in which the goal is to apply substantially uniform irradiation to the cornea. All N sources have a line of sight to the apex. Some of the N source can have a line of sight to any particular point on the cornea near the limbus. As a starting point for achieving substantially uniform irradiance as a function of radial position on the cornea, consider one of the N sources having a known (e.g., from a manufacturer's technical specifications) ratio of the intensity at angle θₛ to the intensity along the source axis, I(θₛ)/I(0). For the irradiance received by the cornea at the apex to be approximately equal to that near the limbus,

$$\frac{I ( {θ}_{s} ) cos {θ}_{sc}}{{r}_{apex}^{2}} = \frac{1}{N} \frac{I ( 0 )}{{r}_{limbus}^{2}} \qquad ( 4 )$$

Equation 4 can be used for a simplified case in which the distance from the source to the cornea is much greater than the radius of the cornea, such that rₐₚₑₓ is approximately the same as r_{limbus}; and the axis of the source is approximately normal to the cornea near the limbus, so the cosine term on the right hand side of Equation 4 is approximately 1. Then, the following simplifications provide a useful approximate expression that can be used to find the height at which the light ring should be positioned to minimize the variation in the irradiance as a function of radial position on the cornea. The left hand side of Equation 4 gives the ratio of the height at which the ring should be placed to the radial position of the individual sources in the ring. To apply this equation to solve for the height, the values of N and the radius at which the sources are placed R are selected. Guidance for the selection of those two variables is given in the following paragraph.

$$cos {θ}_{sc} = \frac{1}{N} \frac{I ( 0 )}{I ( {θ}_{s} )} \qquad ( 5 )$$

Continuing with the illustrative example of specifying an inventive device for applying substantially uniform irradiance to the cornea, the azimuthal variation of intensity is considered. The azimuthal variation decreases as N increases. However, the cost of fabrication increases as N increases. Therefore, the smallest N that satisfies an imposed constraint on the magnitude of variations in irradiance is desired to minimize the cost and complexity of the device. For sources distributed uniformly around the ring, the azimuthal variation of intensity observed on the cornea near the limbus can be used to evaluate the magnitude of the deviations from uniformity in the azimuthal direction.

The greatest irradiance on the cornea is at points where the source axis of a particular source intercepts the cornea; aximuthally, minima in the irradiance occur midway between the maxima. In terms of the azimuthal angle α between two flanking sources shown in FIG. 7B, the azimuthal minimum near the limbus occurs at α/2. At such a midpoint, the irradiance due to each of the two sources flanking that minima are equal and the more remote sources make a small contribution (or none at all, if there is no line of sight connecting them to the point of interest on the cornea), so the irradiance at one of the minima can be approximated as twice that due to one of the flanking sources. To evaluate the irradiance due to one of the sources, the known angular distribution of the intensity, I(θₛ)/I(0), is used for the angle θₛ of the ray that connects the source to the midpoint described above, denoted θₘ. Using simple trigonometry to relate the angle alpha and the corneal radius r_{c} to r_{limbus} defined above, the angle θₘ between the axis of the source and the ray that connects the source to the midpoint is approximately

$${θ}_{m} = atan \frac{α π {r}_{c}}{{r}_{limbus}} \qquad ( 6 )$$

The ratio of the corneal irradiance at one of the azimuthal minima to that at one of the aximuthal maxima can be estimated using the same reasoning as above. Given design specifications on the magnitude of variations that can be tolerated (i.e., a requirement that ratio of the azimuthal minimum to the azimuthal maximum be above a certain value), an estimate of the minimum number of sources required to provide the needed degree of uniformity using the above equation, which is implicit in N because alpha=2pi//N. The actual design should be refined using a detailed computation (e.g., using ZEMAX®) and verified by a limited set of experiments.

Selection of the radial position at which the sources should be placed is performed in conjuction with the selection of the sources to meet the required magnitude of the irradiance at the target tissue. The greater the radius at which the sources are placed, the higher the intensity required to provide a specified irradiance (see 1/r̂2 dependence noted above). If the designer wishes to create a more open device, they may choose a brighter source to provide the needed irradiance. If cost constraints dictate that the device be small and use low power sources, a smaller radius can be used. (see Equation 2) Once the designer has chosen the sources, the angular distribution of I(θₛ)/I(0) is set. Once the designer has selected the irradiance, the operating power of the sources and their radial position R can be estimated. Once R is specified, h can be estimated using Equations 3-5, depending of the quality of approximation that is desired.

FIG. 4A shows a light delivery device according to an embodiment of the present disclosure. The light delivery device can comprise a housing portion 100 and an electronics portion 101. By way of example and not of limitation, the housing portion can comprise a housing 102 (e.g., a substantially ring-shaped housing) for housing or mounting a light emitting arrangement (e.g. see Example 1). Although the housing that is shown in FIG. 4A is substantially ring shaped, the housing 102 can have other shapes, such as, for example, circular shaped, square shaped, rectangular shaped, toroidal shaped, etc.

According to an embodiment as shown in FIGS. 4A-4E, the housing 102 can have through-holes 103 along the circumferential extension of the ring shaped housing 102 for placement of the light emitting arrangement. By way of example and not of limitation, the light emitting arrangement can be a plurality of light emitting elements such as light emitting diodes (LEDs) 104, as shown in FIGS. 4A-4E. However, other light sources such as light bulbs, filtered light bulbs, or light sources with optical fiber can also be used. Those skilled in the art would understand that other types of light sources can be used as the light emitting elements according to their desired use of the device. Accordingly, the terms “light emitting arrangement”, “light emitting elements”, “LEDs”, “light bulbs”, “filtered light bulbs”, “lamps” and “light sources with optical fiber” can be used interchangeably throughout the present application.

The LEDs 104 shown in FIG. 4A are positioned in each of the holes 103 such that the illumination end (as shown in FIG. 4C) of the LED is exposed from the interior side 108 of the substantially ring shaped housing 102. The anode 106 and cathode 105 probe side of the LED 104 protrude on the exterior side 109 of the substantially ring shaped housing, which connect to a circuit board 110 as shown in FIGS. 4A and 4E. The circuit board is then ultimately connected to a power source (e.g., battery pack, power adapter) and/or a controller to turn on, turn off, or dim the LEDs. Each of the LEDs can be controlled independently from each of the other LEDs as selected by the user. An exemplary controller is also described that can be connected to the light delivery device, which can be used to turn on, turn off, or dim the LEDs as selected by the user (e.g. see Example 1, 2).

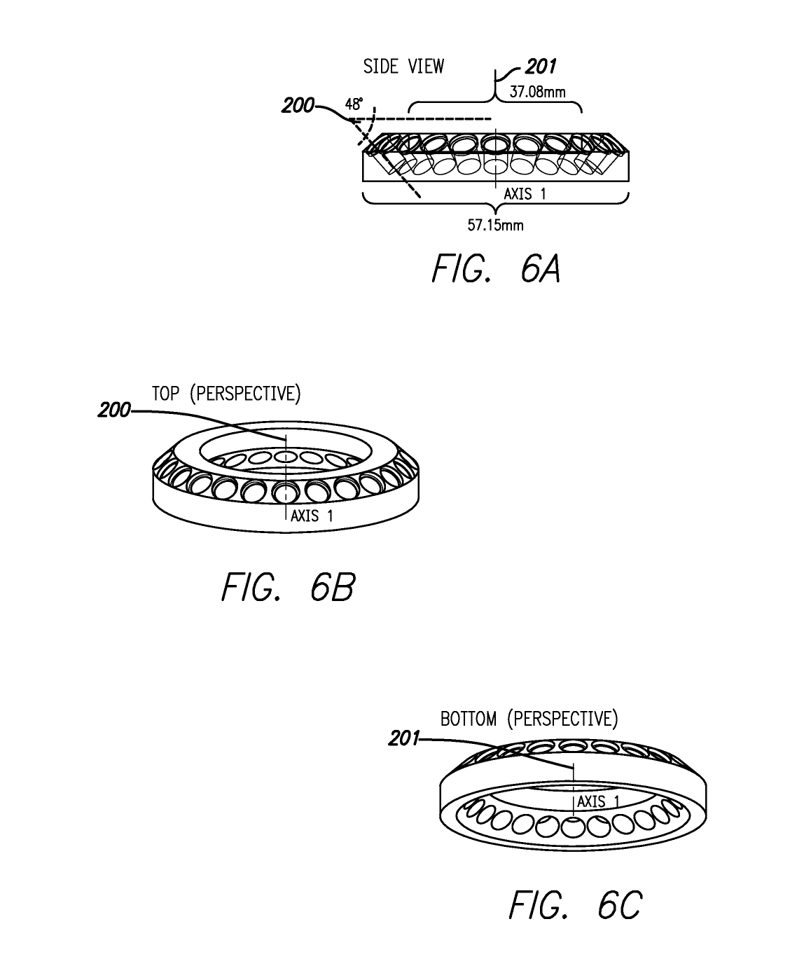

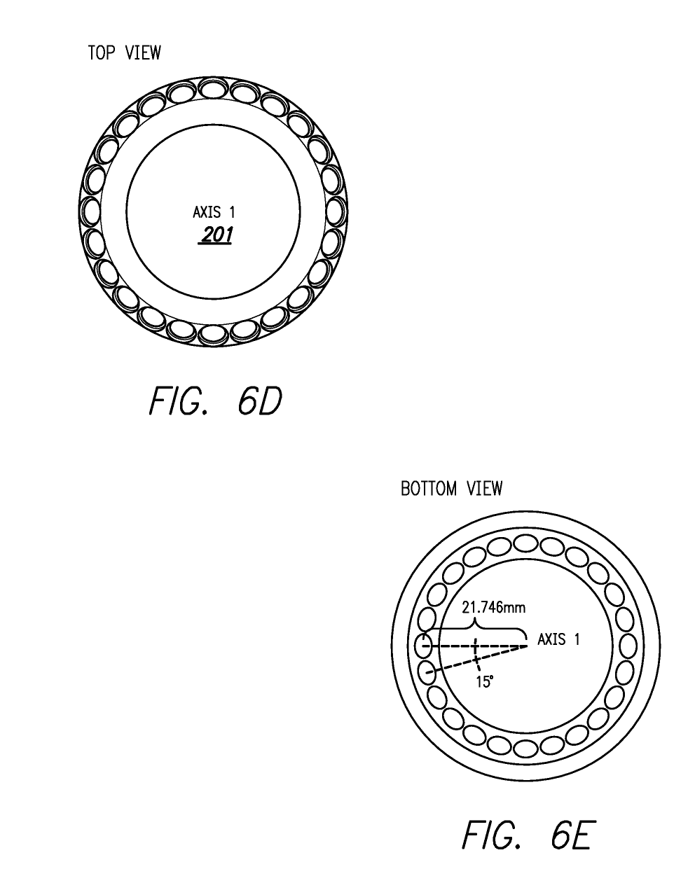

The LEDs 104 are mounted on the housing 102 at an angle 200 as shown in FIGS. 6A-6E such that the center of the illumination of the LED points along the direction of the central axis 201 of the ring shaped housing. The central axis 201 can be defined as an imaginary axis that extends orthogonally to the plane formed by the ring shape of the ring shaped housing. Thus, when a plurality of LEDs are illuminated, and each of the LEDs are angled in the direction of the central axis 201, then the illumination of the LEDs converge somewhere along the central axis 201 depending on the degree of the angle 200. By way of example and not of limitation, the LEDs in FIG. 6A shows the angle to be 48 degrees (e.g, θ shown in FIG. 1). FIG. 6E shows by way of example and without limitation, 24 LEDs around the ring shaped housing at 15 degrees apart. An equivalent angle is also shown in FIG. 7B where each of the 8 lights are spaced apart as angle cc. Thus, those skilled in the art will understand that the angle cc can be computed by dividing 360 degrees by the number of lights (e.g., LEDs), N.

In some embodiments, the center opening of the ring shaped housing can be used for the user of the light delivery device to observe the target, when in use. Thus, the opening can allow the user to see the target directly through the light delivery device by directly viewing (e.g., by looking down the optical axis of the eye) the target region, instead of having to observe the target region from the side. Alternatively, the housing can be shaped and/or configured to be optically transparent to the user such that the user can observe the target region from through the light delivery device. By way of example and not of limitation, a camera or other imaging device can be mounted to the device, and the camera can be connected to a monitor such that the user can see the same view as if there was an opening in the housing (e.g. see Example 1).

FIGS. 7A-7E shows the light delivery device connected with a mounting module 300. The mounting module 300 is connected with the housing portion 100 and the electronics portion 101 (of FIG. 4A) of the light delivery device to form one complete module as shown in FIGS. 7A-7E. The mounting module can comprise an electronic adapter 301 for easily connecting power to each of the LEDs on the housing 102 (of FIG. 4A).

According to another embodiment, a distance indicating device can be connected with the light delivery device (or any portion of the light delivery device module thereof) to measure, determine, set and/or indicate a distance of the light emitting arrangement from the target to be irradiated.

In one embodiment of a distance measuring device, one or more laser sources can be used to measure and/or determine the distance. By way of example and not of limitation, a pair of laser sources 302 can be placed on the mounting module 300. Such laser sources 302 can be positioned at an angle so that the each of the laser beams from each laser source converges at a set distance. Thus, the user of the light delivery device can determine that the light delivery device is at the set distance when the two laser beams converge. FIG. 7D shows the two laser beams 303 on the target, where the target is at a distance such that the two laser beams 303 on the target do not quite converge. Thus, the user can determine that the light delivery device will need to be moved either closer or farther from the target until the two laser beams 303 visible on the target converges. Although FIG. 7D shows dots formed by the laser beams, other distance determining patterns can be use by superpositioning a plurality of sharply focuses patterns and/or reticles (e.g. see Example 1-3). One skilled in the art can choose known reticles that super impose when both distance and orientation are correct.

In another embodiment of a distance measuring device, a spacer can be used to measure and/or determine the distance. By way of example and not of limitation, a spacer can be connected to the light delivery device, configured to provide a distance and relative orientation between a target and the light delivery device when the spacer is placed on the target surface.

FIGS. 7B-7E show the light delivery device 100 and the mounting module connected with an electronic adapter 301 and an arm 304 according to some embodiments. The arm 304 can be connected to some fixed equipment such that the user can move, and precisely position the light delivery device at the desired position and orientation near the target.

In some embodiments, the light delivery device 100 and the mounting module can be fixed and the target can be moved to the desired position.

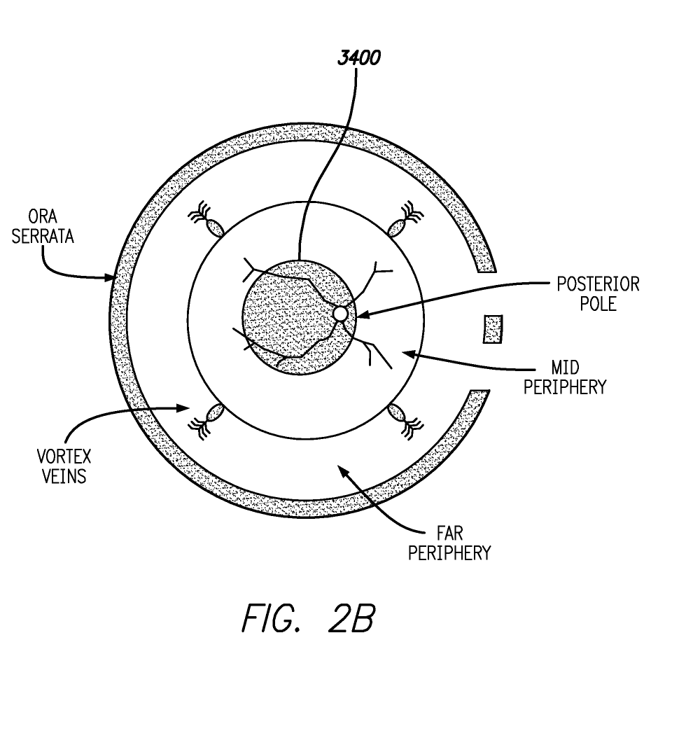

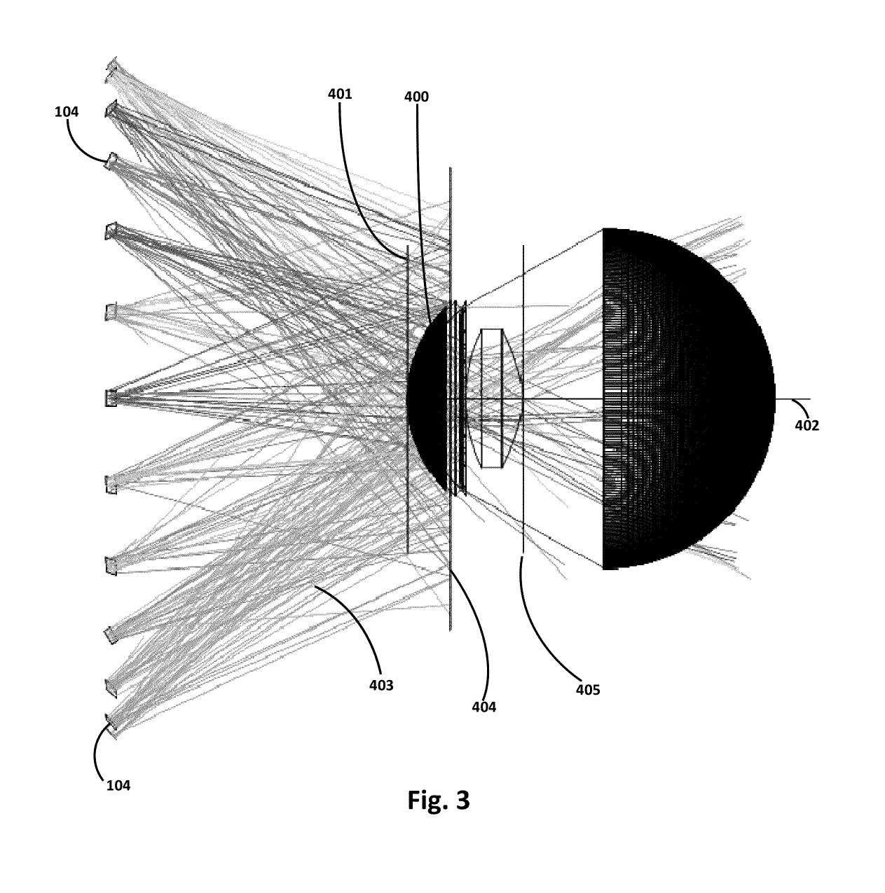

According to an embodiment of the present disclosure as shown in FIG. 3, the light delivery device can be positioned over a target that is desired to be irradiated by the light emitting arrangement (e.g., LEDs) by way of example and not of limitation, the target may be the cornea 400 as illustrated in FIG. 3. In particular, the light delivery device can be positioned such that the central axis (e.g. 201 of FIG. 6A) of the light delivery device is positioned directly over the center of the target region that is desired to be irradiated such that when the LEDs are turned on, the desired target region is irradiated with a therapeutic distribution of irradiance at the target.

According to another embodiment, the target region 400 can have a substantially convex surface as shown by 400 in FIG. 3. In such case, the substantially convex target region can have a central axis 402 can coincide with the axis of the light delivery device using the distance and orientation as herein described. Central axis 402 is aligned to coincide with the optical axis of the eye.

According to an embodiment of the present disclosure, the substantially convex target region can be, for example, an eye (e.g., human eyeball). In particular, an anterior segment of the eye (e.g., cornea, sclera, limbus) can be irradiated by precisely aligning the light delivery device over the eye. In case of irradiating the cornea of an eye, irradiating the cornea oblique to the central axis 402 (which is the optical axis of the eye in case of the cornea) can cause the radiation to penetrate the cornea and avoid the retinal anti-target region of the eye.

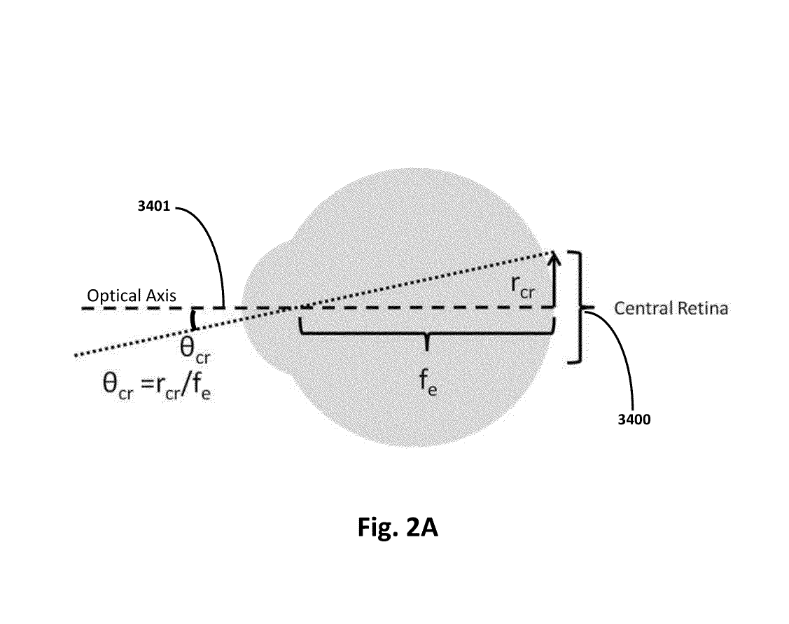

The “retinal anti-target” region indicates a region of the retina that should be substantially avoided by radiation (reached by approximately 10% or less of radiant power delivered to the eye). In several embodiments, the retinal anti-target region comprises a substantially circular central retinal region 3400 around the macula and the fovea as shown in FIG. 2A-2B. By way of example and not of limitation, the average size of the central retinal region 3400 in an eye of an adult person is approximately 12 mm in diameter. Such effect of irradiating the retinal anti-target region can be undesirable because it can cause discomfort to the person or cause damage to the retina. Therefore, by utilizing the light delivery device according to various embodiments of the present disclosure, such irradiation of the cornea can be performed by irradiating the cornea from an angle that is oblique to the optical axis and that substantially avoids the anti-target retinal regions formed by central retinal region 3400. In some embodiments the anti-target retinal region is formed by the macular region within central retinal region 3400. In some embodiments the anti-target retinal region is formed by the fovea within the macular region.

FIG. 2B shows various portions of the retina (e.g., central vs. peripheral retina). The central part of the retina is called the macula, and its very center is the fovea. The fovea is where the finest detail vision is perceived; both the fovea and the surrounding macula perceive color. The peripheral retina refers to that portion outside the central retina. The peripheral retina has lower visual acuity and better low-light sensitivity than the macula.

The optic disc, a part of the retina sometimes called “the blind spot” because it lacks photoreceptors, is where the optic-nerve fibers leave the eye. It appears as an oval white area of 3 mm². Temporal (in the direction of the temples) to this disc is the macula. At its center is the fovea, a pit that is responsible for our sharp central vision but is actually less sensitive to light because of its lack of rods. Around the fovea extends the central retina for about 6 mm and then the peripheral retina

From a clinical perspective, the retina emanates at the optic disc and extends anteriorly to the ora serrata. The optic disc represents the confluence of the retinal nerve fiber layer (NFL) as it exits the globe. The retina is divided into the macular area within the central posterior pole and the peripheral retina.

The lines 403 shown in FIG. 3 show the path of the radiation from the LED 104, penetrating the cornea 400, where a small portion of the penetrated radiation is incident on the retinal anti-target region such that the small portion is below a selected threshold for example, based on published safety guidelines. FIGS. 2A-2B show that if a light is directed toward the cornea from an angle greater than or equal to θ_{cr} that is oblique to the optical axis 3401, then the light avoids hitting the retinal anti-target region (e.g., central retinal region 3400). By way of example and not of limitation, the average focal length of the human eye is approximately 17 mm. Therefore, assuming a radius of the retinal anti-target region to be 6 mm, an angle θ_{cr} of greater than 20 degrees can ensure that a substantial portion (e.g., greater than 90%) of the radiation from the incident light avoids the retinal anti-target region.

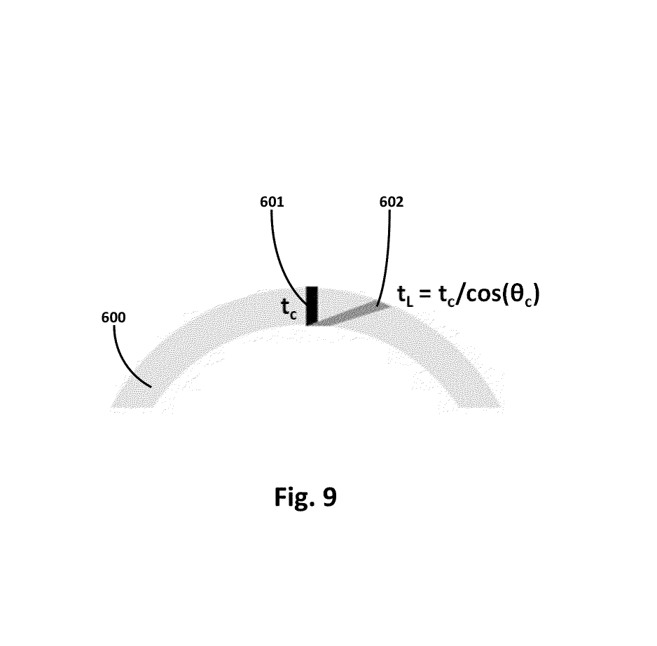

FIG. 9 shows a close up cross sectional view of the corneal tissue 600. If the radiation penetrates the cornea from an angle that is parallel to the optical axis 601 of the cornea, then the radiation is transmitted through the thickness of the cornea, shown as t_{c}. However, if the radiation penetrates the cornea from an angle that is oblique (e.g., θ₀>>θ_{cr} degrees in FIG. 9) to the optical axis of the cornea (e.g., θ_{c}=42 degrees), then the radiation penetrates the corneal tissue at an angle such that the tissue path length is equivalent to t_{c}/cos θ. The greater tissue path length allows for greater absorption of the radiation by the corneal tissue. In some embodiments, a photosensitizing compound (e.g., drugs) can be applied to the eye, and thus absorbed by the corneal tissue. Therefore, the thicker the tissue path length for the radiation to pass through, the more radiation that can be absorbed by the photosensitizing compound. Finally, the greater the absorption of the radiation by the tissue and the photosensitizing compound, the less radiation that is transmitted beyond the cornea to the back of the eye. Thus, by irradiating the corneal tissue from an angle oblique to the optical axis 601, the intensity of the radiation (e.g, light intensity) is reduced and avoids the retinal anti-target region. The intensity of the radiation that is transmitted passed the corneal tissue can be represented by I₀ exp (−μ_{c}t−μ_{d}t), where I₀ is the incident radiation, μ_{c} is the absorption of the radiation by the cornea, μ_{d} is the absorption of the radiation by the photosensitizing compound, and t is the distance of the path length.

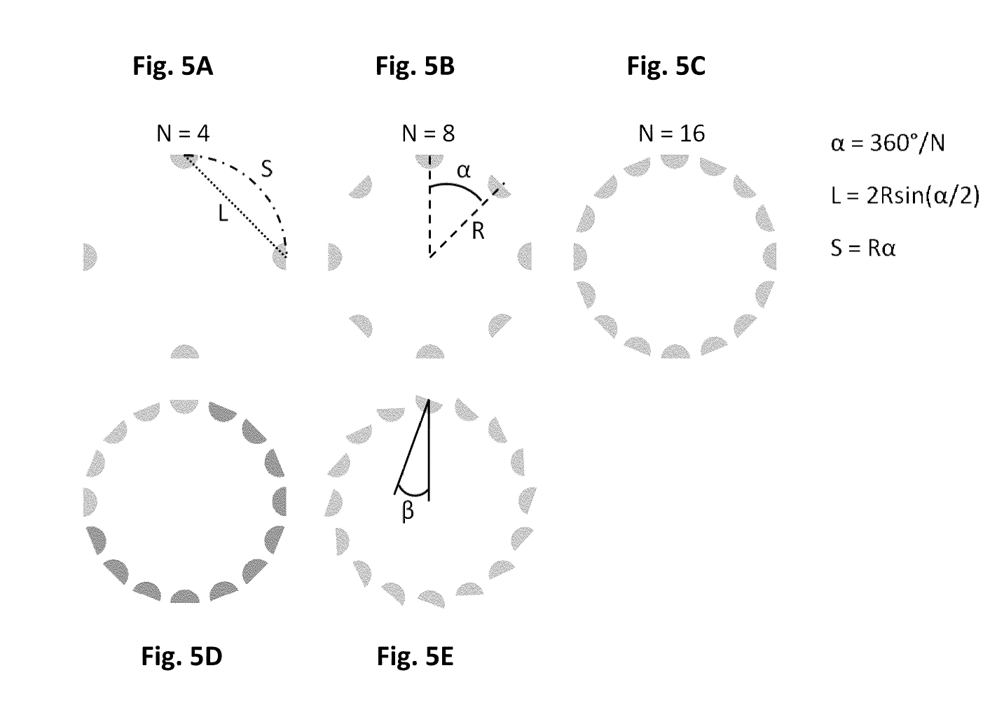

FIG. 5A-5C show that any number of light emitting elements (e.g., 4, 8, 16) can be configured in the light delivery device as described in the various embodiments of the present disclosure. FIG. 5D shows 16 light emitting elements (e.g. 104 of FIG. 4A) where each of the 16 light emitting elements are arranged in a circular pattern that points toward the center of the circular arrangement. Alternatively, FIG. 5E shows 16 light emitting elements (e.g. 104 of FIG. 4A) are arranged in a circular pattern, yet where each of the 16 light emitting elements point off-center of the circular arrangement, shown as angle β (e.g. see Example 1).

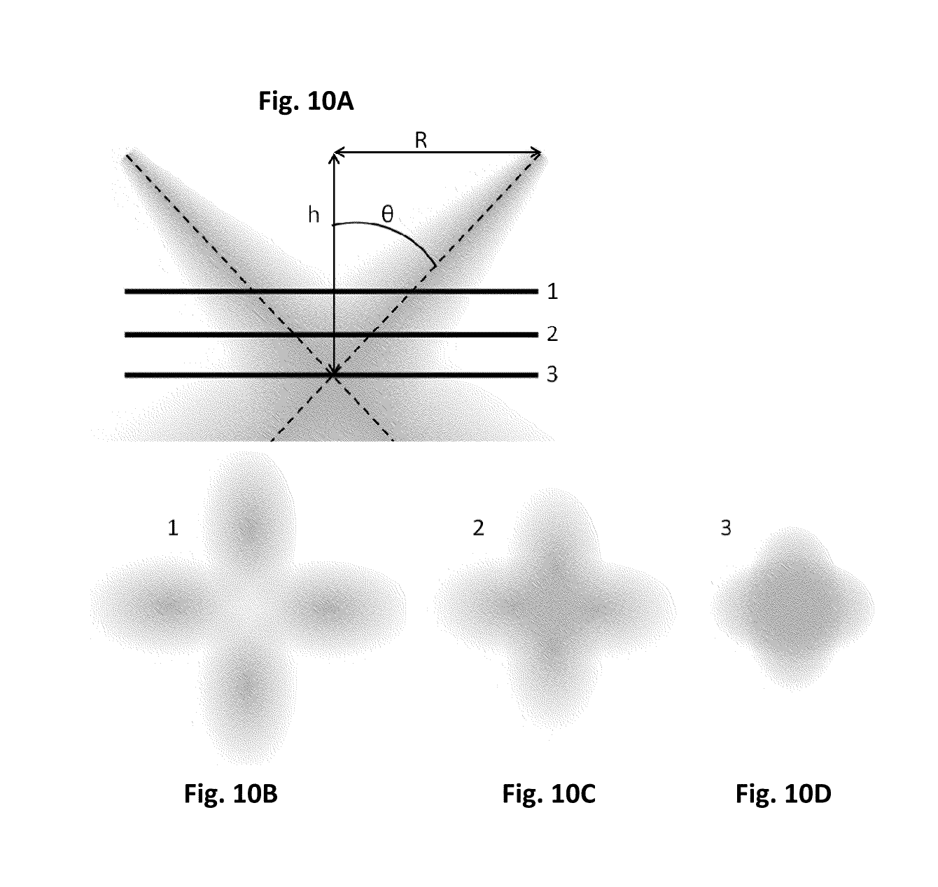

FIGS. 10A-10D illustrate the effects of the pattern formed on the target when there are four light emitting elements (e.g. 104 of FIG. 4A). FIG. 10A shows a side view of the light delivery device with the target. The h shows the position of the apex of the cornea relative to the light delivery device when the central axis of the device is parallel to the optical axis of the eye. For example, the corneal apex can be at line 3 in FIG. 10A and accordingly, would provide a distribution of irradiance on the cornea as shown in FIG. 10D. FIGS. 10B-10D are views looking down onto the cornea, over the light delivery device. By decreasing h (e.g., moving the light delivery device closer to the target), the distribution of the irradiance would change, for example, shown in FIG. 10C (which corresponds to line 2 in FIG. 10A). Further decreasing h would change the distribution of the irradiance as shown, for example, in FIG. 10B (which corresponds to line 1 in FIG. 10A).

Although four light emitting elements are used to describe the effects shown in FIGS. 10A-10D, those skilled in the art would understand that a substantially similar effect can result in configurations with more or less light emitting elements. Lines 1, 2, and 3 represent three different target regions at three different distances (h) from the light source plane (e.g. plane defined, for example, by the plurality of light sources 104 depicted in FIG. 3) of the light emitting elements (e.g. 104 of FIG. 3). In each case, the light emitting elements 104 are directed at the target region from an angle θ. R is the radius of the arrangement of the light emitting elements 104. Thus the relationship between h, R and θ can be shown as h=R/tan θ. In the first scenario where the target region is at line 1, the pattern formed by the light can appear as shown in FIG. 10B where the light pattern from each of the light emitting elements are distinctly visible. In the second scenario where the target region is at line 2, the pattern formed by the light is less distinct than for target region at line 1 since the target distance (h) is greater. It can be seen in FIG. 10A that the light (e.g. whose path is shown via dotted lines) converges approximately at a point on line 3. Thus, as the distance (h) increases, the light becomes more convergent, up to the convergent point on line 3. Consequently, in the third scenario where the target region is at line 3, the patterns formed by the light appear the least distinct as shown in FIG. 10D. Thus, the user of the light delivery device can determine the most suitable distance (h) (e.g., 19.2 mm) for the application being performed, according to the light pattern and/or focus desired.

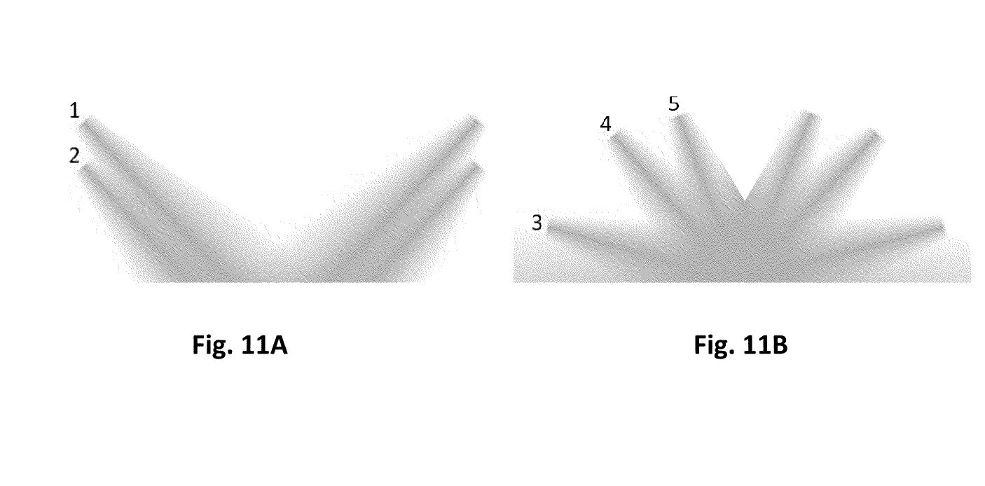

In some embodiments, multiple light delivery device can be used simultaneously to irradiate the target region as shown in FIGS. 11A-11B. Alternatively, a single light delivery device can have more than one set of light emitting arrangements (e.g., two sets of ring-shape patterned LEDs). The multiple sets of light emitting arrangements can all have the same angle, as shown in FIG. 11A, or can have different angles, as shown in FIG. 11B.

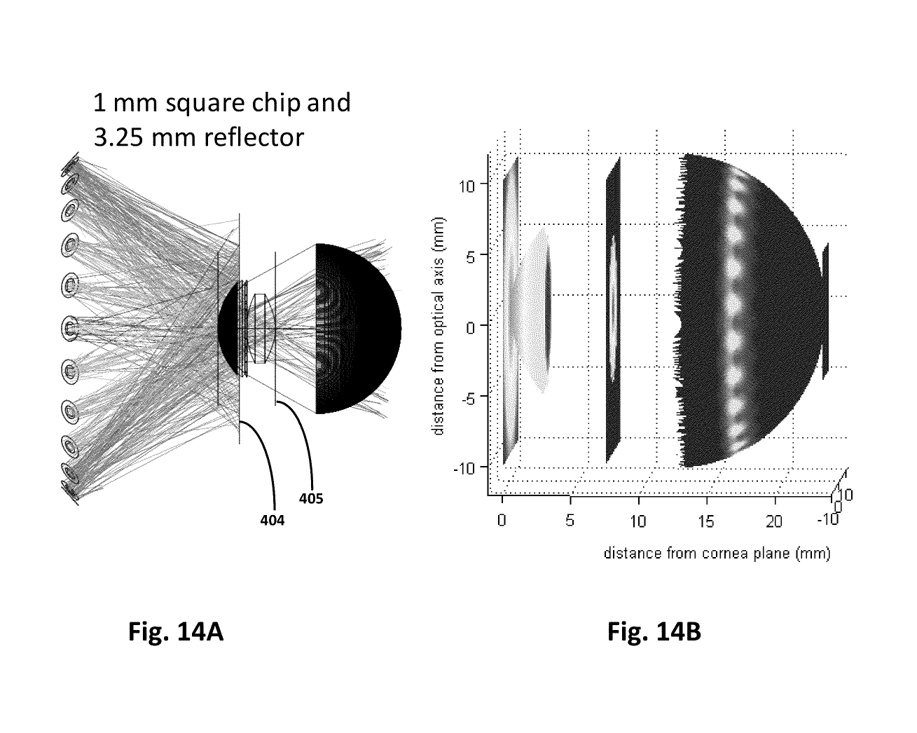

In some embodiments, each of the light emitting elements can comprise a reflector to vary the spread of light from each of the light emitting elements (e.g., LEDs). Further, some LEDs can comprise a square chip (e.g., 1 mm square chip, 3.25 mm square chip) that illuminates as shown in FIG. 14B.

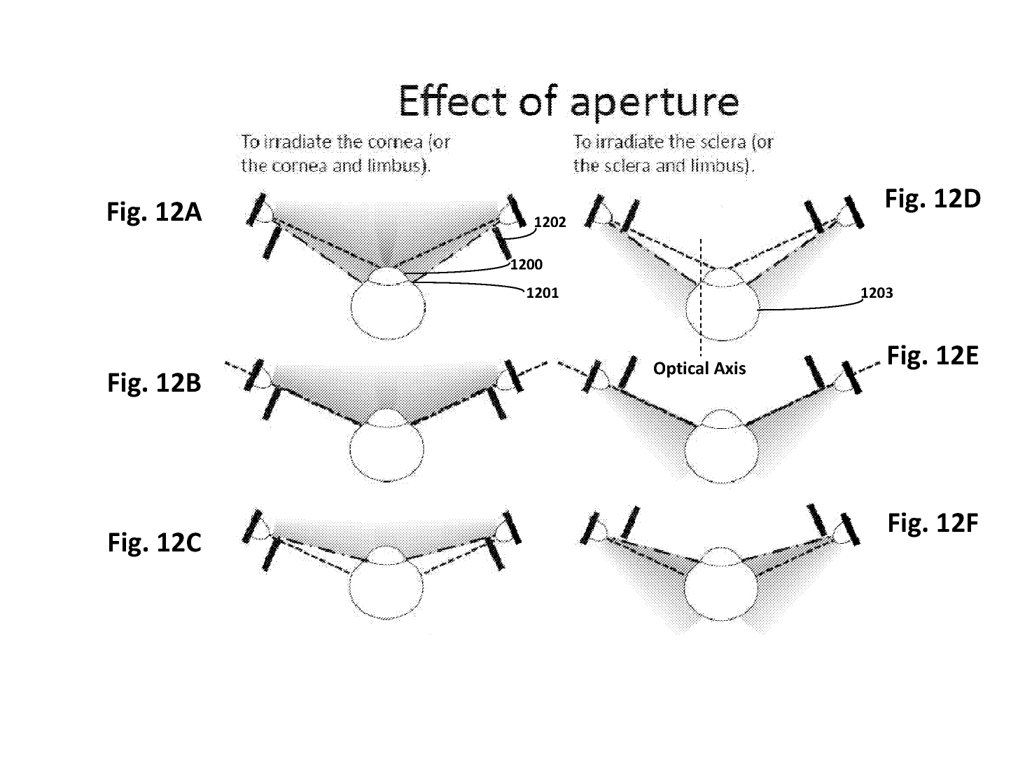

In some embodiments, each of the light emitting elements can comprise an aperture 1202 as shown in FIGS. 12A-12F to control the radiation of light from each of the light emitting elements (e.g., LEDs 104). FIG. 12A shows the aperture 1202 partially blocking the lower portion of the radiation by the LEDs such that the light irradiates, for example, the cornea 1200 and the limbus 1201 of an eye. FIG. 12C shows the aperture 1202 further blocking the radiation of light such that only the cornea 1200 is irradiated. Alternatively, the upper portion of the radiation can be blocked as shown in FIGS. 12D-12F in order to irradiate, for example, only the sclera 1203 of the eye, and not the cornea 1200 or the limbus 1201 (e.g. see Examples 16, 17).

In some embodiments, the light delivery device can be used during a lock-in process of a light adjustable lens. After implantation of a light adjustable lens in a patient, the power of the lens can be adjusted by delivering a dose and profile of light onto the light adjustable lens which causes the lens to change shape and optical performance. When a suitable lens power is determined, the power can be locked in by delivering the light dose yielding the suitable lens power.

In some embodiments, light arrangements herein described can be used in connection with a holder adapted to host a light emitting arrangement and to locate the arrangement at a distance from a target ocular region when in use. In particular, the distance is to allow the radiation from the light emitting arrangement hosted in the holder to be convergently directed towards the target ocular region, along a plurality of irradiating directions each direction of the plurality of irradiating directions being oblique to the optical axis of the eye according to various embodiments herein described. Reference is made in this connection to the illustration of FIGS. 6A-6E and 7A-7E showing various views of a holder according to an embodiment of the present disclosure.

In particular, in the embodiments of FIGS. 7A-7E, the holder hosting a light arrangement is used in combination with an arm shaped support in a light delivery system as will be understood by a skilled person. Additional supports adapted to position a light delivery device herein described at a set distance from the target ocular region in the eye of an individual during use of the light delivery device to allow the radiation to be convergently directed towards the target ocular region, along a plurality of irradiating directions oblique to the optical axis of the eye, will be identifiable by a skilled person. In some embodiments, positioning of a device can be performed using laser arrangement that is configured to identify a position for the device in connection with a target ocular region of interest (see e.g. Example 1-2) Accordingly in some embodiments, light delivering systems can comprise a device together with a laser arrangement for correct position of the device in connection with methods herein described.

In some embodiments, irradiating a target ocular region in an individual can be performed with devices herein described as well as with one or more additional devices directed to emit lights other than the specific devices herein described. In those embodiments, instruments are used to provide a radiation towards a target ocular region along a plurality of irradiating directions, each direction oblique to the optical axis of the eye. In the method, the radiation is provided at a distance from the target ocular region to allow said radiation to be convergently directed towards the target ocular region.

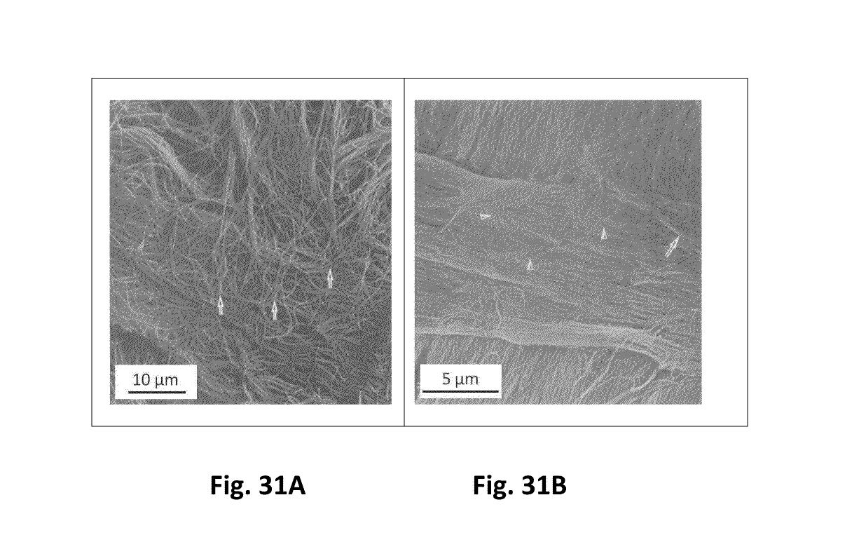

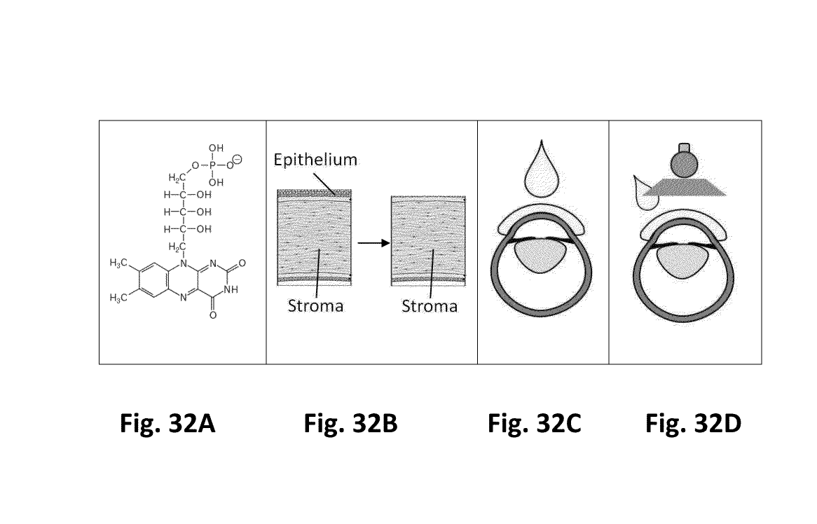

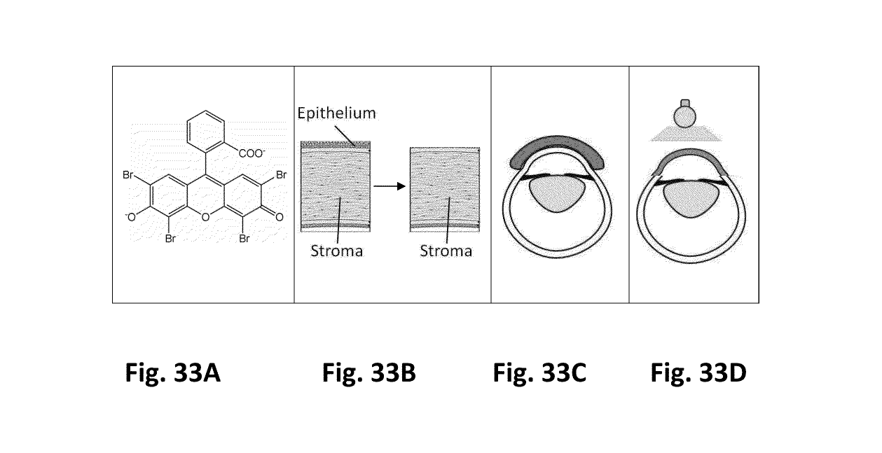

In particular, in several embodiments, irradiating can be performed and controlled in view of different optical properties of the various regions of the eye and in particular of the sclera and the cornea. The difference in optical properties between the sclera and cornea is due to the differences in the size, spacing and orientation of the collagen fibrils. Scleral collagen fibrils vary in both diameter (e.g., 30 to 300 nm) and spacing (e.g., 250 to 280 nm)^{[23]}. They form an interweaving morphology conferring great strength to the sclera to protect the eye (FIG. 31A). The white appearance of the sclera (and the opacity that is vital to its function) is due to light scattering from heterogeneities in fibril diameter, fibril spacing and fibril orientation on length scales from 150 to 600 nm. In contrast, corneal collagen fibrils are very regular in their diameters (e.g., 20 to 33 nm) and spacing (e.g., approximately 60 nm)^{[23]}. The highly regular fibril/proteoglycan structures form sheets (e.g., lamellae) that are stacked such that the collagen fibrils lie in the plane of the tissue, giving the cornea its unique combination of transparency and strength (FIG. 31B).

In some embodiments, oblique directions of radiation with respect to the optical axis in methods herein described can be determined using techniques and approaches identifiable by a skilled person (see e.g. Example 22)

In some embodiments, irradiating the eye of an individual can be performed to perform a photodynamic cross-linking on an eye. The terms “crosslink” or “crosslinking” as used herein refers to a formation of a covalent bond between two molecules and in particular, two polymer molecules. A plurality of crosslinks can provide a network of interlinked polymer molecules held together by covalent linkages. If the polymers are proteins, the crosslink can be referred to as a “protein-protein” crosslink. For example, a collagen molecule can be crosslinked to other collagen molecules to form a network of interlinked collagen molecules held together by covalent linkages. Other proteins such as proteogylcans, for example, can also form cross-links.

The term “photodynamic crosslinking” refers to a method of performing protein-protein cross-linking using photo-activated molecules. For example, the method can be performed by allowing diffusion of a photo-activated molecule to penetrate a desired tissue following by an irradiating of desired locations of a tissue with a wavelength of light suitable to transition the photo-activated molecule from a ground state to an excited state and thus allow crosslinking of proteins to occur by a crosslinking pathway.

Examples of “crosslinking pathways” by which protein-protein crosslinks can be formed when irradiated with light in the presence of a photosensitizer typically include two major photosensitization pathways: type I (direct reaction pathway) and type II (indirect reaction pathway). Both type I and type II photosensitization pathways begin with the photosensitizer absorbing and transitioning from its ground state to an excited state. A second step in the photosensitization type I pathway comprises a reaction of the excited state photosensitizer with a protein molecule, for example by hydrogen or electron transfer. A second step in the photosensitization type II pathway comprises transferring of energy of the excited state photosensitizer to ground state molecular oxygen, thus producing singlet oxygen. Singlet oxygen, some times referred as “reactive oxygen species”, can then oxidize a protein). In some cases, photosensitization reactions by both type I and type II pathways can occur concurrently.

In some embodiments, the method for photodynamic crosslinking comprises providing a photosensitizing compound; topically applying a set quantity of the photosensitizing compound to a target portion of the eye for a set contact time; and irradiating the target portion of the eye with a light source after a set delay time, after removing the excess photosensitizing compound from the eye. In some embodiments, the method can also comprise removing excess photosensitizing compound from the target portion of the eye upon expiration of the set contact time.

The particular photosensitizing compound, the set quantity of the photosensitizing compound to be topically applied to a target portion of the eye, the set contact time, the set delay time after removing excess photosensitizing compound and before irradiating the target portion of the eye, can be used to control a quantity and/or distribution of the photosensitizing compound inside a target tissue and can be set in accordance with a desired effect of photodynamic crosslinking.

The term “contact time” refers to an amount of time that the photosensitizing compound, which is to be topically applied to a target portion of the eye, is allowed to remain on the target portion on the eye before the compound starts diffusion in a depth direction with respect to the surface of the region. Typically, contact time can be set to span between application and removal of any excess of the photosensitizing compound. For example, a longer contact time can provide a higher concentration of the photosensitizing compound in the target tissue compared to a shorter contact time which can provide a lower concentration of the photosensitizing compound in the target tissue. Longer contact times can also lead to a more homogeneous distribution inside a target tissue. For example, a longer contact time can allow the photosensitizing compound to diffuse throughout the surface of a target tissue and thus provide a more homogeneous distribution of the photosensitizing compound.

The term “delay time” refers to a time in which a compound applied to a target region is allowed to diffuse in depth with respect to the surface of the target region. Typically, delay time can be set to span between a removal of the photosensitizing compound and an irradiation of the target tissue and can used to control a distribution of cross-links to be formed in a target tissue upon irradiation. For example, increasing a delay time can allow a concentration of the photosensitizing compound to decrease in the anterior portion of the eye and increase in the posterior portion of the eye. A decrease in the concentration of the photosensitizing compound in the anterior portion of the eye can allow a deeper penetration of light during irradiation.

Corneal tissue generally ranges between approximately 0.3-1 mm in depth varying by individual, and in some individuals the corneal tissue can be less than 0.3 mm depth. Scleral tissue can range between approximately 0.3-1 mm depth and varies in thickness from the posterior pole being approximately 1 mm depth and decreasing in thickness to approximately 0.3 mm towards the equator of the eye, and varying also by individual and in some individuals, portions of the sclera can be less than 0.3 mm depth.

In several embodiments of the methods herein described, the contact time is set to be between approximately 0.01-10 times a diffusion time of the photosensitizing compound, the diffusion time is a ratio of the square of the thickness of the target region divided by the diffusion coefficient of the photosensitizing compound in the target tissue and the contact time and delay time are jointly set such that the sum of the contact time and the delay time is between approximately 0.01-10 times the diffusion time of the photosensitizing compound. Additionally the set quantity of photosensitizing compound is capable of extinguishing the irradiating light by between approximately 10-99%; and the contact time.

The amount of a photosensitizing compound transferred from a formulation into a target tissue is determined using the diffusion coefficient and the partition coefficient of the compound as well as the concentration of the photosensitizing compound in the formulation and the contact time of the formulation with the target tissue (e.g. see Example 33,).

Partition coefficient and diffusion coefficient of a compound in a target tissue can be determined by a number of methods (e.g. see Examples 30-36). For example, a photosensitizing compound can be delivered to an eye and the cornea, sclera, or other target tissue and the tissue can then be isolated to determine a number of photo sensitizer molecules delivered to the tissue as a function of contact time with the drug solution (e.g. see Examples 32-34, Eq.'s 28-33).

More particularly, a determination of a number of photosensitizer molecules delivered to the tissue can be determined by dissecting the eye to obtain a desired cross section and measure an absorbance of the tissue (see e.g. Example 33, Eq.'s 27-29) to determine a number of drug molecule delivered per unit area.

A diffusion model can them be used to determine a partition coefficient, k and diffusion coefficient, D based on the number of drug molecules delivered to the tissue, for example, as shown in Examples 32-34 (e.g. see Eq.'s 28-33).

In some embodiments the fitting of the diffusion model can be performed using for example, a calculator or a computer adapted to perform mathematical operations (e.g. a computer comprising MATLAB® software).

In some embodiments, a partition coefficient and diffusion coefficient of particular photosensitizer are known values identifiable by a skilled person.

Using the partition coefficient and diffusion coefficient of the photosensitizing compound in a target tissue a concentration profile as a function of tissue depth can be generated. (see e.g. Examples 32-34).

In particular, according to some embodiments a concentration profile can be generated given a set concentration of the photosensitizing compound, a profile can be generated based on the observation that a longer contact and delay time can be used to increase an amount of the photosensitizing compound in the target tissue, which can be due to a longer diffusion time on the surface (contact time) and/or in depth (delay time). In particular, a profile can be generated based on an equation that is suitable to calculate diffusion for a certain compound. In particular, in some embodiments, Fick's diffusion equation can be used to calculate a concentration of the photosensitizing compound that will diffuse from the topically applied composition into the target tissue over time, see for example, Example 35 (See Eq.'s 42-47.2).

With particular reference to eosin Y, for a desired concentration of 0.016% in the tissue, using the method exemplified in Example 35, concentration of eosin Y in the composition and contact times capable of achieving the 0.016% concentration in the tissue included, for example, 0.027% concentration in the composition with 1 minute contact time, 0.012% concentration in the composition with 5 minutes contact time, or 0.0088% concentration in the composition for 10 minutes.

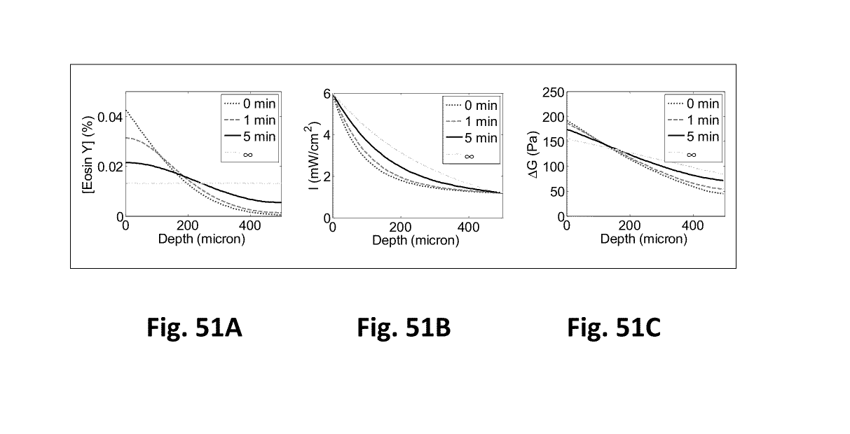

In several embodiments, concentration and distribution of a compound in a tissue is determined by a combination of quantity of compound applied, contact time, and delay time determined based on a concentration profile as will be understood by a skilled person in view of the present disclosure. In particular, for a set quantity applied, concentration in the target region can be determined by controlling the contact time and delay time to achieve a desired final concentration of the tissue. For example, a longer delay time can be used to control a depth up to which the photosensitizing compound penetrates a tissue, while increasing contact time will provide an increased distribution along the surface of the target region. A longer delay time can lead to a deeper penetration of the photosensitizing compound into the target tissue compared to shorter contact time which can lead to a more shallow penetration, as will be understood by a skilled person. Also; for a short contact time, increasing the delay time can result in a more uniform distribution of the photosensitizing compound in the target tissue to give a more uniform concentration profile (see e.g. FIG. 51a) as will be understood by a skilled person. Specific combinations of contact time and delay time for a set quantity of compounds applied can be identified by a skilled person. In some embodiments, the contact time is set to be between approximately 0.01-10 times a diffusion time of the photosensitizing compound. In some embodiments, the contact time and delay time are jointly set such that the sum of the contact time and the delay time is between approximately 0.01-10 times the diffusion time of the photosensitizing compound.

In several embodiments, given a photosensitizing compound concentration and distribution in a tissue, an irradiation intensity and duration can control a quantity of cross-linking as will be understood by a skilled person in view of the present disclosure.

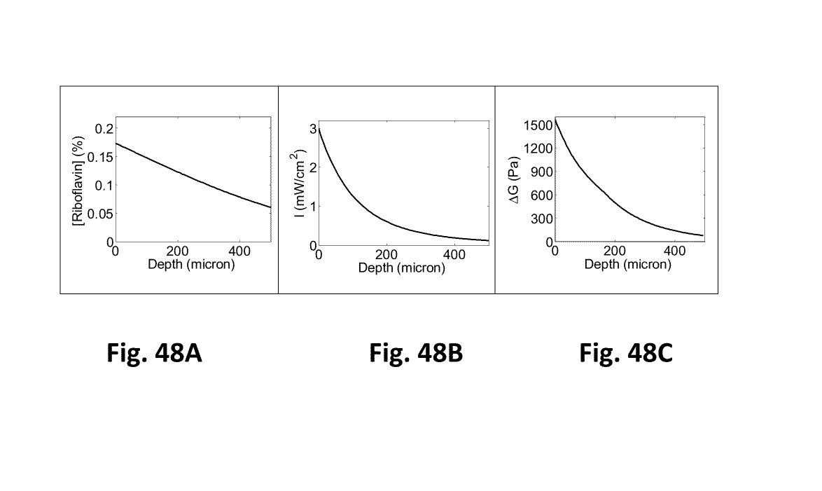

In particular for a given drug concentration profile, a corresponding light intensity profile can be generated for various photosensitizer concentrations in order to determine the light intensity delivered to the target region for the photosensitizer concentrations that are functional to a desired cross-linking effect (e.g. see Example 35, eq. 48, and FIGS. 48A-C, 49-A-C, 50 A-C, 51-A-C, 52-A-C, 53 A-C, 54 A-C).

In particular a light intensity profile can be generated by indicating for a set concentration, the light intensity detected within a target region at various depths. In general, light intensity profiles show that light intensity decreases within a target region at various depths in view of an extinguishing effect due to concentration of the compound and the optical properties of the particular tissue. (e.g. see Example 35 and FIGS. 48B, 49B, 50B, 51B, 52B, 53B, 54B)

In order to select a desired combination of concentration and light intensity a profile of modulus increase (modulus corresponding to an extent of cross-linking) for a set of photosensitizer concentrations and light intensity can be obtained in order to select a desired concentration and light intensity to obtain a desired cross-linking effect (see e.g. Example 35).

The term “modulus” as used herein refers to a constant or coefficient that represents, for example numerically, the degree to which a substance or body possesses a mechanical property. Such mechanical properties include but are not limited to strength and elasticity. Ranges of modulus can depend on the exact method of measurement, the specific type of modulus being measured, the material being measured, and in the case of the sclera, the condition of the tissue (e.g. due to age or health) and the tissue's location on the ocular globe. Examples of moduli include Young's modulus (also known as the Young modulus, modulus of elasticity, elastic modulus or tensile modulus), the bulk modulus (K) and the shear modulus (G, or sometimes S or /−t) also referred to as the modulus of rigidity.

Photorheology can be used to measure a rate of change of storage and loss moduli. In some embodiments, photorheology can be used to measure modulus (e.g. see Examples 26, 29-31, 35). More particularly, in some embodiments, photorheology is used to make in-situ measurements of a sample's modulus during irradiation.

Thus a cross-linking profile can be generated by plotting the modulus determined for a certain tissue in function of the depth where the modulus is determined (e.g. see Example 35 and FIGS. 48C, 49C, 50C, 51C, 52C, 53C, 54C). In particular for each depth a set concentration and light intensity can be associated based on the concentration profile and light intensity profile

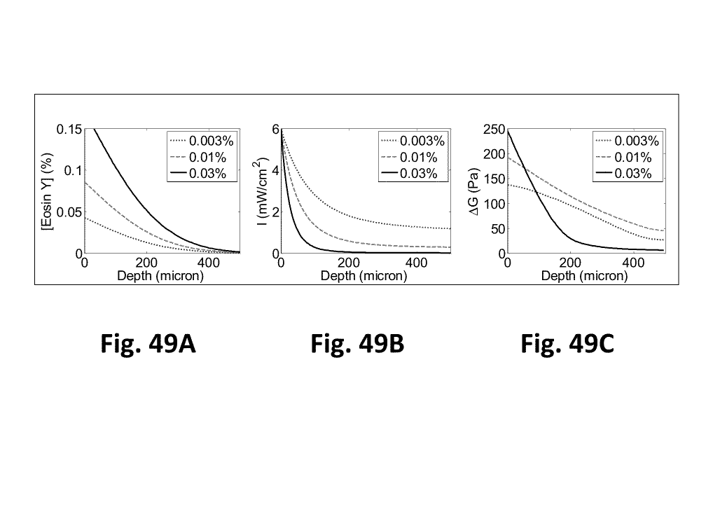

In particular, cross-linking profiles generally show that increasing a concentration of photosensitizer can increase an extent of cross-linking given a fixed set of irradiation conditions (e.g. irradiation time and intensity), however, at some point a concentration of photosensitizer will decrease light penetration in the tissue as a result of light absorbance by the photosensitizer, thus leading to a decrease in an extent of cross-linking. For example, as shown in Example 35 (see e.g. FIG. 49C), increasing the concentration of eosin Y from 0.003% to 0.01% eosin Y, increases the extent of cross-linking everywhere in the tissue, however increasing the concentration from 0.01% to 0.03% can decrease light penetration which in turn decreases an extent of cross-linking.

In particular, a cross-linking profile allows for a determination of an instantaneous local cross-linking rate for each measured depth which is associated with a specific concentration and light intensity as would be understood by a skilled person.

In order to evaluate the instantaneous local cross-linking rate, a tissue can be divided in thin sections along a visual axis so that each section has an approximately uniform concentration and intensity profile. Within each section, an instantaneous cross-linking rate can be obtained from collagen gel photorhelogy data (rate of change in storage modulus) of collagen samples with uniform concentration profiles and approximately uniform light intensity profiles. The local change in storage modulus after a given irradiation time is the sum of the instantaneous changes in modulus at each time step. The instantaneous local cross-linking rate can be quantified by a rate of change in modulus, Ġ′ obtained, for example, from collagen gel photorheology (e.g. see Example 35 and eq.'s 49-50).

Accordingly, for a determined depth a quantified cross linking rate can be determined which is associated to a specific compound concentration and light intensity value.

Accordingly in some embodiments, various steps and conditions of the method for performing photodynamic cross linking can be determined based on the calculated instantaneous local cross-linking rates.

For example, if a certain cross linking effect is desired, an instantaneous local cross-linking rate can be identified that corresponds to the desired cross linking effect and the corresponding concentration and light intensity determined based on the modulus profile. Parameters such as contact time, delay time and amount of compound applied to the target region can then be determined in function of the desired concentration in the target region. Similarly, the irradiation intensity and time can be determined in function of the desired light intensity in the target region.

Variation of those parameters based on additional desired constraints, can be determined by a skilled person by adjusting any of the parameters based on the concentration profile, light intensity profile and modulus profile. For example, if for a certain cross linking effect corresponding to an instantaneous local cross-linking rate, a lower irradiation duration is desired, concentration in the tissue can be increased to give an increase in an extent of cross-linking up to the point at which the photosensitizer decreases light penetration in the tissue as a result of light absorbance by the photosensitizer. On the other hand, if a lower concentration of the photosensitizer is desired, then the irradiation duration can be increased to increase an extent of cross-linking (see e.g Example 35).

Concentrations in the tissue can be controlled by controlling contact time, delay time and amount of compound applied to the tissue. In particular, once a desired concentration in the tissue has been determined based on the quantified instantaneous local crosslinking rate, corresponding contact time and delay time can be determined based on Fick's equation or other equations to determine diffusion of a compound over time, for a set amount of compound applied. In other embodiments, based on a set contact and delay time an amount of compound to be applied to obtain the desired concentration in the tissue can be determined using the same equations (see e.g Example 35).

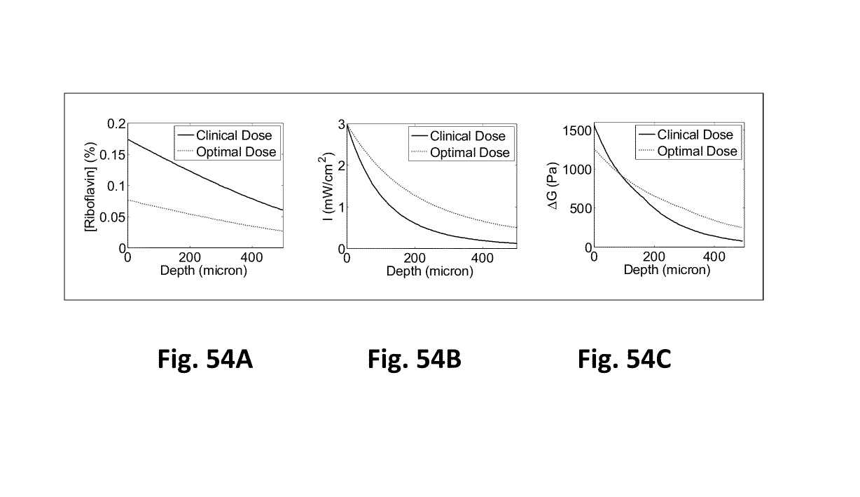

In some embodiments, irradiation time and irradiation intensity can be independently adjusted based on a desired extent of cross-linking for a set concentration of compound in the tissue. For example, for a given concentration profile and irradiation intensity, an extent of cross-linking increases proportionally with irradiation time (see e.g. FIG. 54A-C and Example 35). Thus an irradiation time can be selected in accordance with a desired extent of cross-linking, with longer irradiation times being associated with a greater extent of cross-linking. A lower irradiation intensity with longer duration can result in more cross-linking than a high intensity and shorter duration.

In the method, contact time, the delay time, the quantity of photosensitizing compound to be applied and the irradiating are controllable to vary an effect of the photodynamic crosslinking and can be selected in view of the specific effect of photodynamic crosslinking that is desired for a particular applications.

Effects of photodynamic crosslinking that can be obtained according to several embodiments of methods herein described include, for example, an extent of crosslinking, a tissue depth up to which crosslinking takes place, a uniformity of crosslinking across a surface of particular tissue and/or uniformity through a cross-section of the tissue, a minimizing of cross-linking in a non-target tissue, and/or a minimizing of side effects associated with performing a photodynamic cross-linking (e.g. side effects associated with an exposing of an ocular tissue to the photosensitizing compound and/or associated with an irradiating of an ocular tissue). In several embodiments, in particular the effects of photodynamic crosslinking can be controlled by appropriate selection of the contact time, delay time, and quantity of photosensitizing compound as will be understood by a skilled person.

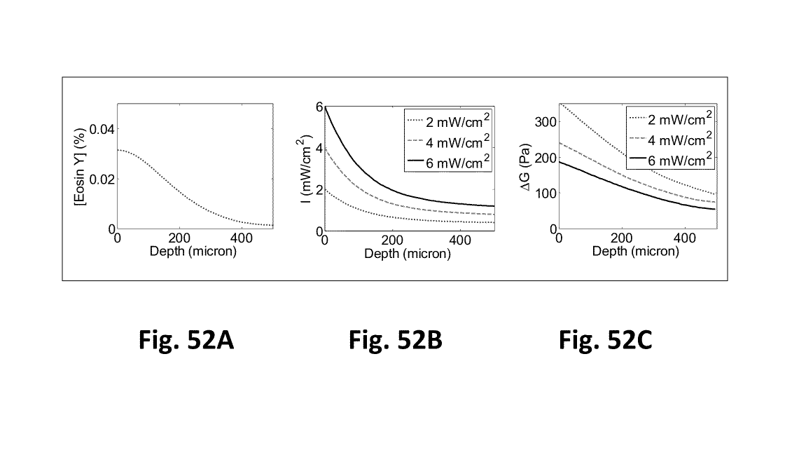

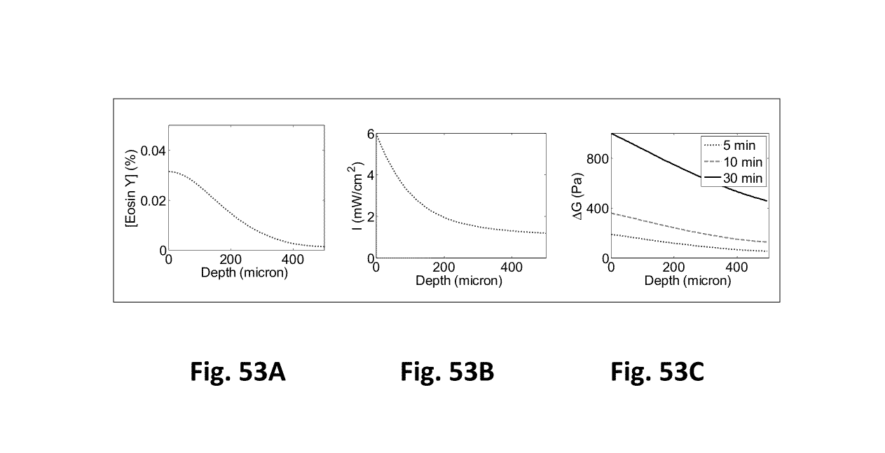

Depending on the extent of cross-linking is desired, a particular irradiation intensity and duration can be selected to obtain a corresponding desired cross-linking effect in the tissue. In particular, in some embodiments, a radiation profile can be used to control a quantity of cross-links in a target tissue. In particular, radiation intensity and irradiation time can be used to control a quantity of cross-links in a target tissue. For example, given a particular light dose to be administered, a combination of a lower intensity radiation and a long irradiation time can lead to a greater extent of cross-linking and an extent of cross-linking can continue to increase, substantially proportionally, with time (see e.g. FIGS. 52A-C, FIGS. 53A-C,). Accordingly, for a given incident light intensity of the light source, an intensity reaching the back of the cornea can depend on a total quantity and distribution of the photosensitizing compound present in the tissue as will be understood by a skilled person.

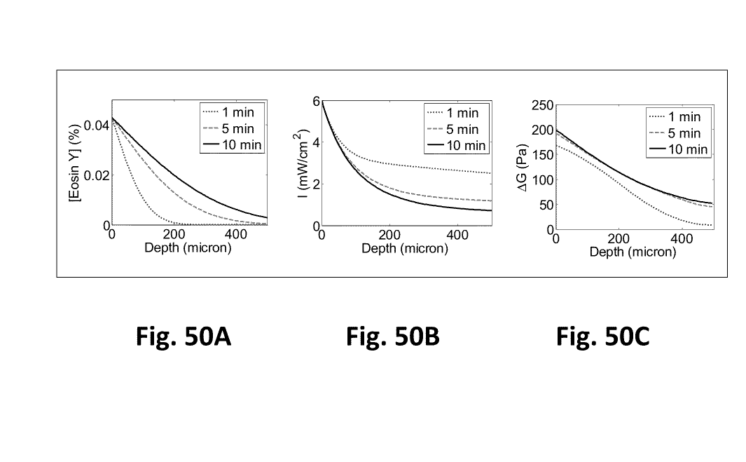

In some embodiments, a compound concentration in a target tissue can be controlled by controlling contact time, delay time and quantity of photosensitizing compound applied to obtain an amount and distribution of a photosensitizing compound throughout a target tissue which provides the desired cross-linking effect in connection with a set irradiation (see e.g. Example 35). As an example made with particular reference to eosin Y as a photosensitizing compound, for a set concentration of eosin Y, increasing the contact time can increase the concentration everywhere in the tissue (provided the contact time is less an amount of time it takes for the photosensitizing molecules to penetrate the entire cornea and can be estimated by L²/(4*D)˜15 minutes for eosin Y in the cornea). In this example the amount Eosin Y in the tissue (see e.g. FIG. 50A), can cause the light intensity to decay more steeply with a longer contact time (FIG. 50B) and for 0.01% eosin Y, light can penetrate the entire thickness of the cornea even with 10 minutes contact time. In this example, increasing the contact time from 1 to 5 minutes, can increase the extent of cross-linking everywhere in the tissue (ΔG′_{avg} increases from 76 to 104 Pa) and increasing the contact time from 5 to 10 minutes, can result in a similar cross-linking profile (FIG. 50C).

In some embodiments, by controlling the distribution of the photosensitizing compound in the target tissue, an intensity profile of light in the eye can be provided that is functional to a desired cross linking effect. In particular, having a more uniform distribution of the photosensitizing compound in the target tissue can provide a light intensity profile having a slower decay in the light intensity as a function of tissue depth compared to a light intensity profile for a tissue having a less uniform distribution of the photosensitive compound. As another example, having higher drug concentration inside the target tissue can provide a light intensity profile having a faster decay, in the light intensity as a function of tissue depth, compared to a light intensity profile for a tissue having a lower concentration of the photosensitive compound.

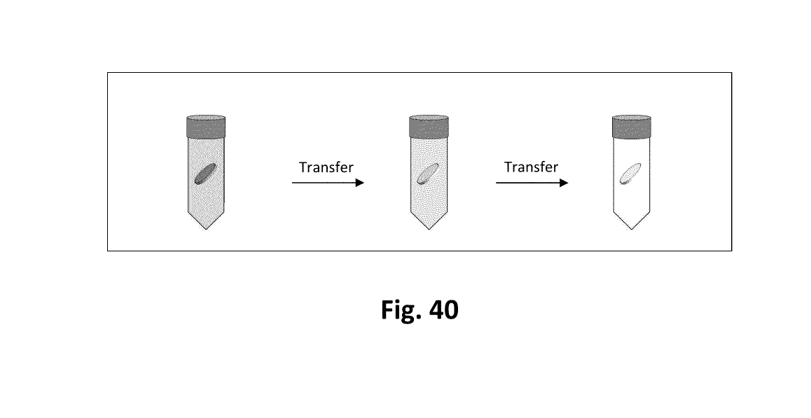

In some embodiments, given an irradiation intensity and duration, a concentration of the photosensitizing compound can be used to control a quantity of cross-links to be formed in a target tissue by controlling of a quantity of the photosensitizing compound inside a target tissue. For example, for a particular set contact time, increasing the concentration of the photosensitizing compound can proportionately increase a concentration of the photosensitizing compound inside a target tissue with which the compound is contacted (see e.g. FIG. 49A) and irradiation of the target tissue having an increased concentration of the photosensitizing compound can lead to a light intensity which decays more steeply (see e.g. FIG. 49B). Therefore, an increase in the concentration can lead to an increased extent of cross-linking and can also lead to a decrease in penetration depth of the light into the target tissue. As an example and with particular reference to eosin Y as a photosensitizing compound, for a set contact time, increasing a concentration from 0.003% to 0.01% eosin Y can lead to an increasing extent of cross-linking in a target tissue (ΔG′_{avg}, see e.g. FIG. 40C). Further increasing the concentration of eosin Y from 0.01% to 0.03% can decrease the light penetration depth from 146 μm to 38 μm (depth at which the intensity is 1/e of the incident intensity), resulting in most of the tissue with very little light for activating the reaction in the posterior side of the tissue (ΔG′_{avg} decreased from 104 Pa to 55 Pa). At 0.03% concentration, 75% of the cross-links form in the anterior 135 μm, compared to 290 μm for 0.01% concentration (see e.g. Example 35).

In some embodiments it is desirable to deliver a quantity of the photosensitizing compound which leads to a light penetration depth approximately equal to the thickness of the tissue to be treated.

In some embodiments, the set quantity of photosensitizing compound is capable of extinguishing the irradiating light by between approximately 10-99%.

In some embodiments, the wavelength of the light source is set to be in a range between +/−10% of the wavelength corresponding to a maximum extinction coefficient of the photosensitizing compound.

In some embodiments, the photosensitizing compound has a permeability in a target tissue which is approximately between 50% to 500% greater than a permeability of riboflavin in a target tissue. (see e.g., Example 34)

In some embodiments, the photosensitizing compound has a partition coefficient (k) between a vehicle for topical application and a target tissue, of approximately greater than 2-20 μm²/s. Having a partition coefficient in this range allows transport of the photosensitizing compound in a concentration sufficient for performing a photodynamic cross-linking. Partition coefficients of a photosensitizing compound can be determined, for example, as seen in Examples 32-34.

In some embodiments, the photosensitizing compound in a particular formulation has a partition coefficient (k)_{PhC} between a vehicle of the formulation and the photosensitizing compound which 1.5 times the partition coefficient of riboflavin (k)_{Rf} a same formulation between a same vehicle with respect and a same target tissue (e.g. where (k)_{PhC}/(k)_{Rf} is approximately greater than between 1.5-30). Such a partition coefficient can allow for transport of the photosensitizing compound in a concentration sufficient for performing a photodynamic cross-linking. (see Example 34 and Table 12)

In some embodiments, permeability can be used as a parameter for selecting a compound suitable for cross-linking in an ocular tissue. Permeability of a particular photosensitizing compound in a particular ocular tissue can be determined, for example, as seen in Examples 32-34.

In some embodiments, the desired portion of the eye is the cornea and the photosensitizing compound has a corneal diffusion coefficient of approximately 40-84 μm²/s. A permeability of greater than approximately 84 μm²/s can allow the photosensitizing compound to permeate the entire thickness of a cornea within less than approximately 26 min.

In some embodiments, the target portion of the eye is the sclera and the photosensitizing compound has a scleral diffusion coefficient of approximately 4-8 μm²/s. A permeability of greater than approximately 8 μm²/s can allow the photosensitizing compound to permeate the entire thickness of a sclera within less than approximately 44 min.

In some embodiments, the target portion of the eye is the limbus and the photosensitizing compound has a limbal diffusion coefficient of approximately 4-84 μm²/s.

In some embodiments, the photosensitizing compound has a phototoxicity which is approximately less than half of a phototoxicity of riboflavin under a set of conditions that provide greater than approximately 80% of the therapeutic crosslinking of riboflavin.

In some embodiments, the photosensitizing compound is eosin Y. Eosin Y has been approved by the FDA for use in the body of a lung and dural sealant (FOCALSEAL™)^{[49, 50]} due to its low toxicity and thus is suitable for use in a medical treatment. More particularly, eosin Y can be suitable for a photodynamic protein-protein cross-linking based on its ability to generate reactive oxygen species (e.g. singlet oxygen). Eosin Y binds to an extracellular matrix of a cell and such binding can substantially prevent eosin Y from entering the cell which can at least, in part, contribute to the non-cytotoxic nature of eosin Y.

In particular, in some embodiments, the photosensitizing compound is eosin Y and the method comprises topically applying a pharmaceutical composition in the form of a gel comprising eosin Y in a concentration ranging between 0.002-8% or 0.03-4.5 mM, and more particularly between 0.03-0.05% or 0.6 mM±5%, and a viscosity enhancer in a concentration ranging between approximately 0-20% and allowing a contact time of the gel with a cornea to be treated, ranging between approximately 10 seconds and 30 minutes. The method further comprises removing excess gel following the contact time. The method further comprises irradiating the cornea with a light source following a delay time between removal of the excess gel from the cornea and before beginning irradiation, the delay time ranging between approximately 0-15 minutes.

In some embodiments of the method, the contact time and delay time are selected to give a combined contact and delay time of approximately 10 seconds and 30 minutes and in particular, approximately 10 minutes. A combined contact time and delay of approximately 10 seconds and 30 minutes can be sufficient to produce a relatively uniform distribution of eosin Y inside the cornea.

In some embodiments, the irradiation of the cornea treated with eosin Y is performed using visible light. In particular, in some embodiments, the visible light is green light, green light having a peak at 514 nm, using green LEDs having a wavelength of approximately 525 nm (see e.g. Example 2).

In some embodiments, the irradiating of the cornea treated with eosin Y is performed using a light dose of approximately 1-10 J/cm² and in particular, in some embodiments, a light dose of approximately 4.2 J/cm² (see e.g. Examples 35, 36).

In some embodiments, the irradiating of the cornea treated with eosin Y is performed using a light dose of approximately 1-10 J/cm². Such a light dose can be achieved with various combinations of light intensities and irradiations times. In some embodiments, the irradiation time ranges from approximately 2-20 minutes and the light intensity ranges from approximately 1-20 mW/cm².

In some embodiments, the combination of light intensity and irradiation times are selected so as not to exceed a selected light dosage. Therefore, longer irradiation times are paired with lower intensity light and shorter irradiation times are paired with higher intensity light. Selection of a particular combination of irradiation time and irradiation power can be selected based on a desired extent of cross-linking. For example, a lower intensity radiation and a long irradiation time can lead to a greater extent of cross-linking and a higher intensity radiation with shorter irradiation time provides an overall shorter treatment duration. Both treatment duration and light intensity can be a consideration of a patient's comfort level. Therefore, an extent of cross-linking as well as a patient's comfort level can be used to select a particular combination of a light intensity.

Other photosensitizing compounds which can be used include, for example, riboflavin and additional compounds identifiable by a skilled person. Riboflavin is a UVA light activated photosensitizer (370 nm ultraviolet irradiation). Eosin Y is a visible light activated photosensitizer having a maximum absorption peak at approximately 514 nm (green light). Additional compounds suitable to be used in methods, systems and compositions herein described are identifiable by a skilled person. (see e.g. Example 24)

In some embodiments, crosslinking can be performed to obtain a “therapeutic cross-linking” in which a disease is treated by triggering protein-protein cross-links. Typically, therapeutic cross-links can be inserted in a controlled manner, both spatially and temporally, for treatment or preventive purposes that range from killing tumors to stabilizing the shape of the eye.

In particular, a desired effect of photodynamic crosslinking can be selected in connection with treatment and or prevention of a particular type ocular condition to be treated, a stage of the ocular condition, and/or a progression of the ocular condition, among other factors identifiable by a skilled person. For example, for a weaker ocular tissue, a greater extent of crosslinking can be desirable.

The term “treatment” as used herein indicates any activity that is part of a medical care for, or deals with, a condition, medically or surgically.

The term “prevention” as used herein indicates any activity which reduces the burden of mortality or morbidity from a condition in an individual. This takes place at primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “condition” as used herein indicates a physical status of the body of an individual (as a whole or as one or more of its parts), that does not conform to a standard physical status associated with a state of complete physical, mental and social well-being for the individual. Conditions herein described include but are not limited to disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

The term “individual” as used herein in the context of treatment includes a single biological organism, including but not limited to, animals and in particular higher animals and in particular vertebrates such as mammals and in particular human beings.

In some embodiments, the ocular condition to be treated comprises ocular diseases which can cause a change in shape of one or more ocular tissues, including but not limited to the cornea and the sclera. By way of example, changes in shape of the cornea can occur as a result of keratoconus, myopic staphyloma, glaucoma, post-LASIK ectasia, and/or other corneal ectasias, and changes in shape of the sclera can occur as a result of degenerative myopia or myopic staphyloma. In particular, Diseases associated with changes in the shape of the sclera (e.g., degenerative myopia), or cornea (e.g., keratoconus and post-LASIK ectasia) can lead to loss of visual acuity due to distortion of the retina or of the refractive surface that is responsible for most of the lens power of the eye, respectively

The term “keratoconus” as used herein refers to an ocular condition in which the cornea develops a cone-like shape from thinning and/or bulging of the cornea. The cone shape can cause irregular refraction of light as it enters the eye on its way to the light-sensitive retina, which can result in distorted vision. Keratoconus is a progressive disease and can occur in one or both of the eyes.

In several embodiments treatment or prevention of an ocular condition can be performed by administering to an individual a photosensitizing compound, the administering comprising applying the photosensitizing compound to a target ocular region for a time and under a condition to allow a suitable concentration of the photosensitizing compound throughout the target ocular region; directing a light source at the target ocular region for a time and under condition to allow a desired extent of cross-linking of a protein to occur in the ocular tissue. In the method the compound: has a partition coefficient (k) in the target ocular region ranging from approximately 2 to 20; has a product of the partition coefficient and a diffusion coefficient (kD) (see Example 34 and Table 12) in the target ocular ranging from approximately 40 to 400 um²/sec; and is capable of generating singlet oxygen upon exposure to a light source of a suitable wavelength.

In particular, in some embodiments, where cross linking is performed to obtain a desired therapeutic effect, contact time and delay time and amount of compounds applied define an administering time and condition that can be used to achieve a concentration in the tissue associated to a desired cross-linking effect and related instantaneous local cross linking rate for certain light intensities. Analogously, irradiation time and intensity define the time and conditions for irradiating the target region by directing a light source towards the target region according to methods and systems herein described, to achieve a light intensity in the tissue associated to a desired cross-linking effect and related instantaneous local cross linking rate for certain concentrations.

A desired effect of photodynamic crosslinking can be selected in connection with a particular type of ocular disease to be treated, a stage of the ocular disease, and/or a progression of the ocular disease, among other factors identifiable by a skilled person. For example, for a weaker the ocular tissue, a greater extent of crosslinking can be desirable.

In some embodiments, irradiating an eye in combination with use of eosin Y or other photosensitizer, can be performed in accordance a method for performing a photodynamic cross-linking treatments using visible light which result in a safer treatment than a treatment involving UV light. Therefore, a photodynamic cross-linking treatment using visible light can allow treatment parameters to be set based on efficacy of a treatment (see e.g. Example 36).

In some embodiments, a compound to be used in connection with treatment or prevention of an ocular condition is a photosensitizer having the following features, has a partition coefficient (k) in a target ocular region ranging from approximately 2 to 20; has a product of the partition coefficient and a diffusion coefficient (kD) (see Example 34 and Table 12) in the target ocular region ranging from approximately 40 to 400 um²/sec, and in particular, 40 to 84 um²/sec in some embodiments (e.g., cornea) and in other embodiments (e.g. sclera) ranging from approximately 4.5 to 7.9 um²/sec; and is capable of generating singlet oxygen upon exposure to a light source of a suitable wavelength.

Pharmaceutical compositions can be identified according to the present disclosure based on the quantified instantaneous local cross-linking rate and related compound concentration and distribution in the tissue.

In particular, a pharmaceutical composition suitable to be used in combination with a light emitting source for performing a photodynamic cross-linking on a target ocular region of an individual, can be identified based on partition coefficient, distribution coefficient and related concentration and intensity light profiles. In particular, in some embodiments a partition coefficient and a diffusion coefficient for a photosensitizing compound in the target ocular region by performing testing on a test tissue thus modifying the tissue. A concentration profile of the photosensitizing compound across the target ocular region can be calculated as a function of time and depth of the ocular region, based on the partition coefficient and the diffusion coefficient of the photosensitizing compound in the target ocular region for one or more set of contact time, delay time and concentration of the photosensitizing compound. A light intensity profile across the target tissue can be calculated as a function of time and tissue depth at a set light dose, based on the concentration profile for the one or more set of contact time, delay time and concentration of the photosensitizing compound. An instantaneous local cross-linking rate can be then quantified based on the concentration profile and the light intensity profile; and selecting a concentration of the photosensitizing compound, a suitable vehicle and the related concentration based on the quantified local cross linking rate, thus providing a pharmaceutical composition comprising the photosensitizing compound and the suitable vehicle.

In particular in some embodiments, instantaneous local cross linking rate can be used to determine concentration of the compound in a composition to be applied to the eye as well as presence of suitable vehicles for delivery conditions and related concentrations in the composition. In particular concentration of the compound and need for inclusion of related vehicles can be calculated in view of a desired concentration in the tissue associated to a desired cross-linking effect. In particular, an instantaneous local cross linking rate for certain light intensities can be determined for the desired cross-linking effect. The corresponding contact and delay time as well as amount of compound to be applied on the tissue can be calculated from the concentration profile based on the desired concentration in the tissue. A corresponding concentration in the composition and need for suitable vehicle can then be determined taking into account partition coefficient and diffusion coefficient.

Various compositions and in particular, pharmaceutical compositions can be identified based on the methods herein described.

In particular in some embodiments a pharmaceutical composition for treatment of an ocular condition, comprising an eosin Y and suitable vehicle is described.

The term “vehicle” as used herein indicates any of various media acting usually as solvents, carriers, binders or diluents for the photosensitizing compound that are comprised in the composition as an active ingredient. In particular, the composition including the photosensitizing compound can be used in one of the methods or systems herein described.

Typical vehicles comprise excipients, diluents and viscosity enhancers. The term “excipient” as used herein indicates an inactive substance used as a carrier for the active ingredients of a medication. Suitable excipients for the pharmaceutical compositions herein described include any substance that enhances the ability of the body of an individual to absorb one or more photosensitizing compounds or combinations thereof. Suitable excipients also include any substance that can be used to bulk up formulations with the peptides or combinations thereof, to allow for convenient and accurate dosage. In addition to their use in the single-dosage quantity, excipients can be used in the manufacturing process to aid in the handling of the peptides or combinations thereof concerned. Depending on the route of administration, and form of medication, different excipients can be used. Exemplary excipients include, but are not limited to, antiadherents, binders, coatings, disintegrants, fillers, flavors (such as sweeteners) and colors, glidants, lubricants, preservatives, sorbents.

The term “diluent” as used herein indicates a diluting agent which is issued to dilute or carry an active ingredient of a composition. Suitable diluents include any substance that can decrease the viscosity of a medicinal preparation.

The term “viscosity enhancer” as used herein refers to a substance capable of increasing a viscosity of a composition. For example, addition of a viscosity enhancer to a composition can give the composition a gel-like behavior. For example, viscosity enhancers can be used in ophthalmic compositions to increase viscosity and thus lead to an increased contact time with the eye when the composition is topically applied to the eye. Examples of viscosity enhancers include but are not limited to natural hydrocolloids (e.g. acacia, tragacanth, alginic acid, carrageenan, locust bean gum, guar gum, gelatin), semisynthetic hydrocolloids (e.g. methylcellulose, sodium carboxymethylcellulose), synthetic hydrocolloids (e.g. CARBOPOL®), and clays (e.g. Bentonite, VEEGUM®).

In some embodiments the suitable vehicle comprises a viscosity enhancer. A viscosity enhancer can provide a pharmaceutical composition with an increased viscosity which can allow the pharmaceutical composition to remain in the eye for a longer period of time thus providing more time for the pharmaceutical composition to undergo absorption. Examples of viscosity enhancers include but are not limited to polymers such as polyvinyl alcohol (PVA), polyvinylpyrrolidone (PVP), methylcellulose (MC), hydroxyethyl cellulose, hydroxypropyl methylcellulose (HPMC), hydroxypropyl cellulose, carboxymethylcellulose (CMC), hyaluronic acid (HA), and sodium alginate (SA).

The pharmaceutical compositions can comprise between approximately 0-20% of a viscosity enhancer. In some embodiments the pharmaceutical composition comprises between approximately 1-6% of a viscosity enhancer. In some embodiments the viscosity enhancer is a carboxymethylcellulose gel. A suitable viscosity enhancer can be selected based on its viscoelastic properties.

An amount of a viscosity enhancer to be used can be selected based on a desired viscosity of the pharmaceutical composition. For example, if a longer contact time of the pharmaceutical composition with the eye is desired then a higher amount of the viscosity enhancer can be used. If a faster contact time of the pharmaceutical composition with the eye is desired then a lower amount (or none) of the viscosity enhancer can be used. For example, if a pharmaceutical composition comprises a higher concentration of a photosensitizing compound then a shorter contact time can be used and thus a lower amount of a viscosity enhancer can be used in the composition compared to a composition with a lower concentration.

Other suitable vehicles for use in the pharmaceutical composition for treatment of an ocular condition comprising an eosin Y include ocular sponges, bandage contact lenses, and other suitable ocular delivery vehicles identifiable by a skilled person.

An amount of eosin Y in the pharmaceutical composition can be selected based on an amount which desired rate of protein-protein cross-linking a desired uniformity of a light profile, and a desired light penetration.

In some embodiments, the pharmaceutical composition comprises eosin Y is between 0.002-8% or 0.03-4.5 mM, and more particularly between approximately 0.03 to 0.05% or 0.6 mM±5%. In concentrations greater than approximately 8% eosin Y, the concentration of drug can be such that light does not fully penetrate a target tissue. In concentrations below approximately 0.001%, a rate of protein-protein cross-linking can be relatively slow and thus would be associated with longer treatment times.

In some embodiments, the pharmaceutical composition can be selected to result in a concentration of eosin Y in a target tissue ranging from approximately 0.002% to approximately 0.4% after application of the composition to the ocular tissue for a set period of time. More particularly, in some embodiments, a concentration of eosin Y in the pharmaceutical composition can be selected to result in a concentration of eosin Y in an ocular tissue of approximately 0.02%±0.01%. In some embodiments the target tissue is corneal tissue or scleral tissue. Various combinations of eosin Y concentration and contact time can be selected to obtain a concentration of eosin Y in a target tissue ranging from approximately 0.002% to approximately 0.4% (see e.g. Example 35, table 2).

In some embodiments, the pharmaceutical composition comprising eosin Y is in an aqueous solution and in particular a buffered saline solution. In some embodiments the composition further comprises deuterium oxide (D₂O) which can increase the crosslinking rate and therefore enhance the treatment effect. Other additives can be used the enhance crosslinking in the compositions described in the present disclosure identifiable by a person skilled in the art.

In some embodiments, compounds other than eosin Y, having similar properties to eosin Y can be used for treating an ocular condition. For example, compounds having similar properties to eosin Y can comprise other photosensitizing compounds which are capable of producing reactive oxygen species; which are capable of binding to an extracellular matrix of a target ocular tissue such as a cornea, sclera, and/or limbus and/or are relatively non-cytotoxic; which have a diffusion coefficient (D) in the target ocular tissue ranging from approximately 40 to 400 um²/sec, and in particular, 40 to 84 um²/sec in some embodiments (e.g., cornea) and in other embodiments (e.g. sclera) ranging from approximately 4.5 to 7.9 um²/sec; and which have a partition coefficient (k) in the target ocular tissue ranging from approximately 2 to 20.

As disclosed herein, the photosensitizing compound, suitable vehicles, related compositions, light emitting arrangements or devices, related support herein described can be provided as a part of systems to perform methods for delivering light to the eye, including any of the methods described herein. The systems can be provided in the form of kits of parts.

In a kit of parts, one or more photosensitizer compounds and other reagents elements and a device to perform the methods herein described can be comprised in the kit independently. The photosensitizer compounds can be included in one or more compositions, and each photosensitizer compound can be in a composition together with a suitable vehicle.

Additional components can include compound delivery devices for delivery of the compound to the target ocular region of interest such as gels, ocular sponges, bandage contact lenses and additional components identifiable by a skilled person.

In some embodiments, various measurements related to radiation directed to a target ocular region can be performed with radiometry devices (see Example 3) or additional devices and techniques identifiable by a skilled person upon reading of the present disclosure.

In particular, the components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials.

Further characteristics of the present disclosure will become more apparent hereinafter from the following detailed disclosure by way of illustration only with reference to an experimental section.

### EXAMPLES

The devices, methods and systems herein disclosed are further illustrated in the following examples, which are provided by way of illustration and are not intended to be limiting.

In particular, the device of FIGS. 4A to 4E and related features, use of the device of FIG. 4A to 4E in providing radiation towards target ocular regions of interest, and combined use with eosin Y as well as criteria for selection of additional photosinthesers and associated concentration profile, light intensity profile, instantaneous local crosslinking rate are exemplified in the following Examples 1 to 37.

A person skilled in the art will appreciate the applicability and the necessary modifications to adapt the features described in detail in the present section, to additional photosensitizer compounds, light arrangements, holders, light delivery systems, method for photodynamic crosslinking and related applications according to embodiments of the present disclosure

### Example 1

### An Exemplary Prototype Light Delivery Device

An exemplary prototype light delivery device has been fabricated and is shown in FIGS. 4A-4E for use with Eosin Y to achieve visible-light cross-linking to treat corneal ectasia. In this example, the Light Delivery Device has a laser alignment that is used to accurately position the device. This alignment procedure uses two 532 nm green lasers that overlap to create a single spot on the cornea surface when at the correct distance. After alignment, the patient is exposed to ˜520 nm light from LEDs for a period of ten minutes (600 seconds). The Light Delivery Device of FIGS. 4A to 4E has an annular array of 24 green, 5-mm diameter, light-emitting diodes (LEDs) to provide uniform irradiation of the cornea.

The LEDs are directed from an oblique direction, which reduces exposure levels at the retina, particularly the central macula. Because of the circular arrangement of the LEDs, the surgeon can also view the irradiated corneal area through the center of the Light Delivery Device, as seen in the illustration in FIG. 4A-4E. Examples 2-23 below provide a risk analysis of the prototype instrument, and show that it is technically a Group 2 instrument in accordance with ISO15004-2 but for any realistic exposure condition does not pose an optical radiation risk to an individual and in particular the patient. The 10 minute irradiation can safely be increased to more than 6 hours, or the intensity could be safely increased 30 times the clinical dose.

### Example 2

### LED Details of a Light Delivery Device

The LED emission wavelength used in the device of Example 1 is: 525 nm±15 nm, chosen to match the peak absorption of the photosensitizer, Eosin Y. The spectral irradiance is shown in FIG. 6A-6E. Note that although the peak wavelength is ˜520 nm, the spectral distribution is not symmetrical, and the central mean wavelength is ˜520 nm. Each LED is a Model RL5-G7532.

In the illustration of FIG. 6A-6E the LEDs are spaced every 15 degrees along the ring with each LED axis directed inward at an angle of 48 degrees from an axis parallel to the optical axis of the eye (e.g. 201) shown in FIG. 6A. The LED annulus has an inner diameter of 37 mm and an outer diameter of 57 mm, and the center-line of the LED positions has a radius of 22 mm around the optical axis. The plane of the LEDs is designed to be 19.2 mm from the corneal plane, and the laser alignment procedure ensures the proper distance from the cornea prior to irradiation.

Prior to each treatment, the light output from the LED array is calibrated at the 19.2-mm distance to the corneal plane to assure the proper irradiation (radiant exposure) dose. Other LEDs having different emission wavelengths can be used and can be selected based on a peak absorption of the photosensitizer to be used and the LEDs can be positioned and calibrated according to a particular application.

### Example 3

### Exemplary Methods to Perform Radiometric Detection

Radiometric measurements of the representative instrument of Example 1 were performed with the maximum setting for light output of each individual optical source and using the following primary instruments: International Light Model 1400A Radiometer/Photometer and Gentech Radiometer, Model Ultra UP Series.

In particular, the International Light Model 1400A Radiometer/Photometer, has three detectors: a. Model SEL240 (#3682) Detector with Input Optic T2ACT3 (#18613) that had been calibrated by the manufacturer on 11 Aug. 2010 to read directly in terms of the ACGIH/ICNIRP UV-Hazard effective irradiance. b. Model SEL033 (#3805) Detector with Input Optic W#6874 and Filter F#14299, which had been calibrated on 11 Aug. 2010 to measure irradiance between 380 and 1000 nm. A 2.2-mm circular mask was used to measure radiance along with this detector. c. Model SEL033 (#3805) Detector (with Input Optic W#6874 and Filter UVA#28246), which had been calibrated on 11 Aug. 2010 to measure near-ultraviolet (UV-A) radiation between approximately 315 and 400 nm.

The Gentech Radiometer, Model Ultra UP Series, with a Solo2 readout and detector XLP12-1SH2-D0, Research was also used as a cross-check of the focal zone irradiance. The detector had a circular entrance aperture of 11.3 mm (i.e., an area of 1.0 cm²), to measure irradiance as well as power for large beam sizes, and a spectral range of 190 nm to 11 μm. The manufacturer calibrated the detector on 6 Feb. 2008 and the instrument remained in calibration.

In particular measurements of photometric values such as flash measurement (photometry), LED, germicidal, UV hazard, plant photobiology, photoresist, UV curing, laser and additional parameters, were performed following manufacturer's instructions of the International Light Model 1400A Radiometer/Photometer and Gentech Radiometer, Model Ultra UP Series. Additional devices can be used to perform those measurements as will be understood by a skilled person.

The general methods for the measurements of the device of Example 1, shown herein Example 3 can also be used for obtaining measurements for other embodiments of the light delivery device.

### Example 4

### Radiometric Detections for a Light Delivery Device

Radiometric measurements were performed on the device of Example 1 at the reference position of the eye in front of the LED array according to the methods outlined in Example 2.

In particular the reference position indicated as normal position was—approximately 19 mm from the plane of the LEDs, i.e., at the corneal-treatment plane. The ring of optical sources was moved laterally in the x and y plane and axially along the z axis to achieve the maximal reading. Measurements are summarized in Table 2.

TABLE 2
Summary of Radiometric Measurements at the Corneal Plane (30 mm from Aperture)

| | Optical Source | | | | |
|---|---|---|---|---|---|
| | Normal | Normal | | | Beam |
| | Position (19-mm | Position (19-mm | | | Irradiance at |
| | distance) | distance) | Extreme (20-mm) | Extreme (20-mm) | 20 cm CIE |
| | Irradiance | Radiant | Irradiance | Radiant | S009 Lamp |
| | averaged over | Power within | averaged over | Power within | Safety |
| | 7-mm | 7-mm | 7-mm | 7 mm | Measurement |
| | Aperture | Aperture | Aperture | Aperture | Condition |
| | (mW · cm⁻²) | (mW) | (mW · cm⁻²) | (mW) | (μW · cm⁻²) |
| Green LED | 5.8 | 2.2 | 6.0 | 2.3 | ~50 |
| Array, 525 nm | | | | | |

The radiometric measurements obtained for the device of Example 1 at the Corneal Plane, can be performed in connection with other embodiments of the light delivery device, light delivering and/or photodynamic crosslinking methods of the present disclosure and related methods and systems. The radiometric measurement can also be obtained and interpreted with reference to other parts of the eye (e.g. sclera, lens, etc.) as will be understood by a skilled person.

### Example 5

### Geometrical Measures of Beam Profiles of a Light Delivery Device

The beam spread of each of the green LED emitters of Example 2 was ˜0.56 radian (FWHM) or ˜32°—sufficient to produce a very uniform irradiance profile at the corneal plane (19 mm) from the 5-mm diameter emitters.

With an emission solid angle of 0.25 steradian (sr) the projected emission surface area effective apparent source size was 2.5 mm. Lateral movement of a 1-mm diameter aperture across the beam at that 19-mm distance showed variations less than 15%. The apparent source size was approximately half the full aperture of each 5-mm diameter LED. The measurements shown here in Example 5 can be used to determine a beam spread of the LEDs or of other light emitting devices suitable for obtaining a desired uniformity of an irradiance profile and can be determined with respect to a corneal target plane or another desired target plane in an eye.

### Example 6

### Ultraviolet Radiation Measurements (UV-A) and Actinic Ultraviolet Radiation Measurements

For all positions optical sources the UV-A irradiance was less than 0.002 μW·cm², which is far below the 1 mW·cm⁻² limit for Group 1 instruments (ISO 15004-2:2007).

For all of the sources, the S(λ)-weighted actinic UV irradiance was undetectable at the noise limit of the instrument, at 0.01 μW·cm⁻². As the limit is 0.1 μW·cm⁻² for an 8-hour exposure (ICNIRP/ACGIH), and much lower than the 0.4 μW·cm⁻² limit for Group 1 instruments (ISO 15004-2:2007), there is a very large safety factor in terms of ultraviolet radiation exposure.

The UV measurements and related safety limits shown here in Example 6 were used to evaluate the safety if the device of Example 1 and can be used as a reference for safety limit for other embodiments of the light delivery device, methods and systems as will be understood by a skilled person.

### Example 7

### ZEMAX® Mathematical Simulation of Radiation Profiles

A model eye has been constructed in ZEMAX®, and the LED arrangement has been simulated using radial sources (FIG. 3) to evaluate radiation profiles of the device of Example 1. Detector planes at the plane of the cornea, on the corneal surface, anterior to the iris, behind the lens, on the retina surface, and in a plane at the posterior retina are used to evaluate the light incident on each component. Furthermore, detector planes at different distances are used to evaluate the homogeneity of the irradiation of the device of Example 1, and variability due to misalignment.

Calculations did not include the absorption effects of Eosin Y, thereby providing conservative estimates of the device safety. A ZEMAX® model along with a simulation of other light emitting elements and/or light emitting arrangement can be performed for various embodiments of the light delivery device to calculate lenticular and retinal irradiances which can be used to in selecting specific features of a light delivery device based, for example, on particular use of the light delivery device. The ZEMAX® model can also be used to calculate corneal irradiance profiles as well as irradiance provided to other target portions of an eye that can be used to vary the device in such a way to obtain a desired effect in the eye as would be understood by a skilled person.

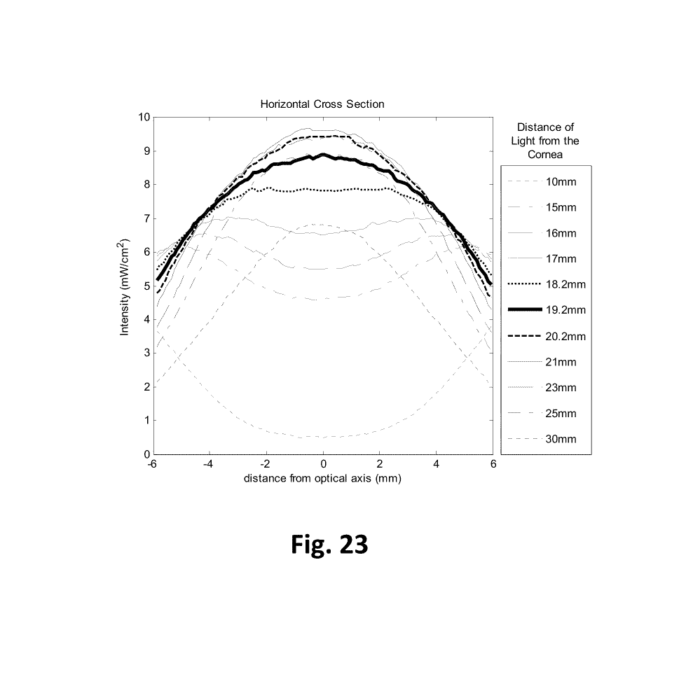

The ZEMAX® simulation provided irradiance values at different planes and provided a measure of the spatial homogeneity. For the distance measurements, a LED radial source power of 1 mW was used for each simulated LED, and the intensity can be considered relative (FIG. 23). It was found that while the LED irradiance patterns overlapped best at 22-23 mm distance from the LEDs, the uniformity was not optimum (i.e., the difference in maximum and minimum was significant) across the cornea and irradiance varied by ˜4.5 mW·cm⁻² and there was a significant central bright spot as shown in FIG. 23. At a distance of ˜16 mm, the irradiance profile at the cornea was fairly uniform, but hot-spots from individual LEDs were evident. Thus, a working distance of 19.2 mm was chosen to ensure a more uniform irradiance pattern. For the light safety simulations, the LED radial source power is adjusted to provide a mean power of 7 mW·cm⁻² on a 0.49 cm² detector located at the corneal plane. Adjusting the simulation to provide the appropriate dose at 19.2 mm shows that the variation across the cornea is ˜3 mW·cm⁻² and if the device distance is misaligned by up to 2 mm, the irradiation (3.7-8.4 mW·cm⁻²) will still provide uniform crosslinking in the cornea (Table 3).

TABLE 3

Irradiance Range Across Cornea Surface

| Distance (mm) | Min (mW · cm−2) | Max (mW · cm−2) |
|---|---|---|
| 17 | 4.7 | 6.1 |
| 18.2 | 4.5 | 6.9 |
| 19.2 | 4.2 | 7.7 |
| 20.2 | 3.9 | 8.2 |
| 21 | 3.7 | 8.4 |

The eye model provided a method to calculate lenticular and retinal irradiances as well as the corneal irradiance profiles, as required for safety calculations in accordance with ISO 15004-2. Irradiance values were calculated at the plane of the corneal surface, anterior to the pupil, posterior to the lens, at the retinal plane, and at a plane posterior to the retina.

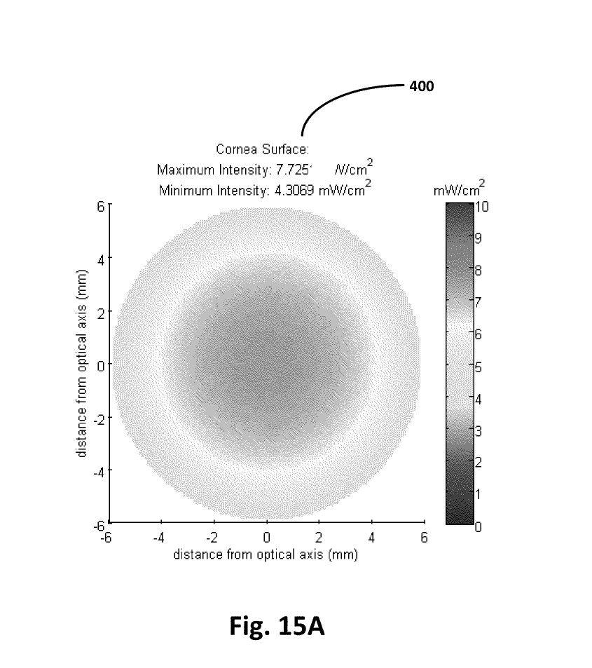

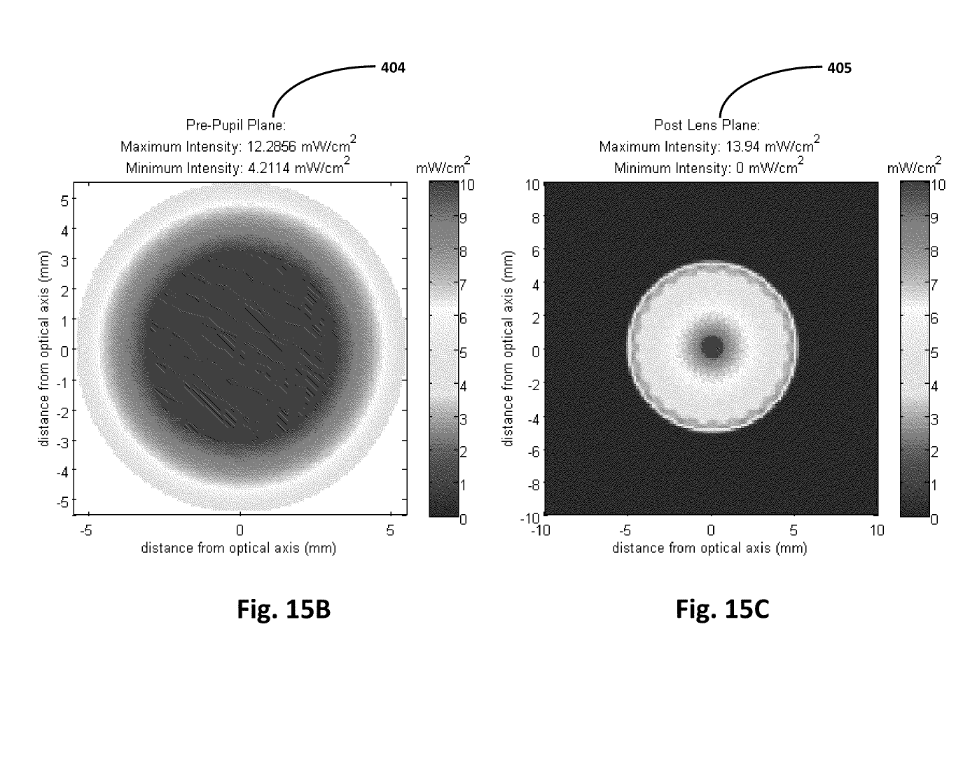

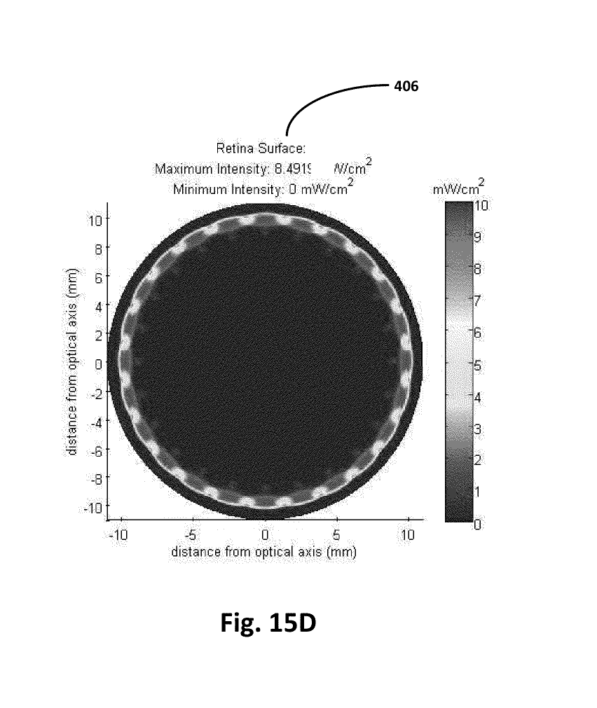

The representation of the irradiance profiles at each plane of interest is illustrated in FIG. 9. There is a uniform cornea irradiance profile (FIG. 15B), and the central macula is devoid of irradiation. The relatively low irradiance falls on the retina ˜12 mm from the center of the macula (FIG. 15D).

The calculated ZEMAX® retinal irradiance profiles of the device of Example 1 are displayed in FIG. 5A-5E. With a maximum exposure to the cornea of 7.7 mW·cm², the maximum retinal irradiance was 8.5 mW·cm⁻², located ˜12 mm from the center of the macula. Thus, for a 600-s treatment duration, the retinal radiant exposure (dose) will be <5.1 J·cm⁻², which will be shown to be safe for this exposure duration and wavelength anticipated for the LED light delivery device. Note that FIG. 9 also demonstrated that exposure of the central macula region of the retina is negligible. Furthermore, the actual light exposure of the retina is further reduced by Eosin Y absorption during the treatment. Eosin Y should reduce the light to ⅓ of the simulated level, thus providing a retinal radiant exposure dose <1.7 J·cm⁻².

The ZEMAX® model as shown in this example can also be used to calculate retinal irradiance profiles of various embodiments of light delivery devices, light arrangements and related methods and systems and can be used to guide a variation the device in such a way to obtain a desired effect in the eye as would be understood by a skilled person.

### Example 8

### Design of a Light Delivery Device Based on Photochemical and Thermal Injury Considerations

The eye is well adapted to protect itself against optical radiation (ultraviolet, visible and infrared radiant energy) from the natural environment and mankind has learned to use protective measures, such as hats and eye-protectors to shield against the harmful effects upon the eye from very intense ultraviolet radiation (UVR) present in sunlight over snow or sand. The eye is also protected against bright light by the natural aversion response to viewing bright light sources. The aversion response normally protects the eye against injury from viewing bright light sources such as the sun, arc lamps and welding arcs, since this aversion limits the duration of exposure to a fraction of a second (about 0.25 s).

There are at least five separate types of hazards to the eye from optical sources: a. Ultraviolet photochemical injury to the cornea (photokeratitis) and lens (cataract) of the eye (180 nm to 400 nm). b. Thermal injury to the retina of the eye (400 nm to 1400 nm). Blue-light photochemical injury to the retina of the eye (principally 400 nm to 550 nm; unless aphakic, 310 to 550 nm).²⁻³ c. Near-infrared thermal hazards to the lens (approximately 800 nm to 3000 nm). d. Thermal injury (burns) of the cornea of the eye (approximately 1400 nm to 1 mm).

For the solid-state-lamp (LED) optical sources used in the device of Example 1 aspect (c) is relevant, since thermal injury requires optical powers in the 100 s′-of-milliwatts-to-watt range. However, in other embodiments of the light delivery device, any one of the other factors can be relevant. Therefore, for the device of Example 1, the photochemical (photoretinopathy) effect was evaluated. In addition, to remove any uncertainty, aspect (a) was measured and confirmed with all sources turned on in order to be assured that there was an absence of ultraviolet radiation. Aspect (d) was also assessed, although this was not considered a realistic concern either in the embodiment of Example 1.

Dosimetric Concepts in Photobiology can also be applied. In particular, the product of the dose-rate and the exposure duration should result in the same exposure dose (in joules-per-square-centimeter at the retina) to produce a threshold injury. For example, blue-light retinal injury (photoretinitis) can result from viewing either an extremely bright light for a short time, or a less bright light for longer exposure periods. This characteristic of photochemical injury mechanisms is termed reciprocity and helps to distinguish these effects from thermal burns, where heat conduction can require a very intense exposure within seconds to cause a retinal coagulation; otherwise, surrounding tissue conducts the heat away from the retinal image. Injury thresholds for acute injury in experimental animals for both corneal and retinal effects have been corroborated for the human eye from accident data. Occupational safety limits for exposure to UVR and bright light are based upon this knowledge. As with any photochemical injury mechanism, one must consider the action spectrum, which describes the relative effectiveness of different wavelengths in causing a photobiological effect. The action spectrum for photochemical retinal injury peaks at approximately 440 nm.

The indications of the present example provide guidance in the design of a light delivery device based on desired effect with respect to photochemical and thermal injury that can result from exposure of an eye to light from a light delivery device.

### Example 9

### Output Characteristics of LEDs of an Exemplary Light Delivery Device

The output characteristics of the LEDs used in the device of Example 1 were compared with known standard to establish potential injury or hazard for the retina. The determination concluded that the output characteristics of the LEDs are far below levels that would pose any potential thermal injury according to the current standard related to retinal hazards.

The principal retinal hazard resulting from viewing bright light sources is photoretinitis, e.g., solar retinitis with an accompanying scotoma, which results from staring at the sun. Solar retinitis was once referred to as “eclipse blindness” and associated “retinal burn.” Only in recent years has it become clear that photoretinitis results from a photochemical injury mechanism following exposure of the retina to shorter wavelengths in the visible spectrum, i.e., violet and blue light. Prior to conclusive animal experiments at that time (Ham, Mueller and Sliney, 1976), it was thought to be a thermal injury mechanism. However, it has been shown conclusively that an intense exposure to short-wavelength light (hereafter referred to as “blue light”) can cause retinal injury.

### Example 10

### Design of a Light Delivery Device Based on Human Exposure Limits

Light delivery devices herein described can be configured according to a design that is functional to set human exposure limits to light.

A number of national and international groups have recommended occupational or public exposure limits (ELs) for optical radiation [i.e., ultraviolet (UV) light, and infrared (IR) radiant energy]. In particular, two principal groups have recommended ELs for visible radiation (i.e., light), and these recommendations are essentially the same. The groups are well known in the field of occupational health—the American Conference of Governmental Hygienists (ACGIH) and radiation protection—the International Commission on Non-Ionizing Radiation Protection (ICNIRP). The ACGIH refers to its ELs as “Threshold Limit Values,” or TLVs and these are issued yearly, so there is an opportunity for a yearly revision. The current ACGIH TLV's for light (400 nm to 760 nm) have been largely unchanged for the last two decades.

The limits are based in large part on ocular injury data from animal studies and from data from human retinal injuries resulting from viewing the sun and welding arcs. The limits also have an underlying assumption that outdoor environmental exposures to visible radiant energy is normally not hazardous to the eye except in very unusual environments such as snow fields and deserts. The ICNIRP publishes Guidelines on limits of exposure to broad-band incoherent optical radiation (0.38 to 3 μm) were published in 1997, and were based upon the ACGIH recommendations to a large extent. The ICNRIP guidelines are developed through collaboration with the World Health Organization (WHO) by jointly publishing criteria documents that provide the scientific database for the exposure limits.

The above-mentioned safety standards, which are described below in more detail, can be used to design light delivery devices in accordance with a desired effect concerning human safety as would be understood by a skilled person.

ICNIRP/ACGIH Limits:

The ACGIH TLV and ICNIRP guidelines are identical for large sources and are designed to protect the human retina against photoretinitis (also referred to as photomaculopathy), “the blue-light hazard” is an effective blue-light radiance L_{B} spectrally weighted against the Blue-Light Hazard action spectrum B(λ) and integrated for t s of 100 J/(cm²·sr), for t<10,000 s, i.e.,

L_{B}·t=ΣL_{λ}·B(λ)·t·Δλ≦100 J/(cm²sr) effective (7)
and for t>10,000 s (2.8 hrs.):

L_{B}≦10 mW/(cm²·sr) for t>10,000 s (8)

To calculate the maximum direct viewing duration when Equation (8) is not satisfied, this maximum “stare time,” t-max, is found by inverting Equation (7) for a CW source with a weighted radiance of L_{B}:

tₘₐₓ=100 J/(cm²·sr)/L_{B} (9)

The radiance values are averaged over a field of view which is not less than 11 mrad=0.011 rad. The blue light hazard is evaluated by mathematically weighting the spectral radiance L_{λ} to obtain L_{B}. Alternatively, the spectral radiant power, Φ_{λ}, against the blue-light hazard function to obtain the fraction of blue light Φ_{B} in the total power entering the eye and then calculate the blue-light retinal irradiance from knowledge of the retinal image size (determined by the cone angle, which is done in this instance). The instrument illuminates far greater areas of the retina than the limiting cone angle applied to consider the spreading of absorbed energy in smaller images by eye movements (0.011 radian) for an unstabilized eye. The individual peak radiance of each LED was ˜0.5 W·cm⁻²·sr⁻¹, which was un-weighted.

The IS0 15004-2:2007 standard uses the aphakic A(λ) spectral weighting function rather than the blue-light hazard B(λ) function. This is largely to deal with operating microscopes where the patient has neither a normal crystalline lens nor an intraocular lens implant briefly during the surgery. When the A(λ) function is used to calculate the effective retinal irradiance, the values increase and any required caution-statement time would decrease slightly. For this spectrum, there is little or no difference between the B(λ) and A(λ) spectral weighting functions.

Product Safety Standards:

In addition to the ACGIH and ICNIRP exposure limits just discussed, other organizations recommend product-safety emission limits. Currently, there are only two sets of different types of product safety standards that apply to the use of lamps—including solid-state lamps (LED's) worldwide. These are:

CIE Standard 5009/E-2002, Photobiological Safety of Lamps and Lamp Systems, which was based upon an earlier edition of the American National Standard, ANSI RP-27.1-2005, Recommended Practice for Photobiological Safety for Lamps and Lamps Systems: General Requirements, published by the Illuminating Engineering Society of North America. These documents are the first in a series of standards, and employ ocular exposure limits that are essentially identical to the guidelines for human exposure published by the International Commission on Non-Ionizing Radiation Protection (ICNIRP), which, in turn, are essentially the same as the Threshold Limit Values (TLVs) for broadband optical radiation published by the American Conference of Governmental Industrial Hygienists (ACGIH). The ACGIH and ICNIRP differ slightly in the UV-A spectral region but not for visible radiation and near infrared. Also, ICNIRP recommends that these incoherent guidelines—and not laser guidelines—be applied to LEDs. One of the IESNA standards included specific guidelines on methods of measurement at realistic viewing distances—not closer than 20 cm—that are not given by the ACGIH, but were adopted by the CIE 5009.

IEC 62471/CIES009-2006, Photobiological Safety of Lamps and Lamp Systems, which is identical to CIE S009/E-2002, but became a joint-logo standard in 2006. It provides guidance to manufacturers on classifying lamps and lamp systems into one of four risk groups, but gives no requirements for labeling, etc. (IEC technical report, IEC TR 62471-2). Photobiological safety of lamps and lamp systems—Part 2: guidance on manufacturing requirements relating to non laser optical radiation was published later in 2009.

ISO 15004-2:2007, Ophthalmic Instruments—Fundamental requirements and test methods—Part 2: Light hazard protection, addresses the photobiological safety of ophthalmic instruments. It provides limits for exposure of the cornea, lens and retina that are based upon ICNIRP guidelines as adjusted for intentional ocular exposure during ophthalmic examination and eye surgery. Special guidance from the ICNIRP on ocular exposure from ophthalmic instruments recognized that the eye might be more stabilized and the pupil could be dilated during ophthalmic examination. Furthermore, an optical beam could be focused or concentrated in the anterior segment and crystalline lens of the eye. This guidance formed part of the basis of the international standard, ISO 15004-2:2007.

Older Product Safety Standards.

In the recent past, there was a period when a laser safety standard issued by the International Electrotechnical Commission, IEC 60825-1:1993 Safety of Laser Products—Part 1: Equipment Classification, Requirements, and Users' Guide, applied to LED products. The inclusion of LEDs by the IEC Technical Committee TC-76 (which developed the standard) in 1993 was largely to treat the specific use of infrared LEDs in optical fiber communication systems. However, it was soon recognized by national and international experts that application of laser limits to incoherent sources was overly conservative, and the IEC TC76 voted to eliminate the inclusion of LEDs in the second edition of IEC 60825-1, which was published in March 2007. Although IEC 60825-1 no longer applies to the LEDs in ophthalmic instruments, IEC 62471:2006 can apply. In the US, the Federal Laser Product Performance Standard (21CFR1040) does not apply to incoherent sources.

The human exposure limits described herein with reference to light delivery devices can be used as considerations regarding safety in designing various embodiments of the light delivery device of the disclosure and/or guidance in using a light delivery device, directed to various treatments in various target portions of an eye.

### Example 11

### Safety Analysis of an Exemplary Light Delivery Device

The values from the previous calculations of corneal and retinal irradiances from the ZEMAX® model simulations (Example 7) can be used to evaluate compliance with ISO 15004-2:2007 (Ophthalmic Instruments—Fundamental Requirements and Test Methods), which is the governing standard for an exemplary light delivery device such as the device of Example 1. In some embodiments of the light delivery device, other standards can be used depending on the particular light source and the particular safety concerns as would be understood by a skilled person.

The absolute maximum irradiance values for the cornea (7.7 mW·cm⁻²) and the retina (9.0 mW·cm⁻²) give a conservative value of the light hazard. The irradiation from the light falls below the limits for a Group 1 device when the pupil is 3 mm in diameter and can be considered an ophthalmic instrument for which no potential light hazard exists. The closest value to the limits is the retinal photochemical aphakic light hazard, which is ˜50% of the maximum permissible exposure (see Table 6 below in Example 19, and sections 5.4.1.3 ISO 15004-2:2007). When the pupil is 7 mm in diameter, retinal irradiation exceeds the limits for a Group 1 device, and it must be treated as a Group 2 device. The irradiation is well below the limits for Group 2—Ophthalmic instruments for which potential light hazard exists (˜2.5% of maximum permissible exposure, see Table 8 below in Example 19, and sections 5.5.1.5 ISO 15004-2:2007). The device can be operated safely as a Group 2 device for up to ˜6 hours. Including Eosin Y in the calculations should decrease the retinal exposure below the levels for a Group 1 device, removing any potential light hazard.

The green-light beam of an exemplary light delivery device such as the device of Example 1 diverges to produce relatively large spots on the retina. The pupil of the patient's eye is the limiting aperture and determines whether all of the energy enters the eye; and since the entire beam does not to enter the eye, the pupil can limit the apparent source size, although at such a close distance the individual LED images are strongly blurred as shown in the ZEMAX® simulation. To test the instrument for comparison with the emission limits to protect the retina, as provided in paragraph 5.4 (Group 1 instruments) in ISO 15004-2:2007, the measurements specified in paragraphs 6.2-6.4 and clarified in Annex C of that standard, provide the simplest method for evaluating the potential retinal hazard by determining the weighted retinal irradiance.

### Example 12

### Example of Determination of Retinal Effective Irradiance

The spectral emission of the green LEDs used in an exemplary light delivery device such as the device of Example 1 are normally spectrally weighted by the spectral weighting factor by the aphakic hazard A(λ) [and B(λ) for the lamp standard] and are the same for the 520-nm band—less than ˜0.04. Hence the spectrally weighted radiance and retinal irradiance calculated values are, in fact, an order of magnitude less than the un-weighted values.

The methods for determining the weighted retinal visible and infrared radiation thermal irradiance, E_{VIR-R}, in accordance with Clause 5.5.1.5 a) in ISO 15004-2, and, weighted retinal radiant exposure, H_{A-R}, in accordance with Clause 5.5.1.6 a) in ISO 15004-2 are similar. For this LED exposure, the retinal irradiance is highly non-uniform, thus the peak calculated irradiance of 9.0 mW·cm⁻² is conservatively applied. The retinal irradiance, in this case may be determined by measuring the sum of the weighted spectral radiant power Φ that enters the eye and determining the area Aᵣₑₜ of retina illuminated, since Eᵣₑₜ=Φ/Aᵣₑₜ. E_{VIR-R} for retinal thermal evaluation is defined by:

$${E}_{VIR - R} = {\sum }_{380}^{{1400}_{2}} {E}_{λ} \cdot R ( λ ) \cdot Δ λ \qquad ( 10 )$$

where,
E_{VIR-R} is the weighted (effective) retinal irradiance for §5.5.2.1 of ISO 15004:2006
E_{λ} is the spectral irradiance
R(λ) is the biological weighting factor (retinal thermal hazard) at wavelength λ for thermal injury to the retina
Δλ is the wavelength summation interval, and the summation is taken over the specified wavelength range from λ₁=380 nm to λ₂=1400 nm.

However, because of the low retinal irradiance it is completely unnecessary to apply the spectral weighting as the limits are far above the calculated retinal irradiances. However, E_{A-R}, the aphakic weighted retinal irradiance is very important, and is given by:

$${E}_{A - R} = {\sum }_{305}^{{700}_{2}} {E}_{λ} \cdot A ( λ ) \cdot Δ λ \qquad ( 11 )$$

where,
E_{A-R} is the weighted (effective) retinal irradiance
E_{λ} is the spectral irradiance
A(λ) is the biological weighting factor at wavelength λ for the photochemical injury to the retina of the aphakic eye as applied in §5.4.1.3, ISO 15004-2. B(λ) for the normal eye is applied in CIE 5009/IEC62471:2006; thus both should be applied.
Δλ is the wavelength summation interval, and the summation is taken over the specified wavelength range from λ₁=305 nm to λ₂=700 nm.

The other wavelength-dependent exposure related quantities, e.g., the weighted radiant exposure, weighted radiance, and weighted integrated radiance all apply similar mathematical expressions with the weighted summation of the spectroradiometric quantity with the biological effectiveness function over the applicable wavelength ranges specified in ISO 15004-2.

The retinal thermal hazard can also be treated in terms of source radiance, which is covered in Clauses 5.4.1.6 b) and 5.5.1.5 b) in ISO 15004-2, where L_{VIR-R} is defined by:

$${L}_{VIR - R} = {\sum }_{380}^{{1400}_{2}} {L}_{λ} \cdot R ( λ ) \cdot Δ λ \qquad ( 12 )$$

And the photochemical retinal hazard is covered in Clauses 5.4.1.3 b) L_{A-R} and 5.5.1.6 b) H_{A-R} in ISO 15004-2 as defined by the equation,

$${tL}_{A - R} = {H}_{A - R} = {\sum }_{305}^{{700}_{2}} {L}_{λ} \cdot t \cdot A ( λ ) \cdot Δ λ \qquad ( 13 )$$

In both cases, the spectral radiance was determined in this approach. The individual peak radiance of each LED was ˜0.5 W·cm⁻²·sr⁻¹, which was un-weighted, and when spectrally weighted, less than ˜0.05 W·cm⁻²·sr⁻¹,

E_{VIR-R} is determined using Equation 10 while E_{A-R} is determined using Equation 11. In both cases, the spectral irradiance would need to be determined for the most accurate determination of retinal effective irradiance, and the spectral weighting factors were no higher than 0.04. An accurate determination of retinal effective irradiance can provide guidance in designing various embodiments of a light delivery device based on a desired level of retinal effective irradiance that is desired.

### Example 13

### Spatially-Average Radiance L of a Light Delivery Device

Spatially-average radiance 1 of the light delivery device of Example 1 was determined for continuous viewing (which would normally not be considered applicable for this instrument), but would be applied for IEC62471/CIE S009:2006 in any case for determining the product's risk group, the B(λ)-weighted radiant power would be used to calculate (or measure) the effective irradiance E_{B-eff} at 200 mm (<5 μW·cm⁻²).

Since for this direct viewing condition, the source size will normally be less that 2.2 mm in diameter, the hazard assessment made at the reference distance of 20 cm will employ an effective cone angle of acceptance of γ=11 mrad (i.e., from a single LED) to determine the spatially-average radiance L. This assessment leads to a product that would be RG-0 (Exempt risk group).

### Example 14

### Control of a Light Delivery Device

Various electronics can be used to prevent the LEDs from being driven at a high irradiance, which will be identifiable by a skilled person. The current can be controlled, and failure in the LEDs can be set to turn off the string of LEDs. The lighting can be controlled by a timer and can be set to turn off automatically after the 10 minute exposure duration. There are stop buttons so the clinician can interrupt the irradiation at any time if necessary.

The peak retinal irradiance levels from the LEDs can give the patient significant after-images, although no reduction in autofluorescence of the retina is expected when used during Eosin Y treatment. Patients can be monitored carefully for up to two days after the exposure to minimize this risk.

### Example 15

### Safety of an Exemplary Device with Reference to Target and Anti-Target Tissues

The exemplary light delivery device of Example 1 was shown to operate at all wavelengths and emission levels that would not produce any ocular injury—even within foreseeable misuse conditions. Requirements for Group 1 instruments were fully met in terms of ultraviolet, infrared and retinal thermal limits, but not met under all conditions for the retinal photo-chemical limits. Because the device does meet the Group 2 criterion, the device can be operated safely, with a warning for maximum exposure duration of ˜6 hours for any one patient in accordance with ISO ISO-15004-2:2007. The 10 minute operation is well below the maximum exposure duration. The instrument is in Exempt Group (RG0, or no realistic risk) with regard to IEC-62471/CIE-S009:2006. Under normal use conditions, individuals should not be at risk. To put this light exposure in perspective, the retinal radiant exposure for this procedure is comparable to viewing bright sunlight reflected from snow for four hours in the middle of the day.

### Example 16

### Example Geometries of an LED Light Emitting Element of a Light Delivery Device

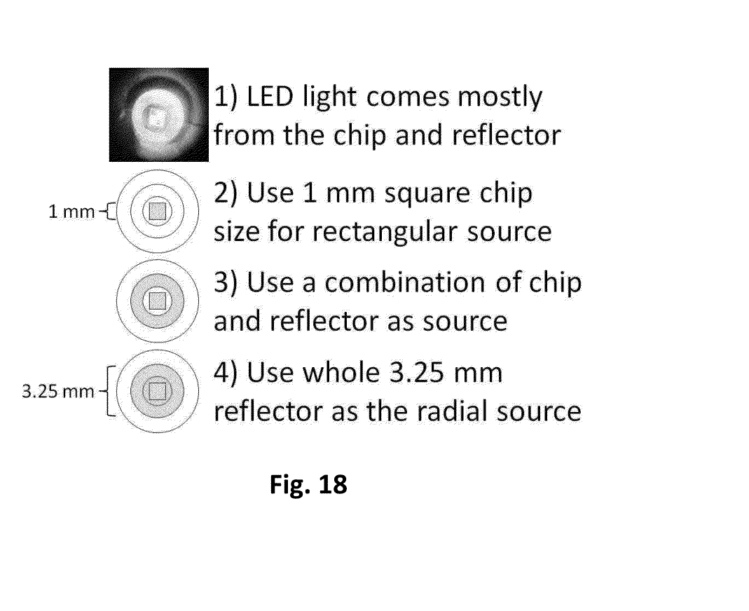

The light source for the simulations was either a 1 mm square chip, a combination of the 1 mm square chip and a 3.25 mm diameter circular reflector, or the whole 3.25 mm diameter reflector. As shown below, the 1 mm square chip produces the brightest retinal spot, and is therefore used for the most conservative estimates of safety. The actual light source more closely resembles a combination of chip and reflector, and it is likely that the retinal exposure more closely resembles the results from that simulation. (see FIG. 18)

### Example 17

### Example of Predicting Light Intensity on a Target Region using a ZEMAX® Setup

The light source for the simulations was either a 1 mm square chip, a combination of the 1 mm square chip and a 3.25 mm diameter circular reflector, or the whole 3.25 mm diameter reflector. As shown below, the 1 mm square chip produces the brightest retinal spot, and is therefore used for the most conservative estimates of safety. The actual light source more closely resembles a combination of chip and reflector, and it is likely that the retinal exposure more closely resembles the results from that simulation.

The ZEMAX® model uses a light source that is modeled after the actual LED. Three different LED source models were used for the calculation of safety: a 1 mm square chip, a combination of the chip and reflector, and a 3.25 mm diameter reflector (see FIG. 18).

To predict light safety of the ring of LED lights, Applicants have created a ZEMAX® model that simulates the transmission of light through a model eye (FIG. 14A). Simulations to measure the effects of distance from the source were all performed using one light power with LED radial source power of 1 mW. Simulations for safety used a calibrated light intensity so that light incident on a circular area of 0.49 mm² at the plane of the cornea had a mean value of 7 mW·cm⁻². Detectors placed in the simulation at the cornea plane, at the cornea surface, in front of the pupil, after the lens, and at the retina provide a comprehensive illustration of how light enters the eye. Detailed detectors placed at the macula, and at the location of the highest incident light on the retina are used for closer analysis.

### Example 18

### Light Exposure of a Target Tissue and Minimizing Light Exposure of an Retinal Anti-Target Region and Variations Base on Spot Size and Shape, Pupil Size, and Distance from LEDs

Light incident on the surface of the cornea is measured using a detector fitted to the corneal surface. Using the ring of LEDs (1 mm square chip simulation) at a distance of 19.2 mm from the cornea, the maximum irradiance is 7.7 mW/cm² (FIG. 23). ISO standards recommend averaging the intensity over a 1 mm circle for safety calculations (white circle). This localized average is 7.6 mW/cm². The black dotted lines in figure are cross sections of the intensity, as illustrated in FIG. 23. The intensity decreases radially, with an intensity of ˜6 mW/cm² at the edge of the treatment (4 mm from the optical axis). A distance of 19.2 mm has been chosen for the treatment in order to reduce the variability due to changes in light source distance, while maintaining uniformity of the corneal irradiation.

The light profile and intensity are dependent on the distance of the LEDs from the cornea (FIG. 23). The light is brightest with a distance of ˜22 mm but, has a central bright spot that is significantly brighter than the edges. The beam profile varies too much from the center to the edge. At a distance of 16 mm, the intensity across the cornea is fairly uniform, but the LEDs created observable bright spots on the surface. To achieve a fairly uniform irradiation without bright spots, a distance of 19.2 mm was selected.

Because of the circular arrangement of the LEDs used in the light source of the exemplary light delivery device of Example 1, and because of the off-axis angles, the light projected onto the retina does not fall on the center of the retina, or the macula. It is also described that the light pattern on the retina with a 7 mm pupil for the ring of 1 mm chips at a distance of 19.2 mm from the cornea. The pattern consists of overlapping LED images and the brightest spot is in the region of overlap (8.5 mW/cm²). The distance of the brightest irradiance from the center of the retina is measured along the retinal surface (12.2 mm from the center).

In order to determine the image spot size, cross sections are taken through the center of the brightest pixel. The cross sections are fit using Gaussian curves, and the full width at half maximum (FWHM) intensity is reported as the critical dimension of the spot. The horizontal and vertical dimensions here are 0.7 mm and 0.5 mm respectively. The fit intensity of 8.4 mW/cm² is very close to the maximum intensity of 8.5 mW/cm². Comparisons using both flat and curved detectors located at the brightest spot were performed to fully characterize the spot.

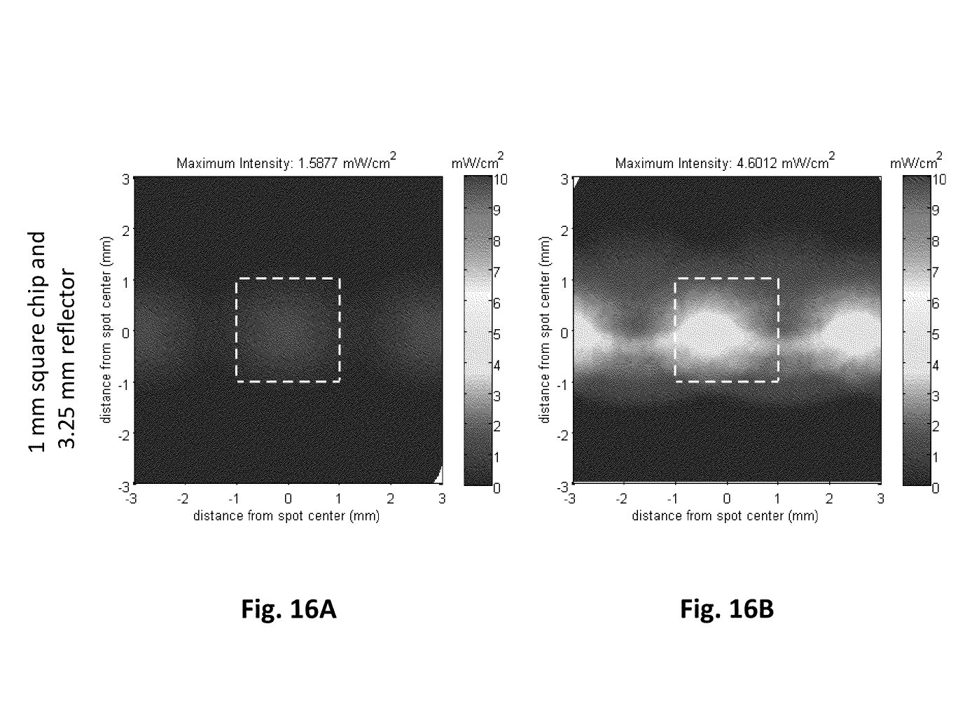

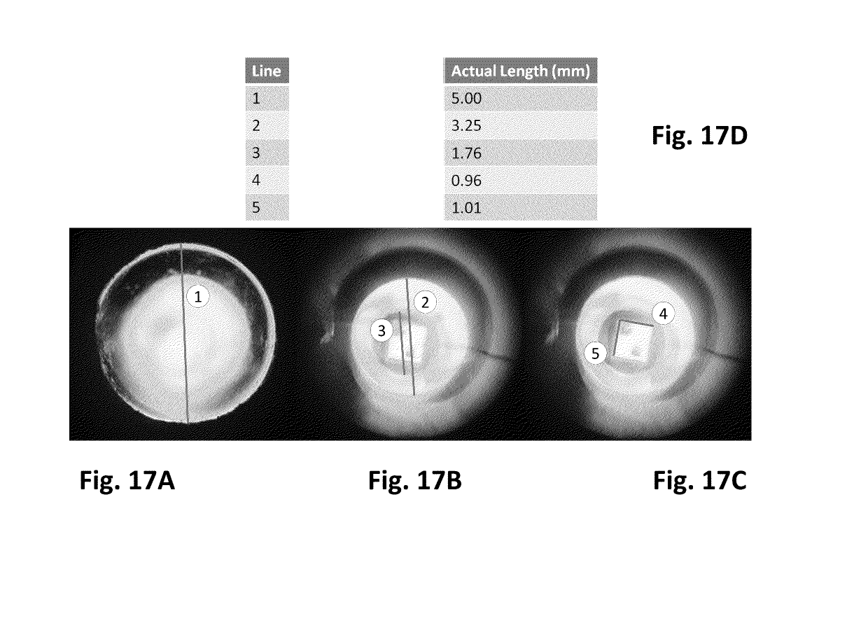

The actual LED sources in the exemplary light delivery device of Example 1 consist of a 1 mm square chip with a reflector and lens (FIG. 18). Running the simulations with just the central chip provides a conservative estimate of the safety, and running the simulations with the reflector included, provides a more accurate estimate. The brightest image spot on the retina is more well defined with just the 1 mm chip, and becomes more spread out with the reflector included (FIGS. 14A-14B; and FIG. 16B). Blurring of the image spot reduces the maximum intensity incident on the retina, and increases the safety.

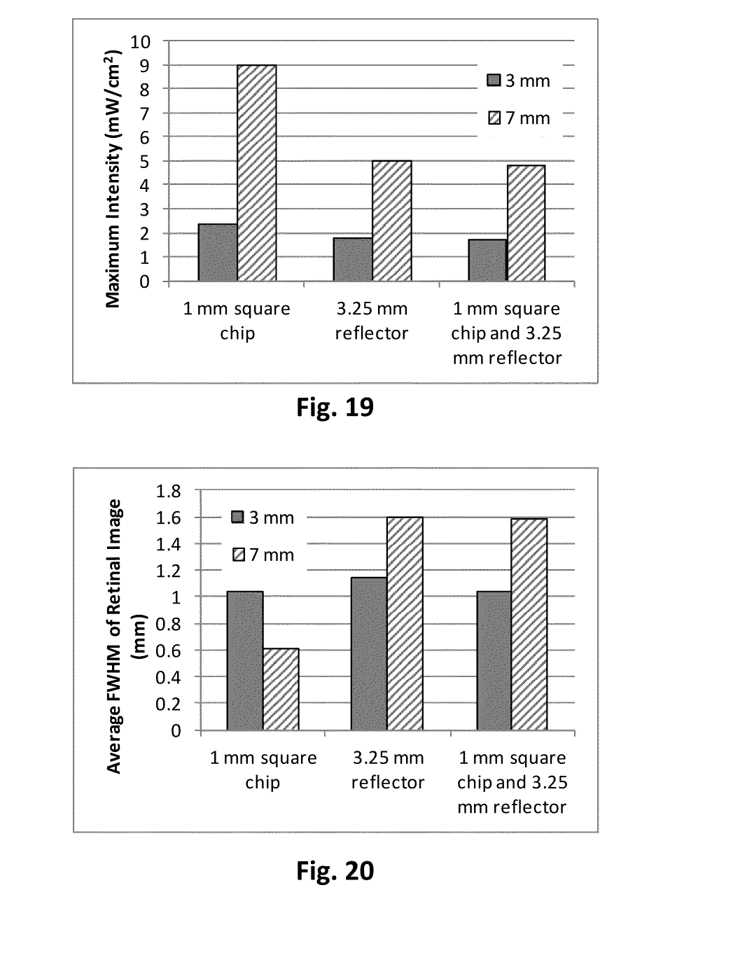

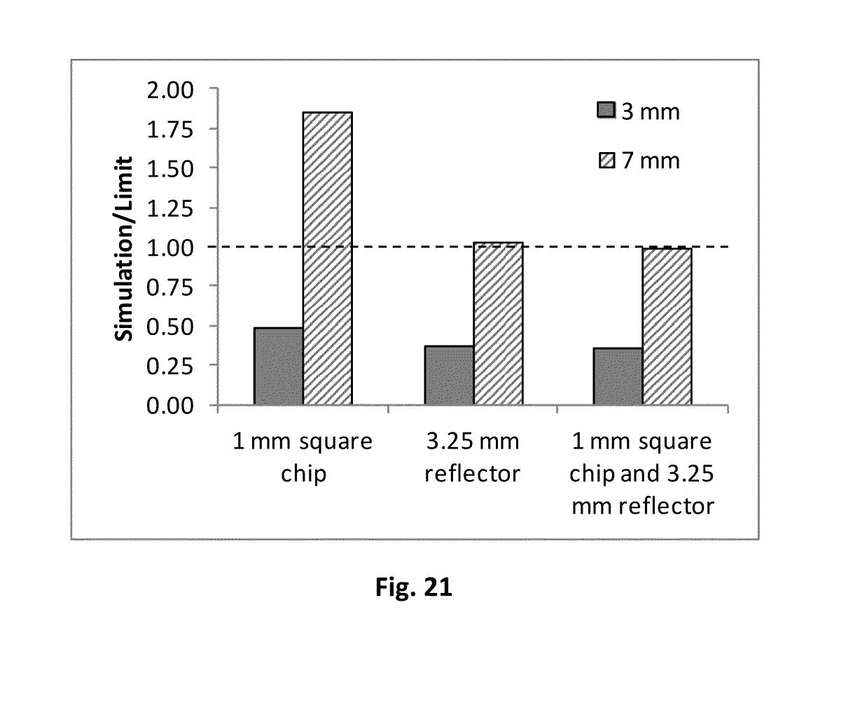

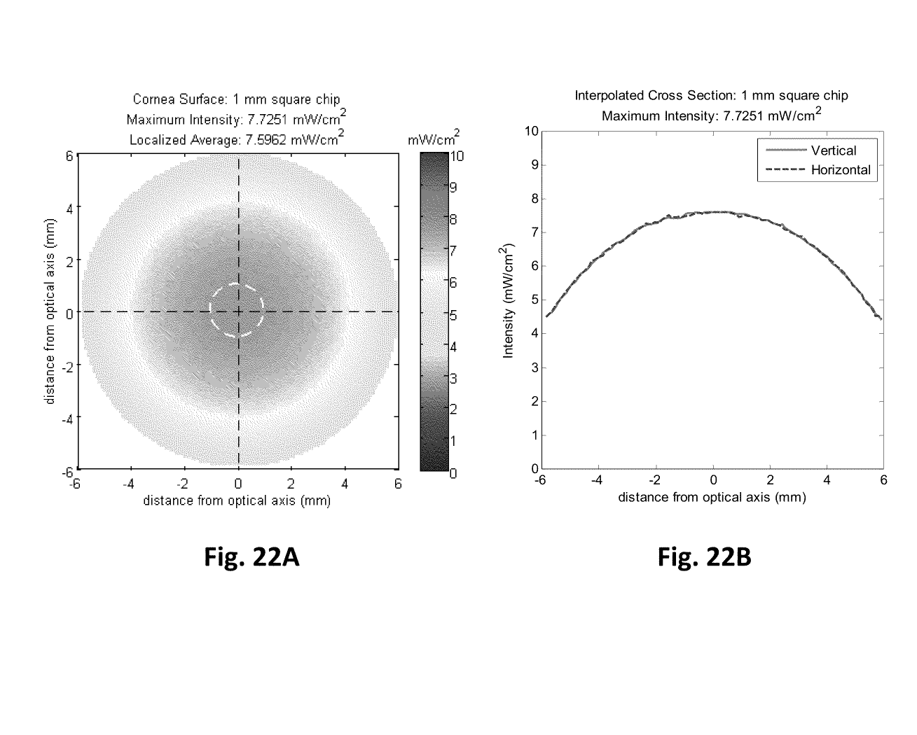

In normal use on normal patients, the pupil is likely to constrict and not remain dilated at 7 mm. Simulations with a 3 mm pupil indicate that the overlap region from the LED image on the retina vanishes, and the brightest spot on the retina is due to the individual LEDs. For the 1 mm square chip geometry, the brightest intensity with a 7 mm pupil is more than three times the intensity with a 3 mm pupil (FIG. 19) Likewise the intensity with the 3 mm pupil is less than half the intensity for a 7 mm pupil when using the other LED source geometries for the simulation. The retinal image size also varies depending on pupil size (FIG. 20). Retinal images for all sources are ˜1 mm in size for the 3 mm pupil. For the 1 mm square chip geometry, retinal image size decreases to ˜0.6 mm, while for the other two source geometries, the image size increases to 1.6 mm.

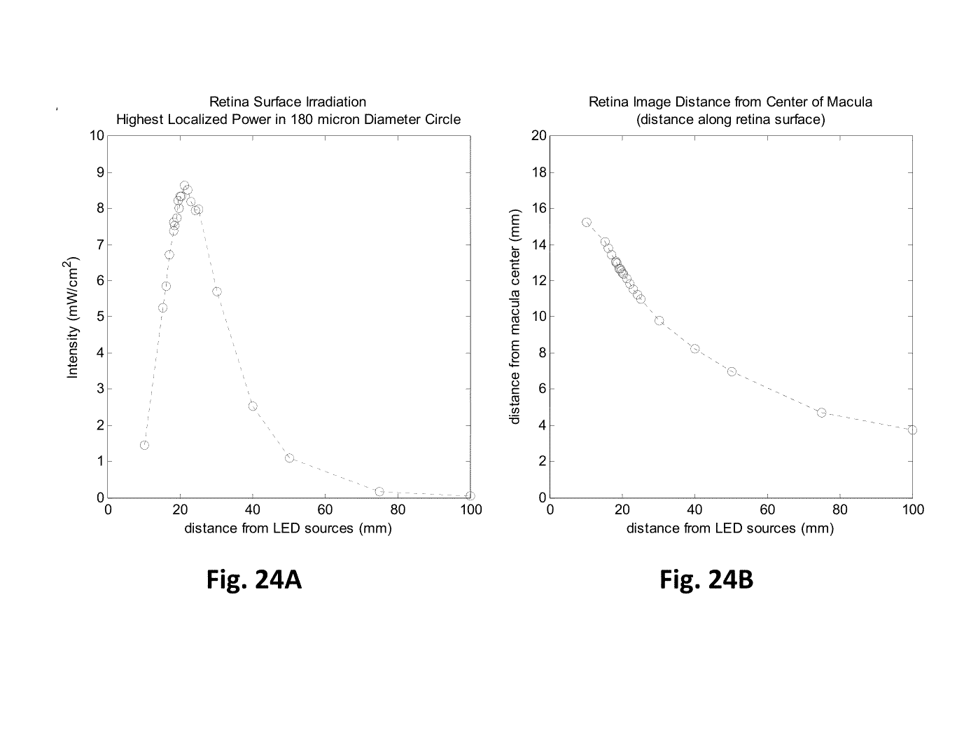

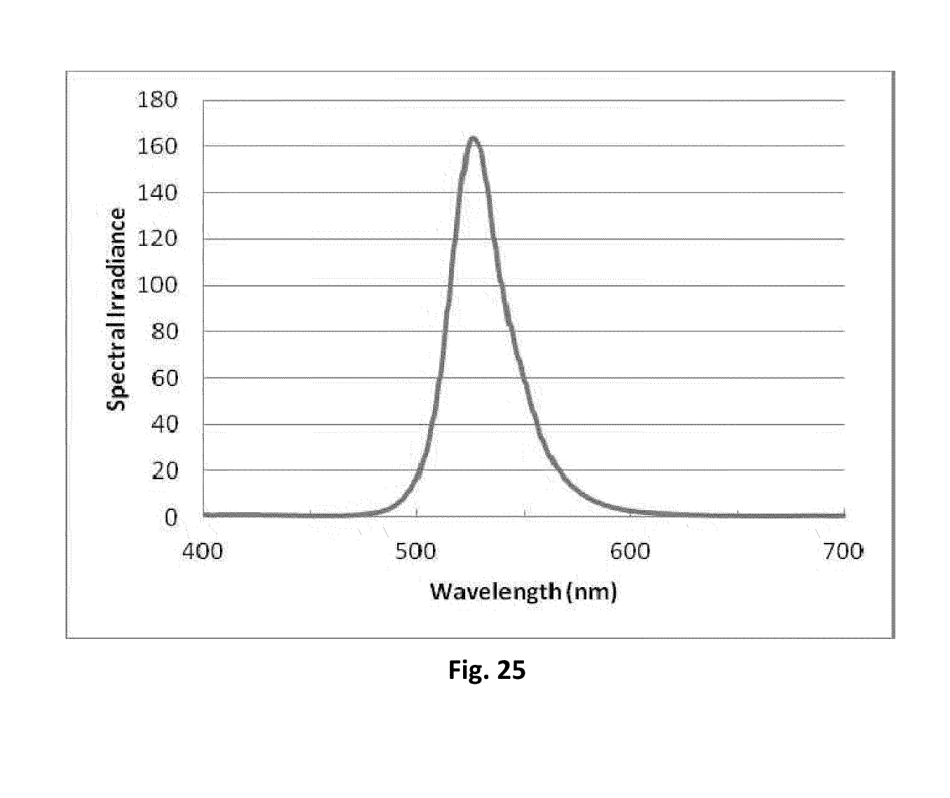

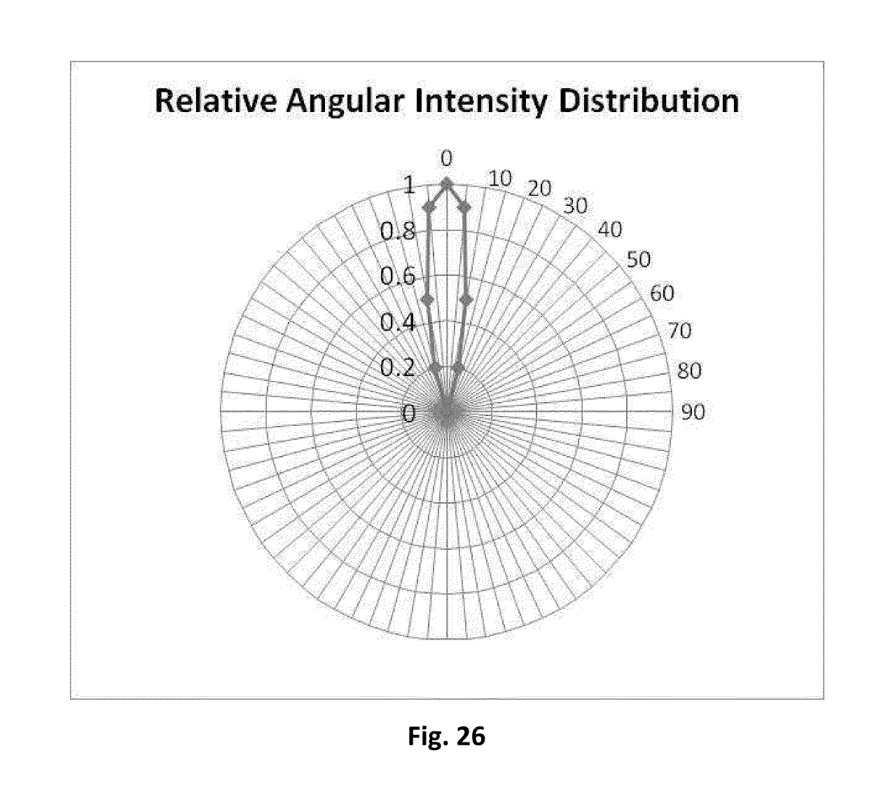

The light source distance from the cornea also affects the image intensity and location on the retina surface. The intensity increases as the light is moved away from the eye, until about 22 mm from the cornea, and then decreases to negligible levels far from the eye (FIGS. 24A-24B). Also, the close the light source is to the eye, the further the retinal image is from the center of the macula.

### Example 19

### Safety Calculations of a Light Device Configured to Deliver Light in a Direction Oblique to a Target Ocular Region

Safety calculations performed using the ISO standards are performed below in Tables 5-8. Results indicate that the light source in the exemplary light delivery device of Example 1 has minimal potential hazard if the pupil diameter is 3 mm. If the pupil diameter is 7 mm, then the exposure levels for the photochemical hazard are higher than the Group 1 safety limits, and Group 2 calculations must be completed.

The retinal exposure falls below all limits for Group 2, indicating that the light source is safe given the current treatment time of 10 minutes (Table 8). Because the light source did not fall below the Group 1 limits, it is required to label the device with a time limit for the exposure. Calculating the exposure time limits based on the photochemical hazard in Group 2, the most conservative estimate for the 1 mm square chip and 7 mm pupil size would be 6.8 hours (Table 4). This time is 40 times greater than that used in the treatment.

TABLE 4
Group 2 Safety Time Limits (7 mm pupil)

| | Exposure Time | Actual Exposure | % of |
|---|---|---|---|
| | Limit (hours) | Time (hours) | Limit |
| 1 mm square chip | 6.8 | 0.17 | 2.4 |
| 3.25 mm reflector | 12.3 | 0.17 | 1.4 |
| 1 mm square chip and 3.25 | 12.8 | 0.17 | 1.3 |
| mm reflector | | | |

Table 5
Group 1 Limits - Ophthalmic Instrument for which no potential light hazard exists

| | | Wavelength | | | |
|---|---|---|---|---|---|
| | Parameter | (nm) | Equation | Limit | |
| 5.4.1.1 | Weighted corneal and lenticular ultraviolet radiation irradiance, E_{S-CL} | 250 to 400 | ${E}_{S - CL} = {\sum }_{250}^{400} {E}_{λ} \times S ( λ ) \times Δλ$ | 0.4 | μW/cm² |
| 5.4.1.2 | Unweighted corneal and lenticular ultraviolet irradianceE_{UV-CL}, | 360 to 400 | ${E}_{UV - CL} = {\sum }_{360}^{400} {E}_{λ} \times Δλ$ | 1 | mW/cm² |
| 5.4.1.3 | Retinal Photochemical aphakic light hazard, Weighted retinal irradiance, E_{A-R} | 305 to 700 | ${E}_{A - R} = {\sum }_{305}^{700} {E}_{λ} \times A ( λ ) \times Δλ$ | 220 | μW/cm² |
| 5.4.1.4 | Unweighted corneal and lenticular infrared radiation irradiance, E_{IR-CL} | 770 to 2500 | ${E}_{IR - CL} = {\sum }_{770}^{2500} {E}_{λ} \times Δλ$ | 20 | mW/cm² |
| 5.4.1.5 | Unweighted anterior segment visible and infrared radiation irradiance, E_{VIS-AS} (convergent beams only) | 380 to 1200 | ${E}_{VIR - AS} = {\sum }_{380}^{1200} {E}_{λ} \times Δλ$ | 4 | W/cm² |
| 5.4.1.6 | Retinal visible and infrared thermal hazard, Weighted retinal visible and infrared radiation thermal irradiance, E_{VIR-R} | 380 to 1400 | ${E}_{VIR - R} = {\sum }_{380}^{1400} {E}_{λ} \times R ( λ ) \times Δλ$ | 0.7 | W/cm² |

TABLE 6
Group 1 Limits—Ophthalmic Instrument for which no potential light hazard exists.

| | | 1 mm Chip | | 1 mm Chip | | Chip and Reflector | | Chip and Reflector | | Whole Reflector | | Whole Reflector | |
|---|---|---|---|---|---|---|---|---|---|---|---|---|---|
| | | (3 mm Pupil) | | (7 mm Pupil) | | (3 mm Pupil) | | (7 mm Pupil) | | (3 mm Pupil) | | (7 mm Pupil) | |
| | | Maximum Corneal Irradiance | | | | | | | | | | | |
| | | 7.71 mW/cm² | | 7.73 mW/cm² | | 7.70 mW/cm² | | 7.76 mW/cm² | | 7.63 mW/cm² | | 7.64 mW/cm² | |
| | | Maximum Retinal Irradiance | | | | | | | | | | | |
| | | 2.35 mW/cm² | | 9.00 mW/cm² | | 1.79 mW/cm² | | 5.00 mW/cm² | | 1.70 mW/cm² | | 4.80 mW/cm² | |
| | | Experiment Exposure Time | | | | | | | | | | | |
| | | 600 sec (10 min) | | 600 sec (10 min) | | 600 sec (10 min) | | 600 sec (10 min) | | 600 sec (10 min) | | 600 sec (10 min) | |
| | | | Simu- | | Simu- | | Simu- | | Simu- | | Simu- | | Simu- |
| | | Simu- | lation/ | Simu- | lation/ | Simu- | lation/ | Simu- | lation/ | Simu- | lation/ | Simu- | lation/ |
| | Parameter | lation | Limit | lation | Limit | lation | Limit | lation | Limit | lation | Limit | lation | Limit |
| 5.5.1.1 | Weighted | 1.0 × | 3.5 × | 1.0 × | 3.5 × | 1.0 × | 3.5 × | 1.0 × | 3.5 × | 1.0 × | 3.4 × | 1.0 × | 3.4 × |
| | Corneal | 10⁻⁵ | 10⁻⁶ | 10⁻⁵ | 10⁻⁶ | 10⁻⁵ | 10⁻⁶ | 10⁻⁵ | 10⁻⁶ | 10⁻⁵ | 10⁻⁶ | 10⁻⁵ | 10⁻⁶ |
| | UV | mJ/cm² | | mJ/cm² | | mJ/cm² | | mJ/cm² | | mJ/cm² | | mJ/cm² | |
| 5.5.1.2 | Un- | 4.9 × | 4.9 × | 4.9 × | 4.9 × | 4.9 × | 4.9 × | 4.9 × | 4.9 × | 4.8 × | 4.8 × | 4.8 × | 4.8 × |
| | weighted | 10⁻⁴ | 10⁻⁴ | 10⁻⁴ | 10⁻⁴ | 10⁻⁴ | 10⁻⁴ | 10⁻⁴ | 10⁻⁴ | 10⁻⁴ | 10⁻⁴ | 10⁻⁴ | 10⁻⁴ |
| | Corneal | mW/cm² | | mW/cm² | | mW/cm² | | mW/cm² | | mW/cm² | | mW/cm² | |
| | UV | | | | | | | | | | | | |
| 5.5.1.3 | Un- | N/A | N/A | N/A | N/A | N/A | N/A | N/A | N/A | N/A | N/A | N/A | N/A |
| | weighted | | | | | | | | | | | | |
| | Corneal | | | | | | | | | | | | |
| | IR | | | | | | | | | | | | |
| 5.5.1.4 | Un- | 7.7 × | 3.9 × | 7.7 × | 3.9 × | 7.7 × | 3.9 × | 7.8 × | 3.9 × | 7.6 × | 3.8 × | 7.6 × | 3.8 × |
| | weighted | 10⁻³ | 10⁻⁴ | 10⁻³ | 10⁻⁴ | 10⁻³ | 10⁻⁴ | 10⁻³ | 10⁻⁴ | 10⁻³ | 10⁻⁴ | 10⁻³ | 10⁻⁴ |
| | Anterior | W/cm² | | W/cm² | | W/cm² | | W/cm² | | W/cm² | | W/cm² | |
| | VIS | | | | | | | | | | | | |
| 5.5.1.5 | Retinal | 2.4 × | 3.4 × | 9.0 × | 1.3 × | 1.8 × | 2.6 × | 5.0 × | 7.1 × | 1.7 × | 2.4 × | 4.8 × | 6.9 × |
| | VIS and | 10⁻³ | 10⁻³ | 10⁻³ | 10⁻² | 10⁻³ | 10⁻³ | 10⁻³ | 10⁻³ | 10⁻³ | 10⁻³ | 10⁻³ | 10⁻³ |
| | IR | W/cm² | | W/cm² | | W/cm² | | W/cm² | | W/cm² | | W/cm² | |
| 5.5.1.6 | Retinal | 6.4 × | 6.4 × | 2.5 × | 2.5 × | 4.9 × | 4.9 × | 1.4 × | 1.4 × | 4.6 × | 4.6 × | 1.3 × | 1.3 × |
| | Photo- | 10⁻² | 10⁻³ | 10⁻¹ | 10⁻² | 10⁻² | 10⁻³ | 10⁻¹ | 10⁻² | 10⁻² | 10⁻³ | 10⁻¹ | 10⁻² |
| | chemical | J/cm² | | J/cm² | | J/cm² | | J/cm² | | J/cm² | | J/cm² | |

| Time Limit | 26.1 hrs | 6.8 hrs | 34.3 hrs | 12.3 hrs | 36.0 hrs | 12.8hrs |
|---|---|---|---|---|---|---|
| Based Retinal | | | | | | |
| Photochemical | | | | | | |

TABLE 7
Group 2 Limits - Ophthalmic Instruments for which potential light hazard exists

| | | Wavelength | | | |
|---|---|---|---|---|---|
| | Parameter | (nm) | Equation | Limit | |
| 5.5.1.1 | Weighted corneal and lenticular ultraviolet radiant exposure, H_{S-CL} | 250 to 400 | ${H}_{S - CL} = {\sum }_{250}^{400} ( {E}_{λ} \times t ) \times S ( λ ) \times Δλ$ | 3 | mJ/cm² |
| 5.5.1.2 | Unweighted corneal and lenticular ultraviolet radiant exposure H_{UV-CL}or irradianceE_{UV-CL}, | 360 to 400 | ${H}_{UV - CL} = {\sum }_{360}^{400} ( {E}_{λ} \times t ) \times Δ λ$ | 1 | J/cm² for t < 1000 s |
| | | | ${E}_{UV - CL} = {\sum }_{360}^{400} {E}_{λ} \times Δ λ$ | 1 | mW/cm² for t > 1000 s |
| 5.5.1.3 | Unweighted corneal and lenticular infrared radiation irradiance, E_{IR-CL} | 770 to 2500 | ${E}_{IR - CL} = {\sum }_{770}^{2500} {E}_{λ} \times Δ λ$ | 100 | mW/cm² |
| 5.5.1.4 | Unweighted anterior segment visible and infrared radiation irradiance, E_{VIS-AS} | 380 to 1200 | ${E}_{VIR - AS} = {\sum }_{380}^{1200} {E}_{λ} \times Δλ$ | 20 | W/cm² |
| | (convergent beams only) | | | | |
| 5.5.1.5 | Retinal visible and infrared radiation thermal hazard, Weighted retinal visible and infrared | 380 to 1400 | ${E}_{VIR - R} = {\sum }_{380}^{1400} {E}_{λ} \times R ( λ ) \times Δλ$ | $\frac{1.2}{{d}_{r}}$ | W/cm² |
| | radiation thermal | | | | |
| | irradiance, E_{VIR-R} | | | | |
| 5.5.1.6 | Retinal radiant exposure guideline (aphakic photochemical light hazard), Weighted retinal | 305-700 | ${H}_{A - R} = {\sum }_{305}^{700} ( {E}_{λ} \times t ) \times A ( λ ) \times Δλ$ | 10 | J/cm² |
| | radiant exposure, H_{A-R} | | | | |

TABLE 8
Group 2 Limits—Ophthalmic Instruments for which potential light hazard exists.

| | | 1 mm Chip | | 1 mm Chip | | Chip and Reflector | | Chip and Reflector | | Whole Reflector | | Whole Reflector | |
|---|---|---|---|---|---|---|---|---|---|---|---|---|---|
| | | (3 mm Pupil) | | (7 mm Pupil) | | (3 mm Pupil) | | (7 mm Pupil) | | (3 mm Pupil) | | (7 mm Pupil) | |
| | | Maximum Corneal Irradiance | | | | | | | | | | | |
| | | 7.71 mW/cm² | | 7.73 mW/cm² | | 7.70 mW/cm² | | 7.76 mW/cm² | | 7.63 mW/cm² | | 7.64 mW/cm² | |
| | | Maximum Retinal Irradiance | | | | | | | | | | | |
| | | 2.35 mW/cm² | | 9.00 mW/cm² | | 1.79 mW/cm² | | 5.00 mW/cm² | | 1.70 mW/cm² | | 4.80 mW/cm² | |
| | | | Simu- | | Simu- | | Simu- | | Simu- | | Simu- | | Simu- |
| | | Simu- | lation/ | Simu- | lation/ | Simu- | lation/ | Simu- | lation/ | Simu- | lation/ | Simu- | lation/ |
| | Parameter | lation | Limit | lation | Limit | lation | Limit | lation | Limit | lation | Limit | lation | Limit |
| 5.4.1.1 | Weighted | 1.7 × 10⁻⁵ | 4.3 × | 1.7 × 10⁻⁵ | 4.3 × | 1.7 × 10⁻⁵ | 4.3 × | 1.7 × 10⁻⁵ | 4.3 × | 1.7 × 10⁻⁵ | 4.3 × | 1.7 × 10⁻⁵ | 4.3 × |
| | Corneal | μW/cm² | 10⁻⁵ | μW/cm² | 10⁻⁵ | μW/cm² | 10⁻⁵ | μW/cm² | 10⁻⁵ | μW/cm² | 10⁻⁵ | μW/cm² | 10⁻⁵ |
| | UV | | | | | | | | | | | | |
| 5.4.1.2 | Unweighted | 4.9 × 10⁻⁴ | 4.9 × | 4.9 × 10⁻⁴ | 4.9 × | 4.9 × 10⁻⁴ | 4.9 × | 4.9 × 10⁻⁴ | 4.9 × | 4.8 × 10⁻⁴ | 4.8 × | 4.8 × 10⁻⁴ | 4.8 × |
| | Corneal | mW/cm² | 10⁻⁴ | mW/cm² | 10⁻⁴ | mW/cm² | 10⁻⁴ | mW/cm² | 10⁻⁴ | mW/cm² | 10⁻⁴ | mW/cm² | 10⁻⁴ |
| | UV | | | | | | | | | | | | |
| 5.4.1.3 | Retinal | 106 | 0.48 | 408 | 1.9 | 81 | 0.37 | 226 | 1 | 77 | 0.35 | 217 | 0.99 |
| | Photo- | μW/cm² | | μW/cm² | | μW/cm² | | μW/cm² | | μW/cm² | | μW/cm² | |
| | chemical | | | | | | | | | | | | |
| 5.4.1.4 | Unweighted | N/A | N/A | N/A | N/A | N/A | N/A | N/A | N/A | N/A | N/A | N/A | N/A |
| | Corneal | | | | | | | | | | | | |
| | IR | | | | | | | | | | | | |
| 5.4.1.5 | Unweighted | 7.7 × 10⁻³ | 1.9 × | 7.7 × 10⁻³ | 1.9 × | 7.7 × 10⁻³ | 1.9 × | 7.8 × 10⁻³ | 1.9 × | 1.6 × 10⁻³ | 1.9 × | 7.6 × 10⁻³ | 1.9 × |
| | Anterior | W/cm2 | 10⁻³ | W/cm² | 10⁻³ | W/cm² | 10⁻³ | W/cm² | 10⁻³ | W/cm² | 10⁻³ | W/cm² | 10⁻³ |
| | VIS | | | | | | | | | | | | |
| 5.4.1.6 | Retinal | 2.3 × 10⁻³ | 3.4 × | 9.0 × 10⁻³ | 1.3 × | 1.8 × 10⁻³ | 2.6 × | 5.0 × 10⁻³ | 7.1 × | 1.7 × 10⁻³ | 2.4 × | 4.8 × 10⁻³ | 6.9 × |
| | VIS and | W/cm² | 10⁻³ | W/cm² | 10⁻² | W/cm² | 10⁻³ | W/cm² | 10⁻³ | W/cm² | 10⁻³ | W/cm² | 10⁻³ |
| | IR | | | | | | | | | | | | |

### Example 20

### Safe Use of a Laser for Alignment of a Light Delivery Device

In the exemplary light delivery device of Example 1, two overlapping laser spots are used to align the light delivery device at a desired distance from the cornea. In this example, a green laser (lambda 0.532 microns) which can cause fluorescence of Eosin Y, is improving visibility on the alignment spots on the cornea. With a laser spot size of ˜1 mm on the cornea, and both lasers with a maximum power of 100 uW, the power incident on the cornea would be ˜25 mW·cm⁻². To ensure minimal activation of Eosin Y, the alignment procedure can be limited to ˜10 seconds (˜2% of total crosslinking time).

Safe light illumination levels on the human eye are regulated through the American National Standards Institute for all types of light sources. For retinal illumination of a stationary spot in the eye at wavelength lambda (in microns) the maximum permissible exposure (MPE) power is (ANSI Z136.1-2000). We can consider the worst case where the collimated beam is directed onto the retina and is considered a “small source.” For lambda 0.500 to 0.700, for times between 10 and 3·10⁴ seconds: 1·10⁻³ W·cm² (From Table 5a of ANSI Z136.1-2000). The limiting aperture is 7 mm in diameter, and the area is 0.385 cm⁻². MPE=385 uW

The alignment power in the exemplary light delivery device of Example 1 is <100 μW, which is less than 30% of the MPE. Since the lasers used for alignment are not directed into the eye, the actual MPE would be lower. Safety checks can be performed before using the device to ensure the subject is not exposed to light levels in excess of the designated safe levels.

Definitions from ANSI Z136.1-2000: Extended source—optical radiation with an angular subtense at the cornea larger than alpha(min). Small source—In this document, a source with an angular subtense at the cornea equal to or less than alpha-min, ie., ≦ than 1.5 mrad. This includes all sources formerly referred to as “point sources” and meeting small-source viewing (formerly called point source or intrabeam viewing conditions. (See section 8.1 of ANSI 2136.1-2000 for criteria). Code of Federal Regulations (CFR) Title 21 regulates the performance standards for light-emitting products (Section 1040.10). Class I levels of laser radiation are not considered to be hazardous. Class I Accessible Emission Limits for Laser Radiation.

For wavelengths >400 nm but ≦1400 nm: Times 10 to 10⁴ seconds: 3.9·10⁻³·k1·k2 (J). k1=1; k2=1. Class I limits for time 10 to 10⁴ seconds: 3.9·10⁻³ (J).

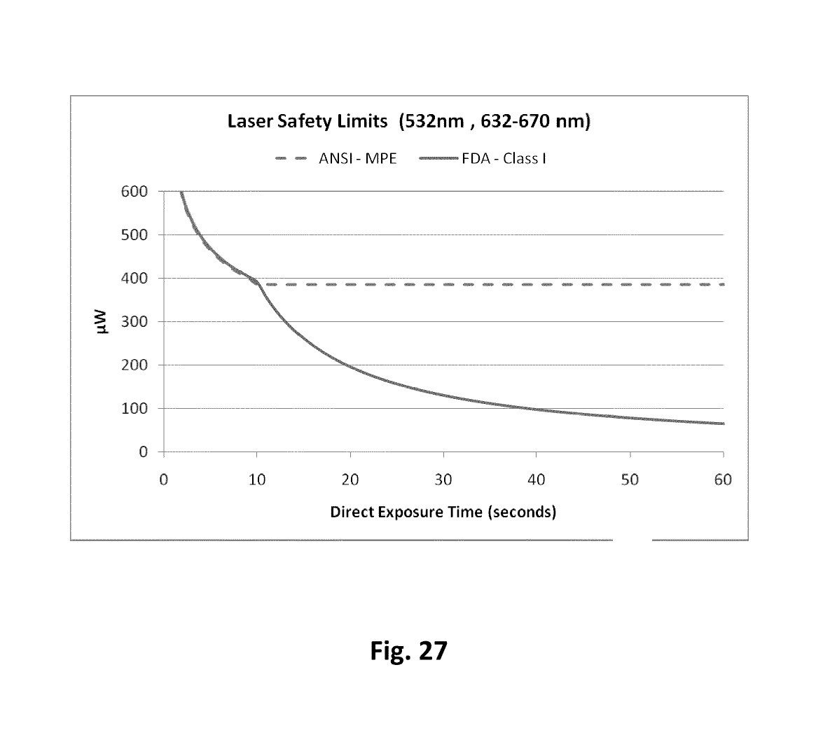

A laser with a power level of 100 μW can be used for 39 seconds without being considered hazardous. Since the alignment process will most likely not exceed 10 seconds, this alignment method will not pose a hazard to the retina. Safety features will ensure that the light levels will not exceed the safe levels. (see FIG. 27)

### Example 21

### Consideration in Designing a Safe Light Delivery Device

Safety of devices can be evaluated with reference to criteria known to a skilled person. For example, reference is made to two papers by Morgan et al [231, 232] discussing safety issues of light delivery devices. A first paper published by Morgan et al in 2008 [231] expresses concerns about those devices because certain devices cause permanent damage to the retinas of macaque while operating near or below the safety standards. Although the devices discussed used a wavelength of 568 nm, the 525 nm light source in the exemplary light delivery device of Example 1 can be compared.

The top of the table summarizes the results of their experiments and the bottom includes values for the light source the exemplary light delivery device of Example 1 (values indicated by the * have been calculated).

The data for Morgan et al [231] indicates that permanent damage occurs above exposures of 233 mW/cm² for exposures of 900 seconds. Below that level, they see no permanent damage, but do see a reversible change in the autofluorescence (AF).

The ZEMAX® simulation of the exemplary light delivery device of Example 1 indicate that there will be a maximum irradiance on the retina of 9 mW·cm⁻² (We include 10 mW/cm² in the table to show conservative estimates of safety). The retinal irradiance the exemplary light delivery device of Example 1 is 23 times less than the safe limit reported by Morgan et al, and ˜50% less than the smallest dose they used. Observations for retinal damage should be performed, but is well below damage thresholds from Morgan et al.[231]

TABLE 9
threshold for observation of retinal damage

| | | | | | | | AF-Ratio | | | | |
|---|---|---|---|---|---|---|---|---|---|---|---|
| | Spot | | Average | Radiant | | | Immediately | | | | |
| | Size | Time | Power | Exposure | Irradiance | | Post- | Permanent Damage | | | |
| Experiment | (°) | (seconds) | (μW) | (J/cm²) | (mW/cm²) | Spots | exposure | RPE | Photoreceptor | Color | FA |

Morgan et al (2008)

| | | | | | | | | | | | |
|---|---|---|---|---|---|---|---|---|---|---|---|
| 1 | 1/2 | 900 | 150 | 788 | 876* | 4 | 0.58 ± 0.03 | 100% | 100% | 100% | 100% |
| 4 | 1/2 | 900 | 150 | 788 | 876* | 3 | 0.51 ± 0.03 | 100% | 100% | 100% | 67% |
| 4 | 1/2 | 900 | 150 | 788 | 876* | 3 | 0.58 ± 0.03 | 100% | 100% | 100% | 100% |
| 1 | 1/2 | 900 | 140 | 735 | 817* | 1 | 0.7 | 100% | 100% | 100% | 100% |
| 1 | 1/2 | 900 | 55 | 289 | 321* | 3 | 0.71 ± 0.02 | 100% | 0% | 100% | 100% |
| 4 | 1/2 | 900 | 55 | 289 | 321* | 3 | 0.58 ± 0.1 | 100% | 33% | 100% | 100% |
| 4 | 1/2 | 900 | 55 | 289 | 321* | 3 | 0.68 ± 0.01 | 100% | 0% | 100% | 100% |
| 1 | 1/2 | 900 | 47 | 247 | 274* | 1 | 0.66 | 100% | 0% | 100% | 100% |
| 4 | 1/2 | 900 | 47 | 247 | 274* | 1 | 0.65 | 100% | 100% | 100% | 100% |
| 4 | 1/2 | 900 | 47 | 247 | 274* | 1 | 0.63 | 100% | 100% | 100% | 0% |
| 1 | 1/2 | 900 | 40 | 210 | 233* | 2 | 0.81 ± 0.02 | 0% | 0% | 0% | 0% |
| 2 | 2 | 900 | 88 | 29 | 32* | 7 | 0.83 | 0% | 0% | 0% | 0% |
| 1 | 1/2 | 900 | 3 | 16 | 18* | 1 | 0.88 | 0% | 0% | 0% | 0% |

Exemplary Light Delivery Device of Example 1 (2011)

| | | | | | | |
|---|---|---|---|---|---|---|
| Simulation | 1/2 | 600 | 2* | 6* | 10 | Various spot sizes have been included here to |
| | 2 | 600 | 27* | 6* | 10 | illustrate the comparable average power if the |
| | 11 | 600 | 79* | 6* | 10 | exemplary light delivery Device of Example 1 light |
| | | | | | | only illuminated such a spot. The actual spot size from |
| | | | | | | ZEMAX ® simulations is on the order of 1 mm, which |
| | | | | | | corresponds to 11°. The energy density (radiant |
| | | | | | | exposure) or power density (irradiance) is used for |
| | | | | | | comparisons. |

The second paper published by Morgan et al in 2009 [232] has more detailed tests of lower radiant exposures. They find that below 105 J·cm⁻² is safe, and above 289 J·cm⁻² caused permanent damage. There was no significant autofluorescence reduction for levels of 1 or 2 J·cm⁻². Levels at 5, 14, and 39 J·cm⁻² had a significant immediate reduction in autofluorescence that vanished several days after exposure.

Because the light source of the exemplary light delivery device of Example 1 is at ˜6 J·cm⁻², patients can experience an immediate reduction in autofluorescence that is restored after several days. This is the same result expressed in the previous paper. Based on the concentration of Eosin Y delivered to the cornea, the absorption of light can be predicted, and it is estimated that less than ⅓ of the light actually penetrates the cornea. This would reduce the retinal radiant exposures to ˜2 J·cm⁻², which should not result in an immediate reduction in autofluorescence.

### Example 22

### Example of Evaluating Irradiance of a Retinal Region and Source Radiance

As explained in Sliney and Wolbarsht (1980) [210], the solid angle formed by an extended source can be used to determine the image size on the retina for reasonably small angles and even for short viewing distances. The reason for this is that for each point on the source there is a corresponding point at the retinal image plane. In this example, the angular subtense a is the linear angle corresponding to the solid angle of the source, Ωs, subtended by either the source or the image on the retina with the apex at the nodal point of the eye is the same. This can be shown by using similar triangles and by assuming that the arc and chord of a circle are approximately the same for reasonably small angles.

With the use of similar triangles, it can be shown that the solid angle of the source is the same as the solid angle of the retinal image, the area of the image on the retina can be determined since the distance of the nodal point to the retinal plane is also known. This distance is usually taken to be 1.7 cm for a relaxed emmetropic eye (focused at infinity). Thus, with knowledge of the solid angle of the source and the distance of the source to the cornea, it is possible to determine the area of the image in the retinal plane.

Sliney and Wolbarsht (1980) [210] show that for small angles, the retinal irradiance is related to the source radiance with the expression,

$${E}_{r} = \frac{π \cdot {d}_{e}^{2} \cdot L \cdot τ}{4 {f}_{e}^{2}} , \qquad ( 14 )$$

where,
Eᵣ is the retinal irradiance,
d_{c} is the diameter of the beam at the pupil for diameters less than 7 mm. For beam diameters greater than or equal to 7 mm, a limiting 7 mm beam diameter is used.
L is the source radiance,
T is the transmittance of the ocular media, and,
fₑ² is the focal length of the eye.

Taking the focal length of the Gulstrand model eye to be 1.7 cm and simplifying, Equation 14 becomes,

$${E}_{r} = 0.79 \frac{{d}_{e}^{2} L \cdot τ}{{f}^{2}} . \qquad ( 15 )$$

Now, the radiance, L, is also equal to the corneal irradiance and the solid angle of the source as given by the expression,

$$L = \frac{{E}_{c}}{{Ω}_{s}} . \qquad ( 16 )$$

### Substituting for L in Equation 15,

$${E}_{r} = \frac{{Φ}_{r}}{{A}_{r}} = 0.79 {d}_{e}^{2} \frac{{E}_{c} τ}{{Ω}_{s} {f}^{2}} = 0.79 {d}_{e}^{2} \frac{{Φ}_{c} τ}{{A}_{c} {f}^{2} {Ω}_{s}} \qquad ( 17 )$$

where,
Φᵣ is equal to the radiant power incident on the retina,
Φ_{c} is the radiant power incident on the cornea, and
A_{c} is the area of the cornea irradiated.

Using the specified 7-mm aperture on the cornea specified in ISO 15004-2, it is clear that all of the radiant power from an ophthalmoscope with a small cone angle will be collected by the 7 mm averaging aperture. Furthermore, all of the radiant power will be transmitted to the retina so that P_{c}=Pᵣ. We can then determine Aᵣ from the expression,

$${A}_{r} = 1.27 \frac{{A}_{c} {f}^{2} {Ω}_{s}}{{d}_{e}^{2} τ} {cm}^{2} \qquad ( 18 )$$

From Equation (18) above, and for a distance of 1 cm, A_{c}=(1)²Ωₛcm²., and,

$${A}_{r} = 1.27 \frac{{f}^{2} {Ω}_{s}^{2}}{{d}_{e}^{2} τ} {cm}^{2} \qquad ( 19 )$$

The retinal irradiance is to be evaluated for hot-spots using an averaging aperture on the retina of ˜25 μm at the retinal plane; but since the beam on the retina should be homogeneous and there should be no hot spots, such a small averaging aperture is unnecessary. Therefore the retinal irradiance, Eᵣ, is equal to the radiant power divided by the area of the beam on the retina since the radiant power divided by the area of the retinal spot size would yield the same result.

The retinal irradiance is then given by,

$${E}_{r} = 0.79 \frac{{P}_{c} {d}_{e}^{2} τ}{{f}^{2} {Ω}_{s}^{2}} W \text{/} {cm}^{2} \qquad ( 20 )$$

It can be shown that Equation (16) yields an equivalent result to the following expression,

$${E}_{r} = \frac{{P}_{c} τ}{{f}_{e}^{2} {Ω}_{s}} W \text{/} {cm}^{2} . \qquad ( 21 )$$

With this formulation, it is only necessary to measure the radiant power on the cornea and the beam solid angle. With the formulation in Equation (16), it is necessary to determine the diameter of the beam on the cornea as well as the radiant power on the cornea and the projected solid angle of the source.

In order to take into account the spectral power distribution, it is necessary to determine the retinal spectral irradiance, and for this case,

$${E}_{λ r} = \frac{{Φ}_{λ}}{{A}_{ret}} , \qquad ( 22 )$$

where,
E_{λ} is the spectral irradiance,
Φ_{λ} is the spectral radiant power, and,
Aᵣₑₜ is the area of the retina illuminated.

E_{VIR-R} is then determined with the use of Equation 10. The weighted retinal irradiance value for E_{VIR-R} determined is then compared to the limit specified in Clauses 5.4.1.5 a, or 5.5.1.5 a from ISO 15004-2. E_{A-R} is determined with the use of Equation. 11. This weighted value is compared to the limit in Clause 5.4.1.3 a, from ISO 15004-2. For H_{A-R} the aphakic weighted retinal irradiance is multiplied times the maximum expected exposure time to determine the aphakic weighted retinal radiant exposure which is compared to the guideline specified in Clause 5.5.1.6 a from ISO 15004-2.

### Example 24

### Selection of Compounds Suitable for Photodynamic Collagen Cross-Linking

There has been great interest in photodynamic protein cross-linking due to its wide range of applications including photodynamic therapy for cancer [62, 63], tissue engineering applications [64, 65] and modification of tissue stiffness [34, 66]. Here, we are interested in the photodynamic cross-linking therapy for enhancing weakened ocular tissues, particularly for diseases including keratoconus [17, 67], post-LASIK ectasia [68, 69], and degenerative myopia[10, 70].

Pioneering work of Wollensak, Seiler, Spoerl, et al has led to the development of a corneal cross-linking treatment for keratoconus [71, 72]. Cross-linking the corneal stroma enhances tissue stiffness and halts progression of the disease. Cross-linking can be achieved by activating riboflavin with UVA light (370 nm ultraviolet irradiation). Collagen cross-links formed by riboflavin/UVA are stable to chemical, heat, and enzymatic treatment[40]. Because the addition of cross-links both enhances tissue strength and provides protection from enzymatic digestion, the treatment stabilizes the cornea over a long period. Clinical trials for riboflavin/UVA cross-linking therapy lasting up to 5 years have demonstrated that the treatment effectively halts the disease progression. The strengthening effect due to cross-linking observed in the cornea is also seen in the sclera[66, 73]. So cross-linking might be possible to halt the progression of degenerative myopia, which is a diseases associated with weakening and thinning of the sclera.

Even though the clinical outcome for keratoconus is promising, there are drawbacks to the riboflavin/UVA protocol. The combination of riboflavin with UVA light can produce cytotoxic effects in the cornea and sclera [44, 48, 74]. The cytotoxic nature of riboflavin and UVA light excludes keratoconus patients with corneas thinner than 400 μm from being able to receive this therapy [47, 75]. Riboflavin/UVA treatment has not yet been successfully demonstrated in treating post-LASIK ectasia or degenerative myopia.

A visible light activated photosensitizer, eosin Y, is described here for cross-linking the cornea and sclera. Eosin Y has a maximum absorption peak at 514 nm (green light). Biocompatibility studies in the cornea show this photosensitizer produces much less cytotoxic effects than riboflavin (Example 37). In order to assess eosin Y's ability to increase tissue stability in the cornea and sclera over a period that is clinically relevant, knowledge of the reaction pathway and chemical nature of the cross-links is necessary. Cross referencing the extensive literature on photodynamic protein cross-linking reaction mechanisms, a small set of data can provide information about the chemical reactions pertinent to therapeutic cross-linking treatments.

Various proteins undergo covalent cross-linking when irradiated with light in the presence of a photosensitizer [76-78]. There are two major photosensitization pathways: type I or direct reaction pathway and type II or indirect reaction pathway. These photodynamic reactions begin with the photosensitizer absorbing light which transitions the molecule from its ground state to an excited state. In type I, the photosensitizer in this excited state reacts with the protein molecule by hydrogen or electron transfer [79]. In type II, the photosensitizer in its excited state transfers its energy to ground state molecular oxygen to produce singlet oxygen. This highly reactive singlet oxygen species then oxidizes the protein [79]. Photosensitization reactions can occur via both type I and type II pathways at the same time. The relative contribution of the two pathways depends on the sensitizer, protein, solvent composition, and other experimental conditions [80, 81]. To determine the relative importance of the two pathways for a specific set of experimental conditions, we assess the involvement of singlet oxygen radicals in the photo-oxidation reaction. A photo-oxidation reaction dominated by the type II pathway would have very different reaction rates in the presence and absence of oxygen [78, 82]. Furthermore, if singlet oxygen is necessary for the reaction, the addition of molecules that quench singlet oxygen radicals (e.g. sodium azide and ascorbic acid) would have an inhibitory effect on the reaction [78, 83-85]. Using this approach, this study examines the role of singlet oxygen radicals in collagen cross-linking induced by riboflavin/UVA and eosin Y/visible light.

Photorheology is used as a tool to make in-situ measurements of a sample's modulus during irradiation. Photo-activated cross-linking of collagen gels are monitored in this manner to determine the effects of adjusting the oxygen in the environment and adjusting the concentration of singlet oxygen quenchers. This provides a simple method of examining the reaction pathway.

### Example 25

### A Method for Preparing a Topical Gel Formulation for Photorheology for Testing a Type of Reaction Pathway

Collagen Gel Preparation—A mixture of 2.5 g gelatin from bovine skin (Sigma Aldrich G6650 Lot #047K0005) and 6.0 mL dulbecco's phosphate buffered saline (DPBS, Sigma D8662) was heated at 75° C. for 30±1 minutes to dissolve all the gelatin. After the gelatin solution was removed from the heat bath, 1 mL of 0.5% riboflavin-5′-monophosphate (riboflavin, Sigma Aldrich R7774) or 1 mL of 0.2% eosin Y (Sigma Aldrich E6003 Lot#022K3692) solution, and 0.5 mL of a quenching reagent (sodium azide or ascorbic acid) stock solution having 20 times the final desired concentration were added to the gelatin solution. The solution was swirled for a few seconds to yield a uniform final mixture containing 25% w/w gelatin, 0.05% riboflavin or 0.02% eosin Y, and the desired quencher concentration. Three conditions were examined for riboflavin: 10 and 100 mM sodium azide and 20 mM ascorbic acid and 4 conditions were examined for eosin Y: 100 mM sodium azide and 10, 20 and 100 mM ascorbic acid.

To produce a uniform layer of gel, a mold was prepared using a Teflon® spacer between two PLEXIGLAS® plates, which were held together with clamps. The Teflon® spacer provided a controlled gap to form 500 μm thick gels. Prior to dispensing the solution into a mold, both the glass Pasteur pipette and the gel mold were warmed using a heat gun (for ˜15 seconds). The warm solution was then dispensed into the warm mold; then the filled mold was wrapped in aluminum foil to prevent dehydration and interaction with light. The gel mold was stored at ˜4° C. for at least 8 hours to form a solid gel. This procedure was used to create eosin Y and riboflavin gels with varying quencher concentrations. All samples were measured within 48 hours of the beginning of gel preparation.

### Example 26

### A Method for Monitoring an Extent of Photodynamic Cross-Linking In-Situ

Collagen gel photorheology was performed on a stress-controlled shear rheometer (TA Instrument AR1000) used as a photorheology apparatus. The lower, stationary tool was modified to deliver light to the sample. A custom-built light delivery device was mounted onto the Peltier plate of the rheometer similar to those described by Khan, Plitz, et al [86] (TA Instrument has similar UV LED accessories for the rheometer). The lower plate was replaced with an aluminum plate with a 50-mm diameter quartz window positioned at the center allowing the transmission of both visible and ultraviolet (UV) light. Irradiation was achieved by placing a cluster of four light emitting diodes directly below the quartz window; two different LED clusters were constructed, one using Luxeon Star LXML-PM01-0080 at 530±15 nm for irradiating gels containing eosin Y and the other using Roithner Lasertechnik UVLED-365-250-SMD at 370±12 nm to irradiate gels containing riboflavin.

To maintain the LED cluster at a steady operating temperature, it was mounted on an aluminum heat sink attached to a tube that provided a steady flow room temperature air over the heat sink. The light intensity (0-6 mW/cm²) was controlled by adjusting the input voltage (0-16 V) provided by a power supply (Hewlett Packard E3620A). The intensity profile as a function of position at the top of the quartz window was characterized using a fiber optic with “cosine corrector” (Ocean Optics Jaz) and was found to vary less than 5% from the value at the center of the 8-mm diameter sample area.

Oscillatory shear storage modulus measurements were then performed as follows. A circular sample 8-mm in diameter was cut from the gel sheet. The sample was placed onto the upper tool (8-mm aluminum parallel plate) to ensure proper alignment. Then the upper tool was lowered to bring the sample in contact with the lower plate. The normal force reading began to register at a gap thickness that was consistent with the spacer's thickness (within 2%). To ensure good contact between the specimen and the tools, the gap was reduced to 90% of the nominal sample thickness with typical initial normal force registering ˜2 N. To prevent gel dehydration, the sample was enclosed in a chamber containing a wet sponge that kept surrounding environment saturated with water vapor. The chamber also had an inlet for gas flow so that the chamber's environment could have oxygen present or absent by purging the chamber with air or argon, respectively.

The temperature of the sample was maintained at 24±1° C. (Omega HH059 thermocouple). Once the sample was in contact with the lower plate, a 15-minute interval was allowed for thermal equilibration before the linear storage modulus was measured at a frequency of 0.3 rad/s using an oscillatory stress amplitude of 30 Pa (in the linear regime). The storage modulus was measured every minute for 50 minutes, including 10 minutes prior to irradiation (to verify that gelation was complete), 30 minutes during irradiation and 10 minutes after cessation of irradiation (to determine if cross-linking continued, i.e., if there is significant “dark reaction”). Each condition was repeated at least 3 times to obtain the reported mean and standard deviation.

### Example 27

### Role of Eosin Y in Photodynamic Cross-Linking

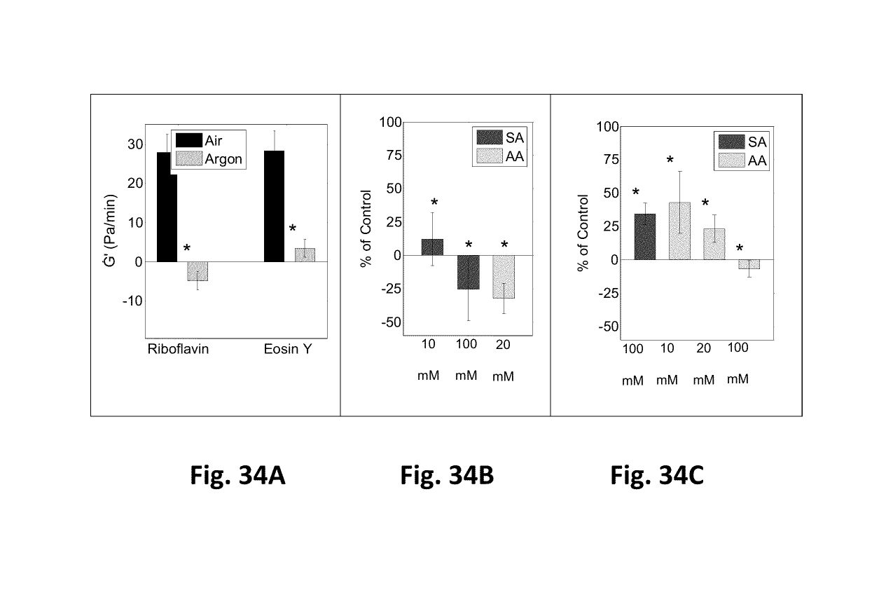

During the 30-minute irradiation period in Example 25, the rate of change in storage modulus, Ġ′, of riboflavin samples increased by 28.0±4.7 Pa/min in the presence of oxygen (in air) and decreased by 4.8±2.3 Pa/min in the absence of oxygen (in argon, FIG. 34A). In the absence of oxygen, no cross-linking occurred. Similarly, Ġ′ of eosin Y samples increased by 28.4±5.1 Pa/min in air and increased by 3.4±2.3 Pa/min in argon. In the absence of oxygen, the cross-linking rate was reduced to 12%.

Addition of singlet oxygen quenchers: sodium azide and ascorbic acid reduced the rate of cross-linking in air. For riboflavin, the cross-linking rate of samples containing: 10 mM sodium azide is 12±20%; 100 mM sodium azide is −26±23%; 20 mM ascorbic is −32±11% of the rate without singlet oxygen quenchers (FIG. 34B). Increasing the concentration of sodium azide increased the inhibitory effect. For eosin Y, the cross-linking rate of samples containing: 100 mM sodium azide is 34±8%; 10 mM ascorbic acid is 43±23%; 20 mM ascorbic is 23±10%; 100 mM ascorbic acid is −7±6% of the rate without singlet oxygen quenchers (FIG. 34C). Increasing the concentration of ascorbic acid increased the inhibitory effect. In both riboflavin and eosin Y samples, ascorbic acid has a greater inhibitory effect than sodium azide.

Riboflavin/UVA clinical treatment for keratoconus relies on the addition of cross-links in the collagen matrix of the cornea to enhance tissue strength and resist enzymatic degradation[39, 87]. The collagen cross-links induced by riboflavin/UVA are stable to chemical, heat, and enzymatic degradation therefore providing a treatment efficacy that lasts for years[40]. A study by McCall, Kraft, et al[84] on the reaction mechanisms of the riboflavin/UVA in the cornea reveals the reaction proceeds via the singlet oxygen pathway. Cross-linking efficacy on the cornea, quantified by the destructive tension of corneal strips, decreased by 76% when sodium azide was added to the riboflavin treatment solution. In accord with this study, we found riboflavin/UVA cross-linking requires oxygen (FIG. 34A), and the addition of singlet oxygen quenchers (sodium azide and ascorbic acid) inhibits cross-linking in the presence of oxygen (FIG. 34B).

Collagen cross-linking activated by eosin Y with visible light exhibits very similar behavior to riboflavin/UVA. Oxygen is required for cross-linking (FIG. 34A) and the addition of singlet oxygen quenchers (sodium azide and ascorbic acid) inhibit cross-linking (FIG. 34B), implying the cross-linking reaction activated by eosin Y/visible light also proceeds via the singlet oxygen pathway. This is also consistent with the fact that eosin Y is known to generate singlet oxygen upon irradiation in the presence of molecular oxygen [88, 89]. Studies by Miskoski and Garcia [90] have also shown eosin photo-sensitized cross-linking of peptides mainly occurs through a process mediated by singlet oxygen. It has been demonstrated that photodynamic reactions proceeding through the singlet oxygen pathway yield similar chemical modifications [91-93]. Based on this understanding, it is expected that the cross-links formed by eosin Y/visible light should be equivalent to the stable ones formed by rifboflavin/UVA light.

### Example 28

### The Role of Eosin Y and Photo-Oxidizable Amino Acids in Collagen Cross-Linking

Extensive studies have shown protein cross-linking can be induced by singlet oxygen in various proteins such as crystallins [77, 85, 94], ribonuclease A [78], spectrin [95], fibrin [96], fibrinogen [96], and collagen [97]. Photo-oxidation of susceptible amino acids in proteins is the primary photodynamic process and the covalent cross-linking is a secondary, light independent reaction [80, 98]. Studies have shown there are only five amino acids: tryptophan, tyrosine, cysteine, methionine and histine, which are susceptible to photo-oxidation [81, 82, 99]. These photo-oxidized amino acids can then interact with other amino acids to form covalent cross-links. Not all interactions result in cross-link formation.

To determine the relative contribution of each photo-oxidizable amino acid in the collagen cross-linking reaction, we can examine the reaction rate of each amino acid with singlet oxygen. The reaction rate between a photo-oxidizable amino acid and a singlet oxygen depends on the chemical rate constant [100, 101] and the concentration of each amino acid present in the gel samples composed of collagen type I [102] (Table 10).

TABLE 11
The quantity of amino acid containing amine group(s)
present in collagen type I.

| Amino Acid | Mole % |
|---|---|
| Glutamine | 0 |
| Asparagine | 0 |
| Arginine | 4.7 |
| Lysine | 2.8 |

TABLE 10
Rate constants for chemical reactions between singlet oxygen
and photo-oxidizable amino acids and the quantity of each
amino found in collagen type I.

| Amino acid | k1_{O}_{₂} × 10⁻⁷ M⁻¹s⁻¹ | Mole % |
|---|---|---|
| Tryptophan | 1.3-3 | 0 |
| Cystei e | 0.89 | 0 |
| Methionine | 1.6 | 0.5 |
| Tyrosine | 0.8 | 0.4 |
| Histidine | 3.2-3.4 | 0.6 |

Cysteine and tryptophan are not present in collagen so they cannot contribute to the observed cross-linking. Methionine has an appreciable rate constant for reacting with singlet oxygen. Even though methionine gets photo-oxidized, different studies have shown it is not involved in cross-linking reactions [78, 80, 103]. Tyrosines can be photo-oxidized to form cross-links with other tyrosines [80, 104]. The formation of dityrosine has been suggested to occur through type I mechanisms [76, 104]. The presence of oxygen actually inhibits tyrosine modification and cross-linking in these reactions. Furthermore, dityrosine formation was not observed in the cross-linking process mediated by singlet oxygen in proteins, peptides, or model tyrosine copolymers [78, 103, 105]. Therefore, tyrosine is not expected to be involved in collagen cross-linking induced by the photosensitizers in this study.

Histidine has been shown to be photo-oxidized via a singlet oxygen mediated process by various independent studies using free histidine amino acid[80, 83, 92, 100], histidine model compounds[78, 82], histidine in peptides[90, 100] and proteins [77, 78, 95, 103, 106]. Photo-oxidation of histidine can lead to cross-linking. Studies using rose bengal as a photosensitizer found histidine residues are necessary for cross-linking; blocking the histidine residues leads to a decrease in cross-link formation in crystalline [77] and ribonuclease A[78]. Proteins without histidine (e.g. melittin and bovine pancreatic trypsin inhibitor) do not form cross-links in the presence of rose bengal and visible light [78]. Even though the exact mechanism of cross-linking involving histidine is not well understood, it has been suggested that cross-link formations occur through an interaction between the photo-oxidized histidine with an amine group [78, 80, 90, 95, 103] or with another histidine[82, 93]. Studies have also shown that modifying amine groups in proteins has an inhibitory effect on photodynamic cross-linking [78, 93, 103]. Because model copolymers containing histidine can react with other copolymer compounds containing lysine[82] or histidine [82, 93] to form photodynamic cross-links through the singlet oxygen pathway, it is likely that both riboflavin/UVA and eosin/visible light react in a similar manner to form cross-links.

Of the five photo-oxidizable amino acids, histidine is the most likely to be involved in collagen cross-linking induced by riboflavin/UVA or eosin Y/visible light. Photo-oxidized histidines can react with other histidines or amino acids containing an amine group in their side chains. Of the four amino acids containing amine group(s) in their side chains, only two are present in collagen type I (Table 3). Based on the quantity present in collagen type I, a photo-oxidized histidine is most likely to react with an asparagine then a lysine, and finally with another histidine. However, the actual rates depend on the proximity of these different amino acids to the photo-oxidized histidine and the degree of “exposure” of the side chains for reaction[81].

Eosin Y is a dye molecule commonly used as a protein staining agent since it unselectively binds to proteins. Studies by Waheed et al[107] found histidine, lysine, and arginine residues of a protein bind electrostatically to eosin Y to produce a stable water-soluble protein-dye complex. Given the proximity due to dye binding between eosin Y and histidine, it further suggests that histidine is likely to react with the near-by singlet oxygen radicals generated by the photosensitizer during irradiation.

Singlet oxygen quenchers inhibit cross-linking by competing with photo-oxidizable amino acids for singlet oxygen. In the two photo-activated cross-linking systems, histidines are expected to be the predominant amino acids being oxidized. Since eosin Y molecules bind to histidines, singlet oxygen generated by these bound photosensitizers are very close to the cross-linking sites. This allows the singlet oxygen molecules to react with the histidines before being quenched by sodium azide or ascorbic acid. For riboflavin, no such binding effect is present (Examples 32-34) to favor the singlet oxygen reaction with histidine over sodium azide or ascorbic acid. Thus, the quenching effects are greater for riboflavin than eosin Y, leading to greater decreases in the cross-linking rates FIG. 34B).

Ascorbic acid has a greater inhibitory effect than sodium azide on the cross-linking rate for both riboflavin/UVA and eosin Y/visible light systems (FIG. 34B). This is in accordance with studies by Zigler et al[85] which also showed ascorbic acid is a better inhibitor of the photo-activated cross-linking reaction in crystallin proteins than sodium azide.

Our experimental results, along with prior literature, suggest collagen cross-linking induced by riboflavin/UVA and eosin Y/visible light are both mediated by singlet oxygen. In addition, histidine is the most likely amino acid to play a major role in the collagen cross-linking reactions in the cornea and sclera. Subsequent reactions with the photo-oxidized histidine residue are likely to involve an arginine, lysine, or another histidine. Cross-links formed via this pathway are found to be stable to chemical treatment using 2-mercaptoethanol, heat treatment by boiling in water for five minutes, and enzymatic degradation by pepsin[40]. These cross-links generated by riboflavin/UVA are found to be stable in the cornea for at least 5 years[71], and since eosin Y generates cross-links via the same reaction pathway they should be stable as well.

### Example 29

### Selecting a Visible Light-Activating Photosensitizing Compound Suitable for Photodynamic Cross-Linking

Keratoconus is a corneal ectasia associated with progressive corneal thinning and protrusion resulting in a conical shaped cornea. This disease has a prevalence of 1 in 2,000 with no race or gender bias[17]. Pioneering research of Wollensak, Seiler, and Spoerl demonstrated that photodynamic corneal collagen cross-linking using riboflavin and UVA can halt the progression of keratoconus[71]. However, the phototoxicity of riboflavin and UVA results in certain limitations and drawbacks. The combination of riboflavin and UVA is toxic to both keratocytes and endothelial cells[44, 108]. The endothelium is responsible for maintaining corneal transparency and the cells do not regenerate in humans. Therefore, the treatment parameters (riboflavin concentration, duration of drug delivery prior to irradiation and frequent reapplication of riboflavin during irradiation) are carefully designed to restrict toxicity to the anterior 350 microns of the stroma[45].

In the current clinical protocol, topical application the drug solution (0.1% riboflavin with 20% dextran) to the cornea is repeated every 2 minutes for 30 minutes before irradiating, and every 5 minutes during the 30 min irradiation with 3 mW/cm2 UVA. The high riboflavin concentration is necessary to prevent significant UVA light from penetrating more than 350 μm. Thirty minutes of topical application prior to irradiation is required to establish the protective riboflavin concentration in the stroma [45]. The treatment typically cannot be used with corneas under 400 μm because it then results in “significant necrosis and apoptosis of endothelial cells”[108]. Almost all of the keratocytes in the anterior 300-350 μm of the stroma undergo apoptosis, which can result in corneal edema [41, 43, 44, 109]. The resulting stromal haze can persist for weeks to months after treatment; full recovery of the keratocyte population requires 6 to 12 months [43, 110].

To retain the benefits of corneal cross-linking and reduce the toxicity of the treatment, it has been suggested that a photosensitizer that is activated by visible light might be used[111]. In vitro results suggest that Eosin Y (a photosensitizer with an absorption peak at 514 nm) can produce cross-linking in the cornea (Example 36) and sclera[1], comparable to riboflavin/UVA treatment. Eosin Y has been approved by the US-FDA for use in the body[51]. Safety studies of eosin Y activated by green light in a rabbit model show little keratocyte apoptosis, using eosin Y concentration and irradiation conditions that produce comparable cross-linking to the riboflavin/UVA treatment for keratoconus (Example 36). No endothelial toxicity was observed with eosin Y/visible light, opening the way to treating patients whose cornea is less than 400 μm thick due to advanced keratoconus or other conditions, such as post-LASIK ectasia[112]. In relation to the application of collagen cross-linking to treat degenerative myopia[48, 66], in vivo studies of eosin Y activated by visible light showed no retinal toxicity in a guinea pig model[1], in contrast to early in vivo results with riboflavin/UVA[108].

### Example 30

### Method of Determine Rates of Photodynamic Cross-Linking for Different Photosensitizing Compounds

This example of relative rates of cross-linking produced by both the clinical therapy (riboflavin/UVA) and the pre-clinical therapy (eosin Y/visible light). The rate of change of the apparent shear modulus is measured as a function of photosensitizer concentration and irradiation intensity using photorheology, which is widely used to study photopolymerization kinetics [86, 113, 114]. Collagen gel is used as a substrate because of its excellent uniformity and reproducibility.

Collagen Gel Preparation was performed using a similar method used in Examples 24-28. Briefly, a mixture of 2.5 g gelatin from bovine skin and 6.5 mL dulbecco's phosphate buffered saline (DPBS) was heated at 75° C. for 30±1 minutes to dissolve all the gelatin. After the gelatin solution was removed from the heat bath, 1 mL of an eosin Y or riboflavin stock solution having 10 times the final desired concentration was added to the 9 mL gelatin solution. The final mixture contained 25% w/w gelatin and the desired concentration of eosin Y or riboflavin. Six concentrations of eosin Y (0.005, 0.01, 0.02, 0.04, 0.1 and 0.2%) and 8 concentrations of riboflavin (0.005, 0.01, 0.03, 0.05, 0.07, 0.1, 0.3, and 0.5%) were examined as well as controls without photosensitizers.

To produce a uniform layer of gel, a mold was prepared using a Teflon® spacer between two PLEXIGLAS® plates, which were held together with clamps. The Teflon® spacer provided a controlled gap with the desired gel thickness; four spacer thicknesses were used (250, 500, 1000 and 1500 μm). The warm solution was dispensed into the warm mold; then the filled mold was wrapped in aluminum foil to prevent dehydration and stored at ˜4° C. for at least 8 hours to form a solid gel. All samples were measured within 48 hours of the beginning of gel preparation.

The same photorheology apparatus was used as in Example 24-28. Briefly, Collagen gel photorheology was performed on a stress-controlled shear rheometer (TA Instrument AR1000). The lower, stationary tool was modified to deliver light to the sample. The lower plate was replaced with an aluminum plate with a 50-mm diameter quartz window positioned at the center allowing the transmission of both visible and ultraviolet (UV) light. The light intensity (0-6 mW/cm²) was controlled by adjusting the input voltage (0-16 V) provided by a power supply. The intensity profile as a function of position at the top of the quartz window was found to vary less than 5% from the value at the center of the 8-mm diameter sample area.

Oscillatory shear storage modulus measurement was performed as follows. An 8-mm diameter sample was cut from the gel sheet. The sample was placed onto the upper tool (8-mm aluminum parallel plate) to ensure proper alignment. Then the upper tool was lowered to bring the sample in contact with the lower plate. To ensure good contact between the specimen and the tools, the gap was reduced to 90% of the nominal sample thickness. To prevent gel dehydration, the sample was enclosed in a chamber containing a wet sponge that kept surrounding air saturated with water vapor.

The temperature of the sample was maintained at 24±1° C. (Omega HH059 thermocouple). Once the sample was in contact with the lower plate, a 15-minute interval was allowed for thermal equilibration before the linear storage modulus was measured at a frequency of 0.3 rad/s using an oscillatory stress amplitude of 30 Pa (in the linear regime). The storage modulus was measured for 50 minutes, including 10 minutes prior to irradiation (to verify that gelation was complete), 30 minutes during irradiation and 10 minutes after cessation of irradiation (to determine if cross-linking continued, i.e., if there is significant “dark reaction”). Each condition was repeated at least 3 times to obtain the reported mean and standard deviation.

### Example 31

### Rates of Photodynamic Cross-Linking for Different Photosensitizing Compounds

The initial modulus was in the range of 3610±760 Pa and, during the ten minutes prior to irradiation, G′ typically decreased slightly, by 50 to 200 Pa (see Appendix for individual G′ curves). The change of the storage modulus G′ during and after irradiation relative to its value at the beginning of irradiation (i.e., end of the first ten minutes, G′₁₀) is

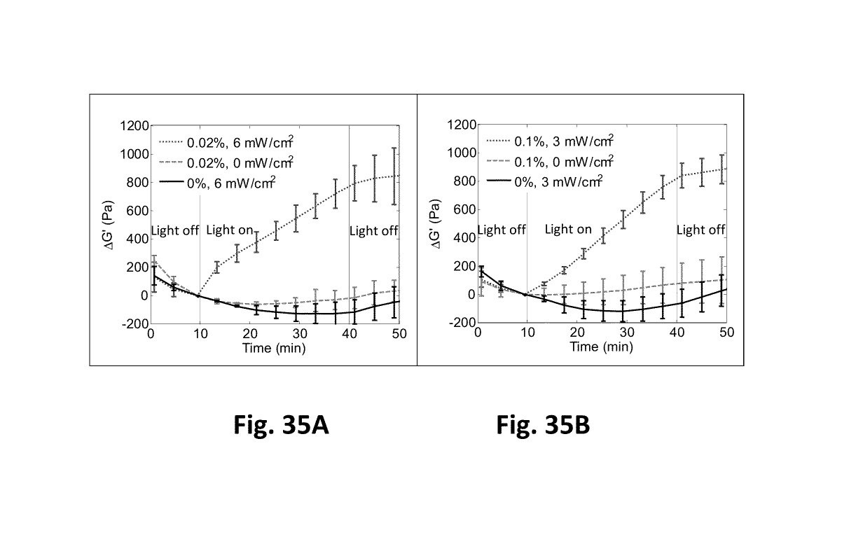

ΔG′=G′ₜ−G′₁₀ Equation (23)

where G′ₜ is the modulus at time t. For example, the storage modulus of a sample containing 0.02% eosin Y increased 793±118 Pa while exposed to 6 mW/cm² at 530±15 nm (FIG. 35A). A similar change in modulus (819±85 Pa) was observed in the gelatin containing 0.1% riboflavin sample over the 30-minute irradiation with 3 mW/cm² at 370±12 nm (FIG. 35B).

Negligible modulus change was observed over the 30-minute period in controls that either received no light or that contained no sensitizer: without irradiation ΔG′ was −23±76 Pa for (0.02% eosin Y, 0 mW/cm²) and 72±136 Pa for (0.1% riboflavin, 0 mW/cm²); and without sensitizer ΔG′ was −125±80 Pa for (0% eosin Y, 6 mW/cm²) and −74±95 Pa for (0% riboflavin, 3 mW/cm²). This demonstrates that both sensitizer and irradiation are necessary to produce the collagen cross-linking that underlies the increase in the storage modulus.

During the 10 minutes after cessation of irradiation, the modulus changes were small and indistinguishable (p-values were >0.05 for all conditions with respect to the average of all of them together) for all six conditions: 61±136 Pa for (0.02% eosin Y, 6 mW/cm²), 59±34 Pa for (0.02% eosin Y, 0 mW/cm²), 84±34 Pa for (0% eosin Y, 6 mW/cm²), 70±19 Pa for (0.1% riboflavin, 3 mW/cm²), 32±36 Pa for (0.1% riboflavin, 0 mW/cm²), and 112±30 Pa for (0% riboflavin, 3 mW/cm²). Therefore, negligible cross-linking occurs after cessation of irradiation in either system.

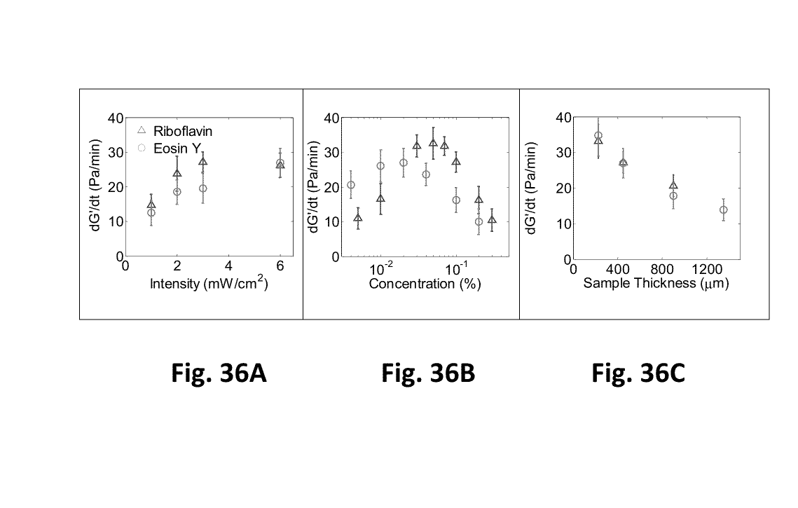

Since the presence of both drug and light are necessary for enhancing the gel's modulus, it is of interest to examine how each of these two factors affects the rate of change of G′. The rate of increase was nearly constant throughout the irradiation period. Therefore, the rate of change of G′, denoted dG′/dt, was estimated by simply dividing the overall change in G′ during irradiation by the irradiation time:

$$\frac{d {G}^{′}}{d t} = \frac{{G}^{′} ( {t}_{f} ) - {G}^{′} ( {t}_{i} )}{{t}_{f} - {t}_{i}} \qquad Equation ( 24 )$$

where tᵢ=10 minute and t_{f}=40 minutes correspond to the beginning and the end of the irradiation period. At a given photosensitizer concentration and sample thickness, the cross-linking rate (manifested by dG′/dt) increases monotonically with irradiation intensity for both eosin Y and riboflavin, approaching a plateau rate (FIG. 36A).

For a fixed sample thickness that is similar to the thickness of the cornea (450 μm) and a light intensity that saturates the cross-linking rate (6 mW/cm2 for eosin Y and 3 mW/cm2 for riboflavin), there is a distinct maximum in dG′/dt as a function of photosensitizer concentration for both eosin Y and riboflavin. The peak values are similar for the two sensitizers (dG′/dt max=27±4.1 Pa/min for eosin Y, and dG′/dt max=33±4.6 Pa/min for riboflavin). The optimal concentrations, 0.02% eosin Y and 0.05% for riboflavin, correlate with the molar absorptivity of the two compounds (below). The shapes of the peaks in dG′/dt as a function of concentration are very similar for eosin Y and riboflavin. For a photosensitizer concentration near the optimal value for a 450 μm thick specimen, dG′/dt decreased with increasing sample thickness over the range from 225 to 1350 μm for both eosin Y and riboflavin (FIG. 36C).

Collagen gel photorheology can be used to efficiently characterize the effects of irradiation intensity, photosensitizer concentration, and sample thickness on the rate of collagen cross-linking. Consistent with previous results, collagen can be cross-linked in the presence of a photosensitizer (e.g. riboflavin [40, 104, 115], eosin Y [52, 116], rose bengal [64, 65, 115, 117], methylene blue [97], and brominated 1,8-naphthalimide [118]) upon irradiation and no cross-linking was observed in the absence of either the sensitizer or irradiation[64, 65].

Collagen cross-linking can also be achieved through non-photo-activated chemical or physical techniques. Chemical agents such as glutaraldehyde and formaldehyde are very effective in cross-linking collagen but they are cytotoxic [64, 65]. Other chemical agents such as carbodiimide and its derivatives are more biocompatible but the reactions are very slow [65]. Collagen cross-linking with physical techniques such as heat, UV irradiation, and gamma irradiation do not form stable cross-links[64]. Photo-activated cross-linking has been demonstrated to be biocompatible [64, 115]. Using photo-activated molecules decouples reaction and diffusion and confers spatial control of cross-linking. Diffusion can occur, then reaction can be initiated by light. Treatment can be targeted to specific locations by delivering the drug and then irradiating selected locations to avoid cross-linking adjacent tissues which can lead to adverse effects. Light activation of the drug also enables control over the depth of cross-linking inside the tissue. The photosensitizing drug can be delivered then allowing time for diffusion to achieve a desirable drug concentration profile before irradiating. In addition, the use of light activation also enables control over the extent of cross-linking by selecting irradiation parameters (intensity and duration). Photo-activated corneal cross-linking efficacy depends on the collagen cross-linking rate.

The non-monotonic concentration dependence of photo-activated reactions is well known in systems ranging from photodynamic therapy to curing polymers via photopolymerization^{[119-122]}. The optimal concentration reflects the trade-off between the number of sensitizer molecules present and the attenuation of light by the sensitizer: at low concentration, the reaction is limited by the amount of photosensitizer present; beyond the optimal concentration, the reaction is limited by the penetration depth of the irradiation.

The fraction of the sample that receives irradiation of the order of that incident on its surface is characterized by Λ, the ratio of the optical penetration depth (Lₚ, at which the intensity has been attenuated by 1/e) to the sample thickness (L), which decreases with increasing photosensitizer concentration in the sample:

$$Λ = \frac{{L}_{p}}{L} \qquad Equation ( 25 )$$

The light intensity profile in a sample with uniform concentration C of photosensitizer is given by:

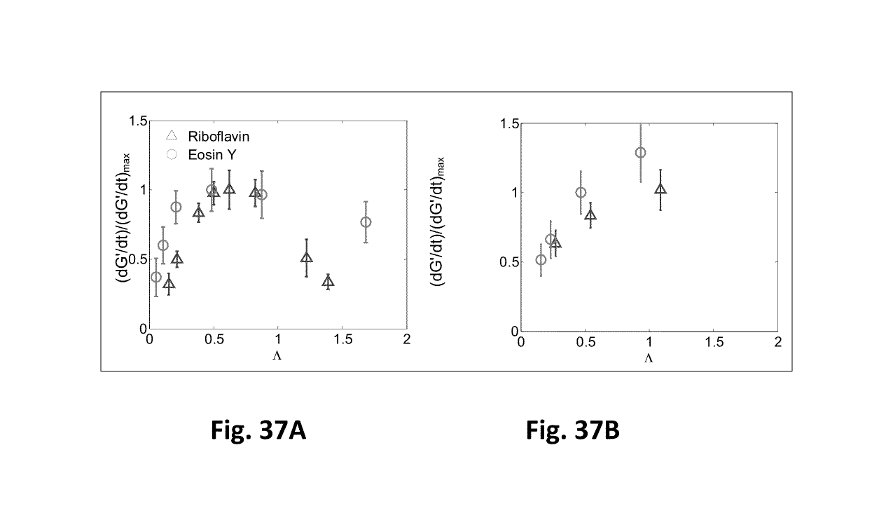

I(z)=Iₒe^{-(μ+Cε)z} Equation (26)

where I(z) is the intensity at depth z, Iₒ is the incident intensity, μ is the sample's absorptivity, and ε is the photosensitizer's molar absorptivity. The normalized cross-linking rate characterized by (dG′/dt)/(dG′/dt)ₘₐₓ initially increases as Λ increases by decreasing concentration at fixed sample thickness until more than half the thickness of the sample receives intensity greater than Iₒ/e (i.e., until Λ>½), where a maximum rate occurs at approximately Λₘₐₓ=0.6 to 0.7 for both eosin Y and riboflavin (FIG. 37A). Beyond Λₘₐₓ the rate decreases with increasing Λ, reflecting the loss of efficacy at low sensitizer concentration. When Λ is increased by decreasing sample thickness, (dG′/dt)/(dG′/dt)ₘₐₓ increases until it saturates at the value associated with uniform light intensity throughout the sample.

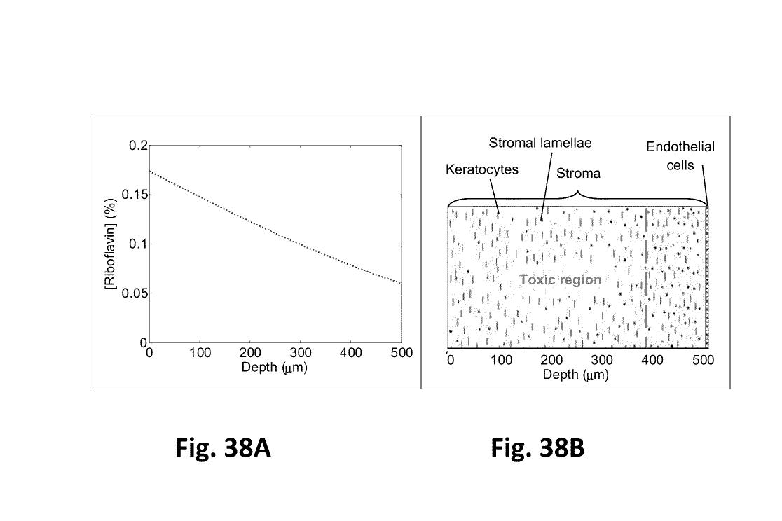

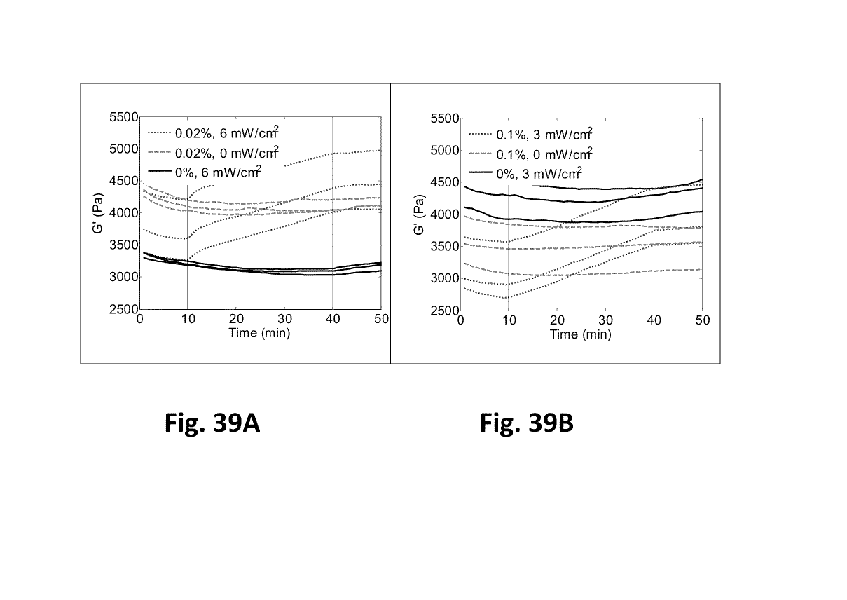

The keratoconus treatment protocol approved for clinical use in Europe and undergoing clinical trials in the United States uses 0.1% riboflavin concentration and 3 mW/cm² at 370 nm. The drug is applied topically every 2 minutes for 30 minutes followed by UV irradiation for 30 minutes while applying drops every five minutes. Using the riboflavin diffusion coefficient (D=79 μm²/s) obtained in Example 32-34, the concentration profile after 30 minutes of drug application yields a relatively uniform concentration throughout the thickness of the cornea, similar to the situation in the collagen gel experiments. Using the riboflavin partition coefficient (k=1.7) obtained in Example 32-34, the average concentration of riboflavin in the cornea is predicted to be 0.12% (varying from 0.17% at the anterior surface to 0.06% at the posterior surface, FIG. 38A). In collagen gel samples with 0.1% riboflavin, the rate increases with intensity up to 3 mW/cm² and then saturates (FIG. 36A). Thus, the clinical irradiation intensity (3 mW/cm²) corresponds to the lowest value which induces the highest cross-linking rate. The clinical protocol uses a riboflavin concentration that is not optimal (by interpolation, 0.12% riboflavin concentration yields a cross-linking rate that is approximately 78% of the optimal rate that would be achieved using 0.05% riboflavin, see FIG. 36B). The selection of a greater-than-optimal concentration of riboflavin may be due to the toxicity of riboflavin and UVA light: the riboflavin concentration is chosen based on the need to attenuate UVA light to a safe level, protecting the endothelium in patients with stromal thickness greater than 400 μm^{[47, 71, 108, 110]}; patients with stromal thickness less than 400 μm are excluded from treatment^{[71]}.

Unlike riboflavin/UVA treatment, collagen cross-linking activated by eosin Y using visible light has relatively low toxicity (Example 36). Therefore, the combination of eosin Y and visible light can be optimized for efficacy (Example 35). The low cytotoxicity of eosin Y and visible light may expand the range of patients who can safely receive corneal cross-linking treatment to include cases of advanced keratoconus or post-LASIK ectasia, in which corneal thickness is frequently less than 400 μm^{[112]}.

Photorheology can be used to efficiently characterize the effects of treatment parameters (including photosensitizer concentration and irradiation intensity) on the cross-linking rate of therapeutic collagen cross-linking. In the specific case of eosin Y activated by green light, photorheology indicates that the rate and extent of collagen cross-linking can match those of riboflavin activated by UVA at the conditions that have proven to be clinically efficacious. The kinetic data provided by photorheology can be used in a predictive model of collagen cross-linking to anticipate the safety and efficacy of proposed treatment protocols (Example 35).

### Example 32

### Using Diffusion Coefficient and Partition Coefficient to Determine Distribution of within a Tissue and Delivery of the Photosensitizing Compound to a Tissue

Topical drug delivery is the dominant route for ocular drug delivery due to the accessibility of the front of the eye, the minimal risk of infection, and the ability to transfer drug into the ocular coat (cornea and sclera), anterior chamber and its associated tissues[123-126]. To reach any of these tissues, topically applied drug must penetrate the ocular coat; therefore, it is important to understand the transport across these tissues. Here, we focus on the transport of drugs through the ocular coat with specific interest in treating diseases associated with progressive thinning and weakening of the ocular coat, including keratoconus, post-LASIK ectasia and degenerative myopia. Photo-activated cross-linking treatments have been proposed for halting the progression of these diseases by strengthening the weakened tissue [48, 71-74, 127]. The safety and efficacy of cross-linking treatments depend on the local drug concentration and light intensity as a function of depth into the tissue. This study aims to characterize the transport of both riboflavin, which is currently being used clinically, and eosin Y, which is a less toxic photosensitizer (Example 36). The development of less toxic routes to tissue cross-linking would enable treatment of patients with post-LASIK ectasia and degenerative myopia, in addition to keratoconus.

Keratoconus is a bilateral corneal thinning disorder with a prevalence of 1 out of 2,000[17]. This eye disease is characterized by progressive corneal thinning and protrusion[17, 67, 128]. Post-LASIK ectasia is a complication of refractive surgery that results in corneal thinning and protrusion, similar to keratoconus [68, 69, 129]. Post-LASIK ectasia has an incidence of 1 out of 2,500 LASIK surgical procedures [130]. Degenerative myopia is associated with the progressive thinning and stretching of the posterior sclera[70, 131]. It is the leading cause of blindness in China and is ranked 7^{th} in the United States[10].

Therapeutic cross-linking using riboflavin activated by UVA irradiation pioneered by Wollensak, Spoerl, and Sieler has been shown to be promising for halting the progression of keratoconus [34, 71]. Riboflavin/UVA treatment has not yet been successfully demonstrated in treating corneal ectasia for patients with cornea thinner than 400 μm [47, 71, 75] or degenerative myopia[48, 66]. Motivated by the need for a safer cross-linking treatment, we have investigated eosin Y, a visible light activated photosensitizer that has been approved by the FDA for use in the body[51].

Even though riboflavin/UVA treatment has been performed on keratoconus patients for almost a decade[41], the transport of riboflavin into the corneal stroma has not yet been quantified. Although two studies examine the concentration profile of riboflavin as a function of depth in the cornea, Sondergaard et al[132] using confocal fluorescence microscopy and Cui et al[133] using two-photon excited fluorescence technique, the reported maximum concentration of riboflavin in the tissue differs by two orders of magnitude. There is also a discrepancy in the reported shapes of the concentration distribution of riboflavin along the tissue depth.

Fundamentally, knowledge of the diffusion and partition coefficient of the molecule in the tissue is needed to predict the time evolution of the amount and distribution of drug transferred into the tissue. We were unable to find any literature on the diffusion and partition coefficient of riboflavin in the cornea or sclera. Riboflavin has a similar molecular structure and molecular weight to fluorescein, a compound used extensively in ophthalmology, so its diffusion and partition coefficient has been measured in various studies [134-136]. A theoretical concentration profile of riboflavin in the cornea has been predicted before using fluorescein's diffusion coefficient (D=65 μm²/s) and assuming a partition coefficient of 1 [45].

Transport through the ocular coat tissues has been commonly studied using an Using chamber to determine the permeability of the compounds of interest across the cornea or sclera[137-139]. Permeability is the product of the partition coefficient and diffusivity divided by the tissue thickness[22]. Other techniques have been developed to determine the partition coefficient and diffusivity. One technique involves applying drug to the end of a strip of cornea or sclera and monitoring the concentration at various positions along the strip as a function of time either by measuring the tissue fluorescence or sectioning the tissue and performing extraction. The concentration profile was fit to a 1-dimensional diffusion model [135, 136, 140, 141] to determine the partition and diffusion coefficient. Another technique entails immersing a cross-section of the sclera in a solution to saturate the tissue, then transferring the cross-section into a solute-free solution and measures the rate of solute leaving the cross-section and then fitting the data to a diffusion model to determine the diffusion coefficient[140].

Our study examined the transport of molecules into an intact globe, which mimics in vivo conditions and avoids damaging the tissue structure from cutting. After drug was delivered to the eye, the cornea or sclera was isolated to extract the molecules delivered to the tissue as a function of contact time with the drug solution. We then applied a diffusion model to fit the data. From this, we were able to determine the partition coefficient, k and diffusion coefficient, D. Absorbance measurements of the tissue cross-sections were performed to check for consistency with the extraction measurements. The absorbance method for quantifying the amount of drug delivered was also used to compare different delivery techniques (drops and different formulations of viscous gels) to determine which would be worthy of pre-clinical evaluation.

### Example 33

### Determine Diffusion Coefficient and Partition Coefficient of Photosensitizing Compounds and their Delivery to the Tissue and Distribution

Drug Diffusion into the Cornea—

Siena for Medical Science supplied porcine eyes from 3-4 month old swine. Eyes were stored in ocular balanced saline solution on ice until use within 48 hours post mortem. Corneal tissues were all clear with no signs of edema. The epithelial cell layer was removed by scraping with a scalpel. Each eye was immersed in 30 mL of drug solution, either 0.289 mM (0.02%) eosin Y (Sigma Aldrich E6003) solution in Dulbecco's phosphate buffered saline (DPBS, Sigma D8662) or 0.289 mM (0.0138%) riboflavin 5′-monophosphate sodium salt (riboflavin, Fluka 77623) solution in DPBS. The drug solution containing the eye was gently agitated using a rocker for a specified “drug contact time” (t_{c,cornea} ranging from 0 to 4 hours). After the drug contact time, the eye was removed from the drug solution, and excess solution on the cornea was dabbed away with a Kimwipe. The eye was dissected using a scalpel blade and a pair of scissors to cut around the corneoscleral limbus to separate out the cornea. The tissue section was placed onto a trephine punch to cut out a 9.5-mm diameter corneal cross-section. The sizes of the cross-sections were very consistent with mass of 95±12 mg for eyes with t_{c,cornea}=0 hr.

Drug Diffusion into the Sclera—

Orbital tissues were removed with scissors to expose the sclera. Each eye was placed into 30 mL of a drug solution (either Eosin Y or riboflavin) and gently agitated using a rocker for a specified contact time (t_{c,sclera} ranging from 0 to 120 hours). After the contact time, the eye was removed from the drug solution, and excess solution on the sclera was dabbed away with a Kimwipe. The eye was dissected using a scalpel blade and a pair of scissors to obtain a posterior scleral section on the temporal side near the optic nerve. The tissue section was placed onto a trephine punch to cut out a 9.5-mm diameter cross-section.

Quantitative assay of the amount of drug delivered—Experiments were performed for five drug contact times (four samples each): for the cornea, t_{c,cornea}=0.25, 0.5, 1, 2, and 4 hrs, and for the sclera, t_{c,sclera}=0.5, 2, 4, 7.5, and 120 hrs. Each experiment was repeated four times. For each experiment, the 9.5-mm diameter tissue cross-section obtained as described above was placed into a 50 mL centrifuge tube and immersed in 50 mL of extractant (doubled-distilled water for cornea or DPBS for sclera) then placed on a rocker to extract the drug molecules (FIG. 40). DPBS was used instead of water to extract drug from the sclera because eosin Y did not partition favorably from the sclera into water. After a first extraction time t_{e,1} of 8 hours, the tissue specimen was then transferred into another 50 mL of fresh extractant and the extract was retained for analysis.

The concentration of the drug in the extract was determined using fluorimetry. Eosin Y was excited at 514 nm and the fluorescence was detected at 534 nm; for riboflavin, the excitation wavelength was 466 nm and detection wavelength was 523 nm. The fluorescence of each extract was measured and concentration was determined based on calibration curves prepared for each compound using a series of solutions with known concentrations. The extraction process was repeated using successively longer extraction times until there was no detectable fluorescence in the supernatant. For the cornea, three extractions were sufficient, with t_{e,1}=8, t_{e,2}=24 and t_{e,3}=48 hours (a total extraction time of 48 hours). For the sclera, five extractions were required with t_{e,1}=8, t_{e,2}=24, t_{e,3}=48, t_{e,4}=72, t_{e,5}=96, and t_{e,6}=120 hours (a total extraction time of 120 hours). The final extraction time was only needed for sclera specimens that had been kept in contact with eosin Y t_{c,sclera}=120 hours.

Assessing residual eosin Y remaining in tissue after extraction—Eosin Y is known to bind to collagen which makes up more than 68% of the cornea's dry mass and more than 80% of the sclera's dry mass. Therefore, light absorption measurements were performed to exclude the possibility that a significant amount of eosin Y remained in the tissue after extraction. One cornea specimen was prepared as above for each of the following drug contact times: t_{c,cornea}=0.25, 1, and 4 hours. Before placing the corneal cross-section into the first extractant, its UV-vis absorption spectrum was measured. There was a distinct peak at 525 nm with absorbance value greater than 2 for all samples (i.e., transmitted intensity was <0.01 of the incident intensity). Eosin Y was then extracted from the corneal cross-sections into double distilled water using three successive extractions with t_{e,1}=8, t_{e,2}=24 and t_{e,3}=48 as described above. After the last extraction, UV-vis absorption spectrum of the corneal cross-section was measured again. Even though the corneas were cloudy after the extraction process, the absorbance values were ˜1.2 (i.e. transmitted intensity was ˜5% of the incident intensity), and the peak at 525 nm was no longer present in any of the three cornea specimen. This demonstrates the amount of eosin Y remaining in the tissue specimen after the extraction procedure is negligible compared to the amount extracted. Riboflavin does not bind to collagen; therefore, none is expected to remain in the tissue after extract is complete.

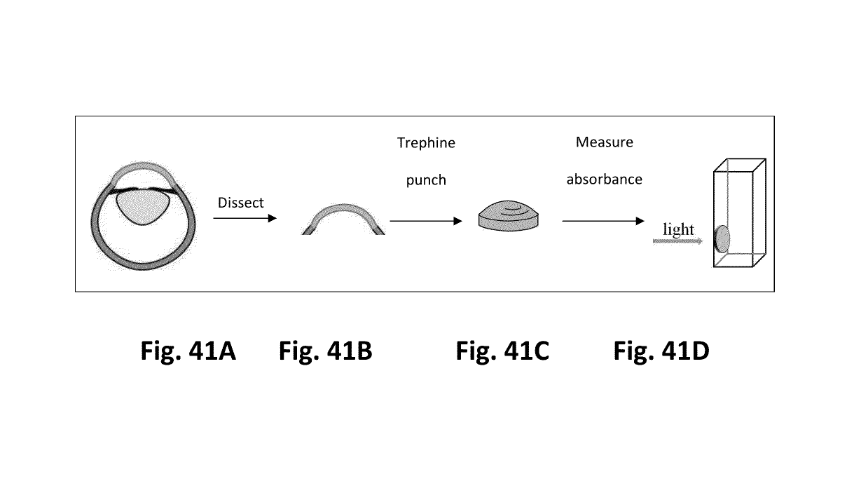

Absorbance measurement to determine the amount of drug delivered—The extraction method provides a quantitative measure of the number of drug molecules delivered; however, the procedure 48 hours to 120 hours. For determining the number of molecules transferred to the cornea as a function of the delivery protocol and delivery vehicle, light absorption measurement suffice to characterize the number of drug molecules delivered to the cornea. Once the drug delivery step is complete, a 9.5-mm diameter cross-section of the central cornea was obtained as described above (FIGS. 41A-D). After taking a “blank” absorbance reading with the empty cuvette, the corneal section is placed into the cuvette and the sample's absorbance was measured at the wavelength of the maximum absorbance of the drug (e.g., at 525 nm for eosin Y) in the tissue. Using the calibration curves described above, the amount of drug delivered was calculated from the absorbance of the corneal section.

The number of molecules delivered to each cornea was calculated from the absorbance using the following equations

Aₒ=εₒ·L Equation (27)

where Aₒ is the apparent absorbance of the control sample soaked in DPBS for 5 minutes, εₒ is the extinction coefficient of the cornea, and L is the thickness of the sample.

A=εₒ·L+ε_{EY}·C·L Equation (28)

where A is the absorbance of the sample with eosin Y and ε_{EY} is the extinction coefficient of eosin Y, C is the of eosin Y concentration inside the tissue. Subtracting Equation 28 from Equation 27 and rearrange yields

$$C \cdot L = \frac{A - {A}_{o}}{{ɛ}_{EY}} \qquad Equation ( 29 )$$

The product of concentration and sample thickness is the number of drug molecules delivered per unit area.

This method was used to compare different drug delivery techniques in vitro to determine which would be worthy of preclinical evaluation in vivo: 1) immersion in drug solution, 2) topical drops of drug solution, and 3) topical application of a gel. These three drug delivery techniques were examined using porcine eyes from 3-4 month old swine (Sierra for Medical Science) stored in saline on ice until use within 48 hours post mortem. The epithelial cell layer was removed from the cornea by scraping with a scalpel. Drug was delivered to the cornea using one of the following techniques (or the corresponding control):

The three drug delivery techniques were studied:

1) Immersion: Each eye was immersed in 30 mL of 0.289 mM eosin Y solution that was gently agitated using a rocker. After 5 minutes, the eye was removed from the eosin Y solution, and excess solution on the cornea was dabbed away with a Kimwipe. Control eyes were placed in 30 mL of DPBS solution instead of eosin Y solution.

2) Topical drops: Drops of 0.289 mM eosin Y in DPBS solution were applied to the cornea every minute for 5 minutes. Excess solution on the cornea was dabbed away with a Kimwipe.

3) Topical gel: Four different viscosity enhancers were examined, each at a concentration such that the gel would remain on the cornea for 5 minutes: 2% hyaluronic acid (HA), 3% carboxymethylcellulose (CMC), 3% sodium alginate (SA), and 3% methylcellulose (MC) each in DPBS. Approximately 0.5 mL of 0.289 mM eosin Y gel was applied to the cornea using a syringe and then the gel was spread evenly over the cornea and limbus using a spatula. Hyaluronic acid provided a gel that was free of bubbles, but it was somewhat difficult to spread into an even layer. Sodium alginate and methylcellulose gels were easy to spread, but retained air bubbles. Carboxymethylcellulose provided a gel free of bubbles that spread easily with a spatula. After 5 minutes, the gel was removed from the cornea with a spatula and the site was quickly rinsed with ˜2 mL of DPBS. Excess solution was then dabbed away with a Kimwipe.

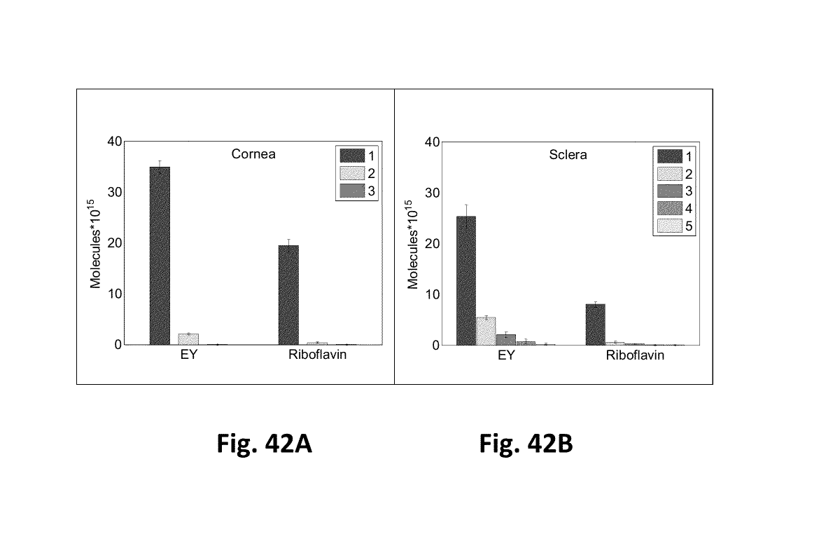

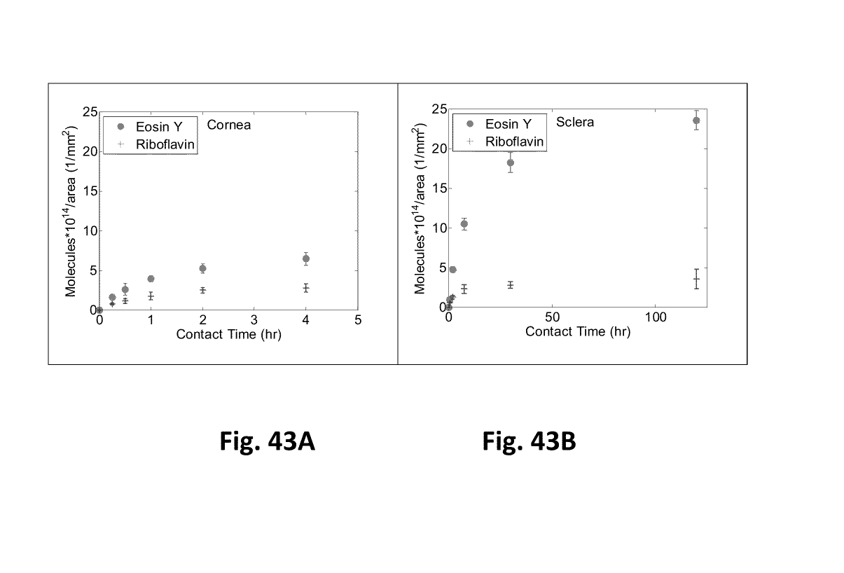

Cornea samples required two extracts to remove the delivered drug molecules (FIGS. 42A-42B). For all contact times examined (0.25, 1, 2, and 4 hours), the second extract contained much less drug than the first (for eosin Y, 3 to 6% and for riboflavin, 1.6 to 2.5% of the first extract) and the third extract had negligible drug (fluorescence value was similar to that of doubled-distilled water, indicating a drug concentration less than 1% of the first extract). Therefore, we approximate the total number of drug molecules delivered to the cornea during t_{c} as the sum of the number of drug molecules in the three extracts (FIGS. 43A-43B).

Sclera samples required more extractions than cornea samples, particularly for eosin Y. For all contact times examined (0.5, 2, 4, 7.5, 30, and 120 hours), the amount of eosin Y in the second extract was a substantial fraction of that in the first extract, between 10 to 45% (e.g., for 2 hours contact time, the second extract contained approximately 20% as much as the first extract, FIG. 42B). As the duration of the extraction step increased, the ratio of the content of eosin Y in the successive extracts approached a constant value of approximately ⅓; specifically, relative to the first extract, the amount of eosin Y in the subsequent extracts was between 3 to 22% in the 3^{rd}, between 2 to 8% in the 4^{th}, between 0-2% in the 5^{th} and ≦1% in the 6^{th}. Riboflavin was much more readily extracted from the sclera: relative to the first extract, the second extract contained only 3 to 6%, the 3^{rd} extract contained between 1 to 3%, and the 4th extract ≦1%. Therefore, we also approximate the total number of drug molecules delivered to the sclera during t_{c} as the sum of the number of drug molecules in all the extracts (FIG. 43B).

The total drug delivered per unit area of contact, estimated as the sum of the number of drug molecules in all extracts, normalized by the cross-sectional area of the sample, increases with drug contact time (FIGS. 43A-43B). The value levels off at long time as the system approaches equilibrium partitioning of drug between the tissue and the solution. For the cornea, the initial increase with drug contact time levels off at 2 hours for both eosin Y and riboflavin, indicating that the two molecules have similar diffusivities in the cornea; the long time asymptotes show that the eosin Y partitions more favorably into the cornea than riboflavin does, by approximately a factor of 2 (FIG. 43A). For the sclera, much longer time was required for the concentration to level off (note different time scales for part a and b of FIGS. 43A-43B). Furthermore, the time scales were not the same for the two drugs (approximately 30 hours for eosin Y and approximately 7.5 hours for riboflavin); the sclera also showed a more dramatic difference in affinity for eosin Y and riboflavin—approximately 7-fold greater for eosin Y than riboflavin at the long time asymptotes (FIG. 43B).

The equilibrium partitioning of riboflavin between the drug solution and the tissue was quite similar for the cornea and the sclera. There is a pronounced difference in the equilibrium partitioning of eosin Y for the cornea and sclera with the sclera being much more favorable than the corneal stroma. Despite, the much greater affinity of the sclera for eosin Y, the rapid rate of transport into the cornea led to greater eosin Y uptake by the cornea than the sclera at short contact times (0.5 and 2 hours). The situation reversed at long contact times (4 hours in the cornea and 120 hours in the sclera), when significantly more eosin Y was absorbed by the sclera than the cornea. In contrast, approximately the same amount of riboflavin was absorbed by both of these tissues at long contact times.

### Example 34

### Evaluation of Partition Coefficients and Diffusion Coefficients

A quantitative description of the number of drug molecules delivered to targeted tissue is crucial in the drug delivery aspect of developing safe and effective therapeutics. In addition to the quantity of drug delivered, photo-activated therapy is sensitive to the distribution of drug inside the tissues. Following Maurice[134], Nagataki et al[135, 136], Prausnitz et al[141, 142] Applicants found that a simplified diffusion model provides a good description of the experiment results (see below) and, therefore, use such diffusion model to evaluate the partition and diffusion coefficients. The observed accord between the model and the present experimental results also indicates that the model can be used to predict the drug concentration profile inside the tissue as a function of treatment parameters (i.e. drug concentration, drug contact time, and the delay time from the drug application to drug activation via irradiation, which is discussed in Example 35).

Porcine eyes closely resemble a sphere with diameter ˜25 mm. The cornea and sclera are the targeted tissues and both have thicknesses that are on the order of 1 mm. Since these tissue thicknesses are less than a tenth of the diameter of the eye, they are modeled as semi-infinite slabs. Each tissue is approximated as a uniform material. Fick's diffusion equation is given by

$$\frac{\partial C ( z )}{\partial t} = D \frac{{\partial }^{2} C ( z )}{\partial {z}^{2}} \qquad Equation ( 30 )$$

where C(z,t) is the drug concentration inside the tissue, t is the time since the exterior surface of the tissue was placed in contact with the drug solution, z is the distance into the tissue from its exterior surface, and D is the diffusion coefficient. An initial condition and two boundary conditions are needed: the initial condition has no drug in the tissue, the concentration just inside the tissue (at z=0) is given by the product of the partition coefficient and the concentration of the drug in the solution, and the concentration falls to zero far from the surface of the tissue. For short contact times, the concentration falls to zero before the profile reaches the endothelium. For longer contact times, the boundary condition that the concentration falls to zero far into the system is still used as a first approximation (neglecting the change in material properties at the endothelium and neglects different transport processes in the aqueous or vitreous).

Initial condition at t=0,C=0 for all z≧0 Equation (30.1)

Boundary condition at z=0,C=k·Cₛₒₗᵤₜᵢₒₙ for t>0 Equation (30.2)

Boundary condition at z=∞,C=0 for all t Equation (30.3)

where k is the partition coefficient and Cₛₒₗᵤₜᵢₒₙ is the concentration of the drug solution applied. Applying the initial and boundary conditions to the diffusion equation, the concentration profile is given by

$$C ( t , z ) = k \cdot {C}_{solution} \cdot erfc ( \frac{z}{\sqrt{4 Dt}} ) \qquad Equation ( 31 )$$

where erfc is the complementary error function. Using this theoretical concentration profile, the total number of drug molecules present in the tissue can be calculated by integrating over the thickness of interest

$$\frac{molecules}{area} = {\int }_{0}^{L} k \cdot {C}_{solution} \cdot erfc ( \frac{z}{\sqrt{4 Dt}} ) \cdot d z \qquad Equation ( 32 )$$

where area represents the cross-sectional area of the tissue sample and L is the thickness of the tissue sample. At long drug contact time, the predicted concentration changes very slowly and approaches a value governed by the partition coefficient. At short drug contact times, the rate of change is strong and largely determined by the diffusivity. The measured number of molecules delivered per area is compared to the model to deduce the values k and D that minimize the mean-square deviation, S, between the predicted and observed values for a given drug-tissue pair:

$$S = {\sum }_{i = 1}^{N} {[ {( \frac{molecules}{area} )}_{i} - {( \frac{molecules}{area} )}_{measured , i} ]}^{2} \qquad Equation ( 33 )$$

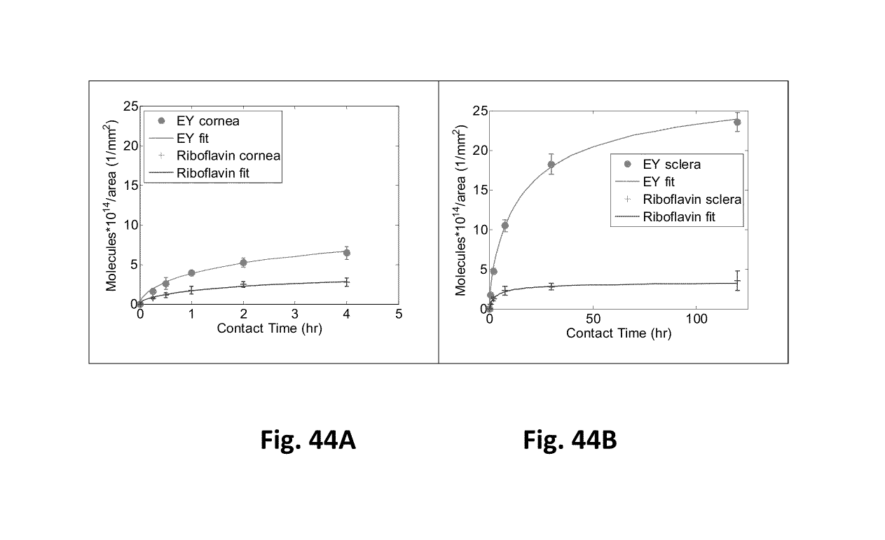

with i being the individual data point and N being the total data points used for the fit. Data fitting was performed by writing a program in MATLAB software. The quality of fit provided by the resulting values of k and D (Table 12) is good (all predicted values are within 15% of the average measured values), suggesting that the approximations made are acceptable (FIGS. 44A-44B).

TABLE 12
Values of diffusivity, D and partition coefficient, k for eosin Y
and riboflavin penetrating into the cornea and sclera from DPBS.

| | | D (μm²/s) | k |
|---|---|---|---|
| Cornea | Eosin Y | 62 ± 22 | 4.3 ± 0.7 |
| | Riboflavin | 79 ± 22 | 1.7 ± 0.2 |
| Sclera | Eosin Y | 6.2 ± 1.7 | 13.0 ± 1.1 |
| | Riboflavin | 27 ± 8.4 | 1.5 ± 0.6 |

For the cornea, eosin Y's partition coefficient is greater than that of riboflavin (k_{c, EY}=4.3, k_{c, riboflavin}=1.7), in accord with the difference noted above in their concentrations observed at long time. Eosin Y's diffusion coefficient in the corneal stroma is similar to that of riboflavin (D_{c, EY}=62 μm²/s, D_{c, riboflavin}=79 μm²/s), in accord with the similar time course for sorption noted above.

In contrast to the cornea, the difference in behavior between the two drugs is pronounced in the sclera. The partition coefficient for eosin Y is almost ten-times greater than that of riboflavin (k_{s, EY}=13, k_{s, riboflavin}=1.5), consistent with the much greater concentration of eosin Y than riboflavin at long times. The diffusion coefficient of eosin Y in the sclera is much less than that of riboflavin (D_{s, EY}=6.2 μm²/s, D_{s, riboflavin}=27 μm²/s), consistent with the much longer time required for eosin Y to reach its long-time asymptotic concentration than riboflavin.

Comparing eosin Y's behavior in the cornea and sclera shows its diffusivity is significantly greater in the cornea than in the sclera (D_{c, EY}=62, D_{s, EY}=6.2) and that it partitions into the cornea significantly less favorably than the sclera (k_{c, EY}=4.3, k_{s, EY}=13.0). In contrast to eosin Y, riboflavin's diffusion coefficient in the cornea is only three-times greater in sclera (D_{c, riboflavin}=79 μm²/s, D_{s, riboflavin}=27 μm²/s), and its partition coefficient is almost the same in the two tissues (k_{c, riboflavin}=1.7, k_{s, riboflavin}=1.5).

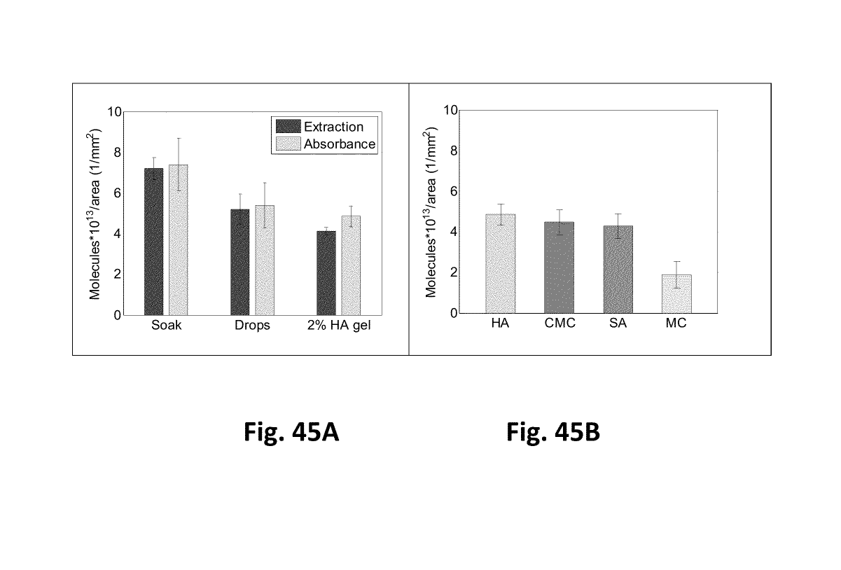

The extraction method and absorbance method for quantifying drug delivery were compared using three different delivery techniques: soak, drops, and 2% hyaluronic acid gel for 5 minutes (as described earlier). The two methods gave consistent results (FIG. 45A), validating the absorption measurement as a tool to study drug delivery.

To facilitate controlled application of the drug formulation on the cornea, it is of interest to increase the viscosity of the solution. Here we compare four clinically relevant options: 2% hyaluronic acid (HA), 3% carboxymethylcellulose (CMC), 3% sodium alginate (SA), and 3% methylcellulose (MC). Three out of the four types of gel (hyaluronic acid, carboxymethyl cellulose, sodium alginate) delivered approximately the same amount as topical drops (FIG. 45B). Although methylcellulose gel delivered less than half as much drug as the other formulations, this may simply be due to the difficulty of removing air bubbles from the resulting gel.

Using the extraction technique to quantify the amount of drug as a function of tissue contact time and fit the data with a diffusion model, we calculated the partition coefficient and diffusion coefficient. These values enable the prediction of quantity and distribution of drug along the tissue depth, which are very important to safety and efficacy in therapeutics. Corneal and scleral transport has been predominantly studied using an Using Chamber to measure the permeability of molecules through these two tissues. Permeability describes the rate of transport at steady state, but steady state is not always reached in clinical treatments so the transient transport is a better description of these systems.

To compare the values obtained in this study to those in the literature, the partition coefficient and diffusion coefficient are related to the permeability, P, by the following[22]

$$P = \frac{kD}{L} \qquad Equation ( 34 )$$

The calculated permeability through porcine cornea for riboflavin and eosin Y are 0.16 and 0.31 μm/s, respectively. Permeability measurements through the corneal stroma have mostly been studied in rabbit corneas[143]. For molecules with similar sizes (4.0 to 5.1 Å), reported permeability through rabbit stroma for 15 compounds range from 0.30 to 0.58 (μm/s)[143]. Permeability is inversely proportional to thickness, and after taking into account porcine stromas are 2.5 times thicker than rabbit stromas[144, 145], corneal permeability values of eosin Y and riboflavin obtained from this study are similar to the range of reported values in the literature (thickness corrected range: 0.13 to 0.25 μm/s).

For the sclera, riboflavin permeability is 0.050 μm/s and eosin Y is 0.099 μm/s. For molecules of similar sizes (3.3 to 4.9 Å), reported permeability through rabbit sclera is 0.25 to 0.71 μm/s for 4 compounds, human sclera is 0.15 to 0.44 μm/s for 6 compounds, and bovine sclera is 0.065 to 0.13 μm/s for 2 compounds[143]. Scleral permeability values are also similar to reported values in the literature if tissue thicknesses [146] are taken into account (thickness corrected range: 0.050 to 0.14 μm/s for rabbit sclera, 0.060 to 0.18 μm/s for human sclera, 0.042 to 0.084 μm/s for bovine sclera).

Permeability describes the transport through the ocular coat once steady state is achieved. In order to quantify the drug transport inside the ocular coat during transient transport, the partition coefficient and diffusion coefficient are necessary. The partition coefficient is the ratio of the drug concentration inside the tissue to the drug concentration in the saline drug solution at equilibrium. In the ocular coat, this coefficient depends on the binding interaction between the tissue and the drug molecule, the drug's lipophilicity and charge[141, 142].

For a given molecule, the binding interaction depends on the tissue properties. The corneal stroma and sclera are very similar in structure. They are both composed of predominantly water, collagen, and proteoglycans. Collagen fibrils are embedded in a gel matrix made up of proteoglycan and water. For hydrated corneal stromas (condition during experiments), the estimated volume fraction of water is 89.7%, collagen is 7.3% and the rest of the volume fraction consists of proteoglycans, non-collagenous free proteins, and salts[22]. For scleras, the estimated volume fraction of water is 77.7%, collagen is 18.4%, and the rest also consists of proteoglycans non-collagenous free proteins, and salts[22].

Eosin Y is known to bind to proteins unselectively including collagen[107, 147]. Its binding affinity for collagen results in a very favorable partitioning into the cornea (4.3±0.7) and sclera (13.0±1.1). Eosin Y's partition coefficient in the cornea is 3 times less than that of the sclera, which correlates with the volume fraction of collagen present in these tissues. The collagen volume fraction of the cornea is 2.5 times less than that of the sclera. Another molecule that is known to bind to collagen is sulforhodamine[148-150]. Its partition coefficient in the sclera is 13.6[141], which is very similar to that of eosin Y. Riboflavin is not known to bind to collagen, resulting in a significantly less favorable partitioning into the cornea (1.7±0.2) and sclera (1.5±0.6) compared to eosin Y. Fluorescein is a molecule that is not known to bind to collagen and its partition coefficient for the cornea has been report to be between 1.20[136] and 1.33[135], which is very similar to that of riboflavin.

Other than binding interactions, a molecule's lipophilicity and charge are also expected to affect the partition coefficient. Both eosin Y and riboflavin readily dissolve in water and both are negatively ionized in solution at physiological pH[107, 147, 151]. Since both of these properties are similar, the binding interaction is predominantly responsible for the differences in partitioning of eosin Y and riboflavin into the cornea and sclera.

The partition coefficient is very important in calculating the transport of drug into the tissue. For a given drug solution and contact time, the concentration everywhere along the tissue is proportional to the partition coefficient (Equation 31). In safety studies, riboflavin concentration was calculated assuming a partition coefficient of 1 [45], which leads to an error of 70% lower than the actual concentration. This can be a significant error in studying the toxic dose of riboflavin combined with UV irradiation.

While, the partition coefficient determines how much molecules prefer being inside the tissue compared to the DPBS solution, the diffusion coefficient determines how rapidly molecules travel through the tissue. With respect to diffusion, the stroma and sclera have been modeled as a matrix consisting of impermeable collagen fibrils embedded in a gel matrix constituted of proteoglycan and water[22]. A molecule's diffusion rate through the stroma and sclera depends on its binding interaction with the tissue, the volume fraction of the impermeable collagen fibrils in the tissue matrix, and its molecular size[22].

The diffusion coefficient, D, evaluated from our model is essentially an effective diffusion coefficient of the molecule diffusing through the tissue including the binding effects. For a given tissue, a higher affinity for protein binding leads to a lower effective diffusion coefficient (Table 12). In order to determine how eosin Y and riboflavin's diffusion coefficients are influenced by the collagen volume fraction in the tissue and the solute's molecular size, we examine diffusion without binding effects. A one-dimensional diffusion model with binding interactions developed by Jiang et al[141] accounts for the binding effect separately from the diffusion process. The result from this model is similar to ours. The effective diffusion coefficient, D is related to the diffusion coefficient without the binding effect, D_{ab} by the following expression

$$D = {D}_{ab} \frac{{K}_{eq}}{1 + {K}_{eq}} \qquad Equation ( 35 )$$

where K_{eq} is the ratio of free-to-bound molecules in the tissue at equilibrium,

$${K}_{eq} = \frac{{C}_{free}}{{C}_{bound}} \qquad Equation ( 36 )$$

At equilibrium, the partition coefficient can be expressed as

$$k = \frac{{C}_{tissue}}{{C}_{solution}} = \frac{{C}_{free} + {C}_{bound}}{{C}_{solution}} \qquad Equation ( 37 )$$

where Cₛₒₗᵤₜᵢₒₙ is the concentration of the bath solution the tissue is immersed in. The model approximates the concentration of the free molecules in the tissue as being equal to the concentration of the bath solution. This yields

$$k = 1 + \frac{1}{{K}_{eq}} \qquad Equation ( 38 )$$

Combining Equation (38) and (35), we can evaluate D_{ab} from our results for each pair of k and D

D_{ab}=kD Equation (39)

TABLE 13
Evaluated values for the diffusion coefficient without the binding
effect, D_{ab} from the effective diffusion coefficient, D and
the partition coefficient, k for eosin Y and riboflavin in the cornea
and sclera.

| | | D (μm²/s) | k | D_{ab} (μm²/s) |
|---|---|---|---|---|
| Cornea | Eosin Y | 62 | 4.3 | 267 |
| | Riboflavin | 79 | 1.7 | 134 |
| Sclera | Eosin Y | 6.2 | 13.0 | 81 |
| | Riboflavin | 27 | 1.5 | 41 |

Interestingly, the diffusion coefficient without the binding interaction is very similar to the permeability except for a factor of 1/L. The diffusion without binding effect is faster than with the binding effect since it is the rate of diffusion of the molecules going through the tissues as if they do not bind to the tissue at all (Table 13). For a given molecule, D_{ab} in the cornea is three times greater than D_{ab} in the sclera. Molecules diffusing through these tissues must diffuse around the impermeable collagen fibrils so the more collagen the tissue has, the more tortuous the diffusion path is expected to be. As stated above, the collagen volume fraction of the cornea is 2.5 times less than that of the sclera which correlates with the difference in the diffusion between these two tissues for a given molecule.

For a given tissue, D_{ab} of riboflavin is two times less than D_{ab} of eosin Y. D_{ab} is proportional to stromal and sclera permeability (Equation (34) and (37)) and they have been determined to be strongly dependent on the molecular radius[143]. Riboflavin's hydrodynamic radius is 5.8 Å[152]. No reported value for Eosin Y's hydrodynamic radius can be found. Based on molecular structure, eosin Y's hydrodynamic radius is expected to be similar to fluorescein's, which has been reported as 4.8 Å[143]. Plotting stromal permeability versus radius on a log-log graph for 19 compiled data points yields a straight line with a negative slope[143]. Permeability decreases with increasing molecular radius and when the radius decreases from 5.8 Å to 4.8 Å, the linear fit indicates an increase in permeability by a factor of 1.7, which correlates with the difference by a factor of two for riboflavin and eosin Y's D_{ab} values.

The effective diffusion coefficient determines how rapidly molecules penetrate through the corneal stroma and sclera, which controls the distribution of drug inside the tissue. In safety studies, riboflavin's concentration was calculated using fluorescein's effective diffusion coefficient, D=65 μm²/s which is similar to riboflavin's value (D=79 μm²/s). The calculated drug distribution in the safety studies using fluorescein's effective diffusion coefficient is an acceptable approximation. The concentration error resulting from the approximated diffusivity value (˜10%) is negligible relative to the error resulting from the partition coefficient (˜70%) used to calculate riboflavin's concentration profile.

The soaking technique is the most effective for delivering drug but it is not applicable for in vivo treatments of the cornea. Application of drops is feasible but the drug solutions can flow and enter other parts of the eye. Viscous gels can be applied onto the cornea and they remain on the targeted tissue without entering into other parts of the eye. The selected viscosity enhancers (hyaluronic acid[153, 154], carboxymethylcellulose[155, 156], sodium alginate[157, 158], and methylcellulose[159, 160] have been widely used in various ocular drug delivery systems. Among the different gels studied, carboxymethylcellulose formulation was clear, smooth, free of air bubbles and the easiest to handle for spreading onto the cornea therefore this gel was selected as the delivery vehicle for in vitro and in vivo corneal treatment (Example 36).

Using the extraction and absorbance techniques to quantify the number of drug molecules delivered to a targeted tissue in the eye, together with a diffusion model to fit the data, we were able to extract two important parameters of the system: the diffusion coefficient and the partition coefficient. With these coefficients, the drug concentration profile can be predicted for different drug concentration, application time, and delay time between drug application and light activation of the drug (Example 35). Knowing the drug concentration profile within the tissue is critical to understanding the quantity and location of cross-link formation inside the tissue.

The ability to quantify the amount of drug delivered to a target tissue as a function of delivery time and with an appropriate model, the partition coefficient and diffusion coefficient of the tissue can be determined. This technique can be extended to other drug molecules and to other tissues as well. With these transport properties, the concentration profiles can be calculated for different treatment conditions.

### Example 35

### A Model for Photodynamic Cross-Linking Treatment

Keratoconus is an ocular disease characterized by progressive corneal thinning, protrusion, and scarring, resulting in irregular astigmatism and myopia. It is a bilateral corneal ectasia with a prevalence of 1 out of 2,000, affecting people of all ethnicities and genders equally[17]. Cornea thinning appears to result from loss of material, but it is unclear how or what causes this to happen. Increases in collagenase and other protease activities have been cited as important in the development of corneal ulcerations and keratoconus[39, 161, 162]. The corneas of keratoconus eyes are found to have fewer collagen lamellae, fewer collagen fibrils per lamella, closer packing of collagen fibrils or various combinations of these factors resulting in a weakened structure.

Wollensak et al has developed a treatment for halting the progression of keratoconus by inducing corneal collagen cross-linking [71, 72]. The treatment uses riboflavin activated by UVA to form cross-links inside the cornea. Cross-links serve two important roles in the treatment: to enhance the tissue strength and to increase resistance to collagen degradation by enzymes [39, 87, 163]. Riboflavin/UVA has shown an ability to halt the progression of keratoconus in patients for studies lasting up to 6 years [71, 72]. However, there are drawbacks to the treatment, including cytotoxicity in the cornea which leads to corneal haze for weeks to months following surgery, and it uses a lengthy surgical procedure (60 minutes per eye). Because the treatment is toxic to both keratocytes and endothelial cells, the treatment was carefully designed to limit cytotoxicity to the anterior 350 μm [75, 108, 110, 164]. A high drug concentration and long drug delivery time prior to cross-linking ensures that there is enough riboflavin in the tissue to block UV light from penetrating to the endothelium, and only patients with corneas thicker than 400 μm can be treated. Thus, there is a need for an improved cross-linking treatment to reduce toxicity and treatment time. Eosin Y/visible light can potentially provide such a treatment (Example 36).

Since treatment efficacy depends on both the quantity and distribution of cross-links formed along the tissue depth, studies have examined various properties of the treated tissue at different depths (i.e. change in biomechanical strength[87], maximum hydrothermal shrinkage temperature[36], collagen fiber diameter[38], and hydration[37]). These comparisons were made using bulk sections of the tissues, so they do not provide information regarding the extent of cross-linking as a function of tissue depth. Here, we create a model to quantify the extent of cross-linking as a function of depth for collagen cross-linking induced by photosensitizers. This model provides a more detailed map of the spatial distribution of cross-links for the riboflavin/UVA treatment. It can also be used as an optimization tool for selecting treatment parameters for the eosin y/visible light treatment.

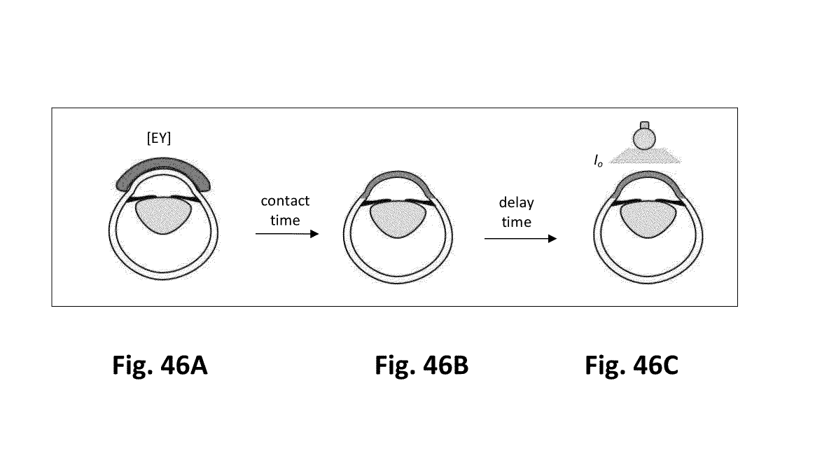

Photodynamic collagen cross-linking treatment has many treatment parameters including drug concentration, drug contact time, delay time between the end of contact time to the beginning of irradiation period, and irradiation intensity and duration (FIGS. 46A-46C). Each parameter affects the safety and efficacy of the treatment and all of the combined parameters yield a very large treatment parameter space. With such a large treatment parameter space, it would be very laborious and costly to optimize the treatment by carrying out experiments. A model can provide insights of how each parameter and combinations of parameters affect the outcome of the treatment.

### Methods

Design of experiments—The collagen gel photorheological technique discussed in Examples 29-31 was used to gather collagen cross-linking rate data in order to model the extent and distribution of cross-link formation in the tissue. Collagen gel samples have uniform drug concentration profiles based on the preparation technique developed Examples 29-31. The protocols for collagen gel preparation and photorheological measurement are described in the Methods Section of Example 29-31. The pairs of drug concentrations and collagen gel thicknesses were selected such that the intensity profile is approximately uniform throughout the sample. Based on light intensity calculations, this is achieved when the ratio of light penetration depth over sample thickness is 1.2. The light intensity profile in the sample is given by:

I(z)=Iₒe^{-(μ+Cε)z} Equation (40)

where I is the intensity, Iₒ is the incident intensity, z is the position inside the collagen gel sample, μ is the collagen gel's absorptivity, C is the drug concentration, and ε is the drug's molar absorptivity. The light enters the sample through a quartz window which is part of the lower plate geometry on the rheometer where the sample sits and travels through the sample up to the upper tool made of aluminum. Some of the light hitting the upper tool gets reflected and some gets scattered. For simplicity, we approximate that all the light hitting the upper aluminum tool gets reflected and travels back down the sample and the profile of the reflected light is also given by Beer's law. Therefore, the total intensity the sample is exposed to at a given position is the sum of the incident light plus the reflected light.

I(z)=Iₒe^{-(μ+Cε)z}+Iₒe^{-(μ+Cε)(L-z)} Equation (41)

Based on this light intensity calculation approach, pairs of drug concentration and sample thickness were selected such that the light intensity profile is approximately uniform throughout the sample. For 450 μm thick riboflavin samples, the light intensity profile is approximately uniform throughout for concentrations less than or equal to 0.03%. For a 225 μm sample, the intensity profile is approximately uniform for concentrations less than or equal to 0.05%. The highest concentration was limited by the minimum thickness of collagen gel samples that could be loaded onto the rheometer. The thinnest collagen gel samples that could be prepared and handled to yield reproducible results were 225 μm thick which corresponds to a 0.05% riboflavin concentration. For 450 μm thick eosin Y samples, the light intensity profile is approximately uniform for concentrations less than or equal to 0.01%. For 225 μm samples, the intensity profile is approximately uniform for concentrations less than or equal to 0.02%. Rate data at these thicknesses and concentrations were used to build a model for cross-linking inside the tissue with non-uniform drug concentration and light intensity profiles.

Design of the model—Photodynamic collagen cross-linking depends on both the local photosensitizer concentration and the light intensity which are functions of treatment parameters: drug concentration, contact time (duration the drug is in contact with the tissue), delay time (period between end of contact time and beginning of irradiation), and irradiation time (FIGS. 46A-46C). Since drug is applied topically to the cornea, the photosensitizer concentration varies along the tissue depth with time. The concentration profile can be calculated using Fick's diffusion equation. The light intensity also varies along the tissue as determined by Beer's law. The cross-linking profile is also expected to vary along the depth since the local cross-linking rate depends on the photosensitizer concentration and light intensity. In order to evaluate the instantaneous local cross-linking rate, the cornea is divided in thin sections along the visual axis so that each section has an approximately uniform concentration and intensity profile. Within each section, the instantaneous cross-linking rate is obtained from collagen gel photorhelogy data (rate of change in storage modulus) of collagen samples with uniform concentration profiles and approximately uniform light intensity profiles. The local change in storage modulus after a given irradiation time is the sum of the instantaneous changes in modulus at each time step.

Using the partition coefficient and diffusion coefficient (Examples 32-34) of the system we can calculate the concentration profile as a function of time for a selected topically applied drug concentration, contact time (duration the drug is in contact with the tissue), delay time (period between end of contact time and beginning of irradiation), and irradiation time.

The cornea is the targeted tissue and it has a thickness on the order of 1 mm. Since the tissue thickness is less than an order of magnitude compared to the diameter of the eye (˜24 mm), it is modeled as a semi-infinite slab of uniform material in which molecules can diffuse. Fick's diffusion equation is given by

$$\frac{\partial C ( z )}{\partial t} = D \frac{{\partial }^{2} C ( z )}{\partial {z}^{2}} \qquad Equation ( 42 )$$

where C is the drug concentration inside the tissue, t is time, z is the position inside the tissue, and D is the diffusion coefficient. An initial condition and two boundary conditions are necessary to solve the equation. During the contact time, the appropriate conditions are

Initial condition at t=0,C=0 for all z≧0 Equation (43.1)

Boundary condition at z=0,C=k·C_{drug} for t>0 Equation (43.2)

Boundary condition at z=∞,C=0 for all t during the contact time Equation (43.3)

where k is the partition coefficient and C_{drug} is the bulk concentration of the drug solution applied. Initially before drug is applied to the tissue, the concentration is zero everywhere inside the tissue (Equation (43.1)). After the drug is applied, the surface of the tissue where drug diffuses into is always in equilibrium with the drug solution (Equation (43.2)). During the drug contact time, the concentration is zero far into the tissue (semi-infinite slab of material approximation, Equation (43.3)). Applying the initial and boundary conditions to the diffusion equation, the concentration profile is given by

$$C ( t , z ) = k \cdot {C}_{drug} \cdot erfc ( \frac{z}{\sqrt{4 Dt}} ) \qquad Equation ( 44 )$$

where erfc is the complementary error function. The concentration profile evolves after the drug solution is removed. The concentration profile after a delay time is also given by Fickian diffusion (Equation (40)) but with a different set of initial and boundary conditions. The concentration profile at the end of the contact time (Equation (44)) is the initial condition for computing the concentration profile during the delay time profile.

Applicants approximate the system with a no flux boundary condition at the anterior surface because drops of balanced saline solution are applied just enough to prevent corneal dehydration during the delay time. Based on this procedure, a negligible quantity of drug would be removed through the anterior surface of the cornea.

$$\frac{d C}{d z} {\vert}_{z = 0} \approx 0 \\ during the delay and irradiation time \qquad Equation ( 45 )$$

The flux at the back of the cornea is given by

J|_{z=L}=hₘ(C|_{z=L}−C_{ac}) Equation (46.1)

where hₘ is the mass transfer coefficient in the anterior chamber, C_{ac} is the concentration in the aqueous chamber. A calculation is done to determine how significant the flux through the back of the cornea into the aqueous chamber is relative to the amount of drug present in the tissue by comparing the flux leaving the cornea to enter the aqueous chamber, Jₒᵤₜ to the average flux of drug entering the cornea, Jᵢₙ during the contact time. As a conservative approximation, we use the greatest flux which is when C_{ac} is 0.

$$\frac{{J}_{out}}{{J}_{i n}} = \frac{{h}_{m} C {\vert}_{z = L}}{{C}_{avg} L / t} \qquad Equation ( 46.2 )$$

where C_{avg} is the average drug concentration in the cornea after a given drug contact time. Reported values of hₘ for fluorescein in the cornea range from 1.15×10⁻⁴ to 2.3×10⁻⁴ cm/min[165]. Using the largest reported value for hₘ, for a 5 minute eosin Y contact time, Jₒᵤₜ/Jᵢₙ is 7.9×10⁻⁴, and for a 30 minutes contact time Jₒᵤₜ/Jᵢₙ is 0.0655. The ratio Jₒᵤₜ/Jᵢₙ is much less than 1 so the flux of drug leaving the cornea to enter the anterior chamber is not significant therefore the no flux boundary condition is also applied at the posterior surface of the cornea.

$$J {\vert}_{z = L} = - D \frac{d C}{d z} {\vert}_{z = L} \approx 0 \qquad Equation ( 46.3 )$$

Apply the initial condition given by Equation (43.1) and the boundary conditions given by Equations (45) and (46.3) to Fick's diffusion equation to solve for the concentration profile with some delay time after the drug contact time. Since the boundary conditions are imposed on surfaces of constant coordinates (z=0 and z=L), and the conditions are homogeneous, the equation can be solved using separation of variables

$$C ( τ , z ) = {\sum }_{1}^{∞} {a}_{n} cos ( n π \frac{z}{L} ) exp ( - \frac{{n}^{2} {π}^{2} D}{{L}^{2}} τ ) \qquad Equation ( 47.1 )$$

where τ is the time since drug solution was removed from the corneal surface and aₙ is

$${a}_{n} = \frac{{\int }_{0}^{L} k \cdot {C}_{bulk} \cdot erfc ( \frac{z}{\sqrt{4 Dt}} ) cos ( n π \frac{z}{L} ) }{{\int }_{0}^{L} {cos}^{2} ( n π \frac{z}{L} )} d z \qquad Equation ( 47.2 )$$

Equation (47.1) and (47.2) give the concentration profile after the drug contact time, and throughout the irradiation time. This model does not take into account the consumption of the photosensitizer as the reaction occurs. This is an acceptable approximation since collagen gel cross-linking experiments show a constant rate for ΔG′ throughout a 30-minute reaction period (Examples 29-31). This implies the fraction of eosin Y consumption is negligible over this time period; therefore, as long as the irradiation period for the treatment is 30 minutes or less, this approximation is reasonable.

For each concentration profile, the corresponding light intensity profile is

I(z)=Iₒ*e^{-[μ+C(z)ε]z} Equation (48)

where I is the intensity, Iₒ is the incident intensity, μ is the tissue's absorptivity, and c is the drug's molar absorptivity.

For a given drug concentration profile and the corresponding light intensity profile, the instantaneous local cross-linking rate is quantified by the rate of change in modulus, Ġ′ obtained from collagen gel photorheology. The total change in local modulus after a given irradiation time, tᵢᵣᵣ is determined by summing over each instantaneous rate of increase in modulus.

ΔG′(z)=∫₀^{t}^{ᵢᵣᵣ}Ġ′(z)dt Equation (49)

The average change in modulus of a sample, ΔG′_{avg}, is determined by

$$Δ {G}_{avg}^{′} = \frac{1}{L} {\int }_{0}^{L} Δ {G}^{′} ( z ) d z \qquad Equation ( 50 )$$

to compare the extent of cross-linking for different treatment conditions.

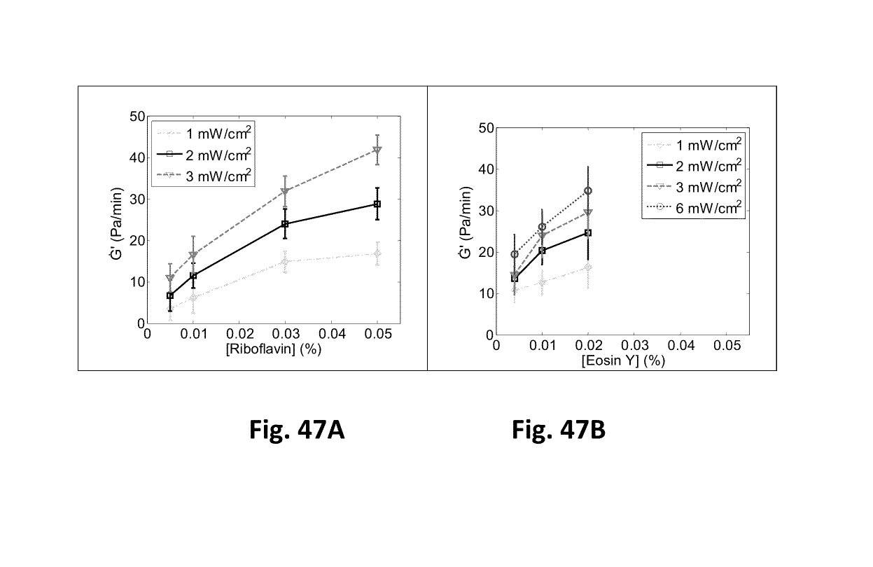

The following results were obtained. The rate of change in oscillatory storage modulus, Ġ′, increased with increasing riboflavin concentration. Increasing the intensity at a given riboflavin concentration increased Ġ′ (FIGS. 47A-47B). The rate of change in modulus also increases with increasing eosin Y concentration. Increasing the intensity at a given eosin Y concentration increased Ġ′. The highest riboflavin and eosin Y concentrations examined were limited by the minimum sample thickness that could be prepared and loaded onto the rheometer. Extrapolations using logarithmic fits provide estimated Ġ′ for concentrations above 0.05% riboflavin and 0.02% eosin Y.

The riboflavin/UVA treatment currently going through clinical trials in the United States uses the procedure where riboflavin drops (0.1% riboflavin, 20% dextran) are applied every 2 minutes for 30 minutes followed UV irradiation (370 nm, 3 mW/cm²) for 30 minutes while adding riboflavin drops every 5 minutes. For the clinical protocol treatment, the concentration profile is approximated for a drug contact time of 30 minutes (FIG. 48A) which yields the corresponding intensity profile (FIG. 48B) and cross-linking profile (ΔG′_{avg} is 503 Pa, FIG. 48C).

Drug concentration, contact time, and delay time determine the quantity and distribution of drug inside the tissue. For a given incident light intensity, the intensity reaching the back of the cornea depends on the total quantity of drug present in the tissue, which is determined by the drug concentration and contact time. How the intensity profile changes inside the tissue depends on the distribution of drug molecules which is determined by the contact time and delay time. For a given contact time, a longer delay time yields a more uniform concentration profile, which results in the intensity decaying slower as a function of tissue depth.

For a given contact time, increasing the drug concentration proportionately increases the concentration inside the tissue (FIG. 49A). In turn, increasing the concentration causes the light intensity to decay more steeply (FIG. 49B). Increasing the concentration from 0.003% to 0.01% eosin Y, increases the extent of cross-linking everywhere in the tissue (average change in modulus, (ΔG′_{avg}, increases from 80 Pa to 104 Pa, FIG. 49C); however, further increasing the concentration from 0.01% to 0.03% decreases the light penetration depth from 146 μm to 38 μm (depth at which the intensity is 1/e of the incident intensity), resulting in most of the tissue with very little light for activating the reaction in the posterior side of the tissue (ΔG′_{avg} decreased from 104 Pa to 55 Pa). At 0.03% concentration, 75% of the cross-links form in the anterior 135 μm, compared to 290 μm for 0.01% concentration.

For a given drug concentration, increasing the contact time increases the concentration everywhere in the tissue (provided the contact time is less than the characteristic time, which is the time it takes for drug molecules to penetrate the entire cornea and is given by L²/(4*D)˜15 minutes for eosin Y in the cornea). The increase in the amount of drug in the tissue (FIG. 50A) causes the light intensity to decay more steeply with longer contact time (FIG. 50A). Nevertheless, for 0.01% eosin Y, light penetrates the entire thickness even if the drug formulation is given 10 minutes contact time. Consequently increasing the contact time from 1 to 5 minutes, increases the extent of cross-linking everywhere in the tissue (ΔG′_{avg} increases from 76 to 104 Pa). Increasing the contact time from 5 to 10 minutes, results in a similar cross-linking profile (FIG. 50C).

For a short contact time (less than the characteristic diffusion time), increasing the delay time between removal of the drug formulation and the inception of irradiation results in a more uniform concentration profile (FIG. 51A). For 5 minutes contact time using 0.01% eosin Y, increasing the delay time, allows the high concentration near the anterior surface to decrease. In turn, this allows light to penetrate more deeply (FIG. 51B), producing a more uniform distribution of cross-links after 5 minutes of irradiation at 6 mW/cm² (FIG. 51C). While increasing the contact time from 0 to 1 to 5 to infinite minutes yields increasingly uniform cross-linking profiles, it has little effect on ΔG′_{avg}: 104 to 108 to 115 to 119 Pa, respectively.

For a given concentration profile (FIGS. 52A-52C) and a selected light dose (1.8 J/cm²), the combination of lower intensity and longer irradiation duration results in a greater ΔG′_{avg}. This example uses the concentration profile predicted for topical application of a 0.01% eosin Y solution for 5 minutes contact time, removing the eosin Y from the surface and allowing 1 minute delay time, the corresponding light intensity profiles for three different irradiation intensities (FIG. 52B), and the resulting cross-linking profiles for a light dose of 1.8 J/cm² (FIG. 52C). The ΔG′_{avg} is 198 Pa for 15 minutes at 2 mW/cm², 139 Pa for 7.5 minutes at 4 mW/cm², and 108 Pa for 5 minutes at 6 mW/cm². The shape of the cross-linking profiles is similar for all irradiation intensities.

For a given concentration profile (FIG. 53A) and a selected irradiation intensity (6 mW/cm²), ΔG′_{avg} increases proportionally with irradiation time. This example uses the concentration profile predicted for topical application of a 0.01% eosin Y solution for 5 minutes contact time, removing the eosin Y from the surface and allowing 1 minutes delay time, the corresponding light intensity profile for 6 mW/cm² incident on the cornea (FIG. 53B), and the resulting cross-linking profiles for three irradiation durations (FIG. 53C). The ΔG′_{avg} is 108 Pa for 5 minutes, 223 Pa for 10 minutes, and 697 Pa for 30 minutes. The shape of the cross-linking profiles is similar for all irradiation intensities.

Combining transport parameters and collagen cross-linking rates, we were able to build a model depicting the collagen cross-linking profile as a function of the depth in the tissue. Various studies have examined the extent of cross-linking in the anterior and posterior corneal stroma resulting from the riboflavin/UVA treatment by comparing changes in the resistance to enzymatic degradation[39], thermomechanical[36], collagen fibril diameter[38], hydration[37], and biomechanical behavior[87].

Enzymatic degradation studies suggested the anterior portion of the stroma was more resistant to degradation compared to the posterior portion since the degradation process started at the posterior and moved toward the anterior portion[39]. This result agrees with the model which predicts a monotonically decreasing cross-linking profile over a 500 μm thick cornea for the riboflavin/UVA treatment (FIG. 49C).

In porcine corneas (800 μm), the anterior portion of treated samples showed significant increase in the maximal hydrothermal shrinkage temperature whereas the posterior portion exhibited a much smaller increase (70.3° C. in control samples, 71.2° C. in the posterior 400 μm, and 75.0° C. in the anterior 400 μm)[36]. The model predicts a ΔG′_{avg} of 609 Pa in the anterior 400 μm compared to 72 Pa in the posterior 400 μm portion of the cornea. This is consistent with the observed behavior where there is a large increase in the shrinkage temperature in the anterior portion due to a greater extent of cross-linking compared to the posterior portion.

Collagen fiber diameter in treated rabbit corneas (400 μm) were found to increase by 12.2% (3.96 nm) in the anterior portion and by 4.6% (1.63 nm) in the posterior portion compared to untreated corneas[38]. The model predicts a ΔG′_{avg} of 916 Pa in the anterior 200 μm compared to 267 Pa in the posterior 200 μm. The change in collagen fiber diameter in the anterior cornea is much greater than that of the posterior cornea which is consistent with the modeling results.

Hydration studies in porcine corneas deduced an intensely cross-linked zone of 242 μm at the anterior surface, an intermediate cross-linked zone of 109 μm, and a non-cross-linked posterior zone of 501 μm [37]. The model predicts a ΔG′_{avg} of 833 Pa in the anterior 242 μm, 307 Pa in the next 109 μm, and 77 Pa in the 501 μm posterior portion.

In human corneas (500 μm), the anterior portion of treated samples showed greater increase in the biomechanical strength compared the posterior portion [87]. At 5% strain for the anterior 200 μm sample, the stress applied was 307×10³ N/m² for treated corneas and was 108×10³ N/m² for control corneas. For the posterior 200 μm sample, the stress applied was 89×10³ N/m² for treated corneas and was 53×10³ N/m² for control corneas.

The anterior flap increased by 254×10³ N/m² whereas the posterior flap increased by 36×10³ N/m². The model predicts a ΔG′_{avg} of 916 Pa in the anterior 200 μm compared to 267 Pa in the posterior 200 μm. Comparison of the results from previous experimental observations with those from the model show very close agreement which suggests the model is a good predictor of the cross-linking profile resulting from the treatment.

Using the model, the predicted riboflavin concentration to maximize cross-linking with the clinical irradiation protocol (3 mW/cm² for 30 minutes) is 0.044%, which yields a ΔG′_{avg} of 618 Pa whereas the clinical concentration (0.1%) only yields a ΔG′_{avg} of 503 Pa. The clinical concentration yields a cross-linking rate that is only 81% of the optimal rate (collagen gel photorheology estimated 78% of the optimal rate, Example 29-31). In addition to providing a greater ΔG′_{avg}, the optimal condition also produces a more uniform cross-linking profile. This is expected to be more advantageous since cross-links serve two purposes when halting the progression of keratoconus: to enhance biomechanical properties and to increase resistance to enzymatic degradation [39, 87, 163]. For an equivalent increase in tissue strength, a more uniform distribution of cross-links is expected to resist enzymatic degradation throughout the cornea better than a less uniform distribution.

Even though an optimal treatment condition exists, it cannot be used due to the cytoxicity nature of riboflavin combined with UVA. The treatment requires a 0.1% riboflavin concentration to prevent a toxic UVA light dose from entering the endothelium[45]. Because the combination of riboflavin and UVA light is cytotoxic, the clinical protocol was optimized for safety rather than efficacy[110]. Given available data on the combination of drug doses and irradiation intensities that are toxic, the model can also be used to predict depth of keratocytes apoptosis and endothelial toxicity for various combinations of treatment parameters.

Unlike riboflavin/UVA treatment, eosin Y/visible light is much more biocompatible (Example 36). Therefore the treatment parameters can be selected based on performance for efficacy instead of safety constraints. The model can be used to examine the role of each treatment parameter and its effect on the overall treatment. In turn, this knowledge can guide selection of treatment conditions that are desirable for clinical use.

The amount of drug transferred from the formulation into the cornea is determined by the drug concentration in the formulation and the contact time (time between topical application and removal of the formulation). The contact time, delay time, and irradiation duration determine how the drug is distributed inside the tissue at any given moment. Results from the model show that low eosin Y concentration (≦0.005% applied for 5 minutes, FIG. 49A) inside the tissue provides a low cross-linking rate yielding a relatively small ΔG′_{avg} for a given irradiation dose (FIG. 49A). A high eosin Y concentration (≧0.03% applied for 5 minutes, FIG. 49A) extinguishes most of the light in the anterior portion of the tissue (FIG. 49B) leaving the posterior section untreated, resulting in a very non-uniform treatment (FIG. 49C) and a lower ΔG′_{avg} both of which are not favorable. Therefore it is desirable to deliver a quantity of drug to the tissue that yields a fast reaction rate and a more uniform light intensity profile, producing a more uniform cross-linking profile. This desirable quantity is the amount such that the average concentration yields a light penetration depth similar to the tissue thickness. The optimal average drug concentration inside the tissue is 0.016%, and concentrations within the 0.016±0.008% are within 90% of the optimal concentration.

Various combinations of eosin Y concentration and contact time can be selected to achieve the optimal quantity of drug inside the tissue: 0.027% with 1 minute contact time, 0.012% with 5 minutes contact time, or 0.0088% for 10 minutes. It is desirable for the treatment to have a short total treatment time and be reproducible. A longer treatment duration increases the risk of infection, increases patients' discomfort, and requires more of a surgeon's time which results in a higher cost. Applying a high drug concentration for a short contact time might have the disadvantage high variability if the delivery time is not carefully monitored (Table 14). Increasing the contact time from 1 to 5 to 10 minutes decreases the variability in the quantity of drug deliver from 29 to 5 to 2%. A 5 minute drug contact time is recommended since it provides a relatively short contact time and low variability.

TABLE 14
Quantity of drug variability resulting from error in
drug contact times by 30 seconds.

| t_{c} (min) | t_{c} error (sec) | Error (%) |
|---|---|---|
| 1 | −30 | −29% |
| | +30 | +22% |
| 5 | −30 | −5% |
| | +30 | +4% |
| 10 | −30 | −2% |
| | +30 | +2% |

Once the desired amount of drug is delivered, adding a delay time before irradiating produces a more uniform concentration profile provided the contact time is less than the characteristic time (15 minutes) (FIG. 51A). A more uniform drug concentration profile provides a more uniform cross-linking profile (FIG. 51C). Given the characteristic time is ˜15 minutes, a 10 minutes total of combined contact time and delay time is sufficient to produce a relatively uniform distribution of drug inside the tissue (FIG. 51A). For a 5 minute contact time and 5 minute irradiation protocol, adding a delay time did not significantly alter the ΔG′_{avg} or the cross-linking distribution (FIG. 51C). For longer irradiation durations, the delay time effect becomes even less significant since the concentration profile continues to evolve during the irradiation period.

Given a drug concentration profile, the irradiation intensity and duration determine the quantity of cross-linking but not the cross-linking distribution. Depending on how much cross-linking is necessary to halt the progression of keratoconus, the irradiation intensity and duration can be selected accordingly. In selecting irradiation intensity and duration, factors that need to be considered are the safety limit of light permissible in the eye, maximum intensity level tolerable for patient comfort, and overall treatment duration. The light intensities and doses considered for corneal irradiation here are much lower than present in other applications such as bonding corneal incisions[166, 167], laser iridectomy and iridoplasty[168]. The light source (514 nm at 640 mW/cm² for 5 minutes or 192 J/cm²) used in bonding corneal incision over a 1-cm diameter area reported no tissue damage to the animals monitored over a 10-week period[166]. This amount of light is 2 orders of magnitude more than the light dose used in the examples above for a 5 minute irradiation period at 6 mW/cm². (Biocompatibility studies in Example 36, shows a 3.6 J/cm² light dose combined with eosin Y is well tolerated by the cornea with no toxicity to the endothelium and very little damage to keratocytes compared to riboflavin/UVA treatment.)

Results show that for the same light dose, selecting a lower irradiation intensity with longer duration results in more cross-linking than a high intensity and shorter duration. However, since the maximum exposure limit is very high, a higher intensity (6 mW/cm²) and shorter irradiation period can be selected to minimize the overall treatment duration. The other factor to consider in selecting the intensity is the level of discomfort patients can tolerate.

The photo-activated collagen cross-linking treatment has multiple parameters that are interdependent and with a model we are able to predict the cross-linking profile resulting from adjusting individual or combinations of different parameters. The parameter space is very large and carrying out experiments to find optimal values would be daunting. This is a powerful tool that can help narrow down the parameter space for selecting optimal values to be used in the clinic.

This model can be used to create customized treatments for individual patients depending on how severely the disease has progressed and how much cross-linking is necessary to treat the patient. Once the amount of cross-linking necessary to halt the progression of the disease in each patient is better understood, this model can also help customize treatments for individual patients so that they are effective, safe and as comfortable for the patients as possible.

### Example 36

### Clinical Comparison of Eosin Y and Riboflavin

Keratoconus is a bilateral corneal disorder with a prevalence of 1 out of 2,000 without racial or gender bias^{[17, 128]}. This eye disease is characterized by progressive corneal thinning, protrusion, and scarring, resulting in irregular astigmatism and myopia. Corneal thinning appears to result from loss of material, partly due to the increased collagen degradation rate^{[31, 110]}. The cornea of keratoconic eyes are found to have fewer collagen lamellae, fewer collagen fibrils per lamella, closer packing of collagen fibrils or various combinations of these factors resulting in a weakened structure^{[17]}.

Corneal thinning results in visual impairment that can be corrected by spectacles in the early stages of the disease. As corneal irregularities increase, eyeglasses are not sufficient to provide clear vision, so contact lenses are used. (Patients who do not tolerate contact lenses, may under surgical procedures, such as thermokeratoplasty^{[19]}, epikertaophakia^{[169]}, and intracorneal ring segments^{[21]} to reduce refractive errors induced by irregular corneal thinning associated with the disease; however, these treatments do not halt the progression of the keratoconus.) When the disease progresses to the stage where contact lensesno longer suffice, a corneal transplant (keratoplasty) is required. About 20% of patients with keratoconus ultimately require keratoplasty^{[17]}.

Pioneering research of Wollensak, Seiler, and Spoerl demonstrated that photodynamic corneal collagen cross-linking using riboflavin and UVA could halt the progression of keratoconus^{[71, 72, 170]}. Five major human clinical trials in different countries ranging from 3 months to 6 years have demonstrated riboflavin/UVA treatment is effective in treating keratoconus^{[111]}. The current protocol requires topical application of drug solution (0.1% riboflavin with 20% dextran) to the cornea every 2 minutes for 30 minutes before irradiating, and every 5 minutes during 30 minutes of irradiation with 3 mW/cm2 UVA light.

The combination of riboflavin and UVA is toxic to both keratocytes and endothelial cells. Since endothelial cells cannot regenerate in human eyes, the treatment was carefully designed to restrict toxicity to the anterior 350 μm of the corneal stroma[45]. This is achieved by selecting a high drug concentration and applying it for an extended duration to limit the amount of UVA light reaching the endothelium. The treatment cannot be used on patients with corneas under 400 μm since it causes “significant necrosis and apoptosis of endothelial cells” in rabbit corneas[108]. Keratocyte apoptosis causes corneal haze until they completely regenerate after 6 months, and in some cases, it takes up to 12 months to recover completely[43, 110, 171]. Even though there is toxicity, patients are willing to risk damaging their eyes to receive this treatment (currently being used in other countries and is going through FDA clinical trials in the U.S.) over the alternative treatment (corneal transplant).

Because riboflavin/UVA treatment has many safety concerns, it has been suggested that selecting a photosensitizer in the visible spectrum might reduce harmful effects[111]. Eosin Y is a photosensitizer with an absorption peak in the visible range (514 nm) which has shown the ability to cross-link collagen[52, 116] and stabilize sclera tissue[1]. It has also been approved for use in the body by the FDA[51].

The following materials and methods were used. In Vitro Treatment—Eyes from New Zealand White Rabbit ranging from 2 to 3 kg were provided by collaborator Dr. Keith Duncan at the University of California at San Francisco. Eyes were shipped and stored in balanced saline on ice until use within 48 hours of enucleation. The epithelial cell layer was removed by scraping with a scalpel until epithelial material could be seen on the scalpel and the surface of the cornea changed from a smooth texture to a matte texture. The eyes were then placed into Dulbecco's phosphate buffer saline (DPBS) until treatment (within 30 minutes). Orbital tissues (muscle, fat, conjunctiva) covering the sclera and corneoscleral limbus were left in place for treatment to simulate the in vivo condition with respect to drug reaching the sclera.

Eosin Y/visible light treatment (EY/vis)—Eosin Y gel (0.04% w/w eosin Y and 3% w/w carboxymethylcellulose in DPBS) was prepared and then transferred into a 10 mL syringe. Using the syringe, ˜0.5 mL of gel was applied onto the cornea. After 5 minutes contact time, the gel was removed from the corneal surface by squirting DPBS onto the cornea. The eye was then placed onto a holder with the cornea facing up to receive irradiation from an array of green light emitting diodes (seven 5-mm LEDs at 525±16 nm, 6 mW/cm² in the plane of the cornea). Irradiation was applied for 10 minutes.

The concentration and contact time were selected based on the results in f showing a cornea immersed in 0.016±0.008% eosin Y solution for 5 minutes delivers the optimal amount of drug. To error on the side of having more drug in the cornea, a concentration of 0.02% was selected. In order to deliver an equivalent amount of drug in a gel form, twice the concentration is necessary in a gel formulation (0.04% eosin Y, 3% carboxymethylcellulose in DPBS) based on measurements using the light absorption technique discussed in Example 24-28. Results from the model in Example 35 show that adding a delay time before irradiation does not significantly affect the cross-linking profile. Therefore, the corneas in these experiments were irradiated immediately after removal of the drug formulation from the corneal surface.

Riboflavin/UVA treatment (R/UVA)—Following the R/UVA protocol used in clinical trials in the United States, the eye was placed onto a holder with the cornea facing up to receive drops of riboflavin (0.1% w/w riboflavin-5′-monophosphate and 20% w/w T-500 dextran in DPBS) and irradiation. Riboflavin drops were applied onto the cornea every 2 minutes for 30 minutes. The eye was then irradiated using a similar light set up described above but with UV LEDs (370±12 nm, 3 mW/cm²). Irradiation was applied for 30 minutes while adding riboflavin drops every 5 minutes.

Control treatment—Nothing was done to the eye other than removal of the epithelium.

After treatment, all eyes were placed into DPBS (Table 15). Orbital tissues were removed with scissors to expose the sclera and ensure accurate analysis of the eye shape.

TABLE 15
In vitro treatment summary

| Treatment | Drug | Light (min) | # of Eyes |
|---|---|---|---|
| EY/vis | Eosin Y gel, 5 min | 10 | 8 |
| R/UVA | Riboflavin drops, 30 min | 30 | 8 |
| Control | None | None | 12 |

Intact Globe Expansion—Intact globe expansion was performed following the procedure described by Mattson, Huynh, et al. Eyes were mounted onto acrylic cylinders inside of a transparent PLEXIGLAS® observation cell filled with DPBS. To minimize bacteria growth during the experiment, several drops of antibiotic eye drops (Bausch & Lomb neomycin, polymyxin B sulfate and gramicidin ophthalmic solution USP) were added to the DPBS solution in the observation cell. The eyes were aligned with the major axis of the equator parallel to the imaging plane. There are two holes sealed with rubber septa used for inserting 30 gauge hypodermic needles to control the intraocular pressure (IOP). The needles were inserted into the eyes through the posterior sclera. The needles were connected to a DPBS reservoir set at a height h above the eyes to impose a desired IOP governed by hydrostatic pressure (IOP=pgh; p=density, g=gravitational acceleration). To minimize activation of any residual photosensitizer present in the tissue, the experiment was performed in the dark except for 15 seconds of illumination from a fluorescent lamp every 15 minutes to provide light for the photographs. For the first hour, the IOP was held at 15 mmHg to restore the shape of the eye (since shipping and handling results in a variable shape). Then IOP was switched to 300 mmHg until the experiment completed (when rupture was observed or the level of fluid in the reservoir began to drop due to leaks in the tissue).

Photographs of the eyes were taken every 15 minutes throughout the experiment then analyzed for changes in ocular dimensions (corneal perimeter—CP, corneal length—CL, and corneal diameter—CD) using a custom MATLAB program created by Dr. Matthew Mattson in the Kornfield Lab. The rate of change for each of the three corneal dimensions was characterized using the difference between their initial value (using the image acquired 15 min after the pressure was changed from 15 to 300 mmHg) and their final value (described below) divided by the elapsed time between the initial and final images. The initial image is selected to be 15 minutes after switching on high pressure to avoid the variability in the transient response during the first few minutes after the large IOP change. For example, the rate of change of the corneal perimeter, d(ΔCP)/dt, is calculated using

$$\frac{d ( Δ CP )}{d t} = \frac{{CP}_{f} - {CP}_{i}}{{CP}_{i}} \times \frac{1}{{t}_{c}} \qquad Equation ( 51 )$$

where CPᵢ is the initial corneal perimeter, CP_{f} is the corneal perimeter measured at end of the creep period. The end of the creep period is selected to be 2 hours before the first eye undergo tissue failure occurred so that calculated rates are due to creep and not tissue defects leading to failure (20 hours for in vitro experiments and 30 hours for in vivo experiments). CL and CD were computed using the same equation replacing CP with the either CL or CD.

In Vivo Treatment—New Zealand White Rabbits ranging from 2 to 3 kg were treated at UCSF in collaboration with Dr. Keith Duncan. Each rabbit was given general anesthesia with 1-5% inhaled isofluorane administered by mask. A speculum was inserted into the rabbit eyelid to keep the eye open for treatment. Drops of 0.5% proparacaine were applied onto the eye followed by sterilization with 5% povidone-iodine (betadyne). The eye was then rinsed with ocular balanced saline solution (BSS). The epithelium was removed by dipping a Weckcell sponge into 40% ethanol solution then rubbing it against the corneal surface until the epithelium came off.

Eosin Y/visible light treatment—Approximately 0.5 mL (between 0.4 to 0.6 mL) eosin Y gel was applied to the cornea using a syringe. After 5 minutes contact time, the gel was removed by rinsing the cornea with BSS. Within 1 minute, the cornea was irradiated with 525±16 nm light at 6 mW/cm² for 10 minutes. The fellow eye served as a control: BSS drops were applied to the cornea for 1 minute then followed by 10 minutes of irradiation as above.

Riboflavin/UVA treatment—Riboflavin drops were applied onto the cornea every 2 minutes for 30 minutes. The eye was then irradiated with 370±12 nm light at 3 mW/cm². Irradiation was applied for 30 minutes while adding riboflavin drops every 5 minutes. The fellow eye served as a control, receiving BSS drops for 1 minute followed by 30 minutes UV irradiation while adding BSS drops every 5 minutes.

TABLE 16
In vivo treatment summary for efficacy study

| Treatment | Drug | Light (min) | # of Eyes |
|---|---|---|---|
| EY/vis Treated | Eosin Y gel, 5 min | 10 | 8 |
| EY/vis Control | BSS, 1 min | 10 | 8 |
| R/UVA Treated | Riboflavin drops, 30 min | 30 | 4 |
| R/UVA Control | BSS, 1 min | 30 | 3* |

*One R/UVA Control eye was damaged during enucleation.

After irradiation, the eye was rinsed with BSS followed by application of antibiotic eye drops. The treatment was completed and the speculum was removed (Table 16). Animals were sacrificed 24 hours after treatment. The eyes were enucleated, stored in BSS on ice, and shipped to Caltech overnight. All eyes were tested within 12 hours of arrival (within 36 hours of enucleation) using the intact globe expansion apparatus described above.

Biocompatibility—Treatments were performed in vivo at UCSF in collaboration with Dr. Keith Duncan according to the procedures described above. After treatment, eyes were observed for inflammation, corneal haze, and epithelial regrowth over a period of 7 days.

Another set of in vivo studies was performed for histology. The treatments used the same in vivo procedure described above. Animals were sacrificed 24 hours after treatment. Eyes were enucleated and fixed in 10% formalin, embedded in paraffin, and sections were cut and stained with eosin and hematoxylin.

TABLE 17
In vivo treatment summary for biocompatibility study

| Animal | Eye | Drug | Light (min) |
|---|---|---|---|
| 1 | OD | Eosin Y gel, 5 min | 0 |
| | OS | None | 0 |
| 2 | OD | Eosin Y gel, 5 min | 10 |
| | OS | None | 10 |
| 3 | OD | Riboflavin drops, 30 min | 30 |
| | OS | BSS, 1 min | 30 |

Results for in vitro treated eyes showed that treated eyes resisted expansion relative controls as measured by all three corneal dimensions (Table 17, * indicates p<0.05). The rate of change of CP, d(ΔCP)/dt, is most closely related to the (approximately biaxial) strain rate during exposure to IOP=300 mmHg. Due to the strength of the sclera, the corneal diameter generally expands less than the corneal perimeter (i.e., d(ΔCD)/dt<d(ΔCP)/dt) and, consequently, CL increases more than CP (i.e., d(ΔCL)/dt>d(ΔCP)/dt).

TABLE 18
10⁴ * Rate of change in corneal dimensions. (CP = corneal
perimeter, CL = corneal length, CD = corneal diameter)

| | | d(ΔCP)/dt | d(ΔCL)/dt | d(ΔCD)/dt |
|---|---|---|---|---|
| In vitro | Control (N = 12) | 23 ± 7 | 59 ± 19 | 16 ± 8 |
| | EY/vis (N = 8) | *3 ± 5 | *2 ± 9 | *5 ± 4 |
| | R/UVA (N = 8) | *13 ± 10 | *15 ± 18 | *13 ± 7 |
| In vitro | Control (N = 11) | 12 ± 6 | 18 ± 11 | 10 ± 4 |
| | EY/vis (N = 8) | *6 ± 5 | 10 ± 8 | *5 ± 5 |
| | R/UVA (N = 4) | *2 ± 3 | 8 ± 3 | *0 ± 3 |

*Indicates statistically significant difference (p < 0.05) from control group for each treatment type (in vitro and in vivo).

By any of these measures, the deformation of the treated corneas is ½ or less that observed for controls (Table 18). The EY/vis treatment is comparable to the riboflavin/UVA treatment; there is no statistically significant difference between these two groups.

Results for in vivo treated eyes show fellow controls respond identically in the EY/vis and R/UVA groups: respectively, the rates of increase of CP were 0.19±0.12%/hr and 0.14±0.07%/hr; of CL were 0.27±0.20%/hr and 0.28±0.09%/hr; and of CD were 0.15±0.08%/hr and 0.08±0.06%/hr. Therefore, the results for the controls are treated in aggregate. Control eyes from the in vivo study resist deformation relative to controls in the in vitro study (cf., top row to bottom row of Table 18). The exact origin of this difference is not yet known.

Results for in vivo treated eyes showed that treated eyes resisted expansion relative to controls (Table 18, * indicates p<0.05). The creep rates of the in vivo treated groups are less than or approximately ½ those of the controls for all three corneal dimensions (Table 18). The EY/vis treatment is comparable to the riboflavin/UVA treatment; there is no statistically significant difference between these two groups.

Two types of biocompatibility studies compare riboflavin/UVA and eosin Y/vis:
examination of corneal recovery during the first week after treatment; and histological examination of acute toxicity during the first 24 hours. In the first, animals were examined on Day 2 and Day 7 after treatment, recording observations of inflammation, corneal haze, and epithelial regrowth (Table 19). The EY/vis treatment was indistinguishable from balanced salt solution (BSS) control in all animals by all measures, with the exception of a delay in re-epithelialization in one animal. R/UVA treated eyes showed moderate inflammation and severe corneal haze on Day 2, which was absent in BSS controls and EY/vis treated eyes. The inflammation resolved and the corneal haze became mild after 7 days. Epithelial regrowth only occurred in patchy areas covering approximately ⅓ of the debrided area after 7 days, in stark contrast to BSS controls or EY/vis treated eyes.

TABLE 19
Eosin Y/visible light treatment biocompatibility

| | | Day 2 | | Day 7 | | |
|---|---|---|---|---|---|---|
| | | Inflam- | Corneal | Inflam- | Corneal | Epithelial |
| Animal | Eye | mation | Haze | mation | Haze | Regrowth |
| 1 | EY/vis | None | None | None | None | 100% |
| | BSS/vis | None | None | None | None | 100% |
| 2 | EY/vis | None | None | None | None | 100% |
| | BSS/vis | None | None | None | None | 100% |
| 3 | EY/vis | None | None | None | None | ~50% |
| | BSS/vis | None | None | None | None | 100% |
| 4 | EY/vis | Mild | Mild | None | None | ~50% |
| | BSS/vis | Mild | Mild | None | None | ~50% |

TABLE 20
Riboflavin/UVA treatment biocompatibility

| | | Day 2 | | Day 7 | | |
|---|---|---|---|---|---|---|
| | | Inflam- | Corneal | Inflam- | Cornea1 | Epithelial |
| Animal | Eye | mation | Haze | mation | Haze | Regrowth |
| 1 | R/UVA | Moderate | Severe | None | Mild | ~33% |
| | BSS/UVA | Mild | Mild | None | None | 100% |
| 2 | R/UVA | Moderate | Severe | None | Mild | ~33% |
| | BSS/UVA | Mild | Mild | None | None | 100% |

Histology performed on corneal cross-sections of animals sacrificed 24 hours after treatment shows that BSS controls are insensitive to irradiation with either visible or UVA light Apoptosis of keratocytes and the presence of some inflammatory cells are observed in the anterior ⅓ of the stroma in all of the BSS controls, in accord with that associated with the response to de-epithelialization[172]. The posterior half of the stroma and the endothelium in all three BSS controls show the usual number and morphology of keratocytes, as well as an intact endothelium. Apoptosis of keratocytes and the presence of some inflammatory cells in the anterior ⅓ of the stroma is also evident in the corneas that received EY (no light) and EY/vis. The number and morphology of keratocytes in the posterior half of the stroma and the intact endothelium observed in both EY (no light) and EY/vis are similar to the BSS controls. The corneas treated with EY (no light) and EY/vis are indistinguishable, indicating that phototoxicity is negligible in the case of EY/vis. The R/UVA treated eye was completely devoid of keratocytes in the stroma and no endothelial cells remained, in accord with prior literature on the phototoxicity of R/UVA^{[108]}. The fellow eye treated with BSS/UVA has an intact endothelial cell layer, a normal distribution of keratocytes in most of the cornea with a few inflammatory cells in the anterior section of the stroma, in accord with prior studies that showed the phototoxicity of riboflavin is not elicited by the UVA irradiation alone^{[109]}.

Eosin Y/vis treatment and R/UVA treatment produce similar stabilization of rabbit cornea as indicated by resistance to creep when challenged by elevated intra-ocular pressure. Similar efficacy is observed both when the treatment is applied in vitro and when treatment is performed in vivo in a rabbit model. The R/UVA treatment is found to be effective in studies lasting up to 6 years due to the stable nature of the cross-links formed. Cross-links induced by EY/vis are expected to be equivalent to the ones formed by R/UVA (Examples 24-28). So they should resist hydrolysis and enzymatic degradation in a similar manner. Therefore, it is worth investigating the expectation that EY/vis would also provide the long term efficacy.

While the efficacy of the two treatments are comparable, the toxicity of R/UVA is much more severe than that of EY/vis as measured by the degree of inflammation, epithelial regrowth, corneal haze (Tables 19 and 20), and cytotoxicity (Table 21). In addition, the total treatment time for the R/UVA protocol (60 minutes) is four times longer than that for the EY/vis treatment (15 min).

Consistent with previous studies, corneal haze was observed after R/UVA treatment[43, 110, 171], which has been attributed to keratocytes apoptosis. Keratocyte apoptosis causes edema formation leading to stromal haze. In accordance, keratocyte apoptosis was observed throughout the rabbit corneas treated with R/UVA. Numerous studies have documented keratocyte apoptosis resulting from R/UVA treatment down to a depth of 300-350 μm, which leads to corneal haze in patients post-operatively ranging from weeks to months until keratocytes repopulate the cornea[44, 71, 164, 173]. Typically, repopulation of keratocytes begins 2-3 months after treatment and reaches a normal density after 6 months[44, 74]. Corneal haze of various degrees in patients has been reported to last for up to 12 months before resolving completely[72].

EY/vis treatment induced little or no corneal haze, which is consistent with histology results showing a normal distribution of keratocytes in most of the stroma (Table 19 and Table 21). Keratocyte apoptosis in the anterior section of the cornea was also observed in the control groups due to removal of the corneal epithelium^{[172]}. Based on these observations in a rabbit model, it is worth investigating the expectation that EY/vis would cause very mild keratocyte toxicity, little corneal haze and faster recovery in patients. If this were borne out in clinical studies, the implication would be that patients could receive corneal cross-linking without the inconvenience of months of corneal haze currently experienced by patients receiving R/UVA treatment.

In addition to keratocyte toxicity, R/UVA treatment also induced endothelial cytotoxicity (Table 21), which has also been observed in previous studies[47, 108]. Endothelial cytotoxicity in rabbit corneas resulting from the treatment has been attributed to their thin corneas (400 μm or less). In such thin corneas, the light intensity reaching the endothelium is high enough to cause damage. Therefore, it has been established that the treatment cannot be performed on patients with corneas thinner than 400 μm[45, 71, 108]. EY/vis treatment was very well tolerated by the endothelial cell layer in rabbit corneas; treated eyes have indistinguishable endothelial cell layers compared to fellow control eyes (Table 21). Based on these observations in a rabbit model, it is worth investigating the expectation that EY/vis treatment would be safe for treatment of advanced keratoconus patients with corneas thinner than 400 μm. If this were borne out in clinical studies, the EY/vis treatment might also be safe for post-LASIK ectasia patients, who tend to have thin corneas due to removal of corneal tissue during LASIK^{[174]}.

TABLE 21
Results from histology of corneal cross-sections 24 hours after treatment. The top
row received treatment with either Eosin Y or riboflavin as indicated, and the bottom row
received controls of balanced saline solution (BSS).

| | Eosin Y with Green Light | | Eosin Y without Light | | Riboflavin with UVA Light | |
|---|---|---|---|---|---|---|
| | Endothelium | Keratocytes | Endothelium | Keratocytes | Endothelium | Keratocytes |
| Treated | Intact— | Apoptosis, | Intact— | Apoptosis, | Devoid— | Apoptosis in |
| | normal | Inflammation | normal | Inflammation | complete | 100% of |
| | | in Anterior | | in Anterior | loss of | Cornea |
| | | 1/3 of Cornea | | 1/3 of Cornea | endothelium | |
| Control | Intact— | Apoptosis, | Intact— | Apoptosis, | Intact— | Apoptosis, |
| | normal | Inflammation | normal | Inflammation | normal | Inflammation |
| | | in Anterior | | in Anterior | | in Anterior |
| | | 1/3 of Cornea | | 1/3 of Cornea | | 1/3 of Cornea |

Corneal collagen cross-linking by production of singlet oxygen upon irradiation of a photosensitizer occurs both using riboflavin (irradiated with UVA) and using eosin Y (irradiated with green light). Cross-links formed by riboflavin are found to be stable in studies lasting up to 6 years and those formed by eosin Y are expected to be equivalent (Examples 24-28) so should produce long-term stability as well. The two approaches are shown to confer similar stabilization of rabbit cornea. Stark differences between the two treatments are seen in corneal toxicity, with little phototoxicity observed for the EY/vis treatment. Of particular interest, no endothelial toxicity was observed with EY/vis in a rabbit model, even though the cornea is less than 400 μm thick. Therefore, future clinical studies are recommended to determine if EY/vis treatment is safe for patients with corneas thinner than 400 μm. Relative to the usual R/UVA clinical protocol, the EY/vis protocol requires ¼ the treatment time (15 minutes). If clinical studies confirm the results seen in a rabbit model, the EY/vis treatment might reduce patient discomfort and treatment cost relative to R/UVA. Clinical studies are highly recommended to further investigate safety and efficacy of EY/vis treatment, which has the ability to retain the benefits of corneal cross-linking demonstrated by R/UVA while significantly reducing toxicity and treatment duration.

### Example 37

### Animal Model for Photodynamic Cross-Linking

In degenerative myopia, the reduction of collagen fibril diameter, enhanced turnover of scleral collagen, and alteration of scleral glycosaminoglycans results in mechanical changes to the sclera.[14] Progressive elongation of the eye in degenerative myopia is thought to be the result of 1) the tissue being inherently weak, 2) the sclera continuously being remodeled, or 3) a combination of these.^{[14, 175]} From studies of human donor tissue, high myopia is associated with weakening and thinning of the sclera, a reduction in matrix material, and reduction in collagen fibril diameter. While refractive errors induced by progressive myopia are readily corrected by spectacles, contact lenses, corneal refractive surgery, or intraocular lenses, these modalities do not prevent visual loss induced by stretching of chorioretinal tissues. Current means to treat choroidal neovascularization in degenerative myopia, such as photodynamic therapy, are minimally effective,[176] and studies have only recently begun to test injections of anti-angiogenic drugs such as bevacizumab (AVASTIN®), or LUCENTIS®.[177-181] Various attempts have been made to treat expansion of the eye due to myopia, including the use of scleroplasty, scleral reinforcement, and even an attempt to polymerize foam around the eye.[182-191] Largely because these modalities remain unproven in well-controlled clinical trials, none have been widely adopted to manage patients with degenerative myopia. Current therapies are essentially palliative, attempting to mitigate visual loss in this condition.

Crosslinking of scleral components has the potential to halt progression of degenerative myopia because it addresses both of the underlying causes that are currently hypothesized: crosslinking increases tissue strength and hinders tissue remodeling.[192-194] Wollensak and Spoerl have reported the use of collagen cross-linking agents, including glutaraldehyde, glyceraldehyde, and riboflavin-UVA treatment, to strengthen both human and porcine sclera in vitro. [66] Glutaraldehyde and glyceraldehyde would be difficult to spatially control, and unwanted crosslinking of collagen in vascular and neural structures might have particularly untoward effects. Use of light-activated riboflavin would seem preferable in this regard; however, when testing on a rabbit model, “serious side-effects were found in the entire posterior globe with almost complete loss of the photoreceptors, the outer nuclear layer and the retinal pigment epithelium (RPE).”[195] While crosslinking near the posterior pole would increase scleral modulus and potentially arrest myopic progression, there remains a need for a non-toxic crosslinking agent that could be activated using short exposure to a less-toxic light source. Applicants of the present disclosure have found that the visible-light-activated co-initiator system of Eosin Y (EY) and triethanolamine (TEOA) has the potential to fill this need.

For transition of this treatment from the lab to clinical practice, biocompatibility and efficacy must be proven in an animal model of myopia. Current state-of-the-art animal models to study the etiology of myopia rely on 1) visual form deprivation and 2) the eye's tendency to correct refractive errors toward emmetropia.[196] During development, eyes tend to grow excessively upon removal of spatial vision. Form-deprivation models use this response to induce myopia either by placing semitransparent occluders over the eye, or by suturing the eyelid shut.[197] The second animal model makes use of emmetropization of the eye, which is the process by which eyes change to focus images on the retina. When minus or plus lenses are placed over the eye, the eye adjusts its growth to bring the image into focus.

As is observed in the human disease, form-deprivation animal models (e.g., tree shrew eyes covered with occluders for 12 days) also exhibit weakened sclearal tissue (e.g., increased scleral creep rates). In these animal models, there is also a measurable change in the amount and type of collagen and proteoglycan present in the tissue, indicating abnormal remodeling of the sclera. Sustained form-deprivation in animals induces changes in collagen fibril diameter and spacing analogous to the distinctive structure observed in human donor tissue of high myopes.

Various animal models exhibit similarities to humans and each other. Eutherian mammals, such as humans, monkeys, tree shrews and guinea pigs, share the trait that “the entire sclera consists of the fibrous, type I collagen-dominated extracellular matrix”.^{[175]} This feature sets them apart from other vertebrates, which have an inner layer of cartilage (e.g., in chicks). Indeed, the mechanism of emmetropization during form-deprivation in eutherian mammals (remodeling of the fibrous sclera) is different from that in other vertebrates (growth of the inner cartilaginous region). Therefore, eutherian mammals provide a better model for testing treatments related to scleral remodeling for potential application in humans. In light of the fact that tree shrews and monkeys are difficult to obtain and monkeys suffer from high variability of the results, researchers have been establishing other mammalian models. Guinea pigs have recently gained acceptance due to the fact that they rapidly develop myopia, the changes are large and reproducible, and they are easy to care for.[198-204] This animal provides a model that is well suited for research requiring significant numbers of animals, and at the same time demonstrates physiological and anatomical similarities to humans.

Despite the fact that the mechanism of degenerative myopia in humans is not completely understood, the animal models of myopia do express the weakened sclera and excessive remodeling typical of the disease. Light activation of Eosin Y/TEOA can strengthen the sclera; and non-enzymatic collagen crosslinking is known to decrease enzymatic degradation. Therefore, treatment with Eosin Y/TEOA has the potential to address both putative mechanisms of degenerative myopia.

Efficacy and biocompatibility of this potential treatment can be demonstrated. Stabilization of ocular shape is demonstrated for in vitro and in vivo drug delivery to rabbit eyes followed by in vitro eye expansion using the intact globe method. Preliminary safety studies in rabbits suggested no ill effect of the treatment. We have also conducted experiments to establish drug and light delivery protocols in guinea pigs and to assess the effect of EY/TEOA on ocular growth and form-deprivation myopia in collaboration with Sally McFadden at the University of Newcastle in Australia. The current results indicate that EY/TEOA has an ability to alter ocular parameters of guinea pig eyes without altering gross ocular function or animal behavior.

The following procedures were used for testing the effect of in vitro treatment of eyes on preventing expansion of intact rabbit kit globes subjected to an elevated intraocular pressure.

Tissue Preparation: Eyes from 2-3 week old New Zealand White Rabbits (University of California at San Francisco) were stored in saline on ice for use within 48 hours of enucleation. Immediately before testing, the extraocular muscles, the conjunctiva, and the episcleral tissues around the eyes were carefully removed to expose the sclera.

Materials: Treatment solutions of 0.0289 mM EY and 90 mM TEOA in DPBS (henceforth called 1×EY) were prepared fresh. These solutions are activated by visible light and have peak absorption at 514 nm. The measured pH was 7.5 for the solution. Glyceraldehyde (GA) solution was prepared by mixing 2% by weight DL-Glyceraldehyde (Sigma) in distilled water. The pH was adjusted to ˜7.5 with HCl and NaOH.

Eosin Procedure: Eyes were soaked for 5 min in 5 mL of treatment (1×EY) or control (DPBS) solution. The eyes were removed from the soak and excess solution was wiped from the surface using a Kimwipe. The treatment was activated by placing the eyes under one of two light sources: a high intensity mercury arc lamp equipped with a 450-550 nm bandpass filter that provided 34 mW/cm², or a panel of seven light emitting diodes (LEDs) with a spectral output at 525±16 nm that provided an irradiance of 7-10 mW/cm², as measured at the center position of the eye. With the arc lamp, the anterior hemisphere of the eye was exposed for 5 minutes and then the eye was flipped and the posterior globe was exposed for 5 minutes. With the LEDs, the entire eye was irradiated at once for 5 minutes. The eyes were placed in a rinse solution of DPBS for 30-45 min and then loaded on the expansion setup which has been described in detail previously.

Glyceraldehyde Procedure: Because of its well-documented effects as a crosslinker, a comparison group was treated with 2% GA solution. To allow GA to penetrate into the cornea (for comparison to keratoconus treatments), the corneal epithelium of enucleated eyes was removed by scraping with a scalpel blade. The eye was then soaked in 5 mL of 2% GA for 12 hours; when it was removed from the soak, excess solution was removed with a Kimwipe. The eyes were rinsed in a 20 mL bath of DPBS for ˜5 seconds, and then put in a fresh 40 mL DPBS bath to rinse for 10 hours. The eyes were then loaded on the expansion setup.

The expansion protocol began with a 1 hour interval at an intraocular pressure (22 mmHg) close to the physiologic value, which allowed the globe to recover from shape distortion that may have occurred during handling post mortem. Then the pressure was raised and held at 85 mmHg for 24 hours. Digital photographs (2272×1704) were acquired every 15 min for the duration of the experiment.

Toxicity studies were performed at UCSF, to determine if the formulation and light exposure selected from in vitro studies would be suitable to use in an animal model for myopia. To test the in vivo response to 1×EY and light exposure, the following experiments were performed using topical application of the drug.

Procedure: Four adult New Zealand White rabbits were given general anesthesia with 1-5% inhaled isofluorane administered by mask and topical 0.5% proparacaine to the right eye (OD). The right eye of each animal was sterilized with 5% povidone-iodine (betadyne). Throughout the procedure the eye was washed with sterile ocular balanced saline solution (BSS). A 15 mm incision was made in the conjunctiva close to the limbus and another incision running anterior to posterior allowed the conjunctiva to be pulled away to expose the sclera over approximately 1 cm² area. The animal was positioned such that the exposed sclera faced upward and a drop of solution placed on it could remain in contact with the tissue for 5 minutes. Rabbits from Group 1 had 200 microliters of 1×EY solution applied directly to the exposed sclera. Rabbits from Group 2 had 200 microliters of DPBS (control) applied directly to the exposed sclera.

After 5 minutes, the treated area was rinsed with 1-2 mL of BSS and then photoactivated by exposure to light from an LW Scientific Alpha 1501 Fiber Light Source (˜34 mW/cm²) for 5 minutes.

The conjunctival incision was closed with 7-0 vicryl suture. All animals received subconjunctival injections of celestone (75-150 microliters) and cepahzolin (75-150 microliters). All animals were given injections of carprofen (5 mg/kg) and buprenorphine (0.05 mg/kg) for pain and 2-3 drops of neomycin, polymixin B sulfates, and gramicidin OD to prevent infection.

Eyes were examined for any signs of pain or inflammation such as redness of the eye, discharge, ptosis of the eyelid, blepharospasm, or photophobia once a day for 1 week then once a week for 3 additional weeks.

Histology: After 4 weeks all animals were anesthetized with 30-50 mg/kg ketamine and 5-10 mg/kg xylazine, euthanized, and the eyes were removed, fixed in 10% formalin, and processed for light microscopic examination (Eosin/hematoxylin stain).

The following experiments used in vivo treatment of the eye followed by in vitro expansion on the intact globe setup to test the ability to deliver drug and treatment in a live animal.

Materials: Although in vivo treatment does not permit soaking of an entire eye and direct access to the sclera is blocked by conjunctiva and tenon, subconjunctival/subtenon injection is a low-impact surgical procedure that permits drug delivery into the space adjacent to the sclera. Literature on the subconjunctival delivery of mitomycin-C to the sclera indicates that only ˜5% of drug present on the surface of sponges is able to diffuse into the sclera, and there is a preferential uptake by the conjunctiva.[205-208] For this reason, a higher drug concentration than that used in vitro was used to achieve the desired dose in the sclera. Literature reports excellent cell viability with Eosin Y concentrations up to 20 mM and TEOA concentrations up to 450 mM.^{[209}] Our in vivo studies used a solution with 0.289 mM Eosin Y concentration, and 90 mM TEOA concentration, denoted 10×EY from here on. Solutions denoted 10×EY w/PEGDM were a mixture of 10×EY with 10% w/w Poly(ethylene glycol) dimethacrylate. All solutions were adjusted to pH 7.5 and passed through a 0.2 micron filter before use.

Surgical Procedure: The procedures for in vivo drug delivery were conducted at UCSF and were performed on 2-3 week old New Zealand White rabbits. The rabbits were given general anesthesia with 1-5% inhaled isofluorane administered by mask and topical 0.5% proparacaine to the eye. The eye of each animal was sterilized with 5% betadyne. Throughout the procedure the eye was washed with sterile ocular balanced saline solution (BSS).

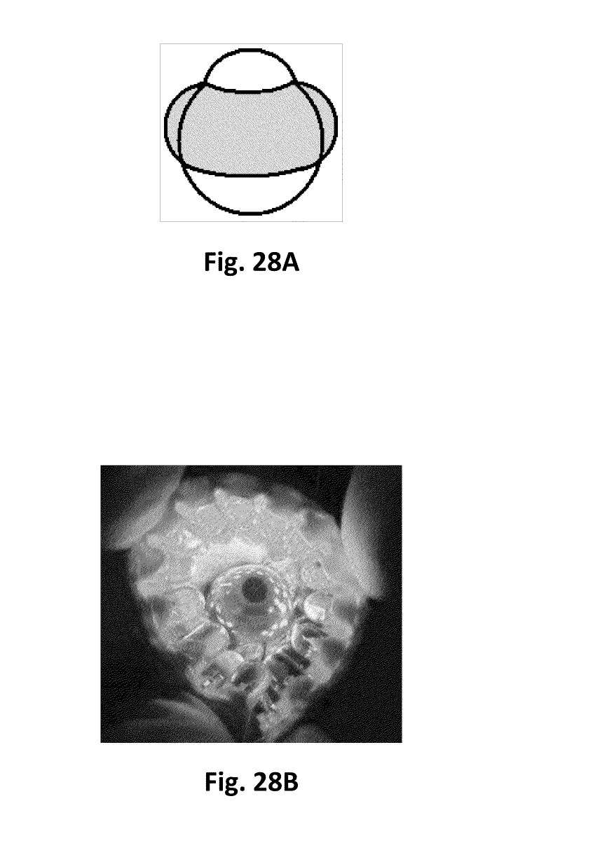

A minimal procedure using subconjunctival injection (0.6-1.2 mL) placed the drug formulation in contact with the sclera. Eight treated eyes were injected with 10×EY, four treated eyes were injected with 10×EY w/PEGDM, and four control eyes received an injection of DPBS (Table 21). The injection formed a pocket of fluid between the conjunctiva and sclera which remained during the 5 minutes given for diffusion (FIG. 28A). During this time, the lids were closed over the eye. After the 5 minute diffusion time, the lids were retracted and the eye slightly prolapsed. A circular array of 525 nm LEDs was held around the eye for 5 minutes (FIG. 28B). The control eyes received irradiation of 2 mW/cm², four 10×EY treated eyes received 2 mW/cm², four 10×EY w/PEGDM treated eyes received 2 mW/cm², and the remaining four 10×EY treated eyes received 6 mW/cm². After irradiation, the animals were sacrificed, and the eyes were enucleated and stored in DPBS on ice until use on the intact globe expansion setup.

TABLE 21
Variations for In Vivo Rabbit Treatments and Ex Vivo Expansion

| Set | Light Protocol | Drug Formulation | # of Rabbits |
|---|---|---|---|
| A | 2 mW/cm² | DPBS | 4 |
| B | 2 mW/cm² | 10x EY | 4 |
| C | 2 mW/cm² | 10x EY w/PEGDM | 4 |
| D | 6 mW/cm² | 10x EY | 4 |

Expansion Testing: Expansion experiments were performed within 48 hours post mortem. The appearance of the eyes (e.g., clarity of the cornea and size of the globe) was unchanged over this time scale. For the expansion experiment, extraocular tissues were carefully removed from the eye and then the eye was placed into DPBS for ˜1 hour to equilibrate to room temperature. The eyes were loaded onto the expansion setup where the intraocular pressure was set to 22 mmHg for 1 hour then increased to 85 mmHg for 24 hours.

These experiments in a guinea pig model were conducted. These tests examine the feasibility and safety of surgery, the safety of drug and irradiation, the effect of treatment on development of form deprivation, and the effect of treatment on normal ocular growth.

Materials: All treatment solutions were prepared at pH 7.5 and passed through a 0.2 micron filter to ensure sterility for surgery. The tests used DPBS, 3×EY (0.1 mM EY & 90 mM TEOA in DPBS), and 10×EY.

Pigmented guinea pigs (Cavia porcellus, n=47) were maternally reared and housed in their natural litters with their mothers in opaque plastic boxes (65×45×20 cm) with wire mesh lids. Water (supplemented with Vitamin C), guinea pig food pellets, and hay were available ad libitum. Light hoods containing incandescent bulbs evenly diffused through a perpex barrier were suspended 30 cm above each box and switched on a 12 h light/12 h dark cycle.

Procedures: Animals were anesthetized with Ketamine (50 mg/kg) and Xylazine (5 mg/kg) and if necessary, administered a small dose of Bupremorphine (0.1 mg/kg). The eyes received topical anesthetic as needed. On the right eye, drug was delivered through subconjunctival injection, which was previously demonstrated as a successful method in rabbits. Some animals received a sham surgery with injection of DPBS instead of drug (Table 22). After subconjunctival injection, 10 minutes was allowed for diffusion of drug formulation into the sclera.

TABLE 22
Treatment Variations for In Vivo Guinea Pig Studies

| | | | | # of | |
|---|---|---|---|---|---|
| | | Drug | Form | Guinea | Day of |
| Set | Light Protocol | formulation | Deprivation | Pigs | Enucleation |
| A | No Irradiation | 10x EY | No | 3 | Immediate |
| B | No Irradiation | No | Yes | 7 | 17 days |
| | | Treatment | | | post |
| C | 3 Trisections | 3x EY | Yes | 14 | surgery |
| D | 3 Trisections | 10x EY | No | 7 | |
| E | Circumferential | 10x EY | No | 8 | 30 days |
| F | Circumferential | DPBS | No | 8 | post |
| | | (sham) | | | surgery |

After the 10 minute diffusion time, the right eyes of Sets C-E were prolapsed and irradiated in two different manners. One group of animals had a superficial suture placed at the limbus for traction while prolapsing the eye (Sets C, D). The eye was irradiated with an LED light source for 5 minutes at each of 3 trisections. The second group of animals had a piece of elastic placed around the eye to hold it prolapsed (Set E). While prolapsed in this manner, a circular array of LEDs was placed around the eye for 5 minutes. We built the light sources from 525±16 nm LEDs to provide 6-8 mW/cm2 at the plane of the sclera; the light for trisection illumination consisted of three 5 mm LEDs aligned to irradiate a 120 degree section of the eye while held a distance of ˜8 mm from the eye, and the light for circumferential illumination consisted of 2 rows of twelve 3 mm LEDs ˜2 mm from the scleral surface that could irradiate 360 degrees of the eye.

After irradiation, the eyes were placed back in the normal position and washed with antibiotic eyedrops. The animals were placed back with their mothers after surgery and monitored to observe behavioral responses. Animals from Set A were immediately euthanized and the eyes were enucleated. The eyes were examined for the presence of Eosin Y in the sclera.

The animals from Sets B and C included form-deprivation studies. Diffusers were secured with velcro over the right eye when the animals were ˜6 days old and the fellow eye was left untreated. This was 2-3 days after surgery of animals in Set C. The animals were exposed to a 12 h/12 h light/dark cycle, and the diffusers were removed for 50-90% of the dark periods overnight. Diffusers were also removed during measurements. Animals from Sets D, E, & F did not receive diffusers and they were monitored to observe normal growth of the eye.

Measurements were made before surgery, and then periodically after surgery to track changes in eye shape throughout form deprivation and normal growth. Corneal power was measured using IR video keratometry. The animals were cyclopleged (e.g., dilated) with 2 drops of 1% cyclopentolate, and refractive error was measured using streak retinoscopy. Finally, the animals were anesthetized with 2% isoflurane in oxygen and the axial ocular parameters were measured using high-frequency ultrasound (20 MHz).

Within 2 days of the last ocular measurements, guinea pigs were euthanized and the eyes were prepared for histology. A strip of tissue was dissected from the eye cup, fixed overnight in 4% glutaldehyde, imbedded in resin, cut in 1 μm sections, and then mounted and stained.

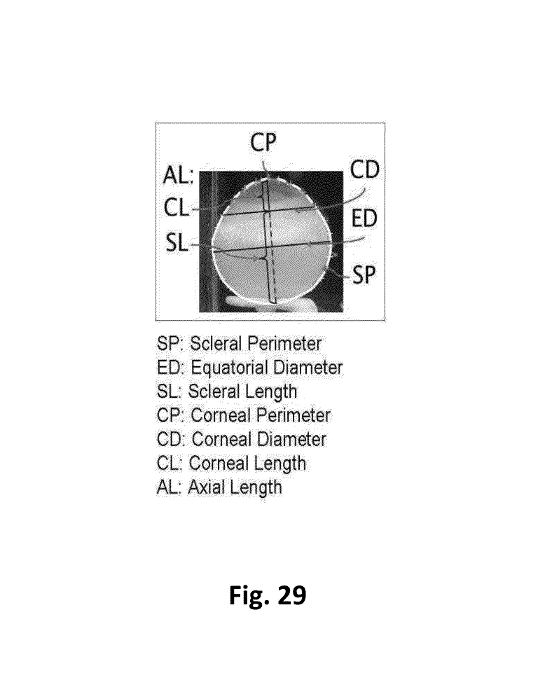

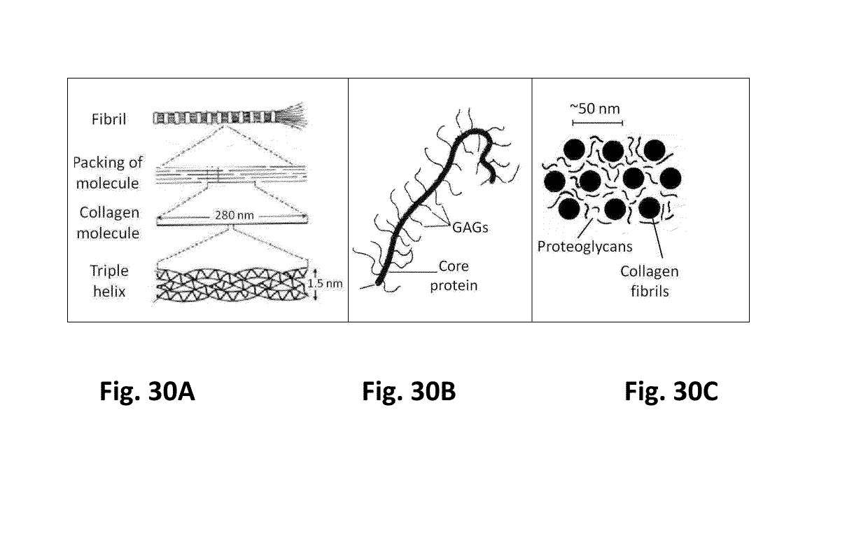

Treatment with GA was performed as a positive control to demonstrate the ability of crosslinking to prevent creep and the results have previously been discussed in prior sections of the present disclosure. Motivated by the advantages of using a visible light activated crosslinking system, we chose EY/TEOA for these studies. Digitized images from the expansion studies were analyzed to measure the ocular dimensions labeled in FIG. 29. Over the 24 hour period, control eyes expand continuously along every dimension. This is expected due to the high pressure which induces creep. The treated eyes resist expansion along SP, ED, and SL—dimensions associated with the sclera. Expansion along dimensions associated with the cornea (CP, CD, and CL) increase in the same manner for treated and control eyes. Because the corneal epithelium remained intact during treatment, it provided a protective layer that prevented treatment of the cornea. Because we are currently interested in the treatment's ability to strengthen sclera for degenerative myopia, we will focus on results of SP, ED, and SL expansion (all components of the sclera).

Treatments tested with a high-intensity, broadband arc lamp source, and with a low-intensity LED light source both show similar results after 24 hours. Further reduction in the intensity may be possible using a light source more in tune with the absorption peak of EY (514 nm). The use of low light doses (5 minutes, 6 mW/cm²) of visible wavelength may avoid the cytotoxic effects on the retina that were seen with larger doses of UV (30 minutes, 3 mW/cm²).

Biocompatibility studies were performed on albino rabbit eyes because the lack of pigmentation in these eyes allows for easy visualization of toxic or inflammatory responses. In all of the eyes we operated on, there was some observed swelling and inflammation for 2 days following the procedure. This was consistent with what would be expected to result from the surgical procedure itself. There were no clinical signs of pain or inflammation in any of the eyes 3 days after the procedure and on each examination thereafter.

Histological examination revealed that there was mild inflammation and scarring along the conjunctival-sclera junction in the surgical area of all the eyes. The irises, retinas, and ciliary bodies were all normal in all experimental groups. There was no significant difference in the sclera of treated and control eyes, indicating that the mild inflammation and scarring which occurs is a result of the surgery and not of the treatment. Likewise, the viability of cells in the nearby tissues of the treated eyes matches that of normal eyes.

Average values of changes along ocular dimensions indicate that all injections except the control decrease the expansion of the sclera. Values for expansion along SP and ED are significantly smaller compared to controls for the low-intensity treatment, while all values for the high-intensity treatment are significantly smaller than controls. Significance with p<0.05 was determined by comparing values from treatment and control groups using an unpaired t-test. After 24 hours of elevated pressure, in vivo treated eyes have an ocular stability comparable to that of the in vitro treated eyes. This proves that the subconjunctival injection delivers drug to the sclera, and the 5 minute diffusion time is sufficient for 10×EY to penetrate into the live sclera. In addition, the circumferential irradiation with LEDs is able to activate the treatment around the eye.

Using a guinea pig model, data was obtained regarding drug delivery, toxicity, and tolerance of surgical procedures. After surgery, there was minimal inflammation of the conjunctiva that disappeared within 2-3 days. The eyes had a normal pupil response and clear ocular media which allowed for streak retinoscopy measurements. Gross ocular function (pupillary reflexes, response to light, blink reflex) appeared normal. Behaviorally, the animals moved about the habitat normally and had normal eating and drinking habits.

Observations of the sham surgery controls indicated that the surgical procedure was well-tolerated by the eyes. The eyes receiving the 3×EY and 10×EY formulations demonstrated no evidence of toxicity problems. Tissue sections from treated and fellow eyes showed that the sclera was structurally normal. The sclera, choroid and retinal pigment epithelium (RPE) had no signs of toxicity from the treatment. There were normal RPE cells and depigmented RPE cells in the treated and untreated eye sections. The sclera of treated and untreated eyes is indistinguishable. Although the retina was removed before fixing the tissue, the overall retinal thickness was within the normal limits and no signs of retinal toxicity were observed. These important findings support our hypothesis that the treatment is safe based on EY/TEOA literature,¹⁻¹¹ light- and drug-penetration calculations, and rabbit histology.

Of the three eyes enucleated immediately after treatment (Set A), all showed pink staining from Eosin Y over the entire sclera, including at the posterior pole, indicating that the formulation can be delivered to the entire sclera following subconjunctival injection.

Results of normal form-deprivation with untreated eyes (Set B) are presented along with results for form-deprivation of 3×EY treated eyes (Set C). Measurements of refractive state before surgery indicate that the guinea pigs are hyperopic, which is expected for their age. Immediately before beginning form-deprivation (2 days after surgery), the treated (3×+FD) and fellow control eyes (3× Fellow) are the same, indicating that surgery had no effect on refractive error. For normal form-deprivation, the differences of refractive error between the form deprived (FD) and fellow eye (Fellow) are −4.04±0.667 D on the first measure (day 7), and −5.12±0.659 D on the second measure (day 11). Nearly identical changes are seen in the treated animals with differences of −4.11±0.675 D and −5.23±0.612 Don the matching days. Measurement of ocular length (from the front of the cornea to the back of the sclera) by ultrasound also reveals similar behavior in the treated and untreated animals. On day 7, the myopic eye was 111±20.4 μm greater in length than the fellow eye. By day 11, this length difference reduced some in the untreated animals, but remained the same in the 3×EY treated animals. The similarities between the 3×EY treated (Set C) and the normal animals (Set B) indicates that this treatment does not have an effect on the eye. We hypothesize that insufficient EY diffused into the tissue, motivating experiments with 10×EY (below).

Although this treatment was not able to prevent myopia, the results were encouraging due to the lack of cytotoxic effects using 3×EY and irradiation, and the resilience of animals to the surgery. Before examining higher doses in form-deprived animals, we began tests of higher doses in normal eyes to observe if they could tolerate the dose (Sets D & E). At this time, analysis from Sets E and F is incomplete and only results from the other sets are presented.

Eyes from Set D received the same irradiation protocol as those from Set C, but were given higher doses of drug (10×EY instead of 3×EY). Measures of refractive error indicate that 2 days after surgery there is a difference between the treated eye and untreated fellow eye of −3.11±0.714 D. The treatment causes the eye to become more myopic. Over the course of the experiment, the treated eye becomes more hyperopic. The fellow eye emmetropizes normally during this period. The 10×EY treatment also causes an increased ocular length, and the difference between treated and untreated fellow eyes reduces over time. These initial differences were not seen in the 3×EY treated eyes and they indicate that significant changes have occurred due to treatment with 10×EY.

The change in ocular length is examined in greater detail using ultrasound biometry to evaluate all the ocular dimensions that contribute to ocular length. The cornea and anterior chamber thickness (CAC) grows normally for both eyes. The lens grows normally despite an initial difference at day 2. The variability in day-to-day lens thickness suggests that the uncertainty in the measurement is greater than the error bars indicate. The vitreous chamber elongates more slowly in the treated eye than in the untreated eye. There is no difference in retinal thickness. The choroid and sclera are both thicker in the treated eye. The sum of these individual components gives the ocular length:

CAC+Lens+Vit+Ret+Scl+Chr=OL.

1.06 mm+3.53 mm+3.02 mm+0.16 mm+0.11 mm+0.11 mm=7.99 mm.

The slight differences in corneal power dissipate over the growth period. The differences in the sclera, choroid, and vitreous chamber of the treated and fellow eyes persist over 15 days of observation. Choroid thickness is known to increase with inhibitory growth signals, and the drug treatment may have triggered an inhibitory response. The initial change in vitreous chamber depth may be explained by crosslinking of the sclera in an extended state. The intraocular pressure increases during prolapsing, which could induce stretching of the sclera. After prolapsing, the pressure decreases, and the sclera relaxes back to normal. However, in a treated eye, the stretched state of the sclera might be crosslinked in place, causing noticeable shape differences. Further tests such as MRI may be capable of examining the shape of the eye before and after prolapsing, with and without treatment.

The data also suggests that the cornea grows in a normal manner in a treated eye despite the abnormal changes in vitreous chamber depth. This is also seen with the normal growth of the lens. The growth of the cornea and lens may not be coupled to axial length. Using this method of crosslinking tissue, whether it is cornea or sclera, might enable researchers to determine if there is a coupled feedback for growth of the ocular components in these animals.

Were it possible to retard or prevent abnormal axial elongation of the globe in degenerative myopia, visual loss might be prevented. Use of the expansion model in this study has allowed us to measure the progressive enlargement of the eye due to creep in the sclera. The ability of 1×EY and 10×EY to halt expansion in vitro in rabbit eyes indicates that the change in tissue properties upon treatment might prevent creep in vivo. Results from the biocompatibility studies in rabbits and guinea pigs show only minor inflammation from the surgery, and no adverse responses due to treatment concentrations up to 10×EY. Results from in vivo guinea pig studies demonstrate that the treatment with 3×EY did not alter ocular shape or prevent form-deprivation myopia. However, the higher dose of 10×EY did substantially alter ocular parameters during normal growth, possibly due to elevated intraocular pressure during prolapsing at the time of irradiation. The experiments establish protocols that may be extended to form-deprivation studies of 10×EY, perhaps with modification of the irradiation step to ensure that the globe is at normal intraocular pressure. Future treatments of the entire eye, or specifically the posterior pole, are also recommended to test their ability to prevent form-deprivation myopia in the guinea pigs.

The examples set forth above are provided to give those of ordinary skill in the art a complete disclosure and description of how to make and use the embodiments of the compositions, arrangements, devices, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which is not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

### LIST OF REFERENCES

1. Mattson, M., Understanding and Treating Eye Diseases: Mechanical Characterization and Photochemical Modification of the Cornea and Sclera. Dissertation (Ph.D), California Institute of Technology, 2008.
2. Oyster, C. W., The Human Eye: Structure and Function. 1999.
3. Wulf, H. C., Skin aging and natural photoprotection. Micron, 2004. 35(3): p. 185.
4. Dyer, D. G., Accumulation of Maillard reaction products in skin collagen in diabetes and aging. The Journal of clinical investigation, 1993. 91(6): p. 2463.
5. MacDonald, E., Advanced glycosylation end products in the mesenteric artery. Clinical Chemistry, 1992. 38(4): p. 530.
6. Sims, T. J., The role of glycation cross-links in diabetic vascular stiffening. Diabetologia, 1996. 39(8): p. 946.
7. Vitek, M. P., Advanced glycation end products contribute to amyloidosis in Alzheimer disease. Proceedings of the National Academy of Sciences of the United States of America, 1994. 91(11): p. 4766.
8. Niwa, T., Amyloid bold beta 2-microglobulin is modified with N alt epsilon-(carboxymethyl) lysine in dialysis-related amyloidosis. Kidney international, 1996. 50(4): p. 1303.
9. Tano, Y., Pathologic myopia: where are we now? American journal of ophthalmology, 2002. 134(5): p. 645-60.
10. Curtin, B. J., The myopias: basic science and clinical management. 1985.
11. Hsu, W. M. and Hsu, Prevalence and causes of visual impairment in an elderly Chinese population in Taiwan: the Shihpai Eye Study. Ophthalmology, 2004. 111(1): p. 62.
12. Iwase, A., Prevalence and Causes of Low Vision and Blindness in a Japanese Adult Population: The Tajimi Study. Ophthalmology, 2006. 113(8): p. 1354.
13. Xu, L. and Xu, Causes of Blindness and Visual Impairment in Urban and Rural Areas in Beijing: The Beijing Eye Study. Ophthalmology, 2006. 113(7).
14. McBrien, N. A. and A. Gentle, Role of the sclera in the development and pathological complications of myopia. Progress In Retinal And Eye Research, 2003. 22(3): p. 307-338.
15. Avetisov, E. E. S., et al., Nonsurgical and surgical methods of sclera reinforcement in progressive myopia. Acta ophthalmologica Scandinavica, 1997. 75(6): p. 618-23.
16. Belyaev, V. V. S. and T. T. S. Ilyina, Late results of scleroplasty in surgical treatment of progressive myopia. Eye, ear, nose & throat monthly, 1975. 54(3): p. 109-12.
17. Rabinowitz, Y. S., Keratoconus. Survey of ophthalmology, 1998. 42(4): p. 297-319.
18. Bron, A. A. J., Keratoconus. Cornea, 1988. 7(3): p. 163-9.
19. Gasset, A. R. and H. E. Kaufman, THERMOKERATOPLASTY IN TREATMENT OF KERATOCONUS. American journal of ophthalmology, 1975. 79(2): p. 226-232.
20. McDonald, M. B., et al., Epikeratophakia for keratoconus. The nationwide study. Archives of ophthalmology, 1986. 104(9): p. 1294-1300.
21. Colin, J., et al., Correcting keratoconus with intracorneal rings. Journal of cataract and refractive surgery, 2000. 26(8): p. 1117-1122.
22. Edwards, A., Fiber matrix model of sclera and corneal stroma for drug delivery to the eye. AIChE journal, 2006. 44(1): p. 214.
23. Fatt, I., Physiology of the eye: an introduction to the vegetative functions. 1992.
24. Zorn, M., et al., COLLAGEN GENE-EXPRESSION IN THE DEVELOPING TREE SHREW SCLERA. Investigative Ophthalmology & Visual Science, 1992. 33(4): p. 1053-1053.
25. McBrien, N. N. A., L. L. M. Cornell, and A. A. Gentle, Structural and ultrastructural changes to the sclera in a mammalian model of high myopia. Investigative Ophthalmology & Visual Science, 2001. 42(10): p. 2179-87.
26. Guggenheim, J. J. A. and N. N. A. McBrien, Form-deprivation myopia induces activation of scleral matrix metalloproteinase-2 in tree shrew. Investigative Ophthalmology & Visual Science, 1996. 37(7): p. 1380-95.
27. Aimes, R. R. T. and J. J. P. Quigley, Matrix metalloproteinase-2 is an interstitial collagenase. Inhibitor-free enzyme catalyzes the cleavage of collagen fibrils and soluble native type I collagen generating the specific ¾- and ¼-length fragments. The Journal of biological chemistry, 1995. 270(11): p. 5872-6.
28. Rada, J. J. A., D. D. L. Nickla, and D. D. Troilo, Decreased proteoglycan synthesis associated with form deprivation myopia in mature primate eyes. Investigative Ophthalmology & Visual Science, 2000. 41(8): p. 2050-8.
29. Curtin, B. B. J., T. T. Iwamoto, and D. D. P. Renaldo, Normal and staphylomatous sclera of high myopia. An electron microscopic study. Archives of ophthalmology, 1979. 97(5): p. 912-5.
30. Kao, W. W. Y., Increased collagenase and gelatinase activities in keratoconus. Biochemical and biophysical research communications, 1982. 107(3): p. 929.
31. Sawaguchi, S., et al., Lysosomal enzyme abnormalities in keratoconus. Archives of ophthalmology, 1989. 107(10): p. 1507-1510.
32. Smith, V. A., et al., Over-expression of a gelatinase A activity in keratoconus. Eye, 1995. 9(4): p. 429-433.
33. Wollensak, G. and E. Spoerl, Collagen crosslinking of human and porcine sclera. Journal of cataract and refractive surgery, 2004. 30(3): p. 689-95.
34. Spoerl, E., Induction of cross-links in corneal tissue. Experimental Eye Research, 1998. 66(1): p. 97.
35. Wollensak, G. and E. Iomdina, Long-term biomechanical properties after collagen crosslinking of sclera using glyceraldehyde. Acta Ophthalmologica, 2008. 86(8): p. 887-893.
36. Spoerl, E., Thermomechanical behavior of collagen-cross-linked porcine cornea. Ophthalmologica, 2004. 218(2): p. 136.
37. Wollensak, G., et al., Hydration behavior of porcine cornea crosslinked with riboflavin and ultraviolet A. Journal of cataract and refractive surgery, 2007. 33(3): p. 516-521.
38. Wollensak, G., et al., Collagen fiber diameter in the rabbit cornea after collagen crosslinking by riboflavin/UVA. Cornea, 2004. 23(5): p. 503-507.
39. Spoerl, E., Increased resistance of crosslinked cornea against enzymatic digestion. Current eye research, 2004. 29(1): p. 35.
40. Wollensak, G., Gel electrophoretic analysis of corneal collagen after photodynamic cross-linking treatment. Cornea, 2008. 27(3): p. 353.
41. Wollensak, G., E. Spoerl, and T. Seiler, Riboflavin/ultraviolet-a-induced collagen crosslinking for the treatment of keratoconus. American journal of ophthalmology, 2003. 135(5): p. 620-627.
42. MÃ¼ller, L. J., Novel aspects of the ultrastructural organization of human corneal keratocytes. Investigative Ophthalmology & Visual Science, 1995. 36(13): p. 2557.
43. Mazzotta, C., Stromal haze after combined riboflavin/UVA corneal collagen cross-linking in keratoconus: in vivo confocal microscopic evaluation. Clinical & experimental ophthalmology, 2007. 35(6): p. 580.
44. Mazzotta, C., Treatment of progressive keratoconus by riboflavin-UVA-induced cross-linking of corneal collagen: ultrastructural analysis by Heidelberg Retinal Tomograph II in vivo confocal microscopy in humans. Cornea, 2007. 26(4): p. 390.
45. Spoerl, E., Safety of UVA-riboflavin cross-linking of the cornea. Cornea, 2007. 26(4): p. 385.
46. Seiler, T., Corneal cross-linking-induced stromal demarcation line. Cornea, 2006. 25(9): p. 1057.
47. Wollensak, G., Corneal endothelial cytotoxicity of riboflavin/UVA treatment in vitro. Ophthalmic research, 2003. 35(6): p. 324.
48. Wollensak, G., Crosslinking of scleral collagen in the rabbit using riboflavin and UVA. Acta ophthalmologica Scandinavica, 2005. 83(4): p. 477.
49. Alleyene, C. C. H., et al., Efficacy and biocompatibility of a photopolymerized, synthetic, absorbable hydrogel as a dural sealant in a canine craniotomy model. Journal of neurosurgery, 1998. 88(2): p. 308-13.
50. Torchiana, D. F. D. F., Polyethylene glycol based synthetic sealants: potential uses in cardiac surgery. Journal of cardiac surgery, 2003. 18(6): p. 504-6.
51. Alleyne Jr, C. H. and Alleyne, Efficacy and biocompatibility of a photopolymerized, synthetic, absorbable hydrogel as a dural sealant in a canine craniotomy model. Journal of neurosurgery, 1998. 88(2): p. 308.
52. Nakayama, Y., Enhancement of visible light-induced gelation of photocurable gelatin by addition of polymeric amine. Journal of photochemistry and photobiology. A, Chemistry, 2006. 177(2-3): p. 205.
53. Janine M. Orban, K. M. F., Richard A. Dluhy, and Elliot L. Chaikof, Cytomimetic Biomaterials. 4. In-Situ Photopolymerization of Phospholipids on an Alkylated Surface. Macromolecules, 2000. 33: p. 4205-4212.
54. Gregory M. Cruise, O. D. H., David S. Scharp, Jeffrey A. Hubbell, A Sensitivity Study of the Key Parameters in the Interfacial Photopolymerization of Poly(ethylene glycol) Diacrylate upon Porcine Islets. Biotechnology and Bioengineering, 1997. 57(6): p. 655-665.
55. Chandrashekhar P. Pathak, A. S. S., and J. A. Hubbell, Rapid Photopolymerization of Immunoprotective Gels in Contact with Cells and Tissue. Journal of American Chemical Society, 1992. 114: p. 8311-8312.
56. A.-I. Desmangles, O. J., F. Marquis-Weible, Interfacial Photopolymerization of b-Cell Clusters: Approaches to Reduce Coating Thickness Using Ionic and Lipophilic Dyes. Biotechnology and Bioengineering, 2000. 72(6): p. 634-641.
57. Elisseeff, J., et al., Transdermal photopolymerization for minimally invasive implantation. Proc. Natl. Acad. Sci. USA, 1999. 96: p. 3104-3107.
58. Nathanael R. Luman, T. K., and Mark W. Grinstaff, Dendritic polymers composed of glycerol and succinic acid: Synthetic methodologies and medical applications. Pure Applied Chemistry, 2004. 76: p. 1375-1385.
59. Michael A. Carnahan, C. M., Jitek Kim, Terry Kim, and Mark W. Grinstaff, Hybrid Dendritic-Linear Polyester-Ethers for in Situ Photopolymerization. Journal of American Chemical Society, 2002. 124: p. 5291-5293.
60. West, J. L. and J. A. Hubbell, Separation of the arterial wall from blood contact using hydrogel barriers reduces intimal thickening after balloon injury in the rat: The roles of medial and luminal factors in arterial healing. Proclomations of the National Academy of Science, 1996. 93: p. 13188-13193.
61. Hill-West, J. L., et al., Inhibition of thrombosis and intimal thickening by in situ photopolymerization of thin hydrogel barriers. Proclomations of the National Academy of Science, 1994. 91: p. 5967-5971.
62. Henderson, B. W., Photodynamic therapy: basic principles and clinical applications. 1992.
63. Henderson, B. W., How does photodynamic therapy work? Photochemistry and photobiology, 1992. 55(1): p. 145.
64. Chan, B. P., Photochemical cross-linking for collagen-based scaffolds: a study on optical properties, mechanical properties, stability, and hematocompatibility. Tissue engineering, 2007. 13(1): p. 73.
65. Chan, B. P., Photochemical crosslinking improves the physicochemical properties of collagen scaffolds. Journal of biomedical materials research. Part A, 2005. 75A(3): p. 689.
66. Wollensak, G. and E. Spoerl, Collagen crosslinking of human and porcine sclera. Journal of cataract and refractive surgery, 2004. 30(3): p. 689-695.
67. Krachmer, J. H., R. S. Feder, and M. W. Belin, Keratoconus and related noninflammatory corneal thinning disorders. Survey of ophthalmology, 1984. 28(4): p. 293-322.
68. Randleman, J. B., Post-laser in-situ keratomileusis ectasia: current understanding and future directions. Current opinion in ophthalmology, 2006. 17(4): p. 406.
69. Binder, P., Ectasia after laser in situ keratomileusis. Journal of cataract and refractive surgery, 2003. 29(12): p. 2419-29.
70. McBrien, N. A., Role of the sclera in the development and pathological complications of myopia. Progress in Retinal and Eye Research, 2003. 22(3): p. 307.
71. Wollensak, G., Crosslinking treatment of progressive keratoconus: new hope. Current opinion in ophthalmology, 2006. 17(4): p. 356.
72. Raiskup-Wolf, F., Collagen crosslinking with riboflavin and ultraviolet-A light in keratoconus: Long-term results. Journal of cataract and refractive surgery, 2008. 34(5): p. 796.
73. Wollensak, G. and E. Iomdina, Long-term biomechanical properties of rabbit sclera after collagen crosslinking using riboflavin and ultraviolet A (UVA). Acta Ophthalmologica, 2009. 87(2): p. 193-198.
74. Kymionis, G. D., One-Year Follow-up of Corneal Confocal Microscopy After Corneal Cross-Linking in Patients With Post Laser In Situ Keratosmileusis Ectasia and Keratoconus. American journal of ophthalmology, 2009. 147(5): p. 774.
75. Wollensak, G. and E. Iomdina, Biomechanical and histological changes after corneal crosslinking with and without epithelial debridement. Journal of Cataract & Refractive Surgery, 2009. 35(3): p. 540-546.
76. Webster, A., A dye-photosensitized reaction that generates stable protein-protein crosslinks. Analytical biochemistry, 1989. 179(1): p. 154.
77. Balasubramanian, D., The reaction of singlet oxygen with proteins, with special reference to crystallins. Photochemistry and photobiology, 1990. 52(4): p. 761.
78. Shen, H. R., Photodynamic crosslinking of proteins II. Photocrosslinking of a model protein-ribonuclease A. Journal of photochemistry and photobiology. B, Biology, 1996. 35(3): p. 213.
79. Foote, C. S., Definition of type I and type II photosensitized oxidation. Photochemistry and photobiology, 1991. 54(5): p. 659.
80. Verweu, H., MODEL STUDIES ON PHOTODYNAMIC CROSS-LINKING. Photochemistry and photobiology, 1982. 35(2): p. 265.
81. Spikes, J. D., DYE-SENSITIZED PHOTOOXIDATION OF PROTEINS*. Annals of the New York Academy of Sciences, 1970. 171(1 International): p. 149.
82. Shen, H. R., Photodynamic crosslinking of proteins. I. Model studies using histidine- and lysine-containing N-(2-hydroxypropyl) methacrylamide copolymers. Journal of photochemistry and photobiology. B, Biology, 1996. 34(2-3): p. 203.
83. Nilsson, R. and Nilsson, Unambiguous evidence for the participation of singlet oxygen (1Î″) in photodynamic oxidation of amino acids. Photochemistry and photobiology, 1972. 16(2): p. 117.
84. McCall, A. S., Mechanisms of Corneal Tissue Cross-linking in Response to Treatment with Topical Riboflavin and Long-Wavelength Ultraviolet Radiation (UVA). Investigative ophthalmology & visual science. 51(1): p. 129.
85. Zigler, J. S., et al., Photodynamic cross-linking of polypeptides in intact rat lens. Experimental Eye Research, 1982. 35(3): p. 239-249.
86. Khan, S. A., In situ technique for monitoring the gelation of UV curable polymers. Rheologica acta, 1992. 31(2): p. 151.
87. Kohlhaas, M. and Kohlhaas, Biomechanical evidence of the distribution of cross-links in corneas treated with riboflavin and ultraviolet A light. Journal of cataract and refractive surgery, 2006. 32(2): p. 279.
88. Hall, R. D., STEADYâ. STATE NEAR INFRARED DETECTION OF SINGLET MOLECULAR OXYGEN: A STERN-VOLMER QUENCHING EXPERIMENT WITH SODIUM AZIDE. Photochemistry and photobiology, 1987. 45(4): p. 459.
89. Amat-Guerri, F., et al., Singlet oxygen photogeneration by ionized and un-ionized derivatives of Rose Bengal and Eosin Y in diluted solutions. Journal of Photochemistry and Photobiology A: Chemistry, 1990. 53(2): p. 199-210.
90. Miskoski, S. and N. A. Garca, Influence of the peptide bond on the singlet molecular oxygen-mediated (02[1 delta g]) photooxidation of histidine and methionine dipeptides. A kinetic study. Photochemistry and photobiology, 1993. 57(3): p. 447-452.
91. Foote, C. S., Singlet Oxygen. A Probable Intermediate in Photosensitized Autoxidations1. Journal of the American Chemical Society, 1964. 86(18): p. 3880.
92. Matheson, I. B. C., Chemical reaction rates of amino acids with singlet oxygen. Photochemistry and photobiology, 1979. 29(5): p. 879.
93. Shen, H. R., Photodynamic cross-linking of proteins: IV. Nature of the His-His bond (s) formed in the rose bengal-photosensitized cross-linking of N-benzoyl—histidine. Journal of photochemistry and photobiology. A, Chemistry, 2000. 130(1): p. 1.
94. Criado, S., Sensitized Photooxidation of Diâ and Tripeptides of Tyrosine*. Photochemistry and photobiology, 1998. 68(4): p. 453.
95. Verweij, H., Photodynamic protein cross-linking. Biochimica et biophysica acta. Biomembranes, 1981. 647(1): p. 87.
96. Hemmendorff, B., Photosensitized labeling of solvent-exposed parts of proteins: Studies on fibrinogen and the fibrinogen-fibrin conversion. Biochimica et biophysica acta. Protein structure, 1981. 667(1): p. 15.
97. Ramshaw, J. A. M., Methylene blue sensitized photo-oxidation of collagen fibrils. Biochimica et biophysica acta. Protein structure and molecular enzymology, 1994. 1206(2): p. 225.
98. Dubbelman, T. M., C. Haasnoot, and J. van Steveninck, Temperature dependence of photodynamic red cell membrane damage. Biochimica et biophysica acta, 1980. 601(1): p. 220-7.
99. Michaeli, A., Reactivity of singlet oxygen toward large peptides. Photochemistry and photobiology, 1995. 61(3): p. 255.
100. Michaeli, A., Reactivity of singlet oxygen toward amino acids and peptides. Photochemistry and photobiology, 1994. 59(3): p. 284.
101. Davies, M. J., Singlet oxygen-mediated damage to proteins and its consequences. Biochemical and biophysical research communications, 2003. 305(3): p. 761.
102. Eastoe, J. E., AMINO ACID COMPOSITION OF MAMMALIAN COLLAGEN AND GELATIN. Biochemical Journal, 1955. 61(4): p. 589-600.
103. Dubbelman, T., Photodynamic effects of protoporphyrin on human erythrocytes. Nature of the cross-linking of membrane proteins. Biochimica et biophysica acta. Biomembranes, 1978. 511(2): p. 141.
104. Kato, Y., Aggregation of collagen exposed to UVA in the presence of riboflavin: a plausible role of tyrosine modification. Photochemistry and photobiology, 1994. 59(3): p. 343.
105. Spikes, J. D., Photodynamic crosslinking of proteins. III. Kinetics of the FMN- and rose bengal-sensitized photooxidation and intermolecular crosslinking of model tyrosine-containing N-(2-hydroxypropyl) methacrylamide copolymers. Photochemistry and photobiology, 1999. 70(2): p. 130.
106. Miranda, M. A., Drug-Photosensitized Protein Modification: Identification of the Reactive Sites and Elucidation of the Reaction Mechanisms with Tiaprofenic Acid/Albumin as Model Systema Chemical research in toxicology, 1998. 11(3): p. 172.
107. Waheed, A. A., Mechanism of dye binding in the protein assay using eosin dyes. Analytical biochemistry, 2000. 287(1): p. 73.
108. Wollensak, G., et al., Endothelial cell damage after riboflavin-ultraviolet-A treatment in the rabbit. Journal of cataract and refractive surgery, 2003. 29(9): p. 1786-90.
109. Wollensak, G., Keratocyte cytotoxicity of riboflavin/UVA-treatment in vitro. Eye, 2004. 18(7): p. 718.
110. Kolli, S., Safety and efficacy of collagen crosslinking for the treatment of keratoconus. Expert Opinion on Drug Safety. 9(6): p. 949.
111. Ashwin, P. T., Collagen cross-linkage: a comprehensive review and directions for future research. British journal of ophthalmology. 94(8): p. 965.
112. Bilgihan, K. and Bilgihan, Excimer laser-assisted anterior lamellar keratoplasty for keratoconus, corneal problems after laser in situ keratomileusis, and corneal stromal opacities. Journal of cataract and refractive surgery, 2006. 32(8): p. 1264.
113. Cook, W. D., Photopolymerization kinetics, photorheology and photoplasticity of thiol-ene-allylic sulfide networks. Polymer international, 2008. 57(3): p. 469.
114. Schmidt, L. E., Photorheology of Fast UV-Curing Multifunctional Acrylates. Macromolecular materials and engineering, 2005. 290(11): p. 1115.
115. Ibusuki, S., Photochemically cross-linked collagen gels as three-dimensional scaffolds for tissue engineering. Tissue engineering, 2007. 13(8): p. 1995.
116. Brinkman, W. T., Photo-cross-linking of type I collagen gels in the presence of smooth muscle cells: mechanical properties, cell viability, and function. Biomacromolecules, 2003. 4(4): p. 890.
117. Son, T., et al., Visible light-induced crosslinkable gelatin. Acta Biomaterialia. 6(10): p. 4005-4010.
118. Judy, M. M., Gel electrophoretic studies of photochemical cross-linking of type I collagen with brominated 1,8-naphthalimide dyes and visible light
Proceedings of SPIE. 1994. 506.
119. Bown, S. G., Photodynamic therapy with porphyrin and phthalocyanine sensitisation: quantitative studies in normal rat liver. British Journal of Cancer, 1986. 54(1): p. 43.
120. Wilson, B. C., Effect of photosensitizer concentration in tissue on the penetration depth of photoactivating light. Lasers in medical science, 1986. 1(4): p. 235.
121. Terrones, G., Effects of optical attenuation and consumption of a photobleaching initiator on local initiation rates in photopolymerizations. Macromolecules, 2001. 34(10): p. 3195.
122. Lee, J. H., Cure depth in photopolymerization: Experiments and theory. Journal of materials research, 2001. 16(12): p. 3536.
123. Lee, V. H. L. and Lee, Topical ocular drug delivery: recent developments and future challenges. Journal of ocular pharmacology and therapeutics, 1986. 2(1): p. 67.
124. Davies, N. M., Biopharmaceutical considerations in topical ocular drug delivery. Clinical and experimental pharmacology & physiology, 2000. 27(7): p. 558.
125. Urtti, A., Challenges and obstacles of ocular pharmacokinetics and drug delivery. Advanced drug delivery reviews, 2006. 58(11): p. 1131.
126. Burstein, N. L., Corneal penetration and ocular bioavailability of drugs. Journal of ocular pharmacology and therapeutics, 1985. 1(3): p. 309.
127. Kohlhaas, M., et al., A new treatment of keratectasia after LASIK by using collagen with riboflavin/UVA light cross-linking. Klinische MonatsblÃ [symbol] tter fÃ¼r Augenheilkunde, 2005. 222(5): p. 430-6.
128. Bron, A. J., Keratoconus. Cornea, 1988. 7(3): p. 163-9.
129. Binder, P., et al., Keratoconus and corneal ectasia after LASIK. Journal of refractive surgery, 2005. 21(6): p. 749-752.
130. Klein, S. R., Corneal ectasia after laser in situ keratomileusis in patients without apparent preoperative risk factors. Cornea, 2006. 25(4): p. 388.
131. Rada, J. A. and Summersrada, The sclera and myopia. Experimental Eye Research, 2006. 82(2): p. 185.
132. SÃ,ndergaard, A. P. and Søndergaard, Corneal Distribution of Riboflavin Prior to Collagen Cross-Linking. Current eye research. 35(2): p. 116.
133. Cui, L., et al., High-resolution, non-invasive two-photon fluorescence measurement of molecular concentrations in corneal tissue. Investigative ophthalmology & visual science.
134. Maurice, D., THE MOVEMENT OF FLUORESCEIN AND WATER IN THE CORNEA. American journal of ophthalmology, 1960. 49(5): p. 1011-1016.
135. Nagataki, S., The diffusion of fluorescein in the stroma of rabbit cornea. Experimental Eye Research, 1983. 36(6): p. 765.
136. Shiraya, K., Movement of fluorescein monoglucuronide in the rabbit cornea. Diffusion in the stroma and endothelial permeability. Investigative ophthalmology & visual science, 1986. 27(1): p. 24.
137. Maren, T. H., The transcorneal permeability of sulfonamide carbonic anhydrase inhibitors and their effect on aqueous humor secretion. Experimental Eye Research, 1983. 36(4): p. 457.
138. Ahmed, I., Physicochemical determinants of drug diffusion across the conjunctiva, sclera, and cornea. Journal of pharmaceutical sciences, 1987. 76(8): p. 583.
139. Ambati, J., Diffusion of high molecular weight compounds through sclera. Investigative ophthalmology & visual science, 2000. 41(5): p. 1181.
140. Boubriak, O. A., The effect of hydration and matrix composition on solute diffusion in rabbit sclera. Experimental Eye Research, 2000. 71(5): p. 503.
141. Jiang, J., Measurement and prediction of lateral diffusion within human sclera. Investigative ophthalmology & visual science, 2006. 47(7): p. 3011.
142. Prausnitz, M. R., Measurement and prediction of transient transport across sclera for drug delivery to the eye. Industrial & engineering chemistry research, 1998. 37(8): p. 2903.
143. Prausnitz, M. R., Permeability of cornea, sclera, and conjunctiva: a literature analysis for drug delivery to the eye. Journal of pharmaceutical sciences, 1998. 87(12): p. 1479.
144. Chan, T., Corneal thickness profiles in rabbits using an ultrasonic pachometer. Investigative ophthalmology & visual science, 1983. 24(10): p. 1408.
145. Bartholomew, L. R., et al., Ultrasound biomicroscopy of globes from young adult pigs. American journal of veterinary research, 1997. 58(9): p. 942-8.
146. Cheruvu, N. P. S., Bovine and porcine transscleral solute transport: influence of lipophilicity and the Choroid-Bruch's layer. Investigative ophthalmology & visual science, 2006. 47(10): p. 4513.
147. Birkedal-Hansen, H., Eosin staining of gelatine. Histochemistry and cell biology, 1973. 36(1): p. 73.
148. Ricard, C., In vivo imaging of elastic fibers using sulforhodamine B. Journal of biomedical optics, 2007. 12(6): p. 064017.
149. Ricard, C., Imaging elastic and collagen fibers with sulforhodamine B and second-harmonic generation
Proceedings of SPIE. 2008. 686700.
150. Chodosh, J., Staining characteristics and antiviral activity of sulforhodamine B and lissamine green B. Investigative ophthalmology & visual science, 1994. 35(3): p. 1046.
151. Anderson, R. F., Energetics of the one-electron reduction steps of riboflavin, FMN and FAD to their fully reduced forms. Biochimica et biophysica acta. Bioenergetics, 1983. 722(1): p. 158.
152. Shin, H. S., Permeation of solutes through interpenetrating polymer network hydrogels composed of poly (vinyl alcohol) and poly (acrylic acid). Journal of applied polymer science, 1998. 69(3): p. 479.
153. Luo, Y., Cross-linked hyaluronic acid hydrogel films: new biomaterials for drug delivery. Journal of controlled release, 2000. 69(1): p. 169.
154. Le Bourlais, C., Ophthalmic drug delivery systems—recent advances. Progress in Retinal and Eye Research, 1998. 17(1): p. 33.
155. Le Bourlais, C. A., New ophthalmic drug delivery systems. Drug development and industrial pharmacy, 1995. 21(1): p. 19.
156. Kaur, I. P., Penetration enhancers and ocular bioadhesives: two new avenues for ophthalmic drug delivery. Drug development and industrial pharmacy, 2002. 28(4): p. 353.
157. Lin, H. R., In situ gelling of alginate/pluronic solutions for ophthalmic delivery of pilocarpine. Biomacromolecules, 2004. 5(6): p. 2358.
158. Mathews, M. F., The ocular impression: a review of the literature and presentation of an alternate technique. Journal of prosthodontics, 2000. 9(4): p. 210.
159. Kumar, S., In situ-forming gels for ophthalmic drug delivery. Journal of ocular pharmacology and therapeutics, 1994. 10(1): p. 47.
160. Trueblood, J. H., et al., Corneal contact times of ophthalmic vehicles. Evaluation by microscintigraphy. Archives of ophthalmology, 1975. 93(2): p. 127-30.
161. Berman, Collagenase inhibitors: rationale for their use in treating corneal ulceration. Int Ophthalmol Clin, 1975. 15(4): p. 49-66.
162. Rehany, U., M. Lahav, and S. Shoshan, Collagenolytic activity in keratoconus. Annals of ophthalmology, 1982. 14(8): p. 751-754.
163. Wollensak, G., E. Spoerl, and T. Seiler, Stress-strain measurements of human and porcine corneas after riboflavin-ultraviolet-A-induced cross-linking. Journal of cataract and refractive surgery, 2003. 29(9): p. 1780-1785.
164. Wollensak, G., et al., Keratocyte apoptosis after corneal collagen cross-linking using riboflavin/UVA treatment. Cornea, 2004. 23(1): p. 43-49.
165. Coakes, R. L., Method of measuring aqueous humor flow and corneal endothelial permeability using a fluorophotometry nomogram. Investigative ophthalmology & visual science, 1979. 18(3): p. 288.
166. Proano, C. E., Photochemical keratodesmos for bonding corneal incisions. Investigative ophthalmology & visual science, 2004. 45(7): p. 2177.
167. Mulroy, L., Photochemical keratodesmos for repair of lamellar corneal incisions. Investigative ophthalmology & visual science, 2000. 41(11): p. 3335.
168. Ritch, R. and J. M. Liebmann, Argon laser peripheral iridoplasty. Ophthalmic surgery and lasers, 1996. 27(4): p. 289-300.
169. Spitznas, M., Long-term functional and topographic results seven years after epikeratophakia for keratoconus. Graefe's archive for clinical and experimental ophthalmology, 2002. 240(8): p. 639.
170. Wollensak, G., E. Sprl, and T. Seiler, [Treatment of keratoconus by collagen cross linking]. Der Ophthalmologe, 2003. 100(1): p. 44-49.
171. Wollensak, G., Haze or calcific band keratopathy after crosslinking treatment? Ophthalmologe, 2008. 105(9): p. 864-865.
172. Wilson, S. E., S. S. Chaurasia, and F. W. Medeiros, Apoptosis in the initiation, modulation and termination of the corneal wound healing response. Experimental Eye Research, 2007. 85(3): p. 305-311.
173. Mazzotta, C., Corneal healing after riboflavin ultraviolet-A collagen cross-linking determined by confocal laser scanning microscopy in vivo: early and late modifications. American journal of ophthalmology, 2008. 146(4): p. 527.
174. Hjortdal, J., Corneal power, thickness, and stiffness: Results of a prospective randomized controlled trial of PRK and LASIK for myopia. Journal of cataract and refractive surgery, 2005. 31(1): p. 21.
175. Rada, J. A. S., S. Shelton, and T. T. Norton, The sclera and myopia. Experimental Eye Research, 2006. 82(2): p. 185-200.
176. Blinder, K. J., et al., Verteporfin therapy of subfoveal choroidal neovascularization in pathologic myopia—2-year results of a randomized clinical Trial—VIP report no. 3. Ophthalmology, 2003. 110(4): p. 667-673.
177. Chan, W. M., et al., Choroidal neovascularisation in pathological myopia: an update in management. British Journal of Ophthalmology, 2005. 89(11): p. 1522-1528.
178. Buys, Y. M., Bevacizumab: the need for controlled studies to move forward. Canadian Journal of Ophthalmology-Journal Canadien D Ophtalmologie, 2007. 42(6): p. 789-789.
179. Rosenfeld, P. J., Intravitreal Avastin for choroidal neovascularisation in pathological myopia: the controversy continues. British Journal of Ophthalmology, 2007. 91(2): p. 128-130.
180. Yamamoto, I., et al., Intravitreal bevacizumab (Avastin) as treatment for subfoveal choroidal neovascularisation secondary to pathological myopia. British Journal of Ophthalmology, 2007. 91(2): p. 157-160.
181. Sakaguchi, H., et al., Intravitreal injection of bevacizumab for choroidal neovascularisation associated with pathological myopia. British Journal of Ophthalmology, 2007. 91(2): p. 161-165.
182. Avetisov, E. S., et al., Nonsurgical and surgical methods of sclera reinforcement in progressive myopia. Acta Ophthalmologica Scandinavica, 1997. 75(6): p. 618-623.
183. Chua, W. H., et al., Progression of childhood myopia following cessation of atropine treatment. Investigative Ophthalmology & Visual Science, 2005. 46
184. Tarutta, Y. P., et al., Sclera Fortification In Children At A High-Risk Of Progressive Myopia. Vestnik Oftalmologii, 1992. 108(2): p. 14-17.
185. Politzer, M., Experiences In Medical-Treatment Of Progressive Myopia. Klinische Monatsblatter Fur Augenheilkunde, 1977. 171(4): p. 616-619.
186. Belyaev, V. S. and T. S. Ilyina, Late Results Of Scleroplasty In Surgical Treatment Of Progressive Myopia. Eye Ear Nose And Throat Monthly, 1975. 54(3): p. 109-113.
187. Chauvaud, D., M. Assouline, and F. Perrenoud, Scleral reinforcement. Journal Francais D Ophtalmologie, 1997. 20(5): p. 374-382.
188. Jacob, J. T., J. J. Lin, and S. P. Mikal, Synthetic scleral reinforcement materials 0.3. Changes in surface and bulk physical properties. Journal Of Biomedical Materials Research, 1997. 37(4): p. 525-533.
189. Korobelnik, J. F., et al., Expanded polytetrafluoroethylene episcleral implants used as encircling scleral buckling—An experimental and histopathological study. Ophthalmic Research, 2000. 32(2-3): p. 110-117.
190. Mortemousque, B., et al., S/e-PTFE episcleral buckling implants: An experimental and histopathologic study. Journal Of Biomedical Materials Research, 2002. 63(6): p. 686-691.
191. Jacoblabarre, J. T., et al., Effects Of Scleral Reinforcement On The Elongation Of Growing Cat Eyes. Archives Of Ophthalmology, 1993. 111(7): p. 979-986.
192. Bailey, A. J., Molecular mechanisms of ageing in connective tissues. Mechanisms of Ageing and Development, 2001. 122(7): p. 735-755.
193. Singh, R., et al., Advanced glycation end-products: a review. Diabetologia, 2001. 44(2): p. 129-146.
194. Spoerl, E., G. Wollensak, and T. Seiler, Increased resistance of crosslinked cornea against enzymatic digestion. Current Eye Research, 2004. 29(1): p. 35-40.
195. Wollensak, G., et al., Cross-linking of scleral collagen in the rabbit using riboflavin and UVA. Acta Ophthalmologica Scandinavica, 2005. 83(4): p. 477-482.
196. Norton, T. T., Animal Models of Myopia: Learning How Vision Controls the Size of the Eye. ILAR, 1999. 40(2): p. 59-77.
197. Gao, Q. Y., et al., Effects of direct intravitreal dopamine injections on the development of lid-suture induced myopia in rabbits. Graefes Archive For Clinical And Experimental Ophthalmology, 2006. 244(10): p. 1329-1335.
198. Howlett, M. H. C. and S. A. McFadden, Emmetropization and schematic eye models in developing pigmented guinea pigs. Vision Research, 2007. 47(9): p. 1178-1190.
199. Zhou, X. T., et al., Normal development of refractive state and ocular dimensions in guinea pigs. Vision Research, 2006. 46(18): p. 2815-2823.
200. Lu, F., et al., Axial myopia induced by a monocularly-deprived facemask in guinea pigs: A non-invasive and effective model. Experimental Eye Research, 2006. 82(4): p. 628-636.
201. Howlett, M. H. C. and S. A. McFadden, Form-deprivation myopia in the guinea pig (Cavia porcellus). Vision Research, 2006. 46(1-2): p. 267-283.
202. McFadden, S. A., M. H. C. Howlett, and J. R. Mertz, Retinoic acid signals the direction of ocular elongation in the guinea pig eye. Vision Research, 2004. 44(7): p. 643-653.
203. McFadden, S. A., Partial occlusion produces local form deprivation myopia in the guinea pig eye. Investigative Ophthalmology & Visual Science, 2002. 43: p. U34.
204. Lodge, A., T. Peto, and S. A. McFadden, Form deprivation myopia and emmetropization in the guinea pig. Proceedings of the Australian Neuroscience Society, 1994. 5: p. 123.
205. Georgopoulos, M., et al., In vitro diffusion of mitomycin-C into human sclera after episcleral application: Impact of diffusion time. Experimental Eye Research, 2000. 71(5): p. 453-457.
206. Vass, C., et al., Intrascleral concentration vs depth profile of mitomycin-C after episcleral application: Impact of applied concentration and volume of mitomycin-C solution. Experimental Eye Research, 2000. 70(5): p. 571-575.
207. Vass, C., et al., Intrascleral concentration vs depth profile of mitomycin-C after episcleral application: Impact of irrigation. Experimental Eye Research, 2000. 70(2): p. 139-143.
208. Vass, C., et al., Impact of Mitomycin-C application time on the scleral Mitomycin-C concentration. Journal of Ocular Pharmacology and Therapeutics, 2001. 17(2): p. 101-105.
209. Cruise, G. M., et al., A sensitivity study of the key parameters in the interfacial photopolymerization of poly(ethylene glycol) diacrylate upon porcine islets. Biotechnology and Bioengineering, 1998. 57(6): p. 655-665.
210. Sliney D. H. and M. L. Wolbarsht (1980) Safety with Lasers and Other Optical Sources, A Comprehensive Handbook, New York, Plenum (Kluwer).
211. Ham, W. T., Jr., H. S. Mueller and D. H. Sliney (1976) Retinal sensitivity to damage by short-wavelength light. Nature, 260(5547): 153-155.
212. Ham, W. T., Jr. (1989) The photopathology and nature of the blue-light and near-UV retinal lesion produced by lasers and other optical sources (M. L. Wolbarsht, ed.) Laser Applications in Medicine and Biology, New York, Plenum Publishing Corp.
213. Pitts D. G. and A. P. Cullen (1981) Determination of infrared radiation levels for acute ocular cataractogenesis, Albrecht von Graefes Arch Klin Ophthalmol, 217:285-297.
214. American Conference of Governmental Industrial Hygienists (ACGIH) (2009), TLV's, Threshold Limit Values and Biological Exposure Indices for 2009, American Conference of Governmental Industrial Hygienists, Cincinnati, Ohio
215. ACGIH (2007), Documentation for the Threshold Limit Values, American Conference of Governmental Industrial Hygienists, Cincinnati, Ohio.
216. International Commission on Non-Ionizing Radiation Protection, Guidelines on limits of exposure to broad-band incoherent optical radiation (0.38 to 3 μm). Health Phys. 73(3): 539554; 1997.
217. World Health Organization [WHO], (1982), Environmental Health Criteria No. 23, Lasers and Optical Radiation, joint publication of the United Nations Environmental Program, the International Radiation Protection Association and the World Health Organization, Geneva.
218. American National Standards Institute/Illuminating Engineering Society of North America (ANSI/IESNA) (2005) Photobiological Safety of Lamps and Lighting Systems—General Requirements, RP27.1-05, New York, IESNA.
219. American National Standards Institute/Illuminating Engineering Society of North America (ANSI/IESNA) (2000) Photobiological Safety of Lamps and Lighting System—Measurement Systems—Techniques, RP27.2-00, New York, IESNA.
220. American National Standards Institute/Illuminating Engineering Society of North America (ANSI/IESNA) (2007) Photobiological Safety of Lamps and Lighting Systems—Risk Group Classification and Labeling, RP27.3-07, New York, IESNA.
221. CIE (Commission International de l'Eclairage, the International Commission on Illumination) (2002) CIE Standard S-009E-2002, Photobiological Safety of Lamps and Lamp Systems, Vienna, CIE. Adopted as a joint-logo standard by the International Electrotechnical Commission (IEC) in 2006, as IEC 62471/CIES009-2006.
222. International Standards Institute (ISO) (2007), Ophthalmic Instruments—Fundamental requirements and test methods—Part 2: Light hazard protection, ISO 15004-2:2007, Geneva, ISO.
223. Sliney, D. H., Aron-Rosa, D., DeLori, F., Fankhauser, F., Landry, R., Mainster, M., Marshall, J Rassow, B., Stuck, B., Trokel, S., Motz-West, T., and Wolffe, M. (2005), Adjustment of guidelines for exposure of the eye to optical radiation from ocular instruments: statement from a task group of the International Commission on Non-Ionizing Radiation Protection (ICNIRP), Applied Optics, 44:11.
224. Okuno, T., Kojima M., Hata I., and Sliney, D. H. (2005) Temperature rises in the crystalline lens experienced in Maxwellian-view illumination, Health Physics, 88(3): 214-222.
225. International Electrotechnical Commission (IEC) (1993), IEC 60825-1, First Edition (with 2 Amendments)—Safety of laser products—Part 1: Equipment classification and requirements, Geneva, International Electrotechnical Commission; now superseded by 2^{nd} Edition, 2007.
226. Center for Devices and Radiological Health (CDRH, 1985), Laser Product Performance Standard, Title 21, Code of Federal Regulations, Part 1040, Washington, D.C., Government Printing Office.
227. International Commission on Non-Ionizing Radiation Protection (ICNIRP) (2000) Light-emitting diodes (LEDs) and laser diodes: implications for hazard assessment. Health Phys. 78(6): 744-752.
228. James, R. H., Bostrom, R. G., Remark, D., and Sliney, D. H. (1988), Handheld ophthalmoscopes for hazards analysis: An evaluation, Applied Optics, 27:5072-5076.
229. Norrby, S., Piers, P., Campbell, C., and van der Mooren, M., (2007), Model eyes for evaluation of intraocular lenses, Appl. Opt. 46, 6595-6605.
230. Sliney D. H., and B. C. Freasier (1973) The evaluation of optical radiation hazards, Applied Opt, 12(1):1-24.
231. Morgan, J. I. W., J. J. Hunter, B. Masella, R. Wolfe, D. C. Gray, W. H. Merigan, F. C. Delori, D. R. Williams (2008) Light-induced retinal changes observed using high-resolution autofluorescence imaging of the retinal pigment epithelium. Investigative Ophthalmology & Visual Science, 49(8): 3715-3729.
232. Morgan, J. I. W., J. J. Hunter, W. H. Merigan, and D. R. Williams (2009) The Reduction of Retinal Autofluorescence Caused by Light Exposure. Investigative Ophthalmology & Visual Science, 50(12): 6015-6022.

1. A method for photodynamic cross-linking of a target tissue in an eye, the method comprising:
applying a set quantity of a photosensitizing compound to a target ocular region of the eye for a set contact time;
allowing diffusion of the photosensitizing compound in the target ocular region for a set delay time, following expiration of the contact time; and
irradiating the target ocular region of the eye with a light source upon expiration of the set delay time,
wherein:
the contact time is set to be between approximately 0.01-10 times a diffusion time of the photosensitizing compound, wherein the diffusion time is a ratio of the square of the thickness of the target tissue divided by the diffusion coefficient of the photosensitizing compound in the target tissue;
the contact time and delay time are jointly set such that the sum of the contact time and the delay time is between approximately 0.01-10 times the diffusion time of the photosensitizing compound;
the set quantity of photosensitizing compound is capable of extinguishing the irradiating light by between approximately 10-99%; and
the contact time, the delay time, and the quantity of photosensitizing compound are controllable to vary an effect of the photodynamic crosslinking.

2. The method of claim 1, further comprising removing excess photosensitizing compound from the target ocular region of the eye upon expiration of the set contact time and before allowing diffusion of the photosensitizing compound.

3. The method of claim 1, wherein the irradiating is performed at a wavelength in a range near the wavelength corresponding a maximum extinction coefficient of the photosensitizing compound such that the extinction coefficient is at least 10% of the maximum extinction.

4. The method of claim 1, wherein the photosensitizing compound has a permeability in a target tissue which is approximately between 50% to 500% that of riboflavin in that tissue.

5. The method of claim 1, wherein the photosensitizing compound has a partition coefficient (k) between a vehicle for topical application and a target tissue, of approximately greater than 3 μm²/s between an aqueous vehicle for topical application and a target tissue.

6. The method of claim 1, wherein the photosensitizing compound has a partition coefficient (k)_{PhC} between a vehicle for topical application and a target tissue, where (k)_{PhC}/(k)_{Rf} is approximately greater than between 1.5-30, where (k)_{Rf} is a partition coefficient of riboflavin between a same vehicle for topical application and a same target tissue.

7. The method of claim 1, wherein the desired portion of the eye is the cornea and the photosensitizing compound has a corneal diffusion coefficient of 40-84 μm²/s.

8. The method of claim 1, wherein the target ocular region of the eye is the sclera and the photosensitizing compound has a scleral diffusion coefficient of approximately 4-8 μm²/s.

9. The method of claim 1, wherein the target ocular region of the eye is the limbus and the photosensitizing compound has a limbal diffusion coefficient of approximately 4-84 μm²/s.

10. The method of claim 1, wherein the photosensitizing compound has a phototoxicity which is approximately less than half of a phototoxicity of riboflavin under a set of conditions that provide greater than approximately 80% of the therapeutic crosslinking of riboflavin.

11. The method of claim 1, wherein the photosensitizing compound is eosin Y.
